(19) United States
(12) Patent Application Publication
(10) Pub. No.: US 2004/0037809 A1
(43) Pub. Date: Feb. 26, 2004

(54) Compositions and methods for enhanced mucosal delivery of interferon beta

(75) Inventors: Steven C. Quay, Edmonds, WA (US); Malini Gupta, Dix Hills, NY (US); Jorge C. de Meireles, Syosset, NY (US); Mohammed Abd El-Shafy, Hauppauge, NY (US)

Correspondence Address: PAUL G. LUNN, ESQ. NASTECH PHARMACEUTICAL COMPANY, INC., 3450 MONTE VILLA PARKWAY, BOTHELL, WA, 98021-8906, US

(73) Assignee: Nastech Pharmaceutical Company Inc.

(21) Appl. No.: 10/462,452

(22) Filed: Jun. 16, 2003

Related U.S. Application Data
(60) Provisional application No. 60/393,066, filed on Jun. 28, 2002.

(51) Int. Cl. A61K 38/21

(52) U.S. Cl. 424/85.6

(57) ABSTRACT

Compositions and methods are provided for intranasal delivery of interferon-β yielding improved pharmacokinetic and pharmacodynamic results. In certain aspects of the invention, the interferon-β is delivered to the intranasal mucosa along with one or more intranasal delivery-enhancing agent(s) to yield substantially increased absorption and/or bioavailability of the interferon-β and/or a substantially decreased time to maximal concentration of interferon-β in a tissue of a subject as compared to controls where the interferon-β is administered to the same intranasal site alone or formulated according to previously disclosed reports. The enhancement of intranasal delivery of interferon-β according to the methods and compositions of the present invention allows for the effective pharmaceutical use of these agents to treat a variety of diseases and conditions in mammalian subjects.

This claims benefit under 35 U.S.C. §119(e) of U.S. Provisional Application No. 60/393,066 filed on Jun. 28, 2002, the entire contents of which are incorporated herein by reference.

### BACKGROUND OF THE INVENTION

The teachings of all of the references cited in the present specification are incorporated in their entirety by reference.

A major disadvantage of drug administration by injection is that trained personnel are often required to administer the drug. For self-administered drugs, many patients are reluctant or unable to give themselves injections on a regular basis. Injection is also associated with increased risks of infection. Some drugs, like beta interferon, can cause tissue necrosis when injected subcutaneously or even intramuscularly to the point of requiring surgical debridement of the wounds created. Other disadvantages of drug injection include variability of delivery results between individuals, as well as unpredictable intensity and duration of drug action.

Mucosal administration of therapeutic compounds may offer certain advantages over injection and other modes of administration, for example in terms of convenience and speed of delivery, as well as by reducing or eliminating compliance problems and side effects that attend delivery by injection. However, mucosal delivery of biologically active agents is limited by mucosal barrier functions and other factors. For these reasons, mucosal drug administration typically requires larger amounts of drug than administration by injection. Other therapeutic compounds, including large molecule drugs, peptides and proteins, are often refractory to mucosal delivery.

The ability of drugs to permeate mucosal surfaces, unassisted by delivery-enhancing agents, appears to be related to a number of factors, including molecular size, lipid solubility, and ionization. Small molecules, less than about 300-1,000 Daltons, are often capable of penetrating mucosal barriers, however, as molecular size increases, permeability decreases rapidly. Lipid-soluble compounds are generally more permeable through mucosal surfaces than are non-lipid-soluble molecules. Peptides and proteins are poorly lipid soluble, and hence exhibit poor absorption characteristics across mucosal surfaces.

In addition to their poor intrinsic permeability, large macromolecular drugs, including proteins and peptides, are often subject to limited diffusion, as well as lumenal and cellular enzymatic degradation and rapid clearance at mucosal sites. These mucosal sites generally serve as a first line of host defense against pathogens and other adverse environmental agents that come into contact with the mucosal surface. Mucosal tissues provide a substantial barrier to the free diffusion of macromolecules, while enzymatic activities present in mucosal secretions can severely limit the bioavailability of therapeutic agents, particularly peptides and proteins. At certain mucosal sites, such as the nasal mucosa, the typical residence time of proteins and other macromolecular species delivered is limited, e.g., to about 15-30 minutes or less, due to rapid mucociliary clearance.

While many penetration enhancing methods and additives have been reported to be effective in improving mucosal drug delivery, few penetration-enhanced products have been developed and approved for mucosal delivery of drugs. This failure can be attributed to a variety of factors, including poor safety profiles relating to mucosal irritation, and undesirable disruption of mucosal barrier functions.

In view of the foregoing, there remains a substantial unmet need in the art for new methods and tools to facilitate mucosal delivery of biotherapeutic compounds. Related to this need, there is a compelling need in the art for methods and formulations to facilitate mucosal delivery of biotherapeutic compounds that have heretofore proven refractory to delivery via this route, to avail the medical community of the numerous potential advantages of mucosal drug delivery.

One group of therapeutic compounds of interest for mucosal delivery is interferon-β IFN-β exhibits a potent antiviral function. IFN-β also mediates a variety of immunoregulatory effects.

Interferon β has been reported for treatment of relapsing forms of multiple sclerosis (MS). MS is a chronic, often disabling disease of the central nervous system. It is caused by the autoimmune destruction of myclin. Myclin is the fatty tissue that surrounds and protects central nervous system nerve fibers and facilitates the flow of nerve impulses to and from the brain. The loss of myelin disrupts the conduction of nerve impulses, producing the symptoms of MS. Symptoms may be mild numbness in the limbs, or severe paralysis or loss of vision.

IFN-β, alone or in combination with IFN- [symbol] has also been reported for treating active or chronic hepatitis B. IFN-β can be used for treatment and prevention of condyloma acuminata (genital or venereal warts caused by papilloma virus infection), papillomavirus warts of the larynx and skin (common warts). The antiviral activity of IFN-β is also reported to be useful in the treatment of severe childhood viral encephalitis.

Three forms of IFN-β approved for treatment of multiple sclerosis (MS) in the United States are IFN-β-1a (Avonex®, Biogen, Inc and Rebif®: Serono, Inc.) and IFN-β-1b (Betaseron®, Berlex Laboratories). IFN-β-1a differs from IFN-β-1b in several respects. IFN-β-1a is generated in mammalian cell culture (Chinese hamster ovary cells) whereas IFN-β-1b is produced in bacterial cells (Escherichia coli). IFN-β-1a amino acid sequence is identical to naturally occurring interferon. IFN-β-1b amino acid sequence substitutes serine for cysteine at position 17 of the 165-amino acid interferon protein.

Previous attempts to successfully deliver IFN-β for therapeutic purposes have suffered from a number of important and confounding deficiencies. These deficiencies point to a long-standing unmet need in the art for pharmaceutical formulations and methods of administering IFN-β using compositions of one or more active agents that are stable and well tolerated and that provide enhanced delivery of IFN-β to desired target sites such as the CNS or serum.

### DESCRIPTION OF THE INVENTION

The present invention fulfills the foregoing needs and satisfies additional objects and advantages by providing novel, effective methods and compositions for intranasal delivery of interferon-β yielding improved pharmacokinetic and pharmacodynamic results. In certain aspects of the invention, the interferon-β is delivered to the intranasal mucosa along with one or more intranasal delivery-enhancing agent(s) to yield substantially increased absorption and/or bioavailability of the interferon-β and/or a substantially decreased time to maximal concentration of interferon-β in a tissue of a subject as compared to controls where the interferon-β is administered to the same intranasal site alone or formulated according to previously disclosed reports. The enhancement of intranasal delivery of interferon-β according to the methods and compositions of the present invention allows for the effective pharmaceutical use of these agents to treat a variety of diseases and conditions in mammalian subjects.

The methods and compositions provided herein provide for enhanced delivery of interferon-β across nasal mucosal barriers to reach novel target sites for drug action yielding an enhanced, therapeutically effective rate or concentration of delivery. In certain aspects, employment of one or more intranasal delivery-enhancing agents facilitates the effective delivery of interferon-β to a targeted, extracellular or cellular compartment, for example the systemic circulation, a selected cell population, tissue or organ. Exemplary targets for enhanced delivery in this context are target physiological compartments, tissues, organs and fluids (e.g., within the blood serum, central nervous system (CNS) or cerebral spinal fluid (CSF)) or selected tissues or cells of the liver, bone, muscle, cartilage, pituitary, hypothalamus, kidney, lung, heart, testes, skin, or peripheral nervous system.

The enhanced delivery methods and compositions of the present invention provide for therapeutically effective mucosal delivery of interferon-β for prevention or treatment of a variety of diseases and conditions in mammalian subjects. Interferon-β can be administered via a variety of mucosal routes, for example by contacting interferon-β to a nasal mucosal epithelium, a bronchial or pulmonary mucosal epithelium, an oral, gastric, intestinal or rectal mucosal epithelium, or a vaginal mucosal epithelium. Typically, the methods and compositions are directed to or formulated for intranasal delivery (e.g., nasal mucosal delivery or intranasal mucosal delivery).

In one aspect of the invention, pharmaceutical formulations suitable for intranasal administration are provided that comprise a therapeutically effective amount of interferon-β and one or more intranasal delivery-enhancing agents as described herein, which formulations are effective in a nasal mucosal delivery method of the invention to prevent the onset or progression of disease related to autoimmune disease, viral infection, or cancer, e.g., a solid tumor, in a mammalian subject, or to alleviate one or more clinically recognized symptoms of autoimmune disease, viral infection or cancer in a mammalian subject.

In another aspect of the invention, pharmaceutical formulations suitable for intranasal administration are provided that comprise a therapeutically effective amount of interferon-β and one or more intranasal delivery-enhancing agents as described herein, which formulation is effective in a nasal mucosal delivery method of the invention to alleviate symptoms or prevent the onset or lower the incidence or severity of multiple sclerosis, condyloma acuminata (genital or venereal warts caused by papilloma virus infection), papillomavirus warts of the larynx and skin (common warts), chronic hepatitis B, or severe childhood viral encephalitis. Within these and related methods, the IFN-β may be administered alone or in combination with IFN- [symbol] or other immune modifiers such as steroids or glatiramer acetate injection.

In more detailed aspects of the invention, methods and compositions for intranasal delivery of interferon-β incorporate one or more intranasal delivery enhancing agent(s) combined in a pharmaceutical formulation together with, or administered in a coordinate nasal mucosal delivery protocol with, a therapeutically effective amount of IFN-\tilde{β}These methods and compositions provide enhanced nasal transmucosal delivery of the interferon-β, often in a pulsatile delivery mode to maintain continued release of interferon-β to yield more consistent (normalized) or elevated therapeutic levels of interferon-β in the blood serum, central nervous system (CNS), cerebral spinal fluid (CSF), or in another selected physiological compartment or target tissue or organ for treatment of disease. Normalized and elevated therapeutic levels of interferon-β are determined, for example, by an increase in bioavailability (e.g., as measured by maximal concentration (Cₘₐₓ) or the area under concentration vs. time curve (AUC) for an intranasal effective amount of interferon-β) and/or an increase in delivery rate (e.g., as measured by time to maximal concentration (tₘₐₓ), Cₘₐₓ, and or AUC). Normalized and elevated high therapeutic levels of interferon-β in the blood serum, central nervous system (CNS), or cerebral spinal fluid (CSF) may be achieved in part by repeated intranasal administration to a subject within a selected dosage period, for example an 8, 12, or 24 hour dosage period.

To maintain more consistent or normalized therapeutic levels of interferon-β, the pharmaceutical formulations of the present invention are often repeatedly administered to the nasal mucosa of the subject, for example one, two or more times within a 24 hour period, four or more times within a 24 hour period, six or more times within a 24 hour period, or eight or more times within a 24 hour period. The methods and compositions of the present invention yield improved pulsatile delivery to maintain normalized and/or elevated therapeutic levels of interferon- [symbol] e.g., in the blood serum. The methods and compositions of the invention enhance transnasal mucosal delivery of interferon-β to a selected target tissue or compartment by at least a two- to five-fold increase, more typically a five- to ten-fold increase, and commonly a ten- to twenty-five- up to a fifty-fold increase (e.g., as measured by tₘₐₓ Cₘₐₓ, and/or AUC, in the blood serum, central nervous system, cerebral spinal fluid, or in another selected physiological compartment or target tissue or organ for delivery), compared to the efficacy of delivery of interferon-β administered alone or using a previously-described delivery method, for example a previously-described mucosal delivery, intramuscular delivery, subcutaneous delivery, intravenous delivery, and/or parenteral delivery method.

Nasal mucosal delivery of interferon-βaccording to the methods and compositions of the invention will often yield effective delivery and bioavailability that approximates dosing achieved by continuous administration methods. In other aspects, the invention provides enhanced nasal mucosal delivery that permits the use of a lower systemic dosage and significantly reduces the incidence of interferon-\overline{β}related side effects. Because continuous infusion of interferon-β outside the hospital setting is otherwise impractical, mucosal delivery of interferon-β as provided herein yields unexpected advantages that allow sustained delivery of interferon-β, with the accrued benefits, for example, of improved patient-to-patient dose variability.

In more detailed aspects of the invention, the methods and compositions of the present invention provide improved and/or sustained delivery of interferon-β to the blood serum, lymphatic system, CNS, and/or CSF. In one exemplary embodiment, an intranasal effective amount of interferon-β and one or more intranasal delivery enhancing agent(s) is contacted with a nasal mucosal surface of a subject to yield enhanced mucosal delivery of interferon-β to the central nervous system (CNS) or cerebral spinal fluid (CSF) of the subject, for example to effectively treat autoimmune diseases. In certain embodiments, the methods and compositions of the invention provide improved and sustained delivery of interferon-β to the CNS and will effectively treat one or more symptoms of multiple sclerosis, including in cases where conventional interferon-β therapy yields poor results or unacceptable adverse side effects.

In exemplary embodiments, the methods and compositions of the present invention yield a two- to five-fold decrease, more typically a five- to ten-fold decrease, and commonly a ten- to twenty-five- up to a fifty- to one hundred-fold decrease in the time to maximal concentration (tₘₐₓ) of the interferon-β in blood serum, central nervous system, cerebral spinal fluid, and/or in another selected physiological compartment or target tissue or organ for delivery—as compared to delivery rates for interferon-β administered alone or in accordance with previously-described drug delivery methods.

In further exemplary embodiments, the methods and compositions of the invention yield a two- to five-fold increase, more typically a five- to ten-fold increase, and commonly a ten- to twenty-five- up to a fifty- to one hundred-fold increase in the area under concentration vs. time curve, AUC, of the interferon-β in blood serum, central nervous system, cerebral spinal fluid, and/or in another selected physiological compartment or target tissue or organ for delivery—as compared to delivery rates for the interferon-β administered alone or in accordance with previously-described administration methods.

In further exemplary embodiments, the methods and compositions of the present invention yield a two- to five-fold increase, more typically a five- to ten-fold increase, and commonly a ten- to twenty-five- up to a fifty- to one hundred-fold increase in the maximal concentration, Cₘₐₓ, of the interferon-β in blood serum, central nervous system, cerebral spinal fluid, and/or in another selected physiological compartment or target tissue or organ for delivery—as compared to delivery rates for the interferon-β administered alone or in accordance with previously-described administration methods.

The methods and compositions of the invention will often serve to improve interferon-β dosing schedules and thereby maintain normalized and/or elevated, therapeutic levels of interferon-β in the subject. In certain embodiments, the invention provides compositions and methods for intranasal delivery of interferon-β, wherein interferon-β dosage normalized and sustained by repeated, typically pulsatile, delivery to maintain more consistent, and in some cases elevated, therapeutic levels. In exemplary embodiments, the time to maximum concentration (tₘₐₓ) of interferon-β in the blood serum will be from about 0.1 to 4.0 hours, alternatively from about 0.4 to 1.5 hours, and in other embodiments from about 0.7 to 1.5 hours, or from about 1.0 to 1.3 hours. Thus, repeated intranasal dosing with the formulations of the invention, on a schedule ranging from about 0.1 to 2.0 hours between doses, will maintain normalized, sustained therapeutic levels of interferon-βto maximize clinical benefits while minimizing the risks of excessive exposure and side effects.

In alternative embodiments, the invention achieves enhanced delivery of normalized and/or elevated, improved therapeutic levels of interferon-β by combining mucosal administration of one dosage amount of interferon-β formulated with one or more intranasal delivery-enhancing agents, with a separate dosage amount of interferon-β delivered by a non-mucosal route, for example by intramuscular administration. In one exemplary embodiment, intranasal delivery of interferon-β according to the compositions and methods herein yields normalized and/or elevated, high therapeutic levels of interferon-β in the blood serum of the subject for a time period between approximately 0.1 and 3 hours following intranasal administration. Coordinate administration of interferon-β by a non-mucosal route (before, simultaneous with, or after mucosal administration) provides more consistent, elevated therapeutic levels of interferon-β in the blood serum of the subject for an effective time period of between approximately 2 to 24 hours, more often between about 4-16 hours, and in certain embodiments between about 6-8 hours. Within these coordinate administration methods, improving clinical benefit while minimizing the risks of excessive exposure facilitates the aims of the treating physician.

In other aspects of the invention, the methods and formulations for intranasally administering interferon-β described herein yield a significantly enhanced rate or level of delivery (e.g., decreased tₘₐₓ, increased AUC, and/or increased Cₘₐₓ) of the interferon-β into the serum, or to selected tissues or cells, of the subject. This includes enhanced delivery rates or levels into the serum, or to selected tissues or cells (e.g., blood serum, CNS, or CSF), compared to delivery rates and levels for the interferon-β administered alone or in accordance with previously-described technologies. Thus, in certain aspects of the invention, the foregoing methods and compositions are administered to a mammalian subject to yield enhanced delivery of the interferon-β to a physiological compartment, fluid, tissue or cell within the mammalian subject.

Within more detailed aspects of the invention, bioavailability of interferon-β achieved by the methods and formulations herein (e.g., measured by peak blood plasma levels (Cₘₐₓ) of interferon-β in blood serum, CNS, CSF or in another selected physiological compartment or target tissue) will be, for example, about 5 μg per liter of blood plasma or CSF, typically about 10 μg per liter of blood plasma or CSF, about 20 μg per liter of blood plasma or CSF, about 30 μg per liter of blood plasma or CSF, about 40 μg per liter of blood plasma or CSF, about 50 μg per liter of blood plasma or CSF, or about 60 μg or greater per liter of blood plasma or CSF.

Within other detailed aspects of the invention, bioavailability of interferon-β following administration in accordance with the methods and compositions of the invention is determined by measuring interferon-β “pharmacokinetic markers”. For example, art-accepted pharmacokinetic markers for interferon-β, serum β-2 microglobulin or serum neopterin, may be measured following administration, e.g., as measured by peak blood plasma levels (Cₘₐₓ) of the marker(s) in blood serum, CNS, CSF or in another selected physiological compartment or target tissue. These and other such marker data are accepted in the art as reasonably correlated with pharmakokinetics of interferon-β compounds that may be undetectable directly in vivo. In certain aspects, enhanced bioavailability of interferon-β as measured by interferon-β markers will be demonstrated by, for example, a correlated Cₘₐₓ for serum β-2 microglobulin of approximately 1.7 mg/ml of blood plasma or CSF, or approximately 2.0 mg/ml of blood plasma or CSF, or approximately 4.0 mg/ml or greater of blood plasma or CSF. Cₘₐₓ for serum neopterin of approximately 8 nmol/l of blood plasma or CSF, approximately 10 nmol/l of blood plasma or CSF, approximately 20 nmol/l of blood plasma or CSF, approximately 30 nmol/l of blood plasma or CSF, or approximately 40 nmol/l or greater of blood plasma or CSF.

Within further detailed aspects, the pharmaceutical composition as disclosed herein following mucosal administration to said subject yields a peak concentration (Cₘₐₓ) for pharmacological markers, neopterin or β2-microglobulin in the blood plasma or CNS tissue or fluid of the subject that is typically 25% or greater, or 75% or greater, or 150% or greater, as compared to a peak concentration of neopterin or β2-microglobulin in blood plasma or CNS tissue or fluid following intramuscular injection of an equivalent concentration or dose of interferon-β to said subject, intranasal delivery of interferon-β alone, and/or mucosal delivery of interferon-β using previously-described methods and formulations.

Within other detailed aspects of the invention, bioavailability of interferon-β as will be determined by measuring interferon-β pharmacokinetic markers, for example, serum β-2 microglobulin or serum neopterin, to determine area under the concentration curve (AUC) for the marker(s) in blood serum, CNS, CSF or in another selected physiological compartment or target tissue. Bioavailability of interferon-β as determined by interferon-β markers in this context will be, for example, AUC_{0-96 hr} for serum β-2 microglobulin of approximately 200 μIU·hr/mL of blood plasma or CSF, AUC_{0-96 hr} for β-2 microglobulin up to approximately 500 μIU·hr/mL of blood plasma or CSF, AUC_{0-96 hr} for serum neopterin of approximately 200 ng·hr/ml of blood plasma or CSF, AUC_{0-96 hr} for serum neopterin up to approximately 500 ng·hr/ml of blood plasma or CSF.

Within further detailed aspects, the pharmaceutical composition as disclosed herein following mucosal administration to said subject yields area under the concentration curve (AUC_{0-96 hr}) for pharmacological markers, neopterin or β2-microglobulin, in the blood plasma or CNS tissue or fluid of the subject that is typically 25% or greater, or 75% or greater, or 150% or greater, as compared to an AUC_{0-96 hr} for neopterin or β2-microglobulin in blood plasma or CNS tissue or fluid following intramuscular injection of an equivalent concentration or dose of interferon-β to said subject, intranasal delivery of interferon-β alone, and/or mucosal delivery of interferon-β using previously-described methods and formulations.

Within yet additional detailed aspects of the invention, bioavailability of interferon-β pharmacokinetic markers, for example, serum β-2 microglobulin or serum neopterin, achieved by the methods and formulations herein is measured by time to maximal concentration (tₘₐₓ) in blood serum, CNS, CSF or in another selected physiological compartment or target tissue. tₘₐₓ for serum β-2 microglobulin will be, for example, between about 45 hours or less and about 48 to 60 hours. In other embodiments, these values may be 35 hours or less, or 25 hours or less following intranasal administration of interferon-β by methods and formulations described herein. tₘₐₓ for serum neopterin will be, for example, about 40 hours or less, typically 30 hours or less, or typically 25 hours or less following intranasal administration of interferon-β by methods and formulations described herein.

Within further detailed aspects, the pharmaceutical composition as disclosed herein following mucosal administration to said subject yields a time to maximal plasma concentration (tₘₐₓ) for pharmacological markers, neopterin or β₂-microglobulin, in a blood plasma or CNS tissue or fluid of the subject that is typically between about 25 to 45 hours, or between about 25 to 35 hours.

In exemplary embodiments, administration of one or more interferon-β formulated with one or more intranasal delivery-enhancing agents as described herein yields effective delivery to the blood serum CNS, or CSF to alleviate a selected disease or condition (e.g., multiple sclerosis, or a symptom thereof) in a mammalian subject. In more detailed aspects, the methods and formulations for intranasally administering interferon-β according to the invention yield a significantly enhanced rate or level of delivery (e.g., decreased tₘₐₓ or increased Cₘₐₓ) of the interferon-β into the serum or to selected tissues or cells (e.g., liver), compared to delivery rates and levels for the interferon-β administered alone or in accordance with previously-described technologies.

Within exemplary aspects, the enhanced delivery rate or level of the interferon-β provides for more effective treatment of multiple sclerosis or viral disease in a subject. For example, by using the intranasal administration methods and formulations of the invention, an effective concentration of interferon-β can be delivered to the blood serum CNS, CSF, or peripheral nervous system, usually within about 45 min, 30 min, 20 min, and even 15 min or less following administration, resulting in an enhanced therapeutic effect (e.g., decreased symptoms of MS, or decreased viral load) in the subject with minimal side effects. Side effects that are generally minimized or avoided by the methods and compositions of the invention include progressive damage and bleeding to the mucosal site of drug delivery from repeated administration—that would otherwise result in poor mucosal absorption of interferon-β. Additional side effects that are reduced or avoided by the present invention include flu-like syndrome of headache, fever, malaise, sensations of temperature change myalgias, arthralgias, and severe delivery site reactions such as necrosis, nausea, leucopoenia, and liver enzyme abnormalities.

The enhanced pharmacokinetics of delivery of interferon-β (e.g., increased frequency of dosing possible, increased rate, normalized, sustained delivery, and elevated levels) according to the methods of the invention, provides improved therapeutic efficacy, e.g., to treat autoimmune disease, viral infection, or cancer in a subject, without unacceptable adverse side effects. Thus, for example, pharmaceutical preparations formulated for nasal mucosal delivery are provided for treating multiple sclerosis in a mammalian subject that comprise a therapeutic intranasal effective amount of interferon-β combined with one or more intranasal delivery-enhancing agents as disclosed herein. These preparations surprisingly yield enhanced mucosal absorption of the interferon-β to produce a therapeutic effective concentration of the drug (e.g., for treating acute MS, or relapsing remitting MS in a subject) at a target site or tissue in the subject in about 45 minutes or less, 30 minutes or less, 20 minutes or less, or as little as 15 minutes or less.

Within other detailed embodiments of the invention, the foregoing methods and formulations are administered to a mammalian subject to yield enhanced bioavailability, or enhanced blood plasma concentration of mucosally-administered interferon-β, a cumulative (e.g., ‘per week’) area under the concentration curve (AUC) for interferon-β (e.g., as expressed by the AUC of a single dose multipled by the number of doses per week) in the blood plasma or CSF following mucosal (e.g., intranasal) administration to the subject by methods and compositions of the present invention is about 10% or greater compared to an area under the concentration curve (AUC) for interferon-β in the plasma or CSF following intramuscular injection to the mammalian subject. In exemplary embodiments, an area under the concentration curve (AUC) for interferon-β in the blood plasma or CSF following intranasal administration of one or more interferon-βs formulated with one or more intranasal delivery-enhancing agents as described herein is at leaset about 25%, 40%, or greater compared to an area under the concentration curve (AUC) for interferon-β in the plasma or CSF following intramuscular injection to the mammalian subject. In yet additional exemplary embodiments an area under the concentration curve (AUC) for interferon-β in the blood plasma or CSF following intranasal administration by methods and compositions of the present invention to the subject is at least about 60%, 80%, 100% or greater, up to 150% or greater, compared to an area under the concentration curve (AUC) for interferon-β in the plasma or CSF following intramuscular injection to the mammalian subject. These enhanced rates and levels of delivery are correlated with increased therapeutic efficacy of the methods and formulations of the invention for prophylaxis and treatment of the indicated diseases and conditions in mammalian subjects as compared to relevant clinical control subjects.

Within other detailed embodiments of the invention, the foregoing methods and formulations are administered to a mammalian subject to yield enhanced blood plasma or CSF levels of interferon-β, wherein following mucosal (e.g., intranasal) administration of interferon-βaccording to the methods and compositions herein yields a time to maximal plasma or CSF concentration (tₘₐₓ) for interferon-β between approximately 0.1 to 4.0 hours. In exemplary embodiments a time to maximal plasma or CSF concentration (tₘₐₓ) of interferon-β in the blood plasma following intranasal administration by methods and compositions of the present invention to the subject is between approximately 0.7 to 1.5 hours, or between approximately 1.0 to 1.3 hours. In exemplary embodiments, a time to maximal plasma or CSF concentration (tₘₐₓ) of interferon-βpharmacokinetic markers, serum β-2 microglobulin or serum neopterin, following administration of one or more interferon-β formulated with one or more intranasal delivery-enhancing agents as described herein is between approximately 25 and 45 hours, or between approximately 25 to 30 hours. These enhanced rates and levels of delivery are correlated with increased therapeutic efficacy of the methods and formulations of the invention for prophylaxis and treatment of the indicated diseases and conditions in mammalian subjects as compared to relevant clinical control subjects.

Within other detailed embodiments of the invention, the foregoing methods and formulations are administered to a mammalian subject to yield enhanced blood plasma or CSF levels of interferon-β, whereby said formulation following mucosal (e.g., intranasal) administration to the subject yields a time to maximal plasma concentration (tₘₐₓ) of said interferon-β in a blood plasma or CSF of said subject that is 75%, 50%, 20%, or as short as 10% or less compared to a time to maximal plasma concentration (tₘₐₓ) of interferon-β in the blood plasma or CSF of the subject following administration of an equivalent concentration or dose of interferon-β by intramuscular injection. These enhanced rates and levels of delivery are correlated with increased therapeutic efficacy of the methods and formulations of the invention for prophylaxis and treatment of the indicated diseases and conditions in mammalian subjects as compared to relevant clinical control subjects.

Within other detailed embodiments of the invention, the foregoing methods and formulations are administered to a mammalian subject to yield enhanced blood plasma or CSF levels of mucosally-administered interferon-β, whereby a peak concentration of interferon-β in the blood plasma (Cₘₐₓ) following mucosal (e.g., intranasal) administration to the subject by methods and compositions of the present invention is about 25% or greater compared to a peak concentration of interferon-β in the plasma following intramuscular injection to the mammalian subject. In exemplary embodiments, a peak concentration of interferon-β in the blood plasma (Cₘₐₓ) following intranasal administration of interferon-β formulated with one or more intranasal delivery-enhancing agents as described herein is about 40% or greater compared to a peak concentration of interferon-β in the plasma following intramuscular injection to the mammalian subject. In yet additional exemplary embodiments a peak concentration of interferon-β in the blood plasma (Cₘₐₓ) following intranasal administration by methods and compositions of the present invention to the subject is about 80% or greater, about 100% or greater, up to 150% or greater, compared to a peak concentration of interferon-β in the plasma following intramuscular injection to the mammalian subject. These enhanced rates and levels of delivery are correlated with improved therapeutic efficacy of the methods and formulations of the invention for prophylaxis and treatment of the indicated diseases and conditions in mammalian subjects.

Within other detailed embodiments of the invention, the foregoing methods and formulations are administered to a mammalian subject to yield enhanced CNS, cerebral spinal fluid (CSF) or peripheral nervous system delivery of the interferon-β, whereby the peak interferon-β concentration in a CNS, CSF or peripheral nervous system target site by intranasal delivery (e.g., nasal mucosal delivery) is at least 5% of a related peak interferon-β concentration in the blood plasma following administration of the formulation to the subject. In exemplary embodiments, administration of one or more interferon-βs formulated with one or more intranasal delivery-enhancing agents as described herein yields a peak interferon-β concentration in the CNS, CSF, or peripheral nervous system of about 10% or greater versus the peak interferon-β concentration in the blood plasma following administration of the formulation to the subject. In other exemplary embodiments, the peak interferon-β concentration in the CNS, CSF or peripheral nervous system is about 15% or greater versus the peak interferon-β concentration in the blood plasma. In yet additional exemplary embodiments, the peak interferon-β concentration in the CNS, CSF or peripheral nervous system is about 20% or greater, 30% or greater, 35% or greater, or up to 40% or greater, versus the peak interferon-β concentration in the blood plasma. These enhanced rates and levels of delivery are correlated directly with the efficacy of the nasal mucosal delivery methods and formulations of the invention for prophylaxis and treatment of diseases and conditions in mammalian subjects amenable to prophylaxis and treatment by CNS, CSF or peripheral nervous system delivery of therapeutic levels of selected interferon-β.

Within other detailed embodiments of the invention, the foregoing methods and formulations are administered to a mammalian subject to yield enhanced blood plasma levels, CNS, CSF or other tissue levels of the interferon-β by administering a formulation comprising an intranasal effective amount of interferon-β and one or more intranasal delivery-enhancing agents and one or more sustained release-enhancing agents. The sustained release-enhancing agents, for example, may comprise a polymeric delivery vehicle. In exemplary embodiments, the sustained release-enhancing agent may comprise polyethylene glycol (PEG) coformulated or coordinately delivered with interferon-β and one or more intranasal delivery-enhancing agents. PEG may be covalently bound to interferon-β. The sustained release-enhancing methods and formulations of the present invention will increase residence time (RT) of the interferon-β at a site of administration and will maintain a basal level of the interferon-β over an extended period of time in blood plasma, CNS, CSF, or other tissue in the mammalian subject.

Within other detailed embodiments of the invention, the foregoing methods and formulations are administered to a mammalian subject to yield enhanced blood plasma levels, CNS, CSF or other tissue levels of the interferon-β to maintain basal levels of interferon-β over an extended period of time. Exemplary methods and formulations involve administering a pharmaceutical formulation comprising an intranasal effective amount of interferon-β and one or more intranasal delivery-enhancing agents to a mucosal surface of the subject, in combination with intramuscular administration of a second pharmaceutical formulation comprising interferon-β. Maintenance of basal levels of interferon-β is particularly useful for treatment and prevention of disease, for example, multiple sclerosis, papilloma virus infection, and chronic hepatitis B.

The foregoing mucosal drug delivery formulations and preparative and delivery methods of the invention provide improved mucosal delivery of interferon-β to mammalian subjects. These compositions and methods can involve combinatorial formulation or coordinate administration of one or more interferon- [symbol] s) with one or more mucosal (e.g., intranasal) delivery-enhancing agents. Among the mucosal delivery-enhancing agents to be selected from to achieve these formulations and methods are (a) aggregation inhibitory agents; (b) charge modifying agents; (c) pH control agents; (d) degradative enzyme inhibitors; (e) mucolytic or mucus clearing agents; (f) ciliostatic agents; (g) membrane penetration-enhancing agents (e.g., (i) a surfactant, (ii) a bile salt, (ii) a phospholipid or fatty acid additive, mixed micelle, liposome, or carrier, (iii) an alcohol, (iv) an enamine, (v) an NO donor compound, (vi) a long-chain amphipathic molecule (vii) a small hydrophobic penetration enhancer; (viii) sodium or a salicylic acid derivative; (ix) a glycerol ester of acetoacetic acid (x) a clyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, (x) an inhibitor of cholesterol synthesis; or (xi) any combination of the membrane penetration enhancing agents of (i)-(x)); (h) modulatory agents of epithelial junction physiology, such as nitric oxide (NO) stimulators, chitosan, and chitosan derivatives; (i) vasodilator agents; (j) selective transport-enhancing agents; and (k) stabilizing delivery vehicles, carriers, supports or complex-forming species with which the interferon- [symbol] is/are effectively combined, associated, contained, encapsulated or bound to stabilize the active agent for enhanced nasal mucosal delivery.

In various embodiments of the invention, interferon-β is combined with one, two, three, four or more of the mucosal (e.g., intranasal) delivery-enhancing agents recited in (a)-(k), above. These mucosal delivery-enhancing agents may be admixed, alone or together, with the interferon-β, or otherwise combined therewith in a pharmaceutically acceptable formulation or delivery vehicle. Formulation of interferon-β with one or more of the mucosal delivery-enhancing agents according to the teachings herein (optionally including any combination of two or more mucosal delivery-enhancing agents selected from (a)-(k) above) provides for increased bioavailability of the interferon-β following delivery thereof to a mucosal (e.g., nasal mucosal) surface of a mammalian subject.

In related aspects of the invention, a variety of coordinate administration methods are provided for enhanced mucosal delivery of interferon-β. These methods comprise the step, or steps, of administering to a mammalian subject a mucosally effective amount of at least one interferon-β in a coordinate administration protocol with one or more mucosal delivery-enhancing agents of (a)-(k) above.

To practice a coordinate administration method according to the invention, any combination of one, two or more of the mucosal delivery-enhancing agents recited in (a)-(k), above, may be admixed or otherwise combined for simultaneous mucosal (e.g., intranasal) administration. Alternatively, any combination of one, two or more of the mucosal delivery-enhancing agents recited in (a)-(k) can be mucosally administered, collectively or individually, in a predetermined temporal sequence separated from mucosal administration of the interferon-β (e.g., by pre-administering one or more of the delivery-enhancing agent(s)), and via the same or different delivery route as the interferon-β (e.g., to the same or to a different mucosal surface as the interferon-β, or even via a non-mucosal (e.g., intramuscular, subcutaneous, or intravenous route). Coordinate administration of interferon-β with any one, two or more of the mucosal delivery-enhancing agents according to the teachings herein provides for increased bioavailability of the interferon-β following delivery thereof to a mucosal surface of a mammalian subject.

In additional related aspects of the invention, various “multi-processing” or “co-processing” methods are provided for preparing formulations of interferon-β for enhanced nasal mucosal delivery. These methods comprise one or more processing or formulation steps wherein one or more interferon-β(s) is/are serially, or simultaneously, contacted with, reacted with, or formulated with, one, two or more (including any combination of) of the mucosal delivery-enhancing agents of (a)-(k) above.

To practice the multi-processing or co-processing methods according to the invention, the interferon-β is/are exposed to, reacted with, or combinatorially formulated with any combination of one, two or more of the mucosal delivery-enhancing agents recited in (a)-(k), above, either in a series of processing or formulation steps, or in a simultaneous formulation procedure, that modifies the interferon-β (or other formulation ingredient) in one or more structural or functional aspects, or otherwise enhances mucosal delivery of the active agent in one or more (including multiple, independent) aspect(s) that are each attributed, at least in part, to the contact, modifying action, or presence in a combinatorial formulation, of a specific mucosal delivery-enhancing agent recited in (a)-(k), above.

In certain detailed aspects of the invention, the methods and compositions which comprise a mucosally effective amount of interferon-β and one or more mucosal delivery-enhancing agent(s) (combined in a pharmaceutical formulation together or administered in a coordinate nasal mucosal delivery protocol) provide nasal transmucosal delivery of the interferon-β in a pulsatile delivery mode to maintain more consistent or normalized, and/or elevated levels of interferon-β in the blood serum. In this context, the pulsatile delivery methods and compositions of the invention yield increased bioavailability (e.g., as measured by maximal concentration, (Cₘₐₓ) or area under concentration curve (AUC) of interferon-β and/or an increased mucosal delivery rate (e.g., as measured by time to maximal concentration (tₘₐₓ), Cₘₐₓ and/or AUC compared to other mucosal or non-mucosal delivery method-based controls. For example, the invention provides pulsatile delivery methods and formulations that comprise interferon-β and one or more mucosal delivery-enhancing agent(s), wherein the formulation administered mucosally (e.g., intranasally) to a mammalian subject, yields an area under the concentration curve (AUC) for interferon-β in the blood plasma that is about 10% or greater compared to an area under the concentration curve (AUC) for interferon-β in the plasma following intramuscular injection to the mammalian subject.

Often the formulations of the invention are administered to a nasal mucosal surface of the subject. In certain embodiments, the interferon-β is a human interferon-β-1a, (Avonex®, Biogen, Inc.), human interferon-β-1b (Betaseron®, Berlex Laboratories), or a pharmaceutically acceptable salt or derivative thereof. A mucosally effective dose within the pharmaceutical formulations of the present invention comprises, for example, between about 10 μg and 600 μg of interferon-β. In certain embodiments, an effective dose of the pharmaceutical formulation comprising interferon-β is, for example, 30 μg, 60 μg, 90 μg, 12 [symbol] μg, 20 [symbol] μg, 25 [symbol] μg, 30 [symbol] μg, or 400 μg. In certain embodiments an effective dose within the pharmaceutical formulations of the invention is, for example, between about 30 and 100 μg of interferon-β. The pharmaceutical formulations of the present invention may be administered in a repeated dosing regimen, for example, one or more times daily, 3 times per week, or weekly. In certain embodiments, the pharmaceutical formulations of the invention are administered two times daily, four times daily, or six times daily. In related embodiments, the mucosal (e.g., intranasal) formulations comprising interferon-β(s) and one or more delivery-enhancing agent(s) administered via a repeated dosing regimen yields an area under the concentration curve (AUC) for interferon-β in the blood plasma or CSF following repeated dosing that is about 25% or greater compared to an area under the concentration curve (AUC) for interferon-β in the plasma or CSF following one or more intramuscular injections of the same or comparable amount of interferon-β. In other embodiments, the mucosal formulations of the invention administered via a repeated dosing regimen yields an area under the concentration curve (AUC) for interferon-β in the blood plasma or CSF following repeated dosing that is about 40%, 80%, 100%, 150%, or greater compared to the AUC for interferon-β in the plasma 25% or greater compared to an area under the concentration curve (AUC) for interferon-β in the plasma or CSF following one or more intramuscular injections of the same or comparable amount of interferon-β.

In certain detailed aspects of the invention, a stable pharmaceutical formulation is provided which comprises interferon-β and one or more delivery-enhancing agent(s), wherein the formulation administered intranasally to a mammalian subject yields a time to maximal plasma concentration (tₘₐₓ) for interferon-β between approximately 0.4 to 2.0 hours in a mammalian subject. Often the formulation is administered to a nasal mucosal surface of the subject.

In certain embodiments of the invention, the intranasal formulation of interferon-β and one or more delivery-enhancing agent(s) yields a time to maximal plasma concentration (tₘₐₓ) for interferon-β between approximately 0.4 to 1.5 hours in the mammalian subject. Alternately, the intranasal formulation of the present invention yields a time to maximal plasma concentration (tₘₐₓ) for interferon-β between approximately 0.7 to 1.5 hours, or between approximately 1.0 to 1.3 hours in the mammalian subject.

In certain detailed aspects of the invention, a stable pharmaceutical formulation is provided which comprises interferon-β and one or more intranasal delivery-enhancing agent(s), wherein the formulation administered intranasally to a mammalian subject yields a peak concentration of interferon-β in the blood plasma (Cₘₐₓ) following intranasal administration to the subject by methods and compositions of the present invention that is about 25% or greater compared to a peak concentration of interferon-β in the plasma following intramuscular injection to the mammalian subject. Within related methods, the formulation is administered to a nasal mucosal surface of the subject.

In other detailed embodiments of the invention, the intranasal formulation of the interferon-β(s) and one or more delivery-enhancing agent(s) yields a peak concentration of interferon-β in the blood plasma (Cₘₐₓ) following intranasal administration to the subject that is about 40% or greater compared to a peak concentration of interferon-β in the plasma following intramuscular injection of a comparable dose of interferon-β to the subject. Alternately, the intranasal formulation of the present invention may yield a peak concentration of interferon-β in the blood plasma (Cₘₐₓ) that is about 80%, 100% or 150%, or greater compared to the peak concentration of interferon-β in the plasma following intramuscular injection to the mammalian subject.

Intranasal delivery-enhancing agents are employed which enhance delivery of interferon-β into or across a nasal mucosal surface. For passively absorbed drugs, the relative contribution of paracellular and transcellular pathways to drug transport depends upon the pKₐ, partition coefficient, molecular radius and charge of the drug, the pH of the luminal environment in which the drug is delivered, and the area of the absorbing surface. The intranasal delivery-enhancing agent of the present invention may be a pH control agent. The pH of the pharmaceutical formulation of the present invention is a factor affecting absorption of interferon-β via paracellular and transcellular pathways to drug transport. In one embodiment, the pharmaceutical formulation of the present invention is pH adjusted to between about pH 3.0 to 8.0. In a further embodiment, the pharmaceutical formulation of the present invention is pH adjusted to between about pH 3.5 to 7.5. In a further embodiment, the pharmaceutical formulation of the present invention is pH adjusted to between about pH 4.0 to 5.0. In a further embodiment, the pharmaceutical formulation of the present invention is pH adjusted to between about pH 4.0 to 4.5.

In still other embodiments of the invention, pharmaceutical compositions and methods are provided wherein one or more of the interferon-β compounds or formulations described herein are administered coordinately or in a combinatorial formulation with one or more steroid or corticosteroid compound(s). These compositions in some embodiments are effective following mucosal administration to alleviate one or more symptom(s) of inflammation, nasal irritation, rhinitis, or allergy without unacceptable adverse side effects. In other embodiments, these combinatorial formulations or coordinate administration methods are effective to alleviate one or more symptom(s) of an autoimmune disease, e.g., multiple sclerosis, or a viral infection.

Other combinatorial formulations for use within the invention comprise a stable pharmaceutical composition comprising an effective amount of one or more cytokine(s) or growth factor(s) formulated for mucosal delivery to a mammalian subject in combination with one or more steroid or corticosteroid compound(s), wherein the formulation is effective following mucosal administration to alleviate one or more symptom(s) of inflammation, nasal irritation, rhinitis, or allergy, or one or more symptom(s) of an autoimmune disease, e.g., multiple sclerosis, or a viral infection, without unacceptable adverse side effects. The combinatorial formulations of a cytokine and steroid may or may not contain mucosal delivery-enhancing agent(s) as described herein.

In more detailed embodiments, the combinatorial formulations and coordinate administration methods involving a cytokine or growth factor and steroid employ lymphokines, monokines, and/or hematopoietic factors. In certain embodiments, the cytokine(s) or growth factor(s) is/are selected from interleukins 1 to 21, tumor necrosis factor-a (TNF-a), tumor necrosis factor-b (TNF-b), malignant leukocyte inhibitory factor (LIF), erythropoietin (EPO), granulocyte colony-stimulating factor (G-CSF), granulocyte macrophage colony-stimulating factor (GM-CSF), macrophage colony-stimulating factor (M-CSF), hepatocyte growth factor, interferon a, interferon b, interferon g, nerve growth factor, oncostatin M, prolactin, RANTES, tumor necrosis factor-a (TNF-a), tumor necrosis factor-b (TNF-b, epidermal growth factor (EGF), basic fibroblast growth factor (bFGF), acidic fibroblast growth factor (aFGF), insulin-like growth factor (IGF), transforming growth factor b1 (TGF b1), transforming growth factor b2 (TGF b2, transforming growth factor b3 (TGF b3), platelet-derived cell growth factor (PDGF), and hepatocyte growth factor (HGF). In exemplary embodiments, the cytokine(s) or growth factor(s) include one or more β-interferon(s).

In more detailed embodiments, the combinatorial formulations and coordinate administration methods involving a cytokine or growth factor and steroid employ one or more steroid or corticosteroid compound(s) selected from triamcinolone, methylprednisolone, prednisolone, prednisone, fluticasone, betamethasone, dexamethasone, hydrocortisone, cortisone, flunisolide, beclomethasone dipropionate, budesonide, amcinonide, clobetasol, clobetasone, desoximetasone, diflorasone, diflucortolone, fluocinolone, fluocinonide, flurandrenolide, fluticasone, halcinonide, halobetasol, hydrocortisone butyrate, hydrocortisone valerate, and mometasone.

In related aspects, these compositions upon repeated dosing once or twice a day for a cumulative dosing period between about 7-14 days yield a cumulative area under the concentration curve (AUC) for the steroid or corticosteroid compound(s) in a central nervous system (CNS) tissue or fluid of the subject that is approximately 50%, 75%, 100% or greater compared to a cumulative AUC of the steroid or corticosteroid compound(s) in the CNS tissue or fluid following intramuscular injection of an equivalent, cumulative concentration or dose of the steroid or corticosteroid compound(s) to the subject during the cumulative dosing period. In alternative embodiments, these compositions upon repeated dosing once or twice a day for a cumulative, effective dosing period between about 7-14 days provide for enhanced mucosal delivery of said one or more steroid or corticosteroid compound(s) to a targeted central nervous system (CNS) tissue or fluid to increase efficacy of the compositions for treatment of a CNS-associated autoimmune disease while minimizing systemic delivery to other sites to substantially reduce adverse side effects associated with systemic delivery administrative modes (e.g. via intravenous, intramuscular, or subcutaneous injection) of the subject steroid or corticosteroid compound(s). In more detailed embodiments, the CNS-associated autoimmune disease is multiple sclerosis. In other detailed embodiments, the side effect associated with systemic delivery of the subject steroid or corticosteroid compound(s) include one or more side effects selected from adrenosuppression and weight gain. Typically, the selected CNS tissue or fluid is a tissue or fluid associated with a subarachnoid space or nasopharyngeal lymphatic plexus in the CNS. In related embodiments, the compositions following single or multiple intranasal administration(s) to the subject yield an area under the concentration curve (AUC) of the steroid or corticosteroid compound(s) in a targeted central nervous system (CNS) tissue or fluid of that is about 2-fold, 3-fold, 5-fold, or 10-fold or greater compared to an AUC of the steroid or corticosteroid compound(s) in a blood plasma, adrenal tissue or fluid, or other non-CNS site in the subject. Exemplary targeted CNS tissues or fluids include tissues and fluids associated with a subarachnoid space or nasopharyngeal lymphatic plexus in the subject. These formulations and related methods, including coordinate (e.g., involving simultaneous or sequential administration) delivery methods, typically provide for increased efficacy for treatment of a CNS-associated autoimmune disease compared to injected steroid formulations and delivery methods involving injection of steroids, while minimizing systemic delivery to other sites to substantially reduce adverse side effects associated with systemic delivery of the subject steroid or corticosteroid compound(s).

The foregoing compositions are useful in methods for treating symptoms of inflammation, nasal irritation, rhinitis, allergy, autoimmune disease, and/or viral infection. In yet additional embodiments of the invention, pharmaceutical compositions and related methods are provided employing a composition for administration to a mammalian subject that comprises one or more steroid or corticosteroid compound(s) formulated with a mucosal delivery-enhancing agent. Typically, these formulations and related methods involve combinatorial formulation or coordinate delivery of the subject steroid or corticosteroid compound(s) with one or more mucosal delivery-enhancing agents described herein. In certain embodiments, these steroid compositions upon repeated intranasal dosing once or twice a day for a cumulative dosing period between about 7-14 days yield a cumulative area under the concentration curve (AUC) for said steroid or corticosteroid compound(s) in a central nervous system (CNS) tissue or fluid of the subject that is approximately 50% or greater compared to a cumulative AUC of the steroid or corticosteroid compound(s) in the CNS tissue or fluid following intramuscular injection of an equivalent, cumulative concentration or dose of the steroid or corticosteroid compound(s) to the subject during the cumulative dosing period.

In related aspects, the intranasal steroid compositions and methods yield, upon repeated dosing once or twice a day for a cumulative dosing period between about 7-14 days yield a cumulative area under the concentration curve (AUC) for the steroid or corticosteroid compound(s) in a central nervous system (CNS) tissue or fluid of the subject that is approximately 50%, 75%, 100% or greater compared to a cumulative AUC of the steroid or corticosteroid compound(s) in the CNS tissue or fluid following intramuscular injection of an equivalent, cumulative concentration or dose of the steroid or corticosteroid compound(s) to the subject during the cumulative dosing period. In alternative embodiments, these compositions upon repeated dosing once or twice a day for a cumulative, effective dosing period between about 7-14 days provide for enhanced mucosal delivery of the steroid or corticosteroid compound(s) to a targeted central nervous system (CNS) tissue or fluid to increase efficacy of the compositions for treatment of a CNS-associated autoimmune disease while minimizing systemic delivery to other sites to substantially reduce adverse side effects associated with systemic delivery administrative modes (e.g. via intravenous, intramuscular, or subcutaneous injection) of the subject steroid or corticosteroid compound(s). In more detailed embodiments, the CNS-associated autoimmune disease is multiple sclerosis. In other detailed embodiments, the side effect associated with systemic delivery of the subject steroid or corticosteroid compound(s) include one or more side effects selected from adrenosuppression and weight gain. Typically, the selected CNS tissue or fluid is a tissue or fluid associated with a subarachnoid space or nasopharyngeal lymphatic plexus in the CNS. In related embodiments, the compositions following single or multiple intranasal administration(s) to the subject yield an area under the concentration curve (AUC) of the steroid or corticosteroid compound(s) in a targeted central nervous system (CNS) tissue or fluid of that is about 2-fold, 3-fold, 5-fold, or 10-fold or greater compared to an AUC of the steroid or corticosteroid compound(s) in a blood plasma, adrenal tissue or fluid, or other non-CNS site in the subject. Exemplary targeted CNS tissues or fluids include tissues and fluids associated with a subarachnoid space or nasopharyngeal lymphatic plexus in the subject. These formulations and related methods, including coordinate (e.g., involving simultaneous or sequential administration) delivery methods, typically provide for increased efficacy for treatment of a CNS-associated autoimmune disease compared to injected steroid formulations and delivery methods involving injection of steroids, while minimizing systemic delivery to other sites to substantially reduce adverse side effects associated with systemic delivery of the subject steroid or corticosteroid compound(s).

As noted above, the present invention provides improved methods and compositions for mucosal delivery of interferon-β (IFN-β) to mammalian subjects for treatment or prevention of a variety of diseases and conditions. Examples of appropriate mammalian subjects for treatment and prophylaxis according to the methods of the invention include, but are not restricted to, humans and non-human primates, livestock species, such as horses, cattle, sheep, and goats, and research and domestic species, including dogs, cats, mice, rats, guinea pigs, and rabbits.

In order to provide better understanding of the present invention, the following definitions are provided:

Interferon-β: As used herein, “interferon-β” or “IFN-β” refers to interferon-β in native-sequence or in variant form, and from any source, whether natural, synthetic, or recombinant. Natural IFN-β is a glycoprotein (approximately 20 percent sugar moiety) of 20 kDa and has a length of 166 amino acids. Glycosylation is not required for biological activity in vitro. The protein contains a disulfide bond Cys31/141 required for biological activity. The human gene encoding IFN-β has a length of 777 bp and maps to chromosome 9q22 in the vicinity of the IFN-α gene cluster. The IFN-β gene does not contain introns. A single gene encodes the human IFN-β. At least three different genes have been found encoding bovine IFN-β. IFN-β is also known as: fibroblast interferon, Type 1 interferon, pH2-stable interferon, and R1-GI factor.

IFN-β includes, for example, human interferon-β (h IFN-β) that is a natural or recombinant IFN-β with the human native sequence. Recombinant interferon-β (rIFN-β) refers to any IFN-β or variant produced by means of recombinant DNA technology. Two subtypes of human IFN-β, IFN-β-1a (Avonex®, Biogen, Inc.) and IFN-β-1b (Betaseron®, Chiron Corp.), have been approved for the treatment and prevention of multiple sclerosis, and other diseases.

Additional disclosures teach detailed methods and tools pointing to specific structural and functional characteristics that define effective therapeutic uses of IFN-β, and further disclose a diverse, additional array of IFN-β agents and functional variants and analogs of IFN-β (including, but not limited to, natural or recombinant mutant forms of IFN-β, chemically or biosynthetically modified derivatives or variants of IFN-β and polypeptide and small molecule drug mimetics of IFN-β) that are also useful within the invention.

IFN-β is produced mainly by fibroblasts and some epithelial cell types. The synthesis of IFN-β can be induced by common inducers of interferons including viruses, double-stranded RNA, and micro-organisms. It is also induced by some cytokines such as tumor necrosis factor (TNF) and IL1. In contrast to IFN-α, IFN-β is strictly species-specific. IFN-β derived from other species is inactive in human cells

Within the mucosal delivery formulations and methods of the invention, continuous administration of interferon β to patients with multiple sclerosis permits the use of a lower dose, with subsequent lowering of significant drug related side effects. Because continuous infusion outside the hospital setting is impractical, the mucosal formulations for delivery of IFN-β of the present invention allow one to approximate a continuous administration, with the accrued benefits, including improved patient-to-patient dose variability.

Treatment and Prevention of Multiple Sclerosis by intranasal administration of a cytokine, for example, interferon-β, in combination with a steroid or corticosteroid composition. As noted above, the instant invention provides improved and useful methods and compositions for mucosal delivery of IFN-β to prevent and treat relapsing forms of multiple sclerosis (MS) in mammalian subjects. Within the mucosal delivery formulations and methods of the invention, nasal mucosal administration of interferon β to patients with multiple sclerosis is effective in treatment of MS disease with subsequent lowering of significant drug related side effects. Furthermore, within the mucosal delivery formulations and methods of the invention, nasal mucosal administration of interferon β in combination (i.e., in a combinatorial formulation or coordinate delivery protocol) with a steroid or corticosteroid composition to patients with multiple sclerosis further reduces symptoms, such as inflammation, associated with MS disease.

In one aspect of the invention, pharmaceutical formulations suitable for intranasal administration are provided that comprise a therapeutically effective amount of a cytokine compound, for example, interferon-β, in combination with a steroid compound and one or more intranasal delivery-enhancing agents as described herein, which formulations are effective in a nasal mucosal delivery method of the invention to prevent the onset or progression of disease or to alleviate one or more symptom(s) of multiple sclerosis, inflammation, nasal irritation, rhinitis, or allergy in a mammalian subject. The pharmaceutical formulations and coordinate delivery methods suitable for intranasal administration, as described herein, deliver the cytokine in combination with a steroid directly to the CNS tissue or fluid, while avoiding delivery of the steroid to the blood serum or other organs, and thus avoiding adverse side effects of the steroid composition.

In one aspect of the invention, pharmaceutical formulations suitable for intranasal administration are provided that comprise a therapeutically effective amount of an interferon-β compound in combination with a corticosteroid compound and one or more intranasal delivery-enhancing agents as described herein. The formulations are effective in a nasal mucosal delivery method of the invention to prevent the onset or progression of disease or to alleviate one or more symptom(s) of multiple sclerosis in a mammalian subject. Symptoms of multiple sclerosis include inflammation, tremors, muscle weakness, numbness in the limbs, and lesions in the central and peripheral nervous system that may lead to paralysis or blindness. Treatment of MS may require interferon-β in combination with a high dose corticosteroid, for example, a high potency steroid such as betamethasone or dexamethasone, or for example, a medium potency steroid such as triamcinolone, triamcinolone acetonide, methylprednisolone, prednisolone, or prednisone or a high dose of low potency steroid such as hydrocortisone or cortisone.

In one embodiment, a pharmaceutical formulation suitable for intranasal administration comprising interferon-β and a high dose corticosteroid compound, as described herein, is delivered once or twice per day for between about 7 and about 14 days. An exemplary dosage delivery of a steroid or corticosteroid composition, flunisolide (Nasalide®), is 2 puffs in nose bid, having a relative potency of 3. An exemplary dosage of a steroid or corticosteroid composition, fluticasone (Flonase®), is 2 puffs in nose qd for one week, then 1 puff qd, having a relative potency of 3. An exemplary dosage of a steroid or corticosteroid composition, triamcinolone acetonide (Nasacort®) is 2 puffs qd for 1 week, then 1 puff per day, having a relative potency of 1. A further exemplary dosage of a steroid or corticosteroid composition, beclomethasone dipropionate (Beconase®, Vancenase®) is 2 puffs bid (2 puffs qd for double strength), having a relative potency of 5. A further exemplary dosage of a steroid or corticosteroid composition, Budesonide (Rhinocort®), is 4 puffs qd for 1 week, then 2 puffs qd, having a relative potency of 10.

Mucosal administration of the interferon-β and corticosteroid compositions once or twice per day for 7 to 14 days to the subject yields extended delivery of the interferon-β and corticosteroid compositions. Delivery of the composition is measured by area under the concentration curve (AUC) for interferon-β, for the corticosteroid, or for a pharmacokinetic marker for interferon-β, for example, neopterin or β₂-microglobulin. Mucosal administration of the interferon-β and steroid compositions to the subject yields an AUC of corticosteroid, neopterin or β₂-microglobulin in a central nervous system (CNS) tissue or fluid of the subject that is typically about 50%, about 75% or about 100% or greater compared to an AUC of corticosteroid, neopterin or β₂-microglobulin in CNS tissue or fluid following intramuscular injection of an equivalent concentration or dose of interferon-β to the subject. Area under the concentration curve (AUC) determinations are made by taking samples of CSF hourly or every two to three hours, or longer over a period of 4 to 6 days. Concentrations of interferon-β, corticosteroid, neopterin or β₂-microglobulin are measured in each CSF sample. An additive value for AUC is determined for each compound for a time period, for example, 0 to 96 hours, or 0 to 144 hours.

In an embodiment of the pharmaceutical composition for treatment of multiple sclerosis, a pharmaceutical formulation suitable for intranasal administration comprising interferon-βcompound and a corticosteroid compound, as described herein, has substantially reduced side effects associated with intranasal administration of corticosteroid compared to side effects associated with intramuscular or subcutaneous injection of interferon-β and corticosteroid. Intranasal administration of interferon-β and corticosteroid, as described herein, provides effective mucosal delivery to sites selected from CNS tissue or cerebrospinal fluid, for example, CNS tissue or fluid within the subarachnoid space or nasopharyngeal lymphatic plexi. Compositions as described herein target the CNS tissue or fluid, and these compositions avoid delivery to sites of the body other than the CNS and avoid side effects associated with systemic delivery. Side effects are normally associated with systemic delivery, for example, by drug delivery via intravenous, intramuscular, or subcutaneous injection. Systemic delivery of steroid compounds targets the blood serum and organs, for example, adrenal gland and kidneys. Adverse steroid side effects, such as adrenosuppression and weight gain, are avoided in the pharmaceutical formulations suitable for intranasal administration to a CNS tissue or fluid of the subject, as described herein.

A pharmaceutical formulation suitable for intranasal administration comprising interferon-β and a high dose corticosteroid compound, as described herein, provides therapeutic delivery to the CNS while avoiding delivery to the blood serum and organs, for example, adrenal gland and kidneys. Pharmaceutical compositions as described herein yield an area under the concentration curve (AUC) of a corticosteroid composition in the CNS that is typically about 2-fold, about 3-fold, about 5-fold, or about 10-fold or greater when compared to an AUC for the composition in a blood plasma or other target tissue (adrenal gland or kidney). Pharmaceutical formulations as described herein target corticosteroids to the CNS tissues and fluids thus avoiding adverse steroid side effects as described above.

In one embodiment, an intranasal formulation of interferon-β in combination with a high potency steroid or corticosteroid composition includes, but is not limited to, betamethasone (0.6 to 0.75 mg dosage), or dexamethasone (0.75 mg dosage), typically in a dosage range from approximately 0.5 mg to approximately 0.8 mg, or typically in a dosage range from approximately 0.6 mg to approximately 0.75 mg. In a further embodiment, an intranasal formulation of interferon-β in combination with a medium potency steroid or corticosteroid composition includes, but is not limited to, methylprednisolone (4 mg dosage), triamcinolone (4 mg dosage), or prednisolone (5 mg dosage), typically in a dosage range from approximately 3 mg to approximately 6 mg, or typically in a dosage range from approximately 4 mg to approximately 5 mg. In a further embodiment, an intranasal formulation of interferon-β in combination with a low potency steroid or corticosteroid composition includes, but is not limited to hydrocortisone (20 mg dosage) or cortisone (25 mg dosage), typically in a dosage range from approximately 15 mg to approximately 30 mg, or typically in a dosage range from approximately 20 mg to approximately 25 mg.

The treatment and prevention of disease, for example, hepatitis B, childhood viral encephalitis, condylomata acuminata, malignant tumors and glioma by therapy with intranasal compositions of interferon-β and corticosteroid, as described herein, results in reduction in disease indications while avoiding side effects of drug delivery. Intranasal compositions of interferon-β and corticosteroid results in reduced nasal irritation, reduced rhinitis and a reduced nasal mucosal allergic response by direct delivery to the nasal mucosal tissue and to the CNS tissue or fluid. Direct intranasal delivery to the CNS tissue or fluid avoids systemic responses, for example adrenosuppression and weight gain.

In further aspect of the invention, pharmaceutical formulations suitable for intranasal administration are provided that comprise a therapeutically effective amount of an interferon-β compound in combination with a corticosteroid compound and one or more intranasal delivery-enhancing agents as described herein. The formulations are effective in a nasal mucosal delivery method of the invention to alleviate one or more symptom(s) of inflammation or disease in a mammalian subject. Compositions as described herein target the CNS tissue or fluid. The compositions avoid delivery to sites of the body other than the CNS and avoid side effects, such as adrenosuppression and weight gain, associated with systemic delivery of corticosteroids to the blood serum and organs, for example, the adrenal gland and kidney.

As described above, mucosal administration of the interferon-β and corticosteroid compositions once or twice per day for 7 to 14 days to the subject yields extended delivery of the interferon-β and corticosteroid compositions. Delivery of the composition is measured by area under the concentration curve (AUC) for interferon-β, the corticosteroid, or for a pharmacokinetic marker for interferon-β, for example, neopterin or β₂-microglobulin. Mucosal administration of the interferon-β and steroid compositions to the subject yields an AUC of corticosteroid, neopterin or β₂-microglobulin in a central nervous system (CNS) tissue or fluid of the subject that is typically about 50%, about 75% or about 100% or greater compared to an AUC of corticosteroid, neopterin or β₂-microglobulin in CNS tissue or fluid following intramuscular injection of an equivalent concentration or dose of interferon-β to the subject.

A pharmaceutical formulation suitable for intranasal administration comprising interferon-β and a corticosteroid compound for treatment of inflammation, as described herein, provides therapeutic delivery to the CNS while avoiding delivery to the blood serum and organs, for example, adrenal gland and kidneys. Pharmaceutical compositions yield an area under the concentration curve (AUC) of a corticosteroid composition in the CNS that is typically about 2-fold, about 3-fold, about 5-fold, or about 10-fold or greater when compared to an AUC for the composition in a blood plasma or other target tissue (adrenal gland or kidney). Pharmaceutical formulations, as described herein, target corticosteroids to the CNS tissues and fluids thus avoiding adverse steroid side effects, such as adrenosuppression and weight gain caused by prolonged steroid treatment.

In a further aspect of the invention, pharmaceutical formulations suitable for intranasal administration are provided that comprise a therapeutically effective amount of a steroid compound as described herein. The pharmaceutical formulation suitable for intranasal administration comprises a therapeutically effective amount of a steroid compound in combination with one or more intranasal delivery-enhancing agents. Alternatively, the pharmaceutical formulation suitable for intranasal administration comprises a therapeutically effective amount of a steroid compound without intranasal delivery-enhancing agents. The formulations are effective in a nasal mucosal delivery method of the invention to delivery a steroid composition, for example, a corticosteroid, resulting in reduced inflammation, reduced nasal irritation, reduced rhinitis, and a reduced nasal mucosal allergic response by direct delivery to the nasal mucosal tissue and to the CNS tissue or fluid.

Mucosal administration of steroid compositions for example, a corticosteroid, once or twice per day for 7 to 14 days to the subject yields extended delivery of the corticosteroid composition. Delivery of the composition is measured by area under the concentration curve (AUC) for the corticosteroid. Mucosal administration of the corticosteroid composition to the subject yields an AUC of corticosteroid in a central nervous system (CNS) tissue or fluid of the subject that is typically about 50%, about 75% or about 100% or greater compared to an AUC of corticosteroid in CNS tissue or fluid following intramuscular injection of an equivalent concentration or dose of corticosteroid to the subject.

A pharmaceutical formulation suitable for intranasal administration comprising a corticosteroid compound for treatment of inflammation, rhinitis, allergy, or nasal irritation, as described herein, provides therapeutic delivery to the CNS tissue or fluid while avoiding delivery to the blood serum and organs, for example, adrenal gland and kidneys. Pharmaceutical compositions as described herein yield an area under the concentration curve (AUC) of a corticosteroid composition in the CNS that is typically about 2-fold, about 3-fold, about 5-fold, or about 10-fold or greater when compared to an AUC for the composition in a blood plasma or other organ (for example, adrenal gland or kidney). Pharmaceutical formulations as described herein target corticosteroids to the CNS tissues and fluids thus avoiding adverse side effects, such as adrenosuppression and weight gain caused by prolonged steroid treatment.

Treatment and Prevention of Hepatitis B: As noted above, the instant invention provides improved and useful methods and compositions for mucosal delivery of IFN-β to prevent and treat hepatitis B infection in mammalian subjects. IFN- [symbol] alone or in combination with IFN- [symbol] is useful in the treatment of chronic active hepatitis B.

Treatment and Prevention of Childhood Viral Encephalitis: As noted above, the instant invention provides improved and useful methods and compositions for mucosal delivery of IFN-β to prevent and treat severe childhood viral encephalitis in mammalian subjects. A combination treatment of interferon β with acyclovir is more effective than treatment with acyclovir alone.

Treatment and Prevention of Condylomata Acuminata: As noted above, the instant invention provides improved and useful methods and compositions for mucosal delivery of IFN-β to prevent and treat papilloma virus infection in mammalian subjects. IFN-β is used for treatment of condyloma acuminata (genital or venereal warts caused by papilloma virus infection), papillomavirus warts of the larynx and skin (common warts). It is also suitable for the prophylactic use following surgical removal of large condylomas.

Treatment and Prevention of Malignant Tumors: Within the mucosal delivery formulations and methods of the invention, IFN-β is a lipophilic molecule that is particularly useful for local tumor therapy due to its specific pharmacokinetics. Head and neck squamous carcinomas, mammary and cervical carcinomas, and also malignant melanomas respond well to treatment with IFN-β. IFN-β is useful for the adjuvant therapy of malignant melanomas with a high potential for metastasis. Response rates are improved by combining IFN-β with antineoplastic agents or other cytokines.

Treatment and Prevention of Malignant Glioma: Within the mucosal delivery formulations and methods of the invention, combination therapy with IFN-β, MCNU (Ranimustine), and radiotherapy had a pronounced effect on untreated malignant glioma, with moderate side effects and no substantial effect on patients' general condition. (Wakabayashi, et al., J. Neurooncol, 49: 57-62, 2000)

Methods and Compositions of Delivery: Improved methods and compositions for mucosal administration of interferon-β to mammalian subjects optimize interferon-β dosing schedules. The present invention provides mucosal delivery of interferon-β formulated with one or more mucosal delivery-enhancing agents wherein interferon-β dosage release is substantially normalized and/or sustained for an effective delivery period of interferon-β release ranges from approximately 0.1 to 2.0 hours; 0.4 to 1.5 hours; 0.7 to 1.5 hours; or 1.0 to 1.3 hours; following mucosal administration. The sustained release of interferon-β is achieved may be facilitated by repeated administration of exogenous interferon-β utilizing methods and compositions of the present invention.

Compositions and Methods of Sustained Release: Improved compositions and methods for mucosal administration of interferon-β to mammalian subjects optimize interferon-β dosing schedules. The present invention provides improved mucosal (e.g., nasal) delivery of a formulation comprising interferon-β in combination with one or more mucosal delivery-enhancing agents and an optional sustained release-enhancing agent or agents. Mucosal delivery-enhancing agents of the present invention yield an effective increase in delivery, e.g., an increase in the maximal plasma concentration (Cₘₐₓ) to enhance the therapeutic activity of mucosally-administered interferon-β. A second factor affecting therapeutic activity of interferon-β in the blood plasma and CNS is residence time (RT). Sustained release-enhancing agents, in combination with intranasal delivery-enhancing agents, increase Cₘₐₓ and increase residence time (RT) interferon-β. Polymeric delivery vehicles and other agents and methods of the present invention that yield sustained release-enhancing formulations, for example, polyethylene glycol (PEG), are disclosed herein. The present invention provides an improved interferon-β delivery method and dosage form for treatment of symptoms related to interferon-β deficiency in mammalian subjects.

Maintenance of Basal Levels of Interferon-β: Improved compositions and methods for mucosal administration of interferon-β to mammalian subjects optimize interferon-β dosing schedules. The present invention provides improved nasal mucosal delivery of a formulation comprising interferon-β and intranasal delivery-enhancing agents in combination with subcutaneous and intramuscular administration of interferon-β. Formulations and methods of the present invention maintain relatively consistent basal levels of interferon-β, for example throughout a 2 to 24 hour, 4-16 hour, or 8-12 hour period following a single dose administration or attended by a multiple dosing regimen of 2-6 sequential administrations, often such that biological markers including neopterin and beta-2 microglobulin or 2,5-oligoadenylate synthetase are maintained at therapeutic levels at all times. Maintenance of basal levels of interferon-β is particularly useful for treatment and prevention of disease, for example, multiple sclerosis, without unacceptable adverse side effects.

Interferon β is produced by various cell types including fibroblasts and macrophages. Interferon β exerts its biological effects by binding to specific receptors on the surface of human cells. This binding initiates a complex cascade of intracellular events that leads to the expression of gene products and markers, for example, 2′,5′ oligoadenylate synthetase (2′,5′-OAS), neopterin, and β₂-microglobulin. These markers have been used to monitor the biological activity of interferon β-1a in humans. Induction of the biological response markers roughly correlates with serum activity levels of interferon β. These biological markers roughly peak 48 hours after administering a intramuscular or subcutaneous dose of interferon β and remain elevated for 4 days. After a intramuscular dose serum levels of interferon β peak about 3 to 15 hours after dosing. The elimination half-life is around 10 hours.

The effectiveness of interferon β is related to the increases in these biological markers. The doses chosen for clinical trials of Avonex® were based on the level of increase in β₂-microglobulin. 6 MIU (30 μg). The recommended dose of Avonex® is 30 μg injected intramuscularly once a week.

For example, interferon β at a 30 μg dose given intramuscularly once weekly would typically be an effective initial dose. The improved nasal mucosal delivery of a formulation comprising interferon-β and intranasal delivery-enhancing agents of the present invention at a dose of 60 to 120 μg per day would typically be given to sustain the biological markers beyond 4 days.

Within the mucosal delivery formulations and methods of the invention, the interferon-β is frequently combined or coordinately administered with a suitable carrier or vehicle for mucosal delivery. As used herein, the term “carrier” means a pharmaceutically acceptable solid or liquid filler, diluent or encapsulating material. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories, can be found in the U.S. Pharmacopeia National Formulary, pp. 1857-1859, 1990. Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other non toxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator. Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like. The amount of active ingredient that can be combined with the carrier materials to produce a single dosage form will vary depending upon the particular mode of administration.

The mucosal formulations of the invention are generally sterile, particulate free and stable for pharmaceutical use. As used herein, the term “particulate free” means a formulation that meets the requirements of the USP specification for small volume parenteral solutions. The term “stable” means a formulation that fulfills all chemical and physical specifications with respect to identity, strength, quality, and purity that have been established according to the principles of Good Manufacturing Practice, as set forth by appropriate governmental regulatory bodies.

Within the mucosal delivery compositions and methods of the invention, various delivery-enhancing agents are employed which enhance delivery of interferon-β into or across a mucosal surface. In this regard, delivery of interferon-β across the mucosal epithelium can occur “transcellularly” or “paracellularly”. The extent to which these pathways contribute to the overall flux and bioavailability of the interferon-β depends upon the environment of the mucosa, the physico-chemical properties the active agent, and on the properties of the mucosal epithelium. Paracellular transport involves only passive diffusion, whereas transcellular transport can occur by passive, facilitated or active processes. Generally, hydrophilic, passively transported, polar solutes diffuse through the paracellular route, while more lipophilic solutes use the transcellular route. Absorption and bioavailability (e.g., as reflected by a permeability coefficient or physiological assay), for diverse, passively and actively absorbed solutes, can be readily evaluated, in terms of both paracellular and transcellular delivery components, for any selected interferon-β within the invention. These values can be determined and distinguished according to well known methods, such as in vitro epithelial cell culture permeability assays (see, e.g., Hilgers, et al., Pharm. Res.7:902-910, 1990; Wilson et al., J. Controlled Release 11:25-40,1990; Artursson. I., Pharm. Sci. 79:476-482, 1990; Cogburn et al., Pharm. Res. 8:210-216, 1991; Pade et al., Pharmaceutical Research 14:1210-1215, 1997).

For passively absorbed drugs, the relative contribution of paracellular and transcellular pathways to drug transport depends upon the pKa, partition coefficient, molecular radius and charge of the drug, the pH of the luminal environment in which the drug is delivered, and the area of the absorbing surface. The paracellular route represents a relatively small fraction of accessible surface area of the nasal mucosal epithelium. In general terms, it has been reported that cell membranes occupy a mucosal surface area that is a thousand times greater than the area occupied by the paracellular spaces. Thus, the smaller accessible area, and the size- and charge-based discrimination against macromolecular permeation would suggest that the paracellular route could be a generally less favorable route than transcellular delivery for drug transport. Surprisingly, the methods and compositions of the invention provide for significantly enhanced transport of biotherapeutics into and across mucosal epithelia via the paracellular route. Therefore, the methods and compositions of the invention successfully target both paracellular and transcellular routes, alternatively or within a single method or composition.

As used herein, “mucosal delivery-enhancing agents” include agents which enhance the release or solubility (e.g., from a formulation delivery vehicle), diffusion rate, penetration capacity and timing, uptake, residence time, stability, effective half-life, peak or sustained concentration levels, clearance and other desired mucosal delivery characteristics (e.g., as measured at the site of delivery, or at a selected target site of activity such as the bloodstream or central nervous system) of interferon-β or other biologically active compound(s). Enhancement of mucosal delivery can thus occur by any of a variety of mechanisms, for example by increasing the diffusion, transport, persistence or stability of interferon-β, increasing membrane fluidity, modulating the availability or action of calcium and other ions that regulate intracellular or paracellular permeation, solubilizing mucosal membrane components (e.g., lipids), changing non-protein and protein sulfhydryl levels in mucosal tissues, increasing water flux across the mucosal surface, modulating epithelial junctional physiology, reducing the viscosity of mucus overlying the mucosal epithelium, reducing mucociliary clearance rates, and other mechanisms.

As used herein, a “mucosally effective amount of interferon-β” contemplates effective mucosal delivery of interferon-β to a target site for drug activity in the subject that may involve a variety of delivery or transfer routes. For example, a given active agent may find its way through clearances between cells of the mucosa and reach an adjacent vascular wall, while by another route the agent may, either passively or actively, be taken up into mucosal cells to act within the cells or be discharged or transported out of the cells to reach a secondary target site, such as the systemic circulation. The methods and compositions of the invention may promote the translocation of active agents along one or more such alternate routes, or may act directly on the mucosal tissue or proximal vascular tissue to promote absorption or penetration of the active agent(s). The promotion of absorption or penetration in this context is not limited to these mechanisms.

As used herein “peak concentration (Cₘₐₓ) of interferon-β in a blood plasma”, “area under concentration vs. time curve (AUC) of interferon-β in a blood plasma”, “time to maximal plasma concentration (tₘₐₓ) of interferon-β in a blood plasma” are pharmacokinetic parameters known to one skilled in the art. (Laursen et al., Eur. J. Endocrinology, 135: 309-315, 1996.) The “concentration vs. time curve” measures the concentration of interferon-β in a blood serum of a subject vs. time after administration of a dosage of interferon-β to the subject either by intranasal, subcutaneous, or other parenteral route of administration. “Cₘₐₓ” is the maximum concentration of interferon-β in the blood serum of a subject following a single dosage of interferon-β to the subject. “tₘₐₓ” is the time to reach maximum concentration of interferon-β in a blood serum of a subject following administration of a single dosage of interferon-β to the subject.

As used herein, “area under concentration vs. time curve (AUC) of interferon-β in a blood plasma” is calculated according to the linear trapezoidal rule and with addition of the residual areas. A decrease of 23% or an increase of 30% between two dosages would be detected with a probability of 90% (type II error β=10%). The “delivery rate” or “rate of absorption” is estimated by comparison of the time (tₘₐₓ) to reach the maximum concentration (Cₘₐₓ). Both Cₘₐₓ and tₘₐₓ are analyzed using non-parametric methods. Comparisons of the pharmacokinetics of subcutaneous, intravenous and intranasal interferon-β administrations were performed by analysis of variance (ANOVA). For pairwise comparisons a Bonferroni-Holmes sequential procedure was used to evaluate significance. The dose-response relationship between the three nasal doses was estimated by regression analysis. P<0.05 was considered significant. Results are given as mean values+/−SEM. (Laursen et al., 1996.)

As used herein, “pharmacokinetic markers” include any accepted biological marker that is detectable in an in vitro or in vivo system useful for modeling pharmacokinetics of mucosal delivery of one or more interferon-β compounds, or other biologically active agent(s) disclosed herein, wherein levels of the marker(s) detected at a desired target site following administration of the interferon-β compound(s) according to the methods and formulations herein, provide a reasonably correlative estimate of the level(s) of the interferon-β compound(s) delivered to the target site. Among many art-accepted markers in this context are substances induced at the target site by adminstration of the interferon-β compound(s) or orther biologically active agent(s). For example, nasal mucosal delivery of an effective amount of one or more interferon-β compounds according to the invention stimulates an immunologic response in the subject measurable by production of pharmacokinetic markers that include, but are not limited to, neopterin and β₂-microglobulin.

While the mechanism of absorption promotion may vary with different intranasal delivery-enhancing agents of the invention, useful reagents in this context will not substantially adversely affect the mucosal tissue and will be selected according to the physicochemical characteristics of the particular interferon-β or other active or delivery-enhancing agent. In this context, delivery-enhancing agents that increase penetration or permeability of mucosal tissues will often result in some alteration of the protective permeability barrier of the mucosa. For such delivery-enhancing agents to be of value within the invention, it is generally desired that any significant changes in permeability of the mucosa be reversible within a time frame appropriate to the desired duration of drug delivery. Furthermore, there should be no substantial, cumulative toxicity, nor any permanent deleterious changes induced in the barrier properties of the mucosa with long-term use.

Within certain aspects of the invention, absorption-promoting agents for coordinate administration or combinatorial formulation with interferon-β of the invention are selected from small hydrophilic molecules, including but not limited to, dimethyl sulfoxide (DMSO), dimethylformamide, ethanol, propylene glycol, and the 2-pyrrolidones. Alternatively, long-chain amphipathic molecules, for example, deacylmethyl sulfoxide, azone, sodium lauryl sulfate, oleic acid, and the bile salts, may be employed to enhance mucosal penetration of the interferon-β. In additional aspects, surfactants (e.g., polysorbates) are employed as adjunct compounds, processing agents, or formulation additives to enhance intranasal delivery of the interferon-β. These penetration-enhancing agents typically interact at either the polar head groups or the hydrophilic tail regions of molecules that comprise the lipid bilayer of epithelial cells lining the nasal mucosa (Barry, Pharmacology of the Skin, Vol. 1, pp. 121-137, Shroot et al., Eds., Karger, Basel, 1987; and Barry, J. controlled Release 6:85-97, 1987). Interaction at these sites may have the effect of disrupting the packing of the lipid molecules, increasing the fluidity of the bilayer, and facilitating transport of the interferon-β across the mucosal barrier. Interaction of these penetration enhancers with the polar head groups may also cause or permit the hydrophilic regions of adjacent bilayers to take up more water and move apart, thus opening the paracellular pathway to transport of the interferon-β. In addition to these effects, certain enhancers may have direct effects on the bulk properties of the aqueous regions of the nasal mucosa. Agents such as DMSO, polyethylene glycol, and ethanol can, if present in sufficiently high concentrations in delivery environment (e.g., by pre-administration or incorporation in a therapeutic formulation), enter the aqueous phase of the mucosa and alter its solubilizing properties, thereby enhancing the partitioning of the interferon-β from the vehicle into the mucosa.

Additional mucosal delivery-enhancing agents that are useful within the coordinate administration and processing methods and combinatorial formulations of the invention include, but are not limited to, mixed micelles; enamines; nitric oxide donors (e.g., S-nitroso-N-acetyl-DL-penicillamine, NOR1, NOR4—which are preferably co-administered with an NO scavenger such as carboxy-PITO or doclofenac sodium); sodium salicylate; glycerol esters of acetoacetic acid (e.g., glyceryl-1,3-diacetoacetate or 1,2-isopropylideneglycerine-3-acetoacetate); and other release-diffusion or intra- or trans-epithelial penetration-promoting agents that are physiologically compatible for mucosal delivery. Other absorption-promoting agents are selected from a variety of carriers, bases and excipients that enhance mucosal delivery, stability, activity or trans-epithelial penetration of the interferon-β. These include, inter alia, clyclodextrins and β-cyclodextrin derivatives (e.g., 2-hydroxypropyl-β-cyclodextrin and heptakis(2,6-di-O-methyl-β-cyclodextrin). These compounds, optionally conjugated with one or more of the active ingredients and further optionally formulated in an oleaginous base, enhance bioavailability in the mucosal formulations of the invention. Yet additional absorption-enhancing agents adapted for mucosal delivery include medium-chain fatty acids, including mono- and diglycerides (e.g., sodium caprate—extracts of coconut oil, Capmul), and triglycerides (e.g., amylodextrin, Estaram 299, Miglyol 810).

The mucosal therapeutic and prophylactic compositions of the present invention may be supplemented with any suitable penetration-promoting agent that facilitates absorption, diffusion, or penetration of interferon-β across mucosal barriers. The penetration promoter may be any promoter that is pharmaceutically acceptable. Thus, in more detailed aspects of the invention compositions are provided that incorporate one or more penetration-promoting agents selected from sodium salicylate and salicylic acid derivatives (acetyl salicylate, choline salicylate, salicylamide, etc.); amino acids and salts thereof (e.g. monoaminocarboxlic acids such as glycine, alanine, phenylalanine, proline, hydroxyproline, etc.; hydroxyamino acids such as serine; acidic amino acids such as aspartic acid, glutamic acid, etc; and basic amino acids such as lysine etc—inclusive of their alkali metal or alkaline earth metal salts); and N-acetylamino acids (N-acetylalanine, N-acetylphenylalanine, N-acetylserine, N-acetylglycine, N-acetyllysine, N-acetylglutamic acid, N-acetylproline, N-acetylhydroxyproline, etc.) and their salts (alkali metal salts and alkaline earth metal salts). Also provided as penetration-promoting agents within the methods and compositions of the invention are substances which are generally used as emulsifiers (e.g. sodium oleyl phosphate, sodium lauryl phosphate, sodium lauryl sulfate, sodium myristyl sulfate, polyoxyethylene alkyl ethers, polyoxyethylene alkyl esters, etc.), caproic acid, lactic acid, malic acid and citric acid and alkali metal salts thereof, pyrrolidonecarboxylic acids, alkylpyrrolidonecarboxylic acid esters, N-alkylpyrrolidones, proline acyl esters, and the like.

Within various aspects of the invention, improved nasal mucosal delivery formulations and methods are provided that allow delivery of interferon-β and other therapeutic agents within the invention across mucosal barriers between administration and selected target sites. Certain formulations are specifically adapted for a selected target cell, tissue or organ, or even a particular disease state. In other aspects, formulations and methods provide for efficient, selective endo- or transcytosis of interferon-β specifically routed along a defined intracellular or intercellular pathway. Typically, the interferon-β is efficiently loaded at effective concentration levels in a carrier or other delivery vehicle, and is delivered and maintained in a stabilized form, e.g., at the nasal mucosa and/or during passage through intracellular compartments and membranes to a remote target site for drug action (e.g., the blood stream or a defined tissue, organ, or extracellular compartment). The interferon-β may be provided in a delivery vehicle or otherwise modified (e.g., in the form of a prodrug), wherein release or activation of the interferon-β is triggered by a physiological stimulus (e.g. pH change, lysosomal enzymes, etc.) Often, the interferon-β is pharmacologically inactive until it reaches its target site for activity. In most cases, the interferon-β and other formulation components are non-toxic and non-immunogenic. In this context, carriers and other formulation components are generally selected for their ability to be rapidly degraded and excreted under physiological conditions. At the same time, formulations are chemically and physically stable in dosage form for effective storage.

Peptide and Protein Analogs and Mimetics

Included within the definition of biologically active peptides and proteins for use within the invention are natural or synthetic, therapeutically or prophylactically active, peptides (comprised of two or more covalently linked amino acids), proteins, peptide or protein fragments, peptide or protein analogs, and chemically modified derivatives or salts of active peptides or proteins. For example, a wide variety of these various kinds of peptide or protein analogs or mimetics are known in the art or achieved following know methods for interferon-β. Often, the peptides or proteins of interferon-β or other biologically active peptides or proteins for use within the invention are muteins that are readily obtainable by partial substitution, addition, or deletion of amino acids within a naturally occurring or native (e.g., wild-type, naturally occurring mutant, or allelic variant) peptide or protein sequence. Additionally, biologically active fragments of native peptides or proteins are included. Such mutant derivatives and fragments substantially retain the desired biological activity of the native peptide or proteins. In the case of peptides or proteins having carbohydrate chains, biologically active variants marked by alterations in these carbohydrate species are also included within the invention.

In additional embodiments, peptides or proteins for use within the invention may be modified by addition or conjugation of a synthetic polymer, such as polyethylene glycol, a natural polymer, such as hyaluronic acid, or an optional sugar (e.g. galactose, mannose), sugar chain, or nonpeptide compound. Substances added to the peptide or protein by such modifications may specify or enhance binding to certain receptors or antibodies or otherwise enhance the mucosal delivery, activity, half-life, cell- or tissue-specific targeting, or other beneficial properties of the peptide or protein. For example, such modifications may render the peptide or protein more lipophilic, e.g., such as may be achieved by addition or conjugation of a phospholipid or fatty acid. Further included within the methods and compositions of the invention are peptides and proteins prepared by linkage (e.g., chemical bonding) of two or more peptides, protein fragments or functional domains (e.g., extracellular, transmembrane and cytoplasmic domains, ligand-binding regions, active site domains, immunogenic epitopes, and the like)—for example fusion peptides and proteins recombinantly produced to incorporate the functional elements of a plurality of different peptides or proteins in a single encoded molecule.

Biologically active peptides and proteins for use within the methods and compositions of the invention thus include native or “wild-type” peptides and proteins and naturally occurring variants of these molecules, e.g., naturally occurring allelic variants and mutant proteins. Also included are synthetic, e.g., chemically or recombinantly engineered, peptides and proteins, as well as peptide and protein “analogs” and chemically modified derivatives, fragments, conjugates, and polymers of naturally occurring peptides and proteins. As used herein, the term peptide or protein “analog” is meant to include modified peptides and proteins incorporating one or more amino acid substitutions, insertions, rearrangements or deletions as compared to a native amino acid sequence of a selected peptide or protein, or of a binding domain, fragment, immunogenic epitope, or structural motif, of a selected peptide or protein. Peptide and protein analogs thus modified exhibit substantially conserved biological activity comparable to that of a corresponding native peptide or protein, which means activity (e.g., specific binding to a interferon-β protein, or to a cell expressing such a protein, specific ligand or receptor binding activity, etc.) levels of at least 50%, typically at least 75%, often 85%-95% or greater, compared to activity levels of a corresponding native protein or peptide.

For purposes of the present invention, the term biologically active peptide or protein “analog” further includes derivatives or synthetic variants of a native peptide or protein, such as amino and/or carboxyl terminal deletions and fusions, as well as intrasequence insertions, substitutions or deletions of single or multiple amino acids. Insertional amino acid sequence variants are those in which one or more amino acid residues are introduced into a predetermined site in the protein. Random insertion is also possible with suitable screening of the resulting product. Deletional variants are characterized by removal of one or more amino acids from the sequence. Substitutional amino acid variants are those in which at least one residue in the sequence has been removed and a different residue inserted in its place.

Where a native peptide or protein is modified by amino acid substitution, amino acids are generally replaced by other amino acids having similar, conservatively related chemical properties such as hydrophobicity, hydrophilicity, electronegativity, small or bulky side chains, and the like. Residue positions which are not identical to the native peptide or protein sequence are thus replaced by amino acids having similar chemical properties, such as charge or polarity, where such changes are not likely to substantially effect the properties of the peptide or protein analog. These and other minor alterations will typically substantially maintain biological properties of the modified peptide or protein, including biological activity (e.g., binding to interferon-β, adhesion molecule, or other ligand or receptor), immunoidentity (e.g., recognition by one or more monoclonal antibodies that recognize a native peptide or protein), and other biological properties of the corresponding native peptide or protein.

As used herein, the term “conservative amino acid substitution” refers to the general interchangeability of amino acid residues having similar side chains. For example, a commonly interchangeable group of amino acids having aliphatic side chains is alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Examples of conservative substitutions include the substitution of a non-polar (hydrophobic) residue such as isoleucine, valine, leucine or methionine for another. Likewise, the present invention contemplates the substitution of a polar (hydrophilic) residue such as between arginine and lysine, between glutamine and asparagine, and between threonine and serine. Additionally, the substitution of a basic residue such as lysine, arginine or histidine for another or the substitution of an acidic residue such as aspartic acid or glutamic acid for another is also contemplated. Exemplary conservative amino acids substitution groups are: valine-leucine-isoleucine, phenylalanine-tyrosine, lysine-arginine, alanine-valine, and asparagine-glutamine.

The term biologically active peptide or protein analog further includes modified forms of a native peptide or protein incorporating stereoisomers (e.g., D-amino acids) of the twenty conventional amino acids, or unnatural amino acids such as α,α-disubstituted amino acids, N-alkyl amino acids, lactic acid. These and other unconventional amino acids may also be substituted or inserted within native peptides and proteins useful within the invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, ω-N-methylarginine, and other similar amino acids and imino acids (e.g., 4-hydroxyproline). In addition, biologically active peptide or protein analogs include single or multiple substitutions, deletions and/or additions of carbohydrate, lipid and/or proteinaceous moieties that occur naturally or artificially as structural components of the subject peptide or protein, or are bound to or otherwise associated with the peptide or protein.

To facilitate production and use of peptide and protein analogs within the invention, reference can be made to molecular phylogenetic studies that characterize conserved and divergent protein structural and functional elements between different members of a species, genus, family or other taxonomic group (e.g., between different human interferon-β protein family members, allelic variants, and/or naturally occurring mutants, or between interferon-β proteins found in different species, such as human, murine, rat and/or bovine interferon-β). In this regard, available studies will provide detailed assessments of structure-function relationships on a fine molecular level for modifying the majority of peptides and proteins disclosed herein to facilitate production and selection of operable peptide and protein analogs, including for example, interferon-β, and other biologically active peptides and proteins disclosed herein for use within the invention. These studies include, for example, detailed sequence comparisons identifying conserved and divergent structural elements among, for example, multiple isoforms or species or allelic variants of a subject interferon-β peptide or protein. Each of these conserved and divergent structural elements facilitate practice of the invention by pointing to useful targets for modifying native peptides and proteins to confer desired structural and/or functional changes.

In this context, existing sequence alignments may be analyzed and conventional sequence alignment methods may be employed to yield sequence comparisons for analysis, for example to identify corresponding protein regions and amino acid positions between protein family members within a species, and between species variants of a protein of interest. These comparisons are useful to identify conserved and divergent structural elements of interest, the latter of which will often be useful for incorporation in a biologically active peptide or protein to yield a functional analog thereof. Typically, one or more amino acid residues marking a divergent structural element of interest in a different reference peptide sequence is incorporated within the functional peptide or protein analog. For example, a cDNA encoding a native interferon-β peptide or protein may be recombinantly modified at one or more corresponding amino acid position(s) (i.e., corresponding positions that match or span a similar aligned sequence element according to accepted alignment methods to residues marking the structural element of interest in a heterologous reference peptide or protein sequence, such as an isoform, species or allelic variant, or synthetic mutant, of the subject interferon-β peptide or protein) to encode an amino acid deletion, substitution, or insertion that alters corresponding residue(s) in the native peptide or protein to generate an operable peptide or protein analog within the invention—having an analogous structural and/or functional element as the reference peptide or protein.

Within this rational design method for constructing biologically active peptide and protein analogs, the native or wild-type identity of residue(s) at amino acid positions corresponding to a structural element of interest in a heterologous reference peptide or protein may be altered to the same, or a conservatively related, residue identity as the corresponding amino acid residue(s) in the reference peptide or protein. However, it is often possible to alter native amino acid residues non-conservatively with respect to the corresponding reference protein residue(s). In particular, many non-conservative amino acid substitutions, particularly at divergent sites suggested to be more amenable to modification, may yield a moderate impairment or neutral effect, or even enhance a selected biological activity, compared to the function of a native peptide or protein.

Sequence alignment and comparisons to forecast useful peptide and protein analogs and mimetics will be further refined by analysis of crystalline structure (see, e.g., Löebermann et al., J. Molec. Biol. 177:531-556, 1984; Huber et al., Biochemistry 28:8951-8966, 1989; Stein et al., Nature 347:99-102, 1990; Wei et al., Structural Biology 1:251-255, 1994) of native biologically active proteins and peptides, coupled with computer modeling methods known in the art. These analyses allow detailed structure-function mapping to identify desired structural elements and modifications for incorporation into peptide and protein analogs and mimetics that will exhibit substantial activity comparable to that of the native peptide or protein for use within the methods and compositions of the invention.

Biologically active peptide and protein analogs of the invention typically show substantial sequence identity to a corresponding native peptide or protein sequence. The term “substantial sequence identity” means that the two subject amino acid sequences, when optimally aligned, such as by the programs GAP or BESTFIT using default gap penalties, share at least 65 percent sequence identity, commonly 80 percent sequence identity, often at least 90-95 percent or greater sequence identity. “Percentage amino acid identity” refers to a comparison of the amino acid sequences of two peptides or proteins which, when optimally aligned, have approximately the designated percentage of the same amino acids. Sequence comparisons are generally made to a reference sequence over a comparison window of at least 10 residue positions, frequently over a window of at least 15-20 amino acids, wherein the percentage of sequence identity is calculated by comparing a reference sequence to a second sequence, the latter of which may represent, for example, a peptide analog sequence that includes one or more deletions, substitutions or additions which total 20 percent, typically less than 5-10% of the reference sequence over the window of comparison. The reference sequence may be a subset of a larger sequence, for example, as a segment of interferon-β protein. Optimal alignment of sequences (e.g., alignment of human interferon-β with another mammalian interferon-β protein) for aligning a comparison window may be conducted according to the local homology algorithm of Smith and Waterman (Adv. Appl. Math. 2:482, 1981), by the homology alignment algorithm of Needleman and Wunsch (J. Mol. Biol. 48:443, 1970), by the search for similarity method of Pearson and Lipman (Proc. Natl. Acad. Sci.USA 85:2444, 1988), or by computerized implementations of these algorithms (GAP, BESTFIT, FASTA, and/or TFASTA, e.g., as provided in the Wisconsin Genetics Software Package Release 7.0, Genetics Computer Group, 575 Science Dr., Madison, Wis.).

By aligning a peptide or protein analog optimally with a corresponding native peptide or protein, and by using appropriate assays, e.g., adhesion protein or receptor binding assays, to determine a selected biological activity, one can readily identify operable peptide and protein analogs for use within the methods and compositions of the invention. Operable peptide and protein analogs are typically specifically immunoreactive with antibodies raised to the corresponding native peptide or protein

Within additional aspects of the invention, peptide mimetics are provided which comprise a peptide or non-peptide molecule that mimics the tertiary binding structure and activity of a selected native peptide or protein functional domain (e.g., binding motif or active site). These peptide mimetics include recombinantly or chemically modified peptides, as well as non-peptide agents such as small molecule drug mimetics, as further described below.

In one aspect, peptides (including polypeptides) useful within the invention are modified to produce peptide mimetics by replacement of one or more naturally occurring side chains of the 20 genetically encoded amino acids (or D amino acids) with other side chains, for instance with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclics. For example, proline analogs can be made in which the ring size of the proline residue is changed from 5 members to 4, 6, or 7 members. Cyclic groups can be saturated or unsaturated, and if unsaturated, can be aromatic or non-aromatic. Heterocyclic groups can contain one or more nitrogen, oxygen, and/or sulphur heteroatoms. Examples of such groups include the furazanyl, furyl, imidazolidinyl, imidazolyl, imidazolinyl, isothiazolyl, isoxazolyl, morpholinyl (e.g. morpholino), oxazolyl, piperazinyl (e.g. 1-piperazinyl), piperidyl (e.g. 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g. 1-pyrrolidinyl), pyrrolinyl, pyrrolyl, thiadiazolyl, thiazolyl, thienyl, thiomorpholinyl (e.g. thiomorpholino), and triazolyl. These heterocyclic groups can be substituted or unsubstituted. Where a group is substituted, the substituent can be alkyl, alkoxy, halogen, oxygen, or substituted or unsubstituted phenyl.

Peptides and proteins, as well as peptide and protein analogs and mimetics, can also be covalently bound to one or more of a variety of nonproteinaceous polymers, e.g., polyethylene glycol, polypropylene glycol, or polyoxyalkenes, in the manner set forth in U.S. Pat. Nos. 4,640,835; 4,496,689; 4,301,144; 4,670,417; 4,791,192; or 4,179,337, all of which are incorporated by reference in their entirety herein.

Other peptide and protein analogs and mimetics within the invention include glycosylation variants, and covalent or aggregate conjugates with other chemical moieties. Covalent derivatives can be prepared by linkage of functionalities to groups which are found in amino acid side chains or at the N- or C-termini, by means which are well known in the art. These derivatives can include, without limitation, aliphatic esters or amides of the carboxyl terminus, or of residues containing carboxyl side chains, O-acyl derivatives of hydroxyl group-containing residues, and N-acyl derivatives of the amino terminal amino acid or amino-group containing residues, e.g., lysine or arginine. Acyl groups are selected from the group of alkyl-moieties including C3 to C18 normal alkyl, thereby forming alkanoyl aroyl species. Covalent attachment to carrier proteins, e.g., immunogenic moieties may also be employed.

In addition to these modifications, glycosylation alterations of biologically active peptides and proteins can be made, e.g., by modifying the glycosylation patterns of a peptide during its synthesis and processing, or in further processing steps. Particularly preferred means for accomplishing this are by exposing the peptide to glycosylating enzymes derived from cells that normally provide such processing, e.g., mammalian glycosylation enzymes. Deglycosylation enzymes can also be successfully employed to yield useful modified peptides and proteins within the invention. Also embraced are versions of a native primary amino acid sequence which have other minor modifications, including phosphorylated amino acid residues, e.g., phosphotyrosine, phosphoserine, or phosphothreonine, or other moieties, including ribosyl groups or cross-linking reagents.

Peptidomimetics may also have amino acid residues that have been chemically modified by phosphorylation, sulfonation, biotinylation, or the addition or removal of other moieties, particularly those that have molecular shapes similar to phosphate groups. In some embodiments, the modifications will be useful labeling reagents, or serve as purification targets, e.g., affinity ligands.

A major group of peptidomimetics within the invention comprises covalent conjugates of native peptides or proteins, or fragments thereof, with other proteins or peptides. These derivatives can be synthesized in recombinant culture such as N- or C-terminal fusions or by the use of agents known in the art for their usefulness in cross-linking proteins through reactive side groups. Preferred peptide and protein derivatization sites for targeting by cross-linking agents are at free amino groups, carbohydrate moieties, and cysteine residues.

Fusion polypeptides between biologically active peptides or proteins and other homologous or heterologous peptides and proteins are also provided. Many growth factors and cytokines are homodimeric entities, and a repeat construct of these molecules or active fragments thereof will yield various advantages, including lessened susceptibility to proteolytic degradation. Repeat and other fusion constructs of interferon-β yield similar advantages within the methods and compositions of the invention. Various alternative multimeric constructs comprising peptides and proteins useful within the invention are thus provided. In certain embodiments, biologically active polypeptide fusions are provided as described in U.S. Pat. Nos. 6,018,026, 5,843,725, 6,291,646, 6,300,099, and 6,323,323, for example by linking one or more biologically active peptides or proteins of the invention with a heterologous, multimerizing polypeptide or protein, for example an immunoglobulin heavy chain constant region, or an immunoglobulin light chain constant region. The biologically active, multimerized polypeptide fusion thus constructed can be a hetero- or homo-multimer, e.g., a heterodimer or homodimer comprising one or more interferon-β protein or peptide element(s), which may each comprise one or more distinct biologically active peptides or proteins operable within the invention. Other heterologous polypeptides may be combined with the active peptide or protein to yield fusions that exhibit a combination of properties or activities of the derivative proteins. Other typical examples are fusions of a reporter polypeptide, e.g., CAT or luciferase, with a peptide or protein as described herein, to facilitate localization of the fused peptide or protein (see, e.g., Dull et al., U.S. Pat. No. 4,859,609). Other fusion partners useful in this context include bacterial beta-galactosidase, trpE, Protein A, beta-1actamase, alpha amylase, alcohol dehydrogenase, and yeast alpha mating factor (see, e.g., Godowski et al., Science 241:812-816, 1988).

The present invention also contemplates the use of biologically active peptides and proteins, including interferon-β peptides and proteins, modified by covalent or aggregative association with chemical moieties. These derivatives generally fall into the three classes: (1) salts, (2) side chain and terminal residue covalent modifications, and (3) adsorption complexes, for example with cell membranes. Such covalent or aggregative derivatives are useful for various purposes, for example to block homo- or heterotypic association between one or more interferon-β proteins, as immunogens, as reagents in immunoassays, or in purification methods such as for affinity purification of ligands or other binding ligands. For example, an active peptide or protein can be immobilized by covalent bonding to a solid support such as cyanogen bromide-activated Sepharose, by methods which are well known in the art, or adsorbed onto polyolefin surfaces, with or without glutaraldehyde cross-linking, for use in the assay or purification of antibodies that specifically bind the active peptide or protein. The active peptide or protein can also be labeled with a detectable group, for example radioiodinated by the chloramine T procedure, covalently bound to rare earth chelates, or conjugated to another fluorescent moiety for use in diagnostic assays, including assays involving intranasal administration of the labeled peptide or protein.

Those of skill in the art recognize that a variety of techniques are available for constructing peptide and protein mimetics with the same or similar desired biological activity as the corresponding native peptide or protein but with more favorable activity than the peptide or protein, for example improved characteristics of solubility, stability, and/or susceptibility to hydrolysis or proteolysis (see, e.g., Morgan and Gainor, Ann. Rep. Med. Chem. 24:243-252, 1989). Certain peptidomimetic compounds are based upon the amino acid sequence of the proteins and peptides described herein for use within the invention, including sequences of interferon-β proteins and peptides. Typically, peptidomimetic compounds are synthetic compounds having a three-dimensional structure (of at least part of the mimetic compound) that mimics, e.g., the primary, secondary, and/or tertiary structural, and/or electrochemical characteristics of a selected peptide or protein, or a structural domain, active site, or binding region (e.g., a homotypic or heterotypic binding site, catalytic active site or domain, receptor or ligand binding interface or domain, etc.) thereof. The peptide-mimetic structure or partial structure (also referred to as a peptidomimetic “motif” of a peptidomimetic compound) will share a desired biological activity with a native peptide or protein, e.g., activity to block homo- or heterotypic association between one or more interferon-β proteins, receptor binding and/or activation activities, immunogenic activity (such as binding to MHC molecules of one or multiple haplotypes and activating CD8⁺ and/or CD4⁺ T). Typically, the subject biologically activity of the mimetic compound is not substantially reduced in comparison to, and is often the same as or greater than, the activity of the native peptide on which the mimetic was modeled. In addition, peptidomimetic compounds can have other desired characteristics that enhance their therapeutic application, such as increased cell permeability, greater affinity and/or avidity, and prolonged biological half-life. The peptidomimetics of the invention will sometimes have a “backbone” that is partially or completely non-peptide, but with side groups identical to the side groups of the amino acid residues that occur in the peptide or protein on which the peptidomimetic is modeled. Several types of chemical bonds, e.g. ester, thioester, thioamide, retroamide, reduced carbonyl, dimethylene and ketomethylene bonds, are known in the art to be generally useful substitutes for peptide bonds in the construction of protease-resistant peptidomimetics.

The following describes methods for preparing peptide and protein mimetics modified at the N-terminal amino group, the C-terminal carboxyl group, and/or changing ore or more of the amido linkages in the peptide to a non-amido linkage. It being understood that two or more such modifications can be coupled in one peptide or protein mimetic structure (e.g., modification at the C-terminal carboxyl group and inclusion of a —CH₂-carbamate linkage between two amino acids in the peptide. For N-terminal modifications, peptides typically are synthesized as the free acid but, as noted above, can be readily prepared as the amide or ester. One can also modify the amino and/or carboxy terminus of peptide compounds to produce other compounds useful within the invention. Amino terminus modifications include methylating (i.e., —NHCH₃ or —NH(CH₃)₂), acetylating, adding a carbobenzoyl group, or blocking the amino terminus with any blocking group containing a carboxylate functionality defined by RCOO—, where R is selected from the group consisting of naphthyl, acridinyl, steroidyl, and similar groups. Carboxy terminus modifications include replacing the free acid with a carboxamide group or forming a cyclic lactam at the carboxy terminus to introduce structural constraints. Amino terminus modifications are as recited above and include alkylating, acetylating, adding a carbobenzoyl group, forming a succinimide group, etc. The N-terminal amino group can then be reacted as follows:

(a) to form an amide group of the formula RC(O)NH— where R is as defined above by reaction with an acid halide [e.g., RC(O)Cl] or acid anhydride. Typically, the reaction can be conducted by contacting about equimolar or excess amounts (e.g., about 5 equivalents) of an acid halide to the peptide in an inert diluent (e.g., dichloromethane) preferably containing an excess (e.g., about 10 equivalents) of a tertiary amine, such as diisopropylethylamine, to scavenge the acid generated during reaction. Reaction conditions are otherwise conventional (e.g., room temperature for 30 minutes). Alkylation of the terminal amino to provide for a lower alkyl N-substitution followed by reaction with an acid halide as described above will provide for N-alkyl amide group of the formula RC(O)NR—;

(b) to form a succinimide group by reaction with succinic anhydride. As before, an approximately equimolar amount or an excess of succinic anhydride (e.g., about 5 equivalents) can be employed and the amino group is converted to the succinimide by methods well known in the art including the use of an excess (e.g., ten equivalents) of a tertiary amine such as diisopropylethylamine in a suitable inert solvent (e.g., dichloromethane) (see, for example, Wollenberg, et al., U.S. Pat. No. 4,612,132). It is understood that the succinic group can be substituted with, for example, C₂-C₆ alkyl or —SR substituents that are prepared in a conventional manner to provide for substituted succinimide at the N-terminus of the peptide. Such alkyl substituents are prepared by reaction of a lower olefin (C₂-C₆) with maleic anhydride in the manner described by Wollenberg, et al. (U.S. Pat. No. 4,612,132) and —SR substituents are prepared by reaction of RSH with maleic anhydride where R is as defined above;

(c) to form a benzyloxycarbonyl—NH— or a substituted benzyloxycarbonyl—NH— group by reaction with approximately an equivalent amount or an excess of CBZ-Cl (i.e., benzyloxycarbonyl chloride) or a substituted CBZ-Cl in a suitable inert diluent (e.g., dichloromethane) preferably containing a tertiary amine to scavenge the acid generated during the reaction;

(d) to form a sulfonamide group by reaction with an equivalent amount or an excess (e.g., 5 equivalents) of R—S(O)₂Cl in a suitable inert diluent (dichloromethane) to convert the terminal amine into a sulfonamide where R is as defined above. Preferably, the inert diluent contains excess tertiary amine (e.g., ten equivalents) such as diisopropylethylamine, to scavenge the acid generated during reaction. Reaction conditions are otherwise conventional (e.g., room temperature for 30 minutes);

(e) to form a carbamate group by reaction with an equivalent amount or an excess (e.g., 5 equivalents) of R—OC(O)Cl or R—OC(O)OC₆H₄-p-NO₂ in a suitable inert diluent (e.g., dichloromethane) to convert the terminal amine into a carbamate where R is as defined above. Preferably, the inert diluent contains an excess (e.g., about 10 equivalents) of a tertiary amine, such as diisopropylethylamine, to scavenge any acid generated during reaction. Reaction conditions are otherwise conventional (e.g., room temperature for 30 minutes);

(f) to form a urea group by reaction with an equivalent amount or an excess (e.g., 5 equivalents) of R—N═C═O in a suitable inert diluent (e.g., dichloromethane) to convert the terminal amine into a urea (i.e., RNHC(O)NH—) group where R is as defined above. Preferably, the inert diluent contains an excess (e.g., about 10 equivalents) of a tertiary amine, such as diisopropylethylamine. Reaction conditions are otherwise conventional (e.g., room temperature for about 30 minutes).

In preparing peptide mimetics wherein the C-terminal carboxyl group is replaced by an ester (i.e., —C(O)OR where R is as defined above), resins as used to prepare peptide acids are typically employed, and the side chain protected peptide is cleaved with base and the appropriate alcohol, e.g., methanol. Side chain protecting groups are then removed in the usual fashion by treatment with hydrogen fluoride to obtain the desired ester.

In preparing peptide mimetics wherein the C-terminal carboxyl group is replaced by the amide —C(O)NR₃R₄, a benzhydrylamine resin is used as the solid support for peptide synthesis. Upon completion of the synthesis, hydrogen fluoride treatment to release the peptide from the support results directly in the free peptide amide (i.e., the C-terminus is —C(O)NH₂). Alternatively, use of the chloromethylated resin during peptide synthesis coupled with reaction with ammonia to cleave the side chain protected peptide from the support yields the free peptide amide and reaction with an alkylamine or a dialkylamine yields a side chain protected alkylamide or dialkylamide (i.e., the C-terminus is —C(O)NRR₁ where R and R₁ are as defined above). Side chain protection is then removed in the usual fashion by treatment with hydrogen fluoride to give the free amides, alkylamides, or dialkylamides.

In another alternative embodiments of the invention, the C-terminal carboxyl group or a C-terminal ester of a biologically active peptide can be induced to cyclize by internal displacement of the —OH or the ester (—OR) of the carboxyl group or ester respectively with the N-terminal amino group to form a cyclic peptide. For example, after synthesis and cleavage to give the peptide acid, the free acid is converted to an activated ester by an appropriate carboxyl group activator such as dicyclohexylcarbodiimide (DCC) in solution, for example, in methylene chloride (CH₂Cl₂), dimethyl formamide (DMF) mixtures. The cyclic peptide is then formed by internal displacement of the activated ester with the N-terminal amine. Internal cyclization as opposed to polymerization can be enhanced by use of very dilute solutions. Such methods are well known in the art.

One can cyclize active peptides for use within the invention, or incorporate a desamino or descarboxy residue at the termini of the peptide, so that there is no terminal amino or carboxyl group, to decrease susceptibility to proteases, or to restrict the conformation of the peptide. C-terminal functional groups among peptide analogs and mimetics of the present invention include amide, amide lower alkyl, amide di(lower alkyl), lower alkoxy, hydroxy, and carboxy, and the lower ester derivatives thereof, and the pharmaceutically acceptable salts thereof.

Other methods for making peptide and protein derivatives and mimetics for use within the methods and compositions of the invention are described in Hruby et al. (Biochem J. 268:249-262, 1990). According to these methods, biologically active peptides and proteins serve as structural models for non-peptide mimetic compounds having similar biological activity as the native peptide or protein. Those of skill in the art recognize that a variety of techniques are available for constructing compounds with the same or similar desired biological activity as the lead peptide or protein compound, or that have more favorable activity than the lead with respect a desired property such as solubility, stability, and susceptibility to hydrolysis and proteolysis (see, e.g., Morgan and Gainor, Ann. Rep. Med. Chem. 24:243-252, 1989). These techniques include, for example, replacing a peptide backbone with a backbone composed of phosphonates, amidates, carbamates, sulfonamides, secondary amines, and/or N-methylamino acids.

Peptide and protein mimetics wherein one or more of the peptidyl linkages [—C(O)NH—] have been replaced by such linkages as a —CH₂-carbamate linkage, a phosphonate linkage, a —CH₂-sulfonamide linkage, a urea linkage, a secondary amine (—CH₂NH—) linkage, and an alkylated peptidyl linkage [—C(O)NR₆— where R₆ is lower alkyl] are prepared, for example, during conventional peptide synthesis by merely substituting a suitably protected amino acid analogue for the amino acid reagent at the appropriate point during synthesis. Suitable reagents include, for example, amino acid analogues wherein the carboxyl group of the amino acid has been replaced with a moiety suitable for forming one of the above linkages. For example, if one desires to replace a —C(O)NR— linkage in the peptide with a —CH₂-carbamate linkage (—CH₂OC(O)NR—), then the carboxyl (—COOH) group of a suitably protected amino acid is first reduced to the —CH₂OH group which is then converted by conventional methods to a —OC(O)Cl functionality or a para-nitrocarbonate —OC(O)O—C₆H₄-p-NO₂ functionality. Reaction of either of such functional groups with the free amine or an alkylated amine on the N-terminus of the partially fabricated peptide found on the solid support leads to the formation of a —CH₂OC(O)NR— linkage. For a more detailed description of the formation of such —CH₂-carbamate linkages, see, e.g., Cho et al. (Science 261:1303-1305, 1993).

Replacement of an amido linkage in an active peptide with a —CH₂-sulfonamide linkage can be achieved by reducing the carboxyl (—COOH) group of a suitably protected amino acid to the —CH₂OH group, and the hydroxyl group is then converted to a suitable leaving group such as a tosyl group by conventional methods. Reaction of the derivative with, for example, thioacetic acid followed by hydrolysis and oxidative chlorination will provide for the —CH₂—S(O)₂Cl functional group which replaces the carboxyl group of the otherwise suitably protected amino acid. Use of this suitably protected amino acid analogue in peptide synthesis provides for inclusion of an —CH₂S(O)₂NR— linkage that replaces the amido linkage in the peptide thereby providing a peptide mimetic. For a more complete description on the conversion of the carboxyl group of the amino acid to a —CH₂S(O)₂Cl group, see, e.g., Weinstein and Boris (Chemistry & Biochemistry of Amino Acids, Peptides and Proteins, Vol. 7, pp. 267-357, Marcel Dekker, Inc., New York, 1983). Replacement of an amido linkage in an active peptide with a urea linkage can be achieved, for example, in the manner set forth in U.S. patent application Ser. No. 08/147,805.

Secondary amine linkages wherein a —CH₂NH— linkage replaces the amido linkage in the peptide can be prepared by employing, for example, a suitably protected dipeptide analogue wherein the carbonyl bond of the amido linkage has been reduced to a CH₂ group by conventional methods. For example, in the case of diglycine, reduction of the amide to the amine will yield after deprotection H₂NCH₂CH₂NHCH₂ COOH that is then used in N-protected form in the next coupling reaction. The preparation of such analogues by reduction of the carbonyl group of the amido linkage in the dipeptide is well known in the art.

Operable analogs and mimetics of an interferon-β or other active peptide or protein disclosed herein will retain partial, complete or enhanced activity compared to a native peptide, protein or unmodified compound. For example analogs or mimetics of interferon-β peptide will exhibit partial or complete activity for binding to a known binding partner of interferon- [symbol] such as an interferon-β receptor or a cell or tissue expressing the receptor, for inducing an interferon-β, e.g., serum β-2 microglobulin or serum neopterin, or for eliciting a physiological, e.g., immune, response correlated with interferon-β activity. In this regard, operable analogs and mimetics for use within the invention will retain at least 50%, often 75%, and up to 95-100% or greater levels of one or more selected activities as compared to the same activity observed for a selected native peptide or protein or unmodified compound. These biological properties of altered peptides or non-peptide mimetics can be determined according to any suitable assay disclosed or incorporated herein or generally known in the art.

A variety of additives, diluents, bases and delivery vehicles are provided within the invention that effectively control water content to enhance protein stability. These reagents and carrier materials effective as anti-aggregation agents in this sense include, for example, polymers of various functionalities, such as polyethylene glycol, dextran, diethylaminoethyl dextran, and carboxymethyl cellulose, which significantly increase the stability and reduce the solid-phase aggregation of peptides and proteins admixed therewith or linked thereto. In some instances, the activity or physical stability of proteins can also be enhanced by various additives to aqueous solutions of the peptide or protein drugs. For example, additives, such as polyols (including sugars), amino acids, proteins such as collagen and gelatin, and various salts may be used.

Certain additives, in particular sugars and other polyols, also impart significant physical stability to dry, e.g., lyophilized proteins. These additives can also be used within the invention to protect the proteins against aggregation not only during lyophilization but also during storage in the dry state. For example sucrose and Ficoll 70 (a polymer with sucrose units) exhibit significant protection against peptide or protein aggregation during solid-phase incubation under various conditions. These additives may also enhance the stability of solid proteins embedded within polymer matrices.

Yet additional additives, for example sucrose, stabilize proteins against solid-state aggregation in humid atmospheres at elevated temperatures, as may occur in certain sustained-release formulations of the invention. Proteins such as gelatin and collagen also serve as stabilizing or bulking agents to reduce denaturation and aggregation of unstable proteins in this context. These additives can be incorporated into polymeric melt processes and compositions within the invention. For example, polypeptide microparticles can be prepared by simply lyophilizing or spray drying a solution containing various stabilizing additives described above. Sustained release of unaggregated peptides and proteins can thereby be obtained over an extended period of time.

Various additional preparative components and methods, as well as specific formulation additives, are provided herein which yield formulations for mucosal delivery of aggregation-prone peptides and proteins, wherein the peptide or protein is stabilized in a substantially pure, unaggregated form. A range of components and additives are contemplated for use within these methods and formulations. Exemplary of these anti-aggregation agents are linked dimers of cyclodextrins (CDs), which selectively bind hydrophobic side chains of polypeptides (see, e.g., Breslow, et al., J. Am. Chem. Soc. 120:3536-3537; Maletic, et al., Angew. Chem. Int. Ed. Engl. 35:1490-1492. These CD dimers have been found to bind to hydrophobic patches of proteins in a manner that significantly inhibits aggregation (Leung et al., Proc. Nat.l Acad. Sci. USA 97:5050-5053, 2000). This inhibition is selective with respect to both the CD dimer and the protein involved. Such selective inhibition of protein aggregation provides additional advantages within the intranasal delivery methods and compositions of the invention. Additional agents for use in this context include CD trimers and tetramers with varying geometries controlled by the linkers that specifically block aggregation of peptides and proteins (Breslow et al., J. Am. Chem. Soc. 118:11678-11681, 1996; Breslow et al., PNAS USA 94:11156-11158, 1997; Breslow et al., Tetrahedron Lett. 2887-2890, 1998).

Yet additional anti-aggregation agents and methods for incorporation within the invention involve the use of peptides and peptide mimetics to selectively block protein-protein interactions. In one aspect, the specific binding of hydrophobic side chains reported for CD multimers is extended to proteins via the use of peptides and peptide mimetics that similarly block protein aggregation. A wide range of suitable methods and anti-aggregation agents are available for incorporation within the compositions and procedures of the invention (Zutshi et al., Curr. Opin. Chem. Biol. 2:62-66, 1998; Daugherty et al., J. Am. Chem. Soc. 121:4325-4333, 1999: Zutshi et al., J. Am. Chem. Soc. 119:4841-4845, 1997; Ghosh et al, Chem. Biol. 5:439-445, 1997; Hamuro et al., Angew. Chem. Int. Ed. Engl. 36:2680-2683, 1997; Alberg et al., Science 262:248-250, 1993; Tauton et al., J. Am. Chem. Soc. 118:10412-10422, 1996; Park et al., J. Am. Chem. Soc. 121:8-13, 1999; Prasanna et al., Biochemistry 37:6883-6893, 1998; Tiley et al., J. Am. Chem. Soc. 119:7589-7590, 1997; Judice et al., PNAS, USA 94:13426-13430, 1997; Fan et al., J. Am. Chem. Soc. 120:8893-8894, 1998; Gamboni et al., Biochemistry 37:12189-12194, 1998). Briefly, these methods involve rational design and selection of peptides and mimetics that effectively block interactions between selected biologically active peptides or proteins, whereby the selected peptides and mimetics significantly reduce aggregation of the active peptides or proteins in a mucosal formulation. Anti-aggregation peptides and mimetics thus identified are coordinately administered with, or admixed or conjugated in a combinatorial formulation with, a biologically active peptide or protein to effectively inhibit aggregation of the active peptide or protein in a manner that significantly enhances absorption and/or bioavailability of the active peptide or protein.

Other anti-aggregation agents for use within the invention include chaperonins and analogs and mimetics of such molecules, as well as antibodies and antibody fragments that function in a similar, but often more specific, manner as chaperonins to bind peptide and protein domains and thereby block associative interactions there between. These molecular chaperones were initially recognized as stress proteins produced in cells requiring repair. In particular, studies of heat shock on enzymes showed that molecular chaperones function not only during cellular stress but also to chaperone the process of normal protein folding. Chaperonins comprise a ubiquitous family of proteins that mediate post-translational folding and assembly of other proteins into oligomeric structures. They prevent the formation of incorrect structures, and also act to disrupt incorrect structures that form during these processes. The chaperones non-covalently bind to the interactive surface of a target protein. This binding is reversed under circumstances that favor the formation of the correct structure by folding. Chaperones have not been shown to be specific for only one protein, but rather act on families of proteins that have similar stoichiometric requirements (e.g., specific structural domains that are recognized by the chaperones). Various publications describe the selection and use of chaperonins, antibodies and antibody fragments as aggregation-blocking agents for use within the invention (see, e.g., WO 93/11248; WO 93/13200; WO 94/08012; WO; WO 94/11513; WO 94/08012; and U.S. Pat. No. 5,688,651).

Other techniques in peptide and protein engineering disclosed herein will further reduce the extent of protein aggregation and instability in mucosal delivery methods and formulations of the invention. One example of a useful method for peptide or protein modification in this context is PEGylation. The stability and aggregation problems of polypeptide drugs can be significantly improved by covalently conjugating water-soluble polymers such as PEG with the polypeptide. Another example is modification of a peptide or protein amino acid sequence in terms of the identity or location of one or more residues, e.g., by terminal or internal addition, deletion or substitution (e.g., deletion of cysteine residues or replacement by alanine or serine) to reduce aggregation potential. The improvements in terms of stability and aggregation potential that are achieved by these methods enables effective mucosal delivery of a therapeutically effective polypeptide or protein composition within the methods of the invention.

Charge Modifying and pH Control Agents and Methods

To improve the transport characteristics of biologically active agents (including interferon- [symbol] other active peptides and proteins, and macromolecular and small molecule drugs) for enhanced delivery across hydrophobic mucosal membrane barriers, the invention also provides techniques and reagents for charge modification of selected biologically active agents or delivery-enhancing agents described herein. In this regard, the relative permeabilities of macromolecules is generally be related to their partition coefficients. The degree of ionization of molecules, which is dependent on the pKₐ of the molecule and the pH at the mucosal membrane surface, also affects permeability of the molecules. Permeation and partitioning of biologically active agents, including interferon-β peptides and analogs of the invention, for mucosal delivery may be facilitated by charge alteration or charge spreading of the active agent or permeabilizing agent, which is achieved, for example, by alteration of charged functional groups, by modifying the pH of the delivery vehicle or solution in which the active agent is delivered, or by coordinate administration of a charge- or pH-altering reagent with the active agent.

Muscosal delivery of charged macromolecular species, including interferon-β peptides and other biologically active peptides and proteins, within the methods and compositions of the invention is substantially improved when the active agent is delivered to the mucosal surface in a substantially un-ionized, or neutral, electrical charge state.

Calculation of the isoelectric points of interferon-β peptides and other biologically active peptides, proteins, and peptide analogs and mimetics is readily undertaken to guide the selection of pH and other values for mucosal formulations within the invention, which optionally deliver charged macromolecules in a substantially un-ionized state to the mucosal surface or, alternatively, following mucosal delivery at a target site of drug action. The pI of an amphoteric molecule is defined as the pH at which the net charge is zero. The variation of net charge with pH is of importance in charge-dependent separation methods like electrophoresis, isoelectric focusing, chromatofocusing and ion-exchange chromatography. Thus, methods for estimating isoelectric points (pI) for native peptides and proteins are well known and readily implemented within the methods and compositions of the invention [see, e.g., Cameselle, et al., Biochem. Educ. 14:131-136, 1986; Skoog, et al., Trends Anal. Chem. 5:82-83, 1986; Sillero et al., Anal. Biochem. 179:319-25, 1989; Englund, et al., Biochim. Biophys. Acta, 1065:185-194, 1991; Bjellquist et al., Electrophoresis. 14:1023-1031, 1993; Mosher et al., J. Chromatogr. 638:155-164, 1993; Bjellqvist et al., Electrophoresis 15:529-539, 1994; Watts, et al., Electrophoresis 16:22-27, (1995)].

For determining pI values of peptides and proteins for use within the invention, net charge can be estimated, for example, by the well-known Henderson-Hasselbalch equation. These determinations are based in part on the amino acid composition of the subject peptide or protein, yielding component pI values for specific amino acid side chains and for the N- and C-terminal groups. The individual ionizable side chains of each type of amino acid are typically assumed to have pKa values distributed around the projected pKa, value, simulating the situation in polypeptides and proteins where a given type of ionizable amino acid side chain often appears in several positions in the amino acid sequence and with various individual ionization constants, depending both on the adjacent side chains and on the three-dimensional environment in the protein (see, e.g., Bjellqvist et al., Electrophoresis 15:529-539, 1994; Matthew, Annu. Rev. Biophys. Chem. 14:387-417, 1985). By assuming a distribution of pKa values, the calculated titration curves will be smoothed out. The presence of other charged groups is also taken into account. These analyses yield a set of pKa values, including values for amino acid residues with ionizable side chains. Each particular type of ionizable group is assumed to have pKa values distributed around the chosen value, thereby simulating the situation in intact proteins and polypeptides. According to these known calculation methods, accurate estimates of pI values for peptides and proteins show sufficient agreement with experimental values determined for native proteins, over a wide pH range (3.4-11), particularly when more refined analyses, including such factors as charge contributions of heme groups, sialic acid residues, etc., are taken into account (see, e.g., Henriksson et al., Electrophoresis. 16:1377-1380, 1995).

Thus, for polypeptides of known amino acid composition, a sufficient pH value estimate can be calculated by use of the ionization constant pKa for amino acid side chain groups. Where other types of ionizable groups occur, the charge for each such group at any given pH can also be readily estimated. The total net charge at a selected pH is obtained by summing up the charge for each type of ionizable group times the number of groups. In the present study, suitable average pKa, values were selected for the ionizable amino acid side chains, and for the terminal groups.

Certain interferon-β peptides and other biologically active peptide and protein components of mucosal formulations for use within the invention will be charge modified to yield an increase in the positive charge density of the peptide or protein. These modifications extend also to cationization of peptide and protein conjugates, carriers and other delivery forms disclosed herein. Cationization offers a convenient means of altering the biodistribution and transport properties of proteins and macromolecules within the invention. Cationization is undertaken in a manner that substantially preserves the biological activity of the active agent and limits potentially adverse side effects, including tissue damage and toxicity. In many cases, cationized molecules have higher organ uptake and penetration compared with non-cationized forms (see, e.g., Ekrami et al., Journal of Pharmaceutical Sciences 84:456-461, 1995; Bergman et al., Clin. Sci. 67:35-43, 1984; Triguero et al., J. Pharm. Exp. Ther. 258:186-192, 1991). In some cases, cationized proteins can penetrate physiological barriers considered impenetrable by the native proteins.

Degradative Enzyme Inhibitory Agents and Methods

A major drawback to effective mucosal delivery of biologically active agents, including interferon-β peptides, is that they may be subject to degradation by mucosal enzymes.

In addition to their susceptibility to enzymatic degradation, many therapeutic compounds, particularly relatively low molecular weight proteins, and peptides, introduced into the circulation, are cleared quickly from mammalian subjects by the kidneys. This problem may be partially overcome by administering large amounts of the therapeutic compound through repeated administration. However, higher doses of therapeutic formulations containing protein or peptide components can elicit antibodies that can bind and inactivate the protein and/or facilitate the clearance of the protein from the subject's body. Repeated administration of the formulation containing the therapeutic protein or peptide is essentially ineffective and can be dangerous as it can elicit an allergic or autoimmune response.

The problem of metabolic lability of therapeutic peptides, proteins and other compounds may be addressed in part through rational drug design. However, medicinal chemists have had less success in manipulating the structures of peptides and proteins to achieve high cell membrane permeability while still retaining pharmacological activity. Unfortunately, many of the structural features of peptides and proteins (e.g., free N-terminal amino and C-terminal carboxyl groups, and side chain carboxyl (e.g., Asp, Glu), amino (e.g., Lys, Arg) and hydroxyl (e.g. Ser, Thr, Tyr) groups) that bestow upon the molecule affinity and specificity for its pharmacological binding partner also bestow upon the molecule undesirable physicochemical properties (e.g., charge, hydrogen bonding potential) which limit their cell membrane permeability. Therefore, alternative strategies need to be considered for intranasal formulation and delivery of peptide and protein therapeutics.

More recent research efforts in the area of protease inhibition for enhanced delivery of biotherapeutic compounds, including peptide and protein therapeutics, has focused on covalent immobilization of enzyme inhibitors on mucoadhesive polymers used as drug carrier matrices (see, e.g., Bernkop-Schnurch et al., Drug Dev. Ind. Pharm. 23:733-40, 1997; Bernkop-Schnurch et al., J. Control. Rel. 47:113-21, 1997; Bernkop-Schnurch et al., J. Drug Targ. 7:55-63, 1999). In conjunction with these teachings, the invention provides in more detailed aspects an enzyme inhibitor formulated with a common carrier or vehicle for mucosal delivery of interferon-β peptides and other biologically active peptides, analogs and mimetics, optionally to be administered coordinately one or more additional biologically active or delivery-enhancing agents. Optionally, the enzyme inhibitor is covalently linked to the carrier or vehicle. In certain embodiments, the carrier or vehicle is a biodegradable polymer, for example, a bioadhesive polymer. Thus, for example, a protease inhibitor, such as Bowman-Birk inhibitor (BBI), displaying an inhibitory effect towards trypsin and \acute{α}-chymotrypsin (Birk Y. Int. J. Pept. Protein Res. 25:113-31, 1985, or elastatinal, an elastase-specific inhibitor of low molecular size, may be covalently linked to a mucoadhesive polymer as described herein. The resulting polymer-inhibitor conjugate exhibits substantial utility as a mucosal delivery vehicle for peptides and other biologically active agents formulated or delivered alone or in combination with other biologically active agents or additional delivery-enhancing agents.

Exemplary mucoadhesive polymer-enzyme inhibitor complexes that are useful within the mucosal delivery formulations and methods of the invention include, but are not limited to: Carboxymethylcellulose-pepstatin (with anti-pepsin activity); Poly(acrylic acid)-Bowman-Birk inhibitor (anti-chymotrypsin); Poly(acrylic acid)-chymostatin (anti-chymotrypsin); Poly(acrylic acid)-elastatinal (anti-elastase); Carboxymethylcellulose-elastatinal (anti-elastase); Polycarbophil-elastatinal (anti-elastase); Chitosan-antipain (anti-trypsin); Poly(acrylic acid)-bacitracin (anti-aminopeptidase N); Chitosan-EDTA (anti-aminopeptidase N, anti-carboxypeptidase A); Chitosan-EDTA-antipain (anti-trypsin, anti-chymotrypsin, anti-elastase) (see, e.g., Bernkop-Schnürch, J. Control. Rel. 52:1-16, (1998). As described in further detail below, certain embodiments of the invention will optionally incorporate a novel chitosan derivative or chemically modified form of chitosan. One such novel derivative for use within the invention is denoted as a β-[1→4]-2-guanidino-2-deoxy-D-glucose polymer (poly-GuD).

The present invention provides coordinate administration methods and/or combinatorial formulations directed toward coordinate administration of a biologically active agent, including one or more interferon-β peptides, proteins, analogs and mimetics, with an enzyme inhibitor. Since a variety of degradative enzymes are present in the mucosal environment, the prophylactic and therapeutic compositions and methods of the invention are readily modified to incorporate the addition or coadministration of an enzyme inhibitor, such as a protease inhibitor, with the biologically active agent (e.g., a physiologically active peptide or protein), to thereby improve bioavailability of the active agent. For example, in the case of therapeutically active peptides and proteins, one or more protease inhibiting agent(s) is/are optionally combined or coordinately administered in a formulation or method of the invention with one or more inhibitors of a proteolytic enzyme. In certain embodiments, the enzyme inhibitor is admixed with or bound to a common carrier with the biologically active agent. For example, an inhibitor of proteolytic enzymes may be incorporated in a therapeutic or prophylactic formulation of the invention to protect a biologically active protein or peptide from proteolysis, and thereby enhance bioavailability of the active protein or peptide.

Any inhibitor that inhibits the activity of an enzyme to protect the biologically active agent(s) may be usefully employed in the compositions and methods of the invention. Useful enzyme inhibitors for the protection of biologically active proteins and peptides include, for example, soybean trypsin inhibitor, pancreatic trypsin inhibitor, chymotrypsin inhibitor and trypsin and chrymotrypsin inhibitor isolated from potato (solanum tuberosum L.) tubers. A combination or mixtures of inhibitors may be employed. Additional inhibitors of proteolytic enzymes for use within the invention include ovomucoid-enzyme, gabaxate mesylate, alpha1-antitrypsin, aprotinin, amastatin, bestatin, puromycin, bacitracin, leupepsin, alpha2-macroglobulin, pepstatin and egg white or soybean trypsin inhibitor. These and other inhibitors can be used alone or in combination. The inhibitor(s) may be incorporated in or bound to a carrier, e.g., a hydrophilic polymer, coated on the surface of the dosage form which is to contact the nasal mucosa, or incorporated in the superficial phase of said surface, in combination with the biologically active agent or in a separately administered (e.g., pre-administered) formulation.

The amount of the inhibitor, e.g., of a proteolytic enzyme inhibitor that is optionally incorporated in the compositions of the invention will vary depending on (a) the properties of the specific inhibitor, (b) the number of functional groups present in the molecule (which may be reacted to introduce ethylenic unsaturation necessary for copolymerization with hydrogel forming monomers), and (c) the number of lectin groups, such as glycosides, which are present in the inhibitor molecule. It may also depend on the specific therapeutic agent that is intended to be administered. Generally speaking, a useful amount of an enzyme inhibitor is from about 0.1 mg/ml to about 50 mg/ml, often from about 0.2 mg/ml to about 25 mg/ml, and more commonly from about 0.5 mg/ml to 5 mg/ml of the of the formulation (i.e., a separate protease inhibitor formulation or combined formulation with the inhibitor and biologically active agent).

With the necessary caveat of determining and considering possible toxic and other deleterious side effects, various inhibitors of proteases may be evaluated for use within the mucosal delivery methods and compositions of the invention. In the case of trypsin inhibition, suitable inhibitors may be selected from, e.g., aprotinin, BBI, soybean trypsin inhibitor, chicken ovomucoid, chicken ovoinhibitor, human pancreatic trypsin inhibitor, camostat mesilate, flavonoid inhibitors, antipain, leupeptin, p-aminobenzamidine, AEBSF, TLCK (tosyllysine chloromethylketone), APMSF, DFP, PMSF, and poly(acrylate) derivatives. In the case of chymotrypsin inhibition, suitable inhibitors may be selected from, e.g., aprotinin, BBI, soybean trypsin inhibitor, chymostatin, benzyloxycarbonyl-Pro-Phe-CHO, FK-448, chicken ovoinhibitor, sugar biphenylboronic acids complexes, DFP, PMSF, β-phenylpropionate, and poly(acrylate) derivatives. In the case of elastase inhibition, suitable inhibitors may be selected from, e.g., elastatinal, methoxysuccinyl-Ala-Ala-Pro-Val-chloromethylketone (MPCMK), BBI, soybean trypsin inhibitor, chicken ovoinhibitor, DFP, and PMSF. Other naturally occurring, endogenous enzyme inhibitors for additional known degradative enzymes present in the intranasal environment, or alternatively present in preparative materials for production of intranasal formulations, will be readily ascertained by those skilled in the art for incorporation within the methods and compositions of the invention.

Additional enzyme inhibitors for use within the invention are selected from a wide range of non-protein inhibitors that vary in their degree of potency and toxicity (see, e.g., L. Stryer, Biochemistry, W H Freeman and Company, NY, N.Y., 1988). As described in further detail below, immobilization of these adjunct agents to matrices or other delivery vehicles, or development of chemically modified analogues, may be readily implemented to reduce or even eliminate toxic effects, when they are encountered. Among this broad group of candidate enzyme inhibitors for use within the invention are organophosphorous inhibitors, such as diisopropylfluorophosphate (DFP) and phenylmethylsulfonyl fluoride (PMSF), which are potent, irreversible inhibitors of serine proteases (e.g., trypsin and chymotrypsin). The additional inhibition of acetylcholinesterase by these compounds makes them highly toxic in uncontrolled delivery settings (L. Stryer, Biochemistry, W H Freeman and Company, NY, N.Y., 1988). Another candidate inhibitor, 4-(2-Aminoethyl)-benzenesulfonyl fluoride (AEBSF), has an inhibitory activity comparable to DFP and PMSF, but it is markedly less toxic. (4-Aminophenyl)-methanesulfonyl fluoride hydrochloride (APMSF) is another potent inhibitor of trypsin, but is toxic in uncontrolled settings. In contrast to these inhibitors, 4-(4-isopropylpiperadinocarbonyl)phenyl 1,2,3,4,-tetrahydro-1-naphthoate methanesulphonate (FK-448) is a low toxic substance, representing a potent and specific inhibitor of chymotrypsin. Further representatives of this non-protein group of inhibitor candidates, and also exhibiting low toxic risk, are camostat mesilate (N,N′-dimethyl carbamoylmethyl-p-(p′-guanidino-benzoyloxy)phenylacetate methane-sulphonate).

Solution or powder formulations of IFN-β administered intranasally without surfactants were not absorbed in rabbits. However, absorption occurred after the addition of surfactants (non-ionic, anionic and amphoteric). Maximum concentrations of IFN in plasma were dependent on the surfactant used, sodium glycocholate being the most effective. Total absorption of IFN following nasal administration with sodium glycocholate was 2.2% of that following intravenous administration. Maitani, et al., Drug Design and Delivery, 4: 109-119, 1989.

Yet another type of enzyme inhibitory agent for use within the methods and compositions of the invention are amino acids and modified amino acids that interfere with enzymatic degradation of specific therapeutic compounds. For use in this context, amino acids and modified amino acids are substantially non-toxic and can be produced at a low cost. However, due to their low molecular size and good solubility, they are readily diluted and absorbed in mucosal environments. Nevertheless, under proper conditions, amino acids can act as reversible, competitive inhibitors of protease enzymes. Certain modified amino acids can display a much stronger inhibitory activity. A desired modified amino acid in this context is known as a ‘transition-state’ inhibitor. The strong inhibitory activity of these compounds is based on their structural similarity to a substrate in its transition-state geometry, while they are generally selected to have a much higher affinity for the active site of an enzyme than the substrate itself. Transition-state inhibitors are reversible, competitive inhibitors. Examples of this type of inhibitor are α-aminoboronic acid derivatives, such as boro-leucine, boro-valine and boro-alanine. The boron atom in these derivatives can form a tetrahedral boronate ion that is believed to resemble the transition state of peptides during their hydrolysis by aminopeptidases. These amino acid derivatives are potent and reversible inhibitors of aminopeptidases and it is reported that boro-leucine is more than 100-times more effective in enzyme inhibition than bestatin and more than 1000-times more effective than puromycin. Another modified amino acid for which a strong protease inhibitory activity has been reported is N-acetylcysteine, which inhibits enzymatic activity of aminopeptidase N. This adjunct agent also displays mucolytic properties that can be employed within the methods and compositions of the invention to reduce the effects of the mucus diffusion barrier.

Still other useful enzyme inhibitors for use within the coordinate administration methods and combinatorial formulations of the invention may be selected from peptides and modified peptide enzyme inhibitors. An important representative of this class of inhibitors is the cyclic dodecapeptide, bacitracin, obtained from Bacillus licheniformis. Bacitracin A has a molecular mass of 1423 Da and shows remarkable resistance against the action of proteolytic enzymes like trypsin and pepsin. It has several biological properties inhibiting bacterial peptidoglycan synthesis, mammalian transglutaminase activity, and proteolytic enzymes such as aminopeptidase N. Besides its inhibitory activity, bacitracin also displays absorption-enhancing effects without leading to a serious intestinal mucosal damage (Gotoh et al., Biol. Pharm. Bull. 18:794-796, 1995).

In addition to these types of peptides, certain dipeptides and tripeptides display weak, non-specific inhibitory activity towards some proteases (Langguth et al., J. Pharm. Pharmacol. 46:34-40, 1994). By analogy with amino acids, their inhibitory activity can be improved by chemical modifications. For example, phosphinic acid dipeptide analogues are also ‘transition-state’ inhibitors with a strong inhibitory activity towards aminopeptidases. They have reportedly been used to stabilize nasally administered leucine enkephalin (Hussain et al., Pharm. Res. 9:626-628, 1992). Another example of a transition-state analogue is the modified pentapeptide pepstatin (McConnell et al., J. Med. Chem. 34:2298-2300, 1991), which is a very potent inhibitor of pepsin. Structural analysis of pepstatin, by testing the inhibitory activity of several synthetic analogues, demonstrated the major structure-function characteristics of the molecule responsible for the inhibitory activity (McConnell et al., J. Med. Chem. 34:2298-2300, 1991). Similar analytic methods can be readily applied to prepare modified amino acid and peptide analogs for blockade of selected, intranasal degradative enzymes.

Another special type of modified peptide includes inhibitors with a terminally located aldehyde function in their structure. For example, the sequence benzyloxycarbonyl-Pro-Phe-CHO, which fulfill the known primary and secondary specificity requirements of chymotrypsin, has been found to be a potent reversible inhibitor of this target proteinase. The chemical structures of further inhibitors with a terminally located aldehyde function, e.g. antipain, leupeptin, chymostatin and elastatinal, are also known in the art, as are the structures of other known, reversible, modified peptide inhibitors, such as phosphoramidon, bestatin, puromycin and amastatin Due to their comparably high molecular mass, polypeptide protease inhibitors are more amenable than smaller compounds to concentrated delivery in a drug-carrier matrix. Additional agents for protease inhibition within the formulations and methods of the invention involve the use of complexing agents. These agents mediate enzyme inhibition by depriving the intranasal environment (or preparative or therapeutic composition) of divalent cations which are co-factors for many proteases. For instance, the complexing agents EDTA and DTPA as coordinately administered or combinatorially formulated adjunct agents, in suitable concentration, will be sufficient to inhibit selected proteases to thereby enhance intranasal delivery of biologically active agents according to the invention. Further representatives of this class of inhibitory agents are EGTA, 1,10-phenanthroline and hydroxychinoline. In addition, due to their propensity to chelate divalent cations, these and other complexing agents are useful within the invention as direct, absorption-promoting agents. As noted in more detail elsewhere herein, it is also contemplated to use various polymers, particularly mucoadhesive polymers, as enzyme inhibiting agents within the coordinate administration, multi-processing and/or combinatorial formulation methods and compositions of the invention. For example, poly(acrylate) derivatives, such as poly(acrylic acid) and polycarbophil, can affect the activity of various proteases, including trypsin, chymotrypsin. The inhibitory effect of these polymers may also be based on the complexation of divalent cations such as Ca² and Zn². It is further contemplated that these polymers may serve as conjugate partners or carriers for additional enzyme inhibitory agents, as described above. For example, a chitosan-EDTA conjugate has been developed and is useful within the invention that exhibits a strong inhibitory effect towards the enzymatic activity of zinc-dependent proteases. The mucoadhesive properties of polymers following covalent attachment of other enzyme inhibitors in this context are not expected to be substantially compromised, nor is the general utility of such polymers as a delivery vehicle for biologically active agents within the invention expected to be diminished. On the contrary, the reduced distance between the delivery vehicle and mucosal surface afforded by the mucoadhesive mechanism will minimize presystemic metabolism of the active agent, while the covalently bound enzyme inhibitors remain concentrated at the site of drug delivery, minimizing undesired dilution effects of inhibitors as well as toxic and other side effects caused thereby. In this manner, the effective amount of a coordinately administered enzyme inhibitor can be reduced due to the exclusion of dilution effects.

More recent research efforts in the area of protease inhibition for enhanced delivery of peptide and protein therapeutics has focused on covalent immobilization of protease inhibitors on mucoadhesive polymers used as drug carrier matrices (see, e.g., Bernkop-Schnurch et al., Drug Dev. Ind. Pharm. 23:733-40, 1997; Bernkop-Schnurch et al., J. Control. Rel. 47:113-21, 1997; Bernkop-Schnurch et al., J. Drug Targ. 7:55-63, 1999). In conjunction with these teachings, the invention provides in more detailed aspects an enzyme inhibitor formulated with a common carrier or vehicle for intranasal delivery of a biologically active agent. Optionally, the enzyme inhibitor is covalently linked to the carrier or vehicle. In certain embodiments, the carrier or vehicle is a biodegradable polymer, for example, a bioadhesive polymer. Thus, for example, a protease inhibitor, such as Bowman-Birk inhibitor (BBI), displaying an inhibitory effect towards trypsin and \acute{α}-chymotrypsin (Birk Y. Int. J. Pept. Protein Res. 25:113-31, 1985), or elastatinal, an elastase-specific inhibitor of low molecular size, may be covalently linked to a mucoadhesive polymer as described herein. The resulting polymer-inhibitor conjugate exhibits substantial utility as an intranasal delivery vehicle for biologically active agents according to the methods and compositions of the invention.

Exemplary mucoadhesive polymer-enzyme inhibitor complexes that are useful within the mucosal formulations and methods of the invention include, but are not limited to: Carboxymethylcellulose-pepstatin (with anti-pepsin activity); Poly(acrylic acid)-Bowman-Birk inhibitor (anti-chymotrypsin); Poly(acrylic acid)-chymostatin (anti-chymotrypsin); Poly(acrylic acid)-elastatinal (anti-elastase); Carboxymethylcellulose-elastatinal (anti-elastase); Polycarbophil-elastatinal (anti-elastase); Chitosan-antipain (anti-trypsin); Poly(acrylic acid)-bacitracin (anti-aminopeptidase N); Chitosan-EDTA (anti-aminopeptidase N, anti-carboxypeptidase A); Chitosan-EDTA-antipain (anti-trypsin, anti-chymotrypsin, anti-elastase).

Mucolytic and Mucus-Clearing Agents and Methods

Effective delivery of biotherapeutic agents via intranasal administration must take into account the decreased drug transport rate across the protective mucus lining of the nasal mucosa, in addition to drug loss due to binding to glycoproteins of the mucus layer. Normal mucus is a viscoelastic, gel-like substance consisting of water, electrolytes, mucins, macromolecules, and sloughed epithelial cells. It serves primarily as a cytoprotective and lubricative covering for the underlying mucosal tissues. Mucus is secreted by randomly distributed secretory cells located in the nasal epithelium and in other mucosal epithelia. The structural unit of mucus is mucin. This glycoprotein is mainly responsible for the viscoelastic nature of mucus, although other macromolecules may also contribute to this property. In airway mucus, such macromolecules include locally produced secretory IgA, lgM, IgE, lysozyme, and bronchotransferrin, which also play an important role in host defense mechanisms.

The thickness of mucus varies from organ to organ and between species. However, mucin glycoproteins obtained from different sources have similar overall amino acid and protein/carbohydrate compositions, although the molecular weight may vary over a wide. Mucin consists of a large protein core with oligosaccharide side-chains attached through the O-glycosidic linkage of galactose or N-acetyl glucosamine to hydroxyl groups of serine and threonine residues. Either sialic acid or L-fucose forms the terminal group of the side chain oligosaccharides with sialic acid (negatively charged at pH greater than 2.8) forming 50 to 60% of the terminal groups. The presence of cysteine in the end regions of the mucin core facilitates cross-linking of mucin molecules via disulfide bridge formation.

The presence of a mucus layer that coats all epithelial surfaces has been largely overlooked in the elucidation of epithelial penetration enhancement mechanisms to date. This is partly because the role of mucus in the absorption of peptide and protein drugs has not yet been well established. However, for these and other drugs exhibiting a comparatively high molecular mass, the mucus layer covering the nasal mucosal surfaces may represent an almost insurmountable barrier. According to the conventional formula for calculation of the diffusion coefficient, in which the radius of the molecule indirectly correlates with the diffusion coefficient, the mucus barrier increases tremendously for polypeptide drugs. Studies focusing on this so called ‘diffusion barrier’ have demonstrated that proteins of a molecular mass greater than approximately 5 kDa exhibit minimal or no permeation into mucus layers.

The coordinate administration methods of the instant invention optionally incorporate effective mucolytic or mucus-clearing agents, which serve to degrade, thin or clear mucus from intranasal mucosal surfaces to facilitate absorption of intranasally administered biotherapeutic agents. Within these methods, a mucolytic or mucus-clearing agent is coordinately administered as an adjunct compound to enhance intranasal delivery of the biologically active agent. Alternatively, an effective amount of a mucolytic or mucus-clearing agent is incorporated as a processing agent within a multi-processing method of the invention, or as an additive within a combinatorial formulation of the invention, to provide an improved formulation that enhances intranasal delivery of biotherapeutic compounds by reducing the barrier effects of intranasal mucus.

A variety of mucolytic or mucus-clearing agents are available for incorporation within the methods and compositions of the invention. Based on their mechanisms of action, mucolytic and mucus clearing agents can often be classified into the following groups: proteases (e.g., pronase, papain) that cleave the protein core of mucin glycoproteins; sulfhydryl compounds that split mucoprotein disulfide linkages; and detergents (e.g., Triton X-100, Tween 20) that break non-covalent bonds within the mucus. Additional compounds in this context include, but are not limited to, bile salts and surfactants, for example, sodium deoxycholate, sodium taurodeoxycholate, sodium glycocholate, and lysophosphatidylcholine.

The effectiveness of bile salts in causing structural breakdown of mucus is in the order deoxycholate>taurocholate>glycocholate. Other effective agents that reduce mucus viscosity or adhesion to enhance intranasal delivery according to the methods of the invention include, e.g., short-chain fatty acids, and mucolytic agents that work by chelation, such as N-acylcollagen peptides, bile acids, and saponins (the latter function in part by chelating Ca²⁺ and/or Mg²⁺ which play an important role in maintaining mucus layer structure).

Additional mucolytic agents for use within the methods and compositions of the invention include N-acetyl-L-cysteine (ACS), a potent mucolytic agent that reduces both the viscosity and adherence of bronchopulmonary mucus and is reported to modestly increase nasal bioavailability of human growth hormone in anesthetized rats (from 7.5 to 12.2%). These and other mucolytic or mucus-clearing agents are contacted with the nasal mucosa, typically in a concentration range of about 0.2 to 20 mM, coordinately with administration of the biologically active agent, to reduce the polar viscosity and/or elasticity of intranasal mucus.

Still other mucolytic or mucus-clearing agents may be selected from a range of glycosidase enzymes, which are able to cleave glycosidic bonds within the mucus glycoprotein. α-amylase and β-amylase are representative of this class of enzymes, although their mucolytic effect may be limited In contrast, bacterial glycosidases which allow these microorganisms to permeate mucus layers of their hosts are highly mucolytic active.

For selecting mucolytic agents for use within the methods and compositions of the invention, it is important to consider the chemical nature of both the mucolytic (or mucus-clearing) and biologically active agents. For example, the proteolytic enzyme pronase exhibits a very strong mucolytic activity at pH 5.0, as well as at pH 7.2. In contrast, the protease papain exhibited substantial mucolytic activity at pH 5.0, but no detectable mucolytic activity at pH 7.2. The reason for these differences in activity are explained in part by the distinct pH-optimum for papain, reported to be pH 5. Thus, mucolytic and other enzymes for use within the invention are typically delivered in formulations having a pH at or near the pH optimum of the subject enzyme.

With respect to chemical characterization of the biologically active agent, one notable concern is the vulnerability of peptide and protein molecules to the degradative activities of proteases and sulfhydryl. In particular, peptide and protein drugs can be attacked by different types of mucolytic agents. The presence and number of cysteine residues and disulfide bonds in peptide and protein therapeutics are also important factors to consider in selecting mucolytic or mucus-clearing agents within the invention.

Whereas it is generally contraindicated to use general proteases such as pronase or papain in combination with peptide or protein drugs, the practical use of more specific proteases can be undertaken according to the above principals, as can the use of sulfhydryl compounds. For therapeutic polypeptides that exhibit no cysteine moieties within their primary structure (e.g. cyclosporin), the use of sulfhydryl compounds is not problematic. Moreover, even for protein drugs bearing disulfide bonds the use of sulfthydryl compounds can be achieved, particularly where the disulfide bonds are not accessible for thiol attack due to the conformation of the protein, they should remain stable in the presence of this type of mucolytic agents.

For combinatorial use with most biologically active agents within the invention, including peptide and protein therapeutics, non-ionogenic detergents are generally also useful as mucolytic or mucus-clearing agents. These agents typically will not modify or substantially impair the activity of therapeutic polypeptides.

Ciliostatic Agents and Methods

Because the self-cleaning capacity of certain mucosal tissues (e.g., nasal mucosal tissues) by mucociliary clearance is necessary as a protective function (e.g., to remove dust, allergens, and bacteria), it has been generally considered that this function should not be substantially impaired by mucosal medications. Mucociliary transport in the respiratory tract is a particularly important defense mechanism against infections. To achieve this function, ciliary beating in the nasal and airway passages moves a layer of mucus along the mucosa to removing inhaled particles and microorganisms.

Various reports show that mucociliary clearance can be impaired by mucosally administered drugs, as well as by a wide range of formulation additives including penetration enhancers and preservatives. For example, ethanol at concentrations greater than 2% has been shown to reduce the in vitro ciliary beating frequency. This may be mediated in part by an increase in membrane permeability that indirectly enhances flux of calcium ion which, at high concentration, is ciliostatic, or by a direct effect on the ciliary axoneme or actuation of regulatory proteins involved in a ciliary arrest response. Exemplary preservatives (methyl-p-hydroxybenzoate (0.02% and 0.15%), propyl-p-hydroxybenzoate (0.02%), and chlorobutanol (0.5%)) reversibly inhibit ciliary activity in a frog palate model. Other common additives (EDTA (0.1%), benzalkoniuin chloride (0.01%), chlorhexidine (0.01%), phenylinercuric nitrate (0.002%), and phenylmercuric borate (0.002%), have been reported to inhibit mucociliary transport irreversibly. In addition, several penetration enhancers including STDHF, laureth-9, deoxycholate, deoxycholic acid, taurocholic acid, and glycocholic acid have been reported to inhibit ciliary activity in model systems.

Despite the potential for adverse effects on mucociliary clearance attributed to ciliostatic factors, ciliostatic agents nonetheless find use within the methods and compositions of the invention to increase the residence time of mucosally (e.g., intranasally) administered interferon-β peptides, proteins, analogs and mimetics, and other biologically active agents disclosed herein. In particular, the delivery these agents within the methods and compositions of the invention is significantly enhanced in certain aspects by the coordinate administration or combinatorial formulation of one or more ciliostatic agents that function to reversibly inhibit ciliary activity of mucosal cells, to provide for a temporary, reversible increase in the residence time of the mucosally administered active agent(s). For use within these aspects of the invention, the foregoing ciliostatic factors, either specific or indirect in their activity, are all candidates for successful employment as ciliostatic agents in appropriate amounts (depending on concentration, duration and mode of delivery) such that they yield a transient (i.e., reversible) reduction or cessation of mucociliary clearance at a mucosal site of administration to enhance delivery of interferon-β peptides, proteins, analogs and mimetics, and other biologically active agents disclosed herein, without unacceptable adverse side effects.

Within more detailed aspects, a specific ciliostatic factor is employed in a combined formulation or coordinate administration protocol with one or more interferon-β peptides, proteins, analogs and mimetics, and/or other biologically active agents disclosed herein. Various bacterial ciliostatic factors isolated and characterized in the literature may be employed within these embodiments of the invention. For example, Hingley, et al. (Infection and Immunity. 51:254-262, 1986) have recently identified ciliostatic factors from the bacterium Pseudomonas aeruginosa. These are heat-stable factors released by Pseudomonas aeruginosa in culture supernatants that have been shown to inhibit ciliary function in epithelial cell cultures. Exemplary among these cilioinhibitory components are a phenazine derivative, a pyo compound (2-alkyl-4-hydroxyquinolines), and a rhamnolipid (also known as a hemolysin). Inhibitory concentrations of these and other active components were established by quantitative measures of ciliary motility and beat frequency. The pyo compound produced ciliostasis at concentrations of 50 μg/ml and without obvious ultrastructural lesions. The phenazine derivative also inhibited ciliary motility but caused some membrane disruption, although at substantially greater concentrations of 400 μg/ml. Limited exposure of tracheal explants to the rhamnolipid resulted in ciliostasis which was associated with altered ciliary membranes. More extensive exposure to rhamnolipid was associated with removal of dynein arms from axonemes. It is proposed that these and other bacterial ciliostatic factors have evolved to enable P. aeruginosa to more easily and successfully colonize the respiratory tract of mammalian hosts. On this basis, respiratory bacteria are useful pathogens for identification of suitable, specific ciliostatic factors for use within the methods and compositions of the invention.

Several methods are available to measure mucociliary clearance for evaluating the effects and uses of ciliostatic agents within the methods and compositions of the invention. Nasal mucociliary clearance can be measured by monitoring the disappearance of visible tracers such as India ink, edicol orange powder, and edicol supra orange. These tracers are followed either by direct observation or with the aid of posterior rhinoscopy or a binocular operating microscope. This method simply measures the time taken by a tracer to travel a definite distance. In more modern techniques, radiolabeled tracers are administered as an aerosol and traced by suitably collimated detectors. Alternatively, particles with a strong taste like saccharin can be placed in the nasal passage and assayed to determine the time before the subject first perceives the taste is used as an indicator of mucociliary clearance.

Additional assays are known in the art for measuring ciliary beat activity. For example, a laser light scattering technique to measure tracheobronchial mucociliary activity is based on mono-chromaticity, coherence, and directionality of laser light. Ciliary motion is measured as intensity fluctuations due to the interference of Doppler-shifted scattered light. The scattered light from moving cilia is detected by a photomultiplier tube and its frequency content analyzed by a signal correlator yielding an autocorrelation function of the detected photocurrents. In this way, both the frequency and synchrony of beating cilia can be measured continuously. Through fiberoptic rhinoscopy, this method also allows the measurement of ciliary activity in the peripheral parts of the nasal passages.

In vitro assays for evaluating ciliostatic activity of formulations within the invention are also available. For example, a commonly used and accepted assay in this context is a rabbit tracheal explant system (Gabridge et al., Pediatr. Res. 1:31-35, 1979; Chandler et al., Infect. Immun. 29:1111-1116, 1980). Other assay systems measure the ciliary beat frequency of a single cell or a small number of cells (Kennedy et al., Exp. Cell Res. 135:147-156, 1981; Rutland et al., Lancet ii 564-565, 1980; Verdugo, et al., Pediatr. Res. 13:131-135, 1979).

Surface Active Agents and Methods

Within more detailed aspects of the invention, one or more membrane penetration-enhancing agents may be employed within a mucosal delivery method or formulation of the invention to enhance mucosal delivery of interferon-β peptides, proteins, analogs and mimetics, and other biologically active agents disclosed herein. Membrane penetration enhancing agents in this context can be selected from: (i) a surfactant, (ii) a bile salt, (ii) a phospholipid additive, mixed micelle, liposome, or carrier, (iii) an alcohol, (iv) an enamine, (v) an NO donor compound, (vi) a long-chain amphipathic molecule (vii) a small hydrophobic penetration enhancer; (viii) sodium or a salicylic acid derivative; (ix) a glycerol ester of acetoacetic acid (x) a clyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, or (x) an inhibitor of cholesterol synthesis; or (xi) any combination of the membrane penetration enhancing agents recited in (i)-(x)

Certain surface-active agents are readily incorporated within the mucosal delivery formulations and methods of the invention as mucosal absorption enhancing agents. These agents, which may be coordinately administered or combinatorially formulated with interferon-β peptides, proteins, analogs and mimetics, and other biologically active agents disclosed herein, may be selected from a broad assemblage of known surfactants. Surfactants, which generally fall into three classes: (1) nonionic polyoxyethylene ethers; (2) bile salts such as sodium glycocholate (SGC) and deoxycholate (DOC); and (3) derivatives of fusidic acid such as sodium taurodihydrofusidate (STDHF). The mechanisms of action of these various classes of surface active agents typically include solubilization of the biologically active agent. For proteins and peptides which often form aggregates, the surface active properties of these absorption promoters can allow interactions with proteins such that smaller units such as surfactant coated monomers may be more readily maintained in solution. These monomers are presumably more transportable units than aggregates. A second potential mechanism is the protection of the peptide or protein from proteolytic degradation by proteases in the mucosal environment. Both bile salts and some fusidic acid derivatives reportedly inhibit proteolytic degradation of proteins by nasal homogenates at concentrations less than or equivalent to those required to enhance protein absorption. This protease inhibition may be especially important for peptides with short biological half-lives.

Degradation Enzymes and Inhibitors of Fatty Acid and Cholesterol Synthesis

In related aspects of the invention, interferon-β peptides, proteins, analogs and mimetics, and other biologically active agents for mucosal administration are formulated or coordinately administered with a penetration enhancing agent selected from a degradation enzyme, or a metabolic stimulatory agent or inhibitor of synthesis of fatty acids, sterols or other selected epithelial barrier components (see, e.g., U.S. Pat. No. 6,190,894). In one embodiment, known enzymes that act on mucosal tissue components to enhance permeability are incorporated in a combinatorial formulation or coordinate administration method of instant invention, as processing agents within the multi-processing methods of the invention. For example, degradative enzymes such as phospholipase, hyaluronidase, neuraminidase, and chondroitinase may be employed to enhance mucosal penetration of interferon-β peptides, proteins, analogs and mimetics, and other biologically active agents (see, e.g., Squier Brit. J. Dermatol. 111:253-264, 1984; Aungst and Rogers Int. J. Pharm. 53:227-235, 1989), without causing irreversible damage to the mucosal barrier. In one embodiment, chondroitinase is employed within a method or composition as provided herein to alter glycoprotein or glycolipid constituents of the permeability barrier of the mucosa, thereby enhancing mucosal absorption of interferon-β peptides, proteins, analogs and mimetics, and other biologically active agents disclosed herein.

With regard to inhibitors of synthesis of mucosal barrier constituents, it is noted that free fatty acids account for 20-25% of epithelial lipids by weight. Two rate limiting enzymes in the biosynthesis of free fatty acids are acetyl CoA carboxylase and fatty acid synthetase. Through a series of steps, free fatty acids are metabolized into phospholipids. Thus, inhibitors of free fatty acid synthesis and metabolism for use within the methods and compositions of the invention include, but are not limited to, inhibitors of acetyl CoA carboxylase such as 5-tetradecyloxy-2-furancarboxylic acid (TOFA); inhibitors of fatty acid synthetase; inhibitors of phospholipase A such as gomisin A, 2-(p-amylcinnamyl)amino-4-chlorobenzoic acid, bromophenacyl bromide, monoalide, 7,7-dimethyl-5,8-eicosadienoic acid, nicergoline, cepharanthine, nicardipine, quercetin, dibutyryl-cyclic AMP, R-24571, N-oleoylethanolamine, N-(7-nitro-2,1,3-benzoxadiazol-4-yl) phosphostidyl serine, cyclosporine A, topical anesthetics, including dibucaine, prenylamine, retinoids, such as all-trans and 13-cis-retinoic acid, W-7, trifluoperazine, R-24571 (calmidazolium), 1-hexadocyl-3-trifluoroethyl glycero-sn-2-phosphomenthol (MJ33); calcium channel blockers including nicardipine, verapamil, diltiazem, nifedipine, and nimodipine; antimalarials including quinacrine, mepacrine, chloroquine and hydroxychloroquine; beta blockers including propanalol and labetalol; calmodulin antagonists; EGTA; thimersol; glucocorticosteroids including dexamethasone and prednisolone; and nonsteroidal antiinflammatory agents including indomethacin and naproxen.

Free sterols, primarily cholesterol, account for 20-25% of the epithelial lipids by weight. The rate limiting enzyme in the biosynthesis of cholesterol is 3-hydroxy-3-methylglutaryl (HMG) CoA reductase. Inhibitors of cholesterol synthesis for use within the methods and compositions of the invention include, but are not limited to, competitive inhibitors of (HMG) CoA reductase, such as simvastatin, lovastatin, fluindostatin (fluvastatin), pravastatin, mevastatin, as well as other HMG CoA reductase inhibitors, such as cholesterol oleate, cholesterol sulfate and phosphate, and oxygenated sterols, such as 25-OH— and 26-OH— cholesterol; inhibitors of squalene synthetase; inhibitors of squalene epoxidase; inhibitors of DELTA7 or DELTA24 reductases such as 22,25-diazacholesterol, 20,25-diazacholestenol, AY9944, and triparanol.

Each of the inhibitors of fatty acid synthesis or the sterol synthesis inhibitors may be coordinately administered or combinatorially formulated with one or more interferon-β peptides, proteins, analogs and mimetics, and other biologically active agents disclosed herein to achieve enhanced epithelial penetration of the active agent(s). An effective concentration range for the sterol inhibitor in a therapeutic or adjunct formulation for mucosal delivery is generally from about 0.0001% to about 20% by weight of the total, more typically from about 0.01% to about 5%.

Nitric Oxide Donor Agents and Methods

Within other related aspects of the invention, a nitric oxide (NO) donor is selected as a membrane penetration-enhancing agent to enhance mucosal delivery of one or more interferon-β peptides, proteins, analogs and mimetics, and other biologically active agents disclosed herein. Recently, Salzman et al. (Am. J. Physiol. 268:G361-G373, 1995) reported that NO donors increase the permeability of water-soluble compounds across Caco-2 cell monolayers with neither loss of cell viability nor lactate dehydrogenase (LDH) release. In addition, Utoguchi et al. (Pharm. Res. 15:870-876, 1998) demonstrated that the rectal absorption of insulin was remarkably enhanced in the presence of NO donors, with attendant low cytotoxicity as evaluated by the cell detachment and LDH release studies in Caco-2 cells.

Various NO donors are known in the art and are useful in effective concentrations within the methods and formulations of the invention. Exemplary NO donors include, but are not limited to, nitroglycerine, nitropruside, NOC5 [3-(2-hydroxy-1-(methyl-ethyl)-2-nitrosohydrazino)-1-propanamine], NOC12 [N-ethyl-2-(1-ethyl-hydroxy-2-nitrosohydrazino)-ethanamine], SNAP [S-nitroso-N-acetyl-DL-penicillamine], NORI and NOR4. Efficacy of these and other NO donors, as well as other mucosal delivery-enhancing agents disclosed herein, for enhancing mucosal delivery of interferon-β peptides, proteins, analogs and mimetics, and other biologically active agents can be evaluated routinely according to known efficacy and cytotoxicity assay methods (e.g., involving control coadministration of an NO scavenger, such as carboxy-PIIO) as described by Utoguchi et al., Pharm. Res. 15:870-876, 1998.

Within the methods and compositions of the invention, an effective amount of a selected NO donor is coordinately administered or combinatorially formulated with one or more interferon-β peptides, proteins, analogs and mimetics, and/or other biologically active agents disclosed herein, into or through the mucosal epithelium.

Agents for Modulating Epithelial Junction Structure and/or Physiology

The present invention provides novel pharmaceutical compositions that include a biologically active agent and a permeabilizing agent effective to enhance mucosal delivery of the biologically active agent in a mammalian subject. The permeabilizing agent reversibly enhances mucosal epithelial paracellular transport, typically by modulating epithelial junctional structure and/or physiology at a mucosal epithelial surface in the subject. This effect typically involves inhibition by the permeabilizing agent of homotypic or heterotypic binding between epithelial membrane adhesive proteins of neighboring epithelial cells. Target proteins for this blockade of homotypic or heterotypic binding can be selected from various related junctional adhesion molecules (JAMs), occludins, or claudins.

In more detailed embodiments of the invention, the permeabilizing agent is a peptide or peptide analog or mimetic. Exemplary permeabilizing peptides comprise from about 4-25 contiguous amino acids of an extracellular domain of a mammalian JAM-1, JAM-2, or JAM-3 protein. Alternatively, the permeabilizing peptide may comprise from about 6-15 contiguous amino acids of an extracellular domain of a mammalian JAM-1, JAM-2, or JAM-3 protein. In additional embodiments, the permeabilizing peptide comprises from about 4-25 contiguous amino acids of an extracellular domain of a mammalian JAM-1, JAM-2, or JAM-3 protein, or a sequence of amino acids that exhibits at least 85% amino acid identity with a corresponding reference sequence of 4-25 contiguous amino acids of an extracellular domain of a mammalian JAM-1, JAM-2, or JAM-3 protein. In certain embodiments, the amino acid sequence of the permeabilizing peptide exhibits one or more amino acid substitutions, insertions, or deletions compared to the corresponding reference sequence of the mammalian JAM-1, JAM-2, or JAM-3 protein. For example, the permeabilizing peptide may exhibit one or more conservative amino acid substitutions compared to a corresponding reference sequence of a mammalian JAM-1, JAM-2, or JAM-3 protein. Such functional peptide analogs or variants may, for instance, have one or more amino acid mutations in comparison to a corresponding wild-type sequence of the same human JAM protein (e.g., human JAM-1), wherein the mutation(s) correspond to a divergent amino acid residue or sequence identified in a different human JAM protein (e.g., human JAM-2 or JAM-3) or in a homologous JAM protein found in a different species (e.g. murine, rat, or bovine JAM-1, JAM-2 or JAM-3 protein).

In more detailed embodiments, the methods and compositions of the invention incorporate a permeabilizing peptide that is between about 4-25 amino acids in length, and includes one or more contiguous sequence elements selected from: V R (I, V, A) P, (SEQ ID NO: 1); (V, A, I) K L (S, T) C A Y, (SEQ ID NO: 2); orE D (T, S) G T Y (T, R) C (M, E), (SEQ ID NO: 3). In one such embodiment, the peptide will include a conservative sequence motif V R (I, V, A) P, (SEQ ID NO: 1), wherein the third position of the motif may be represented by one of the alternative amino acid residues I, V, or A. In another such embodiment, the peptide will include a conservative sequence motif (V, A, I) K L (S, T) C A Y, (SEQ ID NO: 2), wherein the first position of the motif may be represented by one of the alternative amino acid residues V, A, or I, and the fourth position of the motif may be represented by one of the alternative amino acid residues S or T. In yet another such embodiment, the peptide will include a conservative sequence motif E D (T, S) G T Y (T, R) C (M, E), (SEQ ID NO: 3), wherein the third position of the motif may be represented by one of the alternative amino acid residues T or S, the seventh position of the motif may be represented by one of the alternative amino acid residues T or R, and the ninth position of the motif may be represented by one of the alternative residues M or E. In exemplary embodiments, the permeabilizing peptide is between about 4-25 amino acids in length and includes one or more contiguous sequence elements selected from wild-type human JAM-1 peptide sequences VRIP, (SEQ ID NO: 4), VKLSCAY, (SEQ ID NO: 5), TGITFKSVT, (SEQ ID NO: 6), ITAS, (SEQ ID NO: 7), SVTR, (SEQ ID NO: 8), EDTGTYTCM, (SEQ ID NO: 9), and/or GFSSPRVEW, (SEQ ID NO: 10).

Within additional aspects of the invention, pharmaceutical compositions and methods are provided which employ a permeabilizing peptide comprising from about 4-25 contiguous amino acids of an extracellular domain of a mammalian occludin protein. In alternate embodiments, the permeabilizing peptide comprises from about 6-15 contiguous amino acids of an extracellular domain of a mammalian occludin protein. In certain aspects, the permeabilizing peptide comprises from about 4-25 contiguous amino acids of an extracellular domain of a mammalian occludin protein or comprises an amino acid sequence that exhibits at least 85% amino acid identity with a corresponding reference sequence of 4-25 contiguous amino acids of an extracellular domain of a mammalian occludin protein. In exemplary embodiments, the permeabilizing peptide exhibits one or more amino acid substitutions, insertions, or deletions compared to a corresponding reference sequence of the mammalian occludin protein. Often, such peptide “analogs” will exhibit one or more conservative amino acid substitutions compared to the corresponding reference sequence of the mammalian occludin protein. In related embodiments, the permeabilizing peptide is a human occludin peptide and the amino acid sequence of the permeabilizing peptide exhibits one or more amino acid mutations in comparison to a corresponding wild-type sequence of the same human occludin protein, wherein the mutation(s) correspond to a structural feature (e.g., a divergent, aligned residue or sequence of residues) identified in a different human occludin protein or a homologous occludin protein found in a different species.

Within other aspects of the invention, pharmaceutical compositions and methods are provided which employ a permeabilizing peptide comprising from about 4-25 contiguous amino acids of an extracellular domain of a mammalian claudin protein. In alternate embodiments, the permeabilizing peptide comprises from about 6-15 contiguous amino acids of an extracellular domain of a mammalian claudin protein. In certain aspects, the permeabilizing peptide comprises from about 4-25 contiguous amino acids of an extracellular domain of a mammalian claudin protein or comprises an amino acid sequence that exhibits at least 85% amino acid identity with a corresponding reference sequence of 4-25 contiguous amino acids of an extracellular domain of a mammalian claudin protein. In exemplary embodiments, the permeabilizing peptide exhibits one or more amino acid substitutions, insertions, or deletions compared to a corresponding reference sequence of the mammalian claudin protein. Often, such peptide “analogs” will exhibit one or more conservative amino acid substitutions compared to the corresponding reference sequence of the mammalian claudin protein. In related embodiments, the permeabilizing peptide is a human claudin peptide and the amino acid sequence of the permeabilizing peptide exhibits one or more amino acid mutations in comparison to a corresponding wild-type sequence of the same human claudin protein, wherein the mutation(s) correspond to a structural feature (e.g., a divergent, aligned residue or sequence of residues) identified in a different human claudin protein or a homologous claudin protein found in a different species.

In related aspects of the invention, the pharmaceutical composition includes the permeabilizing agent and one or more biologically active agent(s) selected from a small molecule drug, a peptide, a protein, and a vaccine agent. See “Biologically Active Agents” above.

In yet additional embodiments, the invention provides methods and pharmaceutical compositions which employ a permeabilizing agent as described above, such as a permeabilizing peptide, and one or more therapeutic protein(s) or peptide(s) that is/are effective as a hematopoietic agent, cytokine agent, antiinfective agent, antidementia agent, antiviral agent, antitumoral agent, antipyretic agent, analgesic agent, antiinflammatory agent, antiulcer agent, antiallergic agent, antidepressant agent, psychotropic agent, cardiotonic agent, antiarrythmic agent, vasodilator agent, antihypertensive agent, antidiabetic agent, anticoagulant agent, cholesterol-lowering agent, hormone agent, anti-osteoporosis agent, antibiotic agent, vaccine agent, and/or bacterial toxoid.

In certain embodiments of the invention, a biologically active agent and a permeabilizing agent as described above are administered in combination with one or more mucosal delivery-enhancing agent(s). In more detailed embodiments of the inventions, the pharmaceutical compositions noted above are formulated for intranasal administration. In exemplary embodiments, the formulations are provided as an intranasal spray or powder. To enhance intranasal administration, these formulations may combine the biologically active agent and permeabilizing agent with one or more intranasal delivery-enhancing agents selected from:

(a) an aggregation inhibitory agent;

(b) a charge modifying agent;

(c) a pH control agent;

(d) a degradative enzyme inhibitory agent;

(e) a mucolytic or mucus clearing agent;

(f) a ciliostatic agent;

(g) a membrane penetration-enhancing agent selected from (i) a surfactant, (ii) a bile salt, (ii) a phospholipid additive, mixed micelle, liposome, or carrier, (iii) an alcohol, (iv) an enamine, (v) an NO donor compound, (vi) a long-chain amphipathic molecule (vii) a small hydrophobic penetration enhancer; (viii) sodium or a salicylic acid derivative; (ix) a glycerol ester of acetoacetic acid (x) a cyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, or (x) an inhibitor of cholesterol synthesis; or (xi) any combination of the membrane penetration enhancing agents recited in (i)-(x);

(h) a second modulatory agent of epithelial junction physiology;

(i) a vasodilator agent;

(j) a selective transport-enhancing agent; and

(k) a stabilizing delivery vehicle, carrier, support or complex-forming species with which the biologically active agent is effectively combined, associated, contained, encapsulated or bound resulting in stabilization of the active agent for enhanced intranasal delivery, wherein said one or more intranasal delivery-enhancing agents comprises any one or combination of two or more of said intranasal delivery-enhancing agents recited in (a)-(k), and wherein the formulation of said biologically active agent with said one or more intranasal delivery-enhancing agents provides for increased bioavailability of the biologically active agent delivered to a nasal mucosal surface of a mammalian subject.

In other related aspects of the invention, the pharmaceutical compositions comprising a permeabilizing agent, e.g., a permeabilizing peptide, and a biologically active agent are effective following mucosal administration to a mammalian subject to yield enhanced bioavailability of the therapeutic compound, for example by yielding a peak concentration (Cₘₐₓ) of the biologically active agent in a blood plasma or cerebral spinal fluid (CNS) of the subject that is about 25% or greater as compared to a peak concentration of the biologically active agent following intramuscular injection of an equivalent concentration or dose of the active agent to the subject. In certain embodiments, the pharmaceutical composition following mucosal administration yields a peak concentration (Cₘₐₓ) of the biologically active agent in the blood plasma or CNS of the subject that is about 50% or greater than the peak concentration of the biologically active agent in the blood plasma or CNS following intramuscular injection of an equivalent concentration or dose of the active agent.

In alternate embodiments of the invention, the pharmaceutical compositions comprising a permeabilizing agent and a biologically active agent are effective following mucosal administration to yield enhanced bioavailability by yielding an area under concentration curve (AUC) of the biologically active agent in a blood plasma or cerebral spinal fluid (CNS) of the subject that is about 25% or greater compared to an AUC of the biologically active agent in blood plasma or CNS following intramuscular injection of an equivalent concentration or dose of the active agent to the subject. In certain embodiments, the pharmaceutical compositions yield an area under concentration curve (AUC) of the biologically active agent in a blood plasma or cerebral spinal fluid (CNS) of the subject that is about 50% or greater compared to an AUC of the biologically active agent in blood plasma or CNS following intramuscular injection of an equivalent concentration or dose of the active agent to the subject.

In additional embodiments of the invention, the pharmaceutical compositions comprising a permeabilizing agent and a biologically active agent are effective following mucosal administration to yield enhanced bioavailability by yielding a time to maximal plasma concentration (tₘₐₓ) of said biologically active agent in a blood plasma or cerebral spinal fluid (CNS) of the subject between about 0.1 to 1.0 hours. In certain embodiments, the compositions yield a time to maximal plasma concentration (tₘₐₓ) of the biologically active agent in a blood plasma or cerebral spinal fluid (CNS) of the subject between about 0.2 to 0.5 hours.

In other embodiments of the invention, the pharmaceutical compositions comprising a permeabilizing agent and a biologically active agent are effective following mucosal administration to yield enhanced bioavailability of the active agent in the CNS, for example by yielding a peak concentration of the biologically active agent in a CNS tissue or fluid of the subject that is 10% or greater compared to a peak concentration of the biologically active agent in a blood plasma of the subject (e.g., wherein the CNS and plasma concentration is measured contemporaneously in the same subject following the mucosal administration). In certain embodiments, compositions of the invention yield a peak concentration of the biologically active agent in a CNS tissue or fluid of the subject that is 20%, 40%, or greater compared to a peak concentration of the active agent in a blood plasma of the subject.

The methods of the invention for treating or preventing a disease or condition in a mammalian subject amenable to treatment by therapeutic administration of one or more of the biologically active agents identified herein generally comprise coordinately, mucosally administering to said subject a pharmaceutical formulation comprising a biologically active agent (e.g., a dopamine receptor agonist) and an effective amount of a permeabilizing agent (e.g., a permeabilizing peptide), as described above, to enhance mucosal delivery of the biologically active agent. Coordinate administration of the permeabilizing agent reversibly enhances mucosal epithelial paracellular transport by modulating epithelial junctional structure and/or physiology in a target mucosal epithelium of the subject. Typically, the permeabilizing agent effectively inhibits homotypic or heterotypic binding of an epithelial membrane adhesive protein selected from a junctional adhesion molecule (JAM), occludin, or claudin. In certain embodiments, the step(s) of coordinate mucosal administration involves delivery of the permeabilizing agent before, after, or simultaneous with (e.g., in a combinatorial formulation) delivery of the biologically active agent to a mucosal surface of the subject. In more detailed embodiments, the permeabilizing agent is coordinately administered with the biologically active agent to a nasal mucosal surface of said subject, for example in a combinatorial or separate nasal spray, gel or powder formulation(s). In exemplary embodiments, the permeabilizing agent is a permeabilizing peptide administered coordinately with the biologically active agent to yield enhanced mucosal epithelial paracellular transport of the biologically active agent. In certain exemplary embodiments, the permeabilizing peptide comprises from about 4-25, or about 6-15, contiguous amino acids of an extracellular domain of a mammalian JAM, occludin or claudin protein as described above, or a comparable length peptide that exhibits at least 85% amino acid identity with a corresponding reference sequence of an extracellular domain of a mammalian JAM, occludin or claudin protein.

In related aspects of the invention, coordinate administration of the permeabilizing agent and biologically active agent yields a peak concentration (Cₘₐₓ) of the biologically active agent in a blood plasma or cerebral spinal fluid (CNS) of the subject that is 25% or greater as compared to a peak concentration of the biologically active agent following intramuscular injection of an equivalent concentration or dose of the active agent to the subject. In additional embodiments, coordinate administration of the permeabilizing agent and biologically active agent yields an area under concentration curve (AUC) of the biologically active agent in a blood plasma or cerebral spinal fluid (CNS) of the subject that is 25% or greater compared to an AUC of the biologically active agent in blood plasma or CNS following intramuscular injection of an equivalent concentration or dose of the active agent to the subject. In other embodiments, coordinate administration of the permeabilizing agent and biologically active agent yields a time to maximal plasma concentration (tₘₐₓ) of the biologically active agent in a blood plasma or cerebral spinal fluid (CNS) of the subject between 0.2 to 0.5 hours. In still other embodiments, coordinate administration of the permeabilizing agent and biologically active agent yields a peak concentration of the biologically active agent in a central nervous system (CNS) tissue or fluid of the subject that is 10% or greater compared to a peak concentration of the biologically active agent in a blood plasma of the subject.

In yet additional detailed embodiments, the invention provides permeabilizing peptides and peptide analogs and mimetics for enhancing mucosal epithelial paracellular transport. The subject peptides and peptide analogs and mimetics typically work within the compositions and methods of the invention by modulating epithelial junctional structure and/or physiology in a mammalian subject. In certain embodiments, the peptides and peptide analogs and mimetics effectively inhibit homotypic and/or heterotypic binding of an epithelial membrane adhesive protein selected from a junctional adhesion molecule (JAM), occludin, or claudin. In more detailed embodiments, the permeabilizing peptide or peptide analog comprises from about 4-25 contiguous amino acids of a wild-type sequence of an extracellular domain of a mammalian JAM-1, JAM-2, JAM-3, occludin or claudin protein, or an amino acid sequence that exhibits at least 85% amino acid identity with a corresponding reference sequence of about 4-25 contiguous amino acids of a wild-type sequence of an extracellular domain of a mammalian JAM-1, JAM-2, JAM-3, occludin or claudin protein. In exemplary embodiments, the permeabilizing peptide or peptide analog is a human JAM peptide (e.g., human JAM-1) having a wild-type amino acid sequence or exhibiting one or more amino acid mutations in comparison to a corresponding wild-type sequence of the same human JAM protein, wherein the mutation(s) correspond to a structural feature identified in a different human JAM protein or a homologous JAM protein found in a different species.

The permeabilizing peptide is between about 4-25 amino acids in length, and includes one or more contiguous sequence elements selected from: V R (I, V, A) P, (SEQ ID NO: 1); (V, A, I) K L (S, T) C A Y, (SEQ ID NO: 2); or E D (T, S) G T Y (T,R) C (M, E), (SEQ ID NO: 3). In one such embodiment, the peptide will include a conservative sequence motif V R (I, V, A) P, (SEQ ID NO: 1), wherein the third position of the motif may be represented by one of the alternative amino acid residues I, V, or A. In another such embodiment, the peptide will include a conservative sequence motif (V, A, I) K L (S, T) C A Y, (SEQ ID NO: 2), wherein the first position of the motif may be represented by one of the alternative amino acid residues V, A, or I, and the fourth position of the motif may be represented by one of the alternative amino acid residues S or T. In yet another such embodiment, the peptide will include a conservative sequence motif E D (T, S) G T Y (T,R) C (M, E), (SEQ ID NO: 3), wherein the third position of the motif may be represented by one of the alternative amino acid residues T or S, the seventh position of the motif may be represented by one of the alternative amino acid residues T or R, and the ninth position of the motif may be represented by one of the alternative residues M or E. In exemplary embodiments, the permeabilizing peptide is between about 4-25 amino acids in length and includes one or more contiguous sequence elements selected from wild-type human JAM-1 peptide sequences VRIP, (SEQ ID NO: 4), VKLSCAY, (SEQ ID NO: 5), and/or EDTGTYTCM, (SEQ ID NO: 9).

Candidate permeabilizing peptides of human JAM-1 include, but are not limited to, SVTVHSSEPE, (SEQ ID NO: 11), VRIPENNPVK, (SEQ ID NO: 12), LSCAYSGFSS, (SEQ ID NO: 13), PRVEWKFDQG, (SEQ ID NO: 14), DTTRLVCYNN, (SEQ ID NO: 15), KITASYEDRV, (SEQ ID NO: 16), TFLPTGITFK, (SEQ ID NO: 17), SVTREDTGTY, (SEQ ID NO: 18), TCMVSEEGGN, (SEQ ID NO: 19), SYGEVKVKLI, (SEQ ID NO: 20), VLVPPSKPTV, (SEQ ID NO: 21), NIPSSATIGN, (SEQ ID NO: 22), RAVLTCSEQD, (SEQ ID NO: 23), GSPPSEYTWF, (SEQ ID NO: 24), KDGIVMPTNP, (SEQ ID NO: 25), KSTRAFSNSS, (SEQ ID NO: 26), YVLNPTTGEL, (SEQ ID NO: 27), VFDPLSASDT, (SEQ ID NO: 28), GEYSCEARNG, (SEQ ID NO: 29), YGTPMTSNAV, (SEQ ID NO: 30), RMEAVERNVG, (SEQ ID NO: 31). Human JAM-1 peptides further include, SVTVH, (SEQ ID NO: 32), SSEPEVRIPE, (SEQ ID NO: 33), NNPVKLSCAY, (SEQ ID NO: 34), SGFSSPRVEW, (SEQ ID NO: 35), KFDQGDTTRL, (SEQ ID NO: 36), VCYNNKITAS, (SEQ ID NO: 37), YEDRVTFLPT, (SEQ ID NO: 38), GITFKSVTRE, (SEQ ID NO: 39), DTGTYTCMVS, (SEQ ID NO: 40), EEGGNSYGEV, (SEQ ID NO: 41), KVKLIVLVPP, (SEQ ID NO: 42), SKPTVNIPSS, (SEQ ID NO: 43), ATIGNRAVLT, (SEQ ID NO: 44), CSEQDGSPPS, (SEQ ID NO: 45), EYTWFKDGIV, (SEQ ID NO: 46), MPTNPKSTRA, (SEQ ID NO: 47), FSNSSYVLNP, (SEQ ID NO: 48), TTGELVFDPL, (SEQ ID NO: 49), SASDTGEYSC, (SEQ ID NO: 50), EARNGYGTPM, (SEQ ID NO: 51), TSNAVRMEAV, (SEQ ID NO: 52), ERNVGVI, (SEQ ID NO: 53). Human JAM-1 peptides further include, SVTVHSSE, (SEQ ID NO: 54), PEVRIPEN, (SEQ ID NO: 55), NPVKLSCA, (SEQ ID NO: 56), YSGFSSPR, (SEQ ID NO: 57), VEWKFDQG, (SEQ ID NO: 58), DTTRLVCY, (SEQ ID NO: 59), NNKITASY, (SEQ ID NO: 60), EDRVTFLP, (SEQ ID NO: 61), TGITFKSV, (SEQ ID NO: 62), TREDTGTY, (SEQ ID NO: 63), TCMVSEEG, (SEQ ID NO: 64), GNSYGEVK, (SEQ ID NO: 65), VKLIVLVP, (SEQ ID NO: 66), PSKPTVNI, (SEQ ID NO: 67), PSSATIGN, (SEQ ID NO: 68), RAVLTCSE, (SEQ ID NO: 69), QDGSPPSE, (SEQ ID NO: 70), YTWFKDGI, (SEQ ID NO: 71), VMPTNPKS, (SEQ ID NO: 72), TRAFSNSS, (SEQ ID NO: 73), YVLNPTTG, (SEQ ID NO: 74), ELVFDPLS, (SEQ ID NO: 75), ASDTGEYS, (SEQ ID NO: 76), CEARNGYG, (SEQ ID NO: 77), TPMTSNAV, (SEQ ID NO: 78), RMEAVERN, (SEQ ID NO: 79), VGVI, (SEQ ID NO: 80). Human JAM-1 peptides further include, SVTV, (SEQ ID NO: 81), HSSEPEVR, (SEQ ID NO: 82), IPENNPVK, (SEQ ID NO: 83), LSCAYSGF, (SEQ ID NO: 84), SSPRVEWK, (SEQ ID NO: 85), FDQGDTTR, (SEQ ID NO: 86), LVCYNNKI, (SEQ ID NO: 87), TASYEDRV, (SEQ ID NO: 88), TFLPTGIT, (SEQ ID NO: 89), FKSVTRED, (SEQ ID NO: 90), TGTYTCMV, (SEQ ID NO: 91), SEEGGNSY, (SEQ ID NO: 92), GEVKVKLI, (SEQ ID NO: 93), VLVPPSKP, (SEQ ID NO: 94), TVNIPSSA, (SEQ ID NO: 95), TIGNRAVL, (SEQ ID NO: 96), TCSEQDGS, (SEQ ID NO: 97), PPSEYTWF, (SEQ ID NO: 98), KDGIVMPT, (SEQ ID NO: 99), NPKSTRAF, (SEQ ID NO: 100), SNSSYVLN, (SEQ ID NO: 101), PTTGELVF, (SEQ ID NO: 102), DPLSASDT, (SEQ ID NO: 103), GEYSCEAR, (SEQ ID NO: 104), NGYGTPMT, (SEQ ID NO: 105), SNAVRMEA, (SEQ ID NO: 106), VERNVGVI, (SEQ ID NO: 107).

Exemplary permeabilizing peptides of human JAM-1 include but are not limited to VR(I,V,A)P, (SEQ ID NO: 1), VR(I)P, (SEQ ID NO: 4), PVR(I)PE, (SEQ ID NO: 108), EPEVR(I)PENN, (SEQ ID NO: 109), SEPEVR(I)PENNP, (SEQ ID NO: 110), SSEPEVR(I)PENNPV, (SEQ ID NO: 111), HSSEPEVR(I)PENNPVK, (SEQ ID NO: 112), VHSSEPEVR(I)PENNPVKL, (SEQ ID NO: 113), TVHSSEPEVR(I)PENNPVKLS, (SEQ ID NO: 114), VR(I)PE, (SEQ ID NO: 115), VR(I)PEN, (SEQ ID NO: 116), VR(I)PENN, (SEQ ID NO: 117), VR(I)PENNP, (SEQ ID NO: 118), VR(I)PENNPV, (SEQ ID NO: 119), VR(I)PENNPVK, (SEQ ID NO: 120), VR(I)PENNPVKL, (SEQ ID NO: 121), VR(I)PENNPVKLS, (SEQ ID NO: 122), EVR(I)P, (SEQ ID NO: 123), PEVR(I)P, (SEQ ID NO: 124), EPEVR(I)P, (SEQ ID NO: 125), SEPEVR(I)P, (SEQ ID NO: 126), SSEPEVR(I)P, (SEQ ID NO: 127), HSSEPEVR(I)P, (SEQ ID NO: 128), VHSSEPEVR(I)P, (SEQ ID NO: 129), TVHSSEPEVR(I)P, (SEQ ID NO: 130) and PEVRIPEN (SEQ ID NO: 789)

Exemplary permeabilizing human JAM-1 peptides further include, VR(V)P, (SEQ ID NO: 131), PVR(V)PE, (SEQ ID NO: 132), PEVR(V)PEN, (SEQ ID NO: 133), EPEVR(V)PENN, (SEQ ID NO: 134), SEPEVR(V)PENNP, (SEQ ID NO: 135), SSEPEVR(V)PENNPV, (SEQ ID NO: 136), HSSEPEVR(V)PENNPVK, (SEQ ID NO: 137), VHSSEPEVR(V)PENNPVKL, (SEQ ID NO: 138), TVHSSEPEVR(V)PENNPVKLS, (SEQ ID NO: 139), VR(V)PE, (SEQ ID NO: 140), VR(V)PEN, (SEQ ID NO: 141), VR(V)PENN, (SEQ ID NO: 142), VR(V)PENNP, (SEQ ID NO: 143), VR(V)PENNPV, (SEQ ID NO: 144), VR(V)PENNPVK, (SEQ ID NO: 145), VR(V)PENNPVKL, (SEQ ID NO: 146), VR(V)PENNPVKLS, (SEQ ID NO: 147), EVR(V)P, (SEQ ID NO: 148), PEVR(V)P, (SEQ ID NO: 149), EPEVR(V)P, (SEQ ID NO: 150), SEPEVR(V)P, (SEQ ID NO: 151), SSEPEVR(V)P, (SEQ ID NO: 152), HSSEPEVR(V)P, (SEQ ID NO: 153), VHSSEPEVR(V)P, (SEQ ID NO: 154), TVHSSEPEVR(V)P, (SEQ ID NO: 155), VR(A)P, (SEQ ID NO: 156), PVR(A)PE, (SEQ ID NO: 157), PEVR(A)PEN, (SEQ ID NO: 158), EPEVR(A)PENN, (SEQ ID NO: 159), SEPEVR(A)PENNP, (SEQ ID NO: 160), SSEPEVR(A)PENNPV, (SEQ ID NO: 161), HSSEPEVR(A)PENNPVK, (SEQ ID NO: 162), VHSSEPEVR(A)PENNPVKL, (SEQ ID NO: 163), TVHSSEPEVR(A)PENNPVKLS, (SEQ ID NO: 164), VR(A)PE, (SEQ ID NO: 165), VR(A)PEN, (SEQ ID NO: 166), VR(A)PENN, (SEQ ID NO: 167), VR(A)PENNP, (SEQ ID NO: 168), VR(A)PENNPV, (SEQ ID NO: 169), VR(A)PENNPVK, (SEQ ID NO: 170), VR(A)PENNPVKL, (SEQ ID NO: 171), VR(A)PENNPVKLS, (SEQ ID NO: 172), EVR(A)P, (SEQ ID NO: 173), PEVR(A)P, (SEQ ID NO: 174), EPEVR(A)P, (SEQ ID NO: 175), SEPEVR(A)P, (SEQ ID NO: 176), SSEPEVR(A)P, (SEQ ID NO: 177), HSSEPEVR(A)P, (SEQ ID NO: 178), VHSSEPEVR(A)P, (SEQ ID NO: 179), TVHSSEPEVR(A)P, (SEQ ID NO: 180).

Exemplary permeabilizing human JAM-1 peptides further include, (V,A,I)KL(S,T)CAY, (SEQ ID NO: 2), (V)KL(S)CAY, (SEQ ID NO: 6), P(V)KL(S)CAYS, (SEQ ID NO: 181), NP(V)KL(S)CAYSG, (SEQ ID NO: 182), NNP(V)KL(S)CAYSGF, (SEQ ID NO: 183), ENNP(V)KL(S)CAYSGFS, (SEQ ID NO: 184), PENNP(V)KL(S)CAYSGFSS, (SEQ ID NO: 185), IPENNP(V)KL(S)CAYSGFSSP, (SEQ ID NO: 186), RIPENNP(V)KL(S)CAYSGFSSPR, (SEQ ID NO: 187), P(V)KL(S)CAY, (SEQ ID NO: 188), NP(V)KL(S)CAY, (SEQ ID NO: 189), NNP(V)KL(S)CAY, (SEQ ID NO: 190), ENNP(V)KL(S)CAY, (SEQ ID NO: 191), PENNP(V)KL(S)CAY, (SEQ ID NO: 192), IPENNP(V)KL(S)CAY, (SEQ ID NO: 193), RIPENNP(V)KL(S)CAY, (SEQ ID NO: 194), (V)KL(S)CAYS, (SEQ ID NO: 195), (V)KL(S)CAYSG, (SEQ ID NO: 196), (V)KL(S)CAYSGF, (SEQ ID NO: 197), (V)KL(S)CAYSGFS, (SEQ ID NO: 198), (V)KL(S)CAYSGFSS, (SEQ ID NO: 199), (V)KL(S)CAYSGFSSP, (SEQ ID NO: 200), (V)KL(S)CAYSGFSSPR, (SEQ ID NO: 201), (V)KL(T)CAY, (SEQ ID NO: 202), (V)KL(T)CAY, (SEQ ID NO: 203), P(V)KL(T)CAYS, (SEQ ID NO: 204), NP(V)KL(T)CAYSG, (SEQ ID NO: 205), NNP(V)KL(T)CAYSGF, (SEQ ID NO: 206), ENNP(V)KL(T)CAYSGFS, (SEQ ID NO: 207), PENNP(V)KL(T)CAYSGFSS, (SEQ ID NO: 208), IPENNP(V)KL(T)CAYSGFSSP, (SEQ ID NO: 209), RIPENNP(V)KL(T)CAYSGFSSPR, (SEQ ID NO: 210), P(V)KL(T)CAY, (SEQ ID NO: 211), NP(V)KL(T)CAY, (SEQ ID NO: 212), NNP(V)KL(T)CAY, (SEQ ID NO: 213), ENNP(V)KL(T)CAY, (SEQ ID NO: 214), PENNP(V)KL(T)CAY, (SEQ ID NO: 215), IPENNP(V)KL(T)CAY, (SEQ ID NO: 216), RIPENNP(V)KL(T)CAY, (SEQ ID NO: 217), (V)KL(T)CAYS, (SEQ ID NO: 218), (V)KL(T)CAYSG, (SEQ ID NO: 219), (V)KL(T)CAYSGF, (SEQ ID NO: 220), (V)KL(T)CAYSGFS, (SEQ ID NO: 221), (V)KL(T)CAYSGFSS, (SEQ ID NO: 222), (V)KL(T)CAYSGFSSP, (SEQ ID NO: 223), (V)KL(T)CAYSGFSSPR, (SEQ ID NO: 224).

Exemplary permeabilizing human JAM-1 peptides further include, (A)KL(S)CAY, (SEQ ID NO: 225), (A)KL(S)CAY, (SEQ ID NO: 226), P(A)KL(S)CAYS, (SEQ ID NO: 227), NP(A)KL(S)CAYSG, (SEQ ID NO: 228), NNP(A)KL(S)CAYSGF, (SEQ ID NO: 229), ENNP(A)KL(S)CAYSGFS, (SEQ ID NO: 230), PENNP(A)KL(S)CAYSGFSS, (SEQ ID NO: 231), IPENNP(A)KL(S)CAYSGFSSP, (SEQ ID NO: 232), RIPENNP(A)KL(S)CAYSGFSSPR, (SEQ ID NO: 233), P(A)KL(S)CAY, (SEQ ID NO: 234), NP(A)KL(S)CAY, (SEQ ID NO: 235), NNP(A)KL(S)CAY, (SEQ ID NO: 236), ENNP(A)KL(S)CAY, (SEQ ID NO: 237), PENNP(A)KL(S)CAY, (SEQ ID NO: 238), IPENNP(A)KL(S)CAY, (SEQ ID NO: 239), RIPENNP(A)KL(S)CAY, (SEQ ID NO: 240), (A)KL(S)CAYS, (SEQ ID NO: 241), (A)KL(S)CAYSG, (SEQ ID NO: 242), (A)KL(S)CAYSGF, (SEQ ID NO: 243), (A)KL(S)CAYSGFS, (SEQ ID NO: 244), (A)KL(S)CAYSGFSS, (SEQ ID NO: 245), (A)KL(S)CAYSGFSSP, (SEQ ID NO: 246), (A)KL(S)CAYSGFSSPR, (SEQ ID NO: 247), (A)KL(T)CAY, (SEQ ID NO: 248), (A)KL(T)CAY, (SEQ ID NO: 249), P(A)KL(T)CAYS, (SEQ ID NO: 250), NP(A)KL(T)CAYSG, (SEQ ID NO: 251), NNP(A)KL(T)CAYSGF, (SEQ ID NO: 252), ENNP(A)KL(T)CAYSGFS, (SEQ ID NO: 253), PENNP(A)KL(T)CAYSGFSS, (SEQ ID NO: 254), IPENNP(A)KL(T)CAYSGFSSP, (SEQ ID NO: 255), RIPENNP(A)KL(T)CAYSGFSSPR, (SEQ ID NO: 256), P(A)KL(T)CAY, (SEQ ID NO: 257), NP(A)KL(T)CAY, (SEQ ID NO: 258), NNP(A)KL(T)CAY, (SEQ ID NO: 259), ENNP(A)KL(T)CAY, (SEQ ID NO: 260), PENNP(A)KL(T)CAY, (SEQ ID NO: 261), IPENNP(A)KL(T)CAY, (SEQ ID NO: 262), RIPENNP(A)KL(T)CAY, (SEQ ID NO: 263), (A)KL(T)CAYS, (SEQ ID NO: 264), (A)KL(T)CAYSG, (SEQ ID NO: 265), (A)KL(T)CAYSGF, (SEQ ID NO: 266), (A)KL(T)CAYSGFS, (SEQ ID NO: 267), (A)KL(T)CAYSGFSS, (SEQ ID NO: 268), (A)KL(T)CAYSGFSSP, (SEQ ID NO: 269), (A)KL(T)CAYSGFSSPR, (SEQ ID NO: 270).

Exemplary permeabilizing human JAM-1 peptides further include, ED(T,S)GTY(T,R)C(M,E), (SEQ ID NO: 3), ED(T)GTY(T)C(M), (SEQ ID NO: 9), RED(T)GTY(T)C(M)V, (SEQ ID NO: 271), TRED(T)GTY(T)C(M)VS, (SEQ ID NO: 272), VTRED(T)GTY(T)C(M)VSE, (SEQ ID NO: 273), SVTRED(T)GTY(T)C(M)VSEE, (SEQ ID NO: 274), KSVTRED(T)GTY(T)C(M)VSEEG, (SEQ ID NO: 275), RED(T)GTY(T)C(M), (SEQ ID NO: 276), TRED(T)GTY(T)C(M), (SEQ ID NO: 277), VTRED(T)GTY(T)C(M), (SEQ ID NO: 278), SVTRED(T)GTY(T)C(M), (SEQ ID NO: 279), KSVTRED(T)GTY(T)C(M), (SEQ ID NO: 280), ED(T)GTY(T)C(M)V, (SEQ ID NO: 281), ED(T)GTY(T)C(M)VS, (SEQ ID NO: 282), ED(T)GTY(T)C(M)VSE, (SEQ ID NO: 283), ED(T)GTY(T)C(M)VSEE, (SEQ ID NO: 284), ED(T)GTY(T)C(M)VSEEG, (SEQ ID NO: 285), ED(T)GTY(T)C(E), (SEQ ID NO: 286), RED(T)GTY(T)C(E)V, (SEQ ID NO: 287), TRED(T)GTY(T)C(E)VS, (SEQ ID NO: 288), VTRED(T)GTY(T)C(E)VSE, (SEQ ID NO: 289), SVTRED(T)GTY(T)C(E)VSEE, (SEQ ID NO: 290), KSVTRED(T)GTY(T)C(E)VSEEG, (SEQ ID NO: 291), RED(T)GTY(T)C(E), (SEQ ID NO: 292), TRED(T)GTY(T)C(E), (SEQ ID NO: 293), VTRED(T)GTY(T)C(E), (SEQ ID NO: 294), SVTRED(T)GTY(T)C(E), (SEQ ID NO: 295), KSVTRED(T)GTY(T)C(E), (SEQ ID NO: 296), ED(T)GTY(T)C(E)V, (SEQ ID NO: 297), ED(T)GTY(T)C(E)VS, (SEQ ID NO: 298), ED(T)GTY(T)C(E)VSE, (SEQ ID NO: 299), ED(T)GTY(T)C(E)VSEE, (SEQ ID NO: 300), ED(T)GTY(T)C(E)VSEEG, (SEQ ID NO: 301), ED(T)GTY(R)C(M), (SEQ ID NO: 302), RED(T)GTY(T)C(M)V, (SEQ ID NO: 303), TRED(T)GTY(T)C(M)VS, (SEQ ID NO: 304), VTRED(T)GTY(T)C(M)VSE, (SEQ ID NO: 305), SVTRED(T)GTY(T)C(M)VSEE, (SEQ ID NO: 306), KSVTRED(T)GTY(T)C(M)VSEEG, (SEQ ID NO: 307), RED(T)GTY(T)C(M), (SEQ ID NO: 308), TRED(T)GTY(T)C(M), (SEQ ID NO: 309), VTRED(T)GTY(T)C(M), (SEQ ID NO: 310), SVTRED(T)GTY(T)C(M), (SEQ ID NO: 311), KSVTRED(T)GTY(T)C(M), (SEQ ID NO: 312), ED(T)GTY(T)C(M)V, (SEQ ID NO: 313), ED(T)GTY(T)C(M)VS, (SEQ ID NO: 314), ED(T)GTY(T)C(M)VSE, (SEQ ID NO: 315), ED(T)GTY(T)C(M)VSEE, (SEQ ID NO: 316), ED(T)GTY(T)C(M)VSEEG, (SEQ ID NO: 317).

Exemplary permeabilizing human JAM-1 peptides further include, ED(T)GTY(R)C(E), (SEQ ID NO: 318), RED(T)GTY(T)C(M)V, (SEQ ID NO: 319), TRED(T)GTY(T)C(M)VS, (SEQ ID NO: 320), VTRED(T)GTY(T)C(M)VSE, (SEQ ID NO: 321), SVTRED(T)GTY(T)C(M)VSEE, (SEQ ID NO: 322), KSVTRED(T)GTY(T)C(M)VSEEG, (SEQ ID NO: 323), RED(T)GTY(T)C(M), (SEQ ID NO: 324), TRED(T)GTY(T)C(M), (SEQ ID NO: 325), VTRED(T)GTY(T)C(M), (SEQ ID NO: 326), SVTRED(T)GTY(T)C(M), (SEQ ID NO: 327), KSVTRED(T)GTY(T)C(M), (SEQ ID NO: 328), ED(T)GTY(T)C(M)V, (SEQ ID NO: 329), ED(T)GTY(T)C(M)VS, (SEQ ID NO: 330), ED(T)GTY(T)C(M)VSE, (SEQ ID NO: 331), ED(T)GTY(T)C(M)VSEE, (SEQ ID NO: 332), ED(T)GTY(T)C(M)VSEEG, (SEQ ID NO: 333), ED(S)GTY(T)C(M), (SEQ ID NO: 334), RED(T)GTY(T)C(M)V, (SEQ ID NO: 335), TRED(T)GTY(T)C(M)VS, (SEQ ID NO: 336), VTRED(T)GTY(T)C(M)VSE, (SEQ ID NO: 337), SVTRED(T)GTY(T)C(M)VSEE, (SEQ ID NO: 338), KSVTRED(T)GTY(T)C(M)VSEEG, (SEQ ID NO: 339), RED(T)GTY(T)C(M), (SEQ ID NO: 340), TRED(T)GTY(T)C(M), (SEQ ID NO: 341), VTRED(T)GTY(T)C(M), (SEQ ID NO: 342), SVTRED(T)GTY(T)C(M), (SEQ ID NO: 343), KSVTRED(T)GTY(T)C(M), (SEQ ID NO: 344), ED(T)GTY(T)C(M)V, (SEQ ID NO: 345), ED(T)GTY(T)C(M)VS, (SEQ ID NO: 346), ED(T)GTY(T)C(M)VSE, (SEQ ID NO: 347), ED(T)GTY(T)C(M)VSEE, (SEQ ID NO: 348), ED(T)GTY(T)C(M)VSEEG, (SEQ ID NO: 349), ED(S)GTY(T)C(E), (SEQ ID NO: 350), RED(S)GTY(T)C(E)V, (SEQ ID NO: 351), TRED(S)GTY(T)C(E)VS, (SEQ ID NO: 352), VTRED(S)GTY(T)C(E)VSE, (SEQ ID NO: 353), SVTRED(S)GTY(T)C(E)VSEE, (SEQ ID NO: 354), KSVTRED(S)GTY(T)C(E)VSEEG, (SEQ ID NO: 355), RED(S)GTY(T)C(E), (SEQ ID NO: 356), TRED(S)GTY(T)C(E), (SEQ ID NO: 357), VTRED(S)GTY(T)C(E), (SEQ ID NO: 358), SVTRED(S)GTY(T)C(E), (SEQ ID NO: 359), KSVTRED(S)GTY(T)C(E), (SEQ ID NO: 360), ED(S)GTY(T)C(E)V, (SEQ ID NO: 361), ED(S)GTY(T)C(E)VS, (SEQ ID NO: 362), ED(S)GTY(T)C(E)VSE, (SEQ ID NO: 363), ED(S)GTY(T)C(E)VSEE, (SEQ ID NO: 364), ED(S)GTY(T)C(E)VSEEG, (SEQ ID NO: 365).

Exemplary permeabilizing human JAM-1 peptides further include, ED(S)GTY(R)C(M), (SEQ ID NO: 366), RED(S)GTY(R)C(M)V, (SEQ ID NO: 367), TRED(S)GTY(R)C(M)VS, (SEQ ID NO: 368), VTRED(S)GTY(R)C(M)VSE, (SEQ ID NO: 369), SVTRED(S)GTY(R)C(M)VSEE, (SEQ ID NO: 370), KSVTRED(S)GTY(R)C(M)VSEEG, (SEQ ID NO: 371), RED(S)GTY(R)C(M), (SEQ ID NO: 372), TRED(S)GTY(R)C(M), (SEQ ID NO: 373), VTRED(S)GTY(R)C(M), (SEQ ID NO: 374), SVTRED(S)GTY(R)C(M), (SEQ ID NO: 375), KSVTRED(S)GTY(R)C(M), (SEQ ID NO: 376), ED(S)GTY(R)C(M)V, (SEQ ID NO: 377), ED(S)GTY(R)C(M)VS, (SEQ ID NO: 378), ED(S)GTY(R)C(M)VSE, (SEQ ID NO: 379), ED(S)GTY(R)C(M)VSEE, (SEQ ID NO: 380), ED(S)GTY(R)C(M)VSEEG, (SEQ ID NO: 381).

Exemplary permeabilizing human JAM-1 peptides further include, ED(S)GTY(R)C(E), (SEQ ID NO: 382), RED(S)GTY(R)C(E)V, (SEQ ID NO: 383), TRED(S)GTY(R)C(E)VS, (SEQ ID NO: 384), VTRED(S)GTY(R)C(E)VSE, (SEQ ID NO: 385), SVTRED(S)GTY(R)C(E)VSEE, (SEQ ID NO: 386), KSVTRED(S)GTY(R)C(E)VSEEG, (SEQ ID NO: 387), RED(S)GTY(R)C(E), (SEQ ID NO: 388), TRED(S)GTY(R)C(E), (SEQ ID NO: 389), VTRED(S)GTY(R)C(E), (SEQ ID NO: 390), SVTRED(S)GTY(R)C(E), (SEQ ID NO: 391), KSVTRED(S)GTY(R)C(E), (SEQ ID NO: 392), ED(S)GTY(R)C(E)V, (SEQ ID NO: 393), ED(S)GTY(R)C(E)VS, (SEQ ID NO: 394), ED(S)GTY(R)C(E)VSE, (SEQ ID NO: 395), ED(S)GTY(R)C(E)VSEE, (SEQ ID NO: 396), ED(S)GTY(R)C(E)VSEEG, (SEQ ID NO: 397).

Candidate permeabilizing peptides of human JAM-2 include, but are not limited to AVNLKSSNRT, (SEQ ID NO: 398), PVVQEFESVE, (SEQ ID NO: 399), LSCIITDSQT, (SEQ ID NO: 400), SDPRIEWKKI, (SEQ ID NO: 401), QDEQTTYVFF, (SEQ ID NO: 402), DNKIQGDLAG, (SEQ ID NO: 403), RAEILGKTSL, (SEQ ID NO: 404), KIWNVTRRDS, (SEQ ID NO: 405), ALYRCEVVAR, (SEQ ID NO: 406), NDRKEIDEIV, (SEQ ID NO: 407), IELTVQVKPV, (SEQ ID NO: 408), TPVCRVPKAV, (SEQ ID NO: 409), PVGKMATLHC, (SEQ ID NO: 410), QESEGHPRPH, (SEQ ID NO: 411), YSWYRNDVPL, (SEQ ID NO: 412), PTDSRANPRF, (SEQ ID NO: 413), RNSSFHLNSE, (SEQ ID NO: 414), TGTLVFTAVH, (SEQ ID NO: 415), KDDSGQYYCI, (SEQ ID NO: 416), ASNDAGSARC, (SEQ ID NO: 417), EEQEMEVYDLN, (SEQ ID NO: 418).

Candidate permeabilizing peptides of human JAM-2 further include AVNLK, (SEQ ID NO: 419), SSNRTPVVQE, (SEQ ID NO: 420), FESVELSCII, (SEQ ID NO: 421), TDSQTSDPRI, (SEQ ID NO: 422), EWKKIQDEQT, (SEQ ID NO: 423), TYVFFDNKIQ, (SEQ ID NO: 424), GDLAGRAEIL, (SEQ ID NO: 425), GKTSLKIWNV, (SEQ ID NO: 426), TRRDSALYRC, (SEQ ID NO: 427), EVVARNDRKE, (SEQ ID NO: 428), IDEIVIELTV, (SEQ ID NO: 429), QVKPVTPVCR, (SEQ ID NO: 430), VPKAVPVGKM, (SEQ ID NO: 431), ATLHCQESEG, (SEQ ID NO: 432), HPRPHYSWYR, (SEQ ID NO: 433), NDVPLPTDSR, (SEQ ID NO: 434), ANPRFRNSSF, (SEQ ID NO: 435), HLNSETGTLV, (SEQ ID NO: 436), FTAVHKDDSG, (SEQ ID NO: 437), QYYCIASNDA, (SEQ ID NO: 438), GSARCEEQEM, (SEQ ID NO: 439), EVYDLN, (SEQ ID NO: 440).

Candidate permeabilizing peptides of human JAM-2 further include AVNLKSSN, (SEQ ID NO: 441), RTPVVQEF, (SEQ ID NO: 442), ESVELSCI, (SEQ ID NO: 443), ITDSQTSD, (SEQ ID NO: 444), QDEQTTYV, (SEQ ID NO: 445), FFDNKIQG, (SEQ ID NO: 446), DLAGRAEI, (SEQ ID NO: 447), LGKTSLKI, (SEQ ID NO: 448), WNVTRRDS, (SEQ ID NO: 449), ALYRCEVV, (SEQ ID NO: 450), ARNDRKEI, (SEQ ID NO: 451), DEIVIELT, (SEQ ID NO: 452), VQVKPVTP, (SEQ ID NO: 453), VCRVPKAV, (SEQ ID NO: 454), PVGKMATL, (SEQ ID NO: 455), HCQESEGH, (SEQ ID NO: 456), PRPHYSWY, (SEQ ID NO: 457), RNDVPLPT, (SEQ ID NO: 458), DSRANPRF, (SEQ ID NO: 459), RNSSFHLN, (SEQ ID NO: 460), SETGTLVF, (SEQ ID NO: 461), TAVHKDDS, (SEQ ID NO: 462), GQYYCIAS, (SEQ ID NO: 463), NDAGSARC, (SEQ ID NO: 464), EEQEMEVY, (SEQ ID NO: 465), DLN, (SEQ ID NO: 466) and PRIEWKKI (SEQ ID NO: 790).

Candidate permeabilizing peptides of human JAM-2 further include AVNL, (SEQ ID NO: 467), KSSNRTPV, (SEQ ID NO: 468), VQEFESVE, (SEQ ID NO: 469), LSCIITDS, (SEQ ID NO: 470), QTSDPRIE, (SEQ ID NO: 471), WKKIQDEQ, (SEQ ID NO: 472), TTYVFFDN, (SEQ ID NO: 473), KIQGDLAG, (SEQ ID NO: 474), RAEILGKT, (SEQ ID NO: 475), SLKIWNVT, (SEQ ID NO: 476), RRDSALYR, (SEQ ID NO: 477), CEVVARND, (SEQ ID NO: 478), RKEIDEIV, (SEQ ID NO: 479), IELTVQVK, (SEQ ID NO: 480), PVTPVCRV, (SEQ ID NO: 481), PKAVPVGK, (SEQ ID NO: 482), MATLHCQE, (SEQ ID NO: 483), SEGHPRPH, (SEQ ID NO: 484), YSWYRNDV, (SEQ ID NO: 485), PLPTDSRA, (SEQ ID NO: 486), NPRFRNSS, (SEQ ID NO: 487), FHLNSETG, (SEQ ID NO: 488), TLVFTAVH, (SEQ ID NO: 489), KDDSGQYY, (SEQ ID NO: 490), CIASNDAG, (SEQ ID NO: 491), SARCEEQE, (SEQ ID NO: 492), MEVYDLN, (SEQ ID NO: 493).

Exemplary permeabilizing peptides of human JAM-3 include, but are not limited to, GFSAPKDQQV, (SEQ ID NO: 494), VTAVEYQEAI, (SEQ ID NO: 495), LACKTPKKTV, (SEQ ID NO: 496), SSRLEWKKLG, (SEQ ID NO: 497), RSVSFVYYQQ, (SEQ ID NO: 498), TLQGDFKNRA, (SEQ ID NO: 499), EMIDFNIRIK, (SEQ ID NO: 500), NVTRSDAGKY, (SEQ ID NO: 501), RCEVSAPSEQ, (SEQ ID NO: 502), GQNLEEDTVT, (SEQ ID NO: 503), LEVLVAPAVP, (SEQ ID NO: 504), SCEVPSSALS, (SEQ ID NO: 505), GTVVELRCQD, (SEQ ID NO: 506), KEGNPAPEYT, (SEQ ID NO: 507), WFKDGIRLLE, (SEQ ID NO: 508), NPRLGSQSTN, (SEQ ID NO: 509), SSYTMNTKTG, (SEQ ID NO: 510), TLQFNTVSKL, (SEQ ID NO: 511), DTGEYSCEAR, (SEQ ID NO: 512), NSVGYRRCPG, (SEQ ID NO: 513), KRMQVDDLN, (SEQ ID NO: 514).

Exemplary permeabilizing peptides of human JAM-3 further include GFSAP, (SEQ ID NO: 515), KDQQVVTAVE, (SEQ ID NO: 516), YQEAILACKT, (SEQ ID NO: 517), PKKTVSSRLE, (SEQ ID NO: 518), WKKLGRSVSF, (SEQ ID NO: 519), VYYQQTLQGD, (SEQ ID NO: 520), FKNRAEMIDF, (SEQ ID NO: 521), NIRIKNVTRS, (SEQ ID NO: 522), DAGKYRCEVS, (SEQ ID NO: 523), APSEQGQNLE, (SEQ ID NO: 524), EDTVTLEVLV, (SEQ ID NO: 525), APAVPSCEVP, (SEQ ID NO: 526), SSALSGTVVE, (SEQ ID NO: 527), LRCQDKEGNP, (SEQ ID NO: 528), APEYTWFKDG, (SEQ ID NO: 529), IRLLENPRLG, (SEQ ID NO: 530), SQSTNSSYTM, (SEQ ID NO: 531), NTKTGTLQFN, (SEQ ID NO: 532), TVSKLDTGEY, (SEQ ID NO: 533), SCEARNSVGY, (SEQ ID NO: 534), RRCPGKRMQV, (SEQ ID NO: 535), DDLN, (SEQ ID NO: 536).

Exemplary permeabilizing peptides of human JAM-3 further include GFSAPKDQ, (SEQ ID NO: 537), QVVTAVEY, (SEQ ID NO: 538), QEAILACK, (SEQ ID NO: 539), TPKKTVSS, (SEQ ID NO: 540), RLEWKKLG, (SEQ ID NO: 541), RSVSFVYY, (SEQ ID NO: 542), QQTLQGDF, (SEQ ID NO: 543), KNRAEMID, (SEQ ID NO: 544), FNIRIKNV, (SEQ ID NO: 545), TRSDAGKY, (SEQ ID NO: 546), RCEVSAPS, (SEQ ID NO: 547), EQGQNLEE, (SEQ ID NO: 548), DTVTLEVL, (SEQ ID NO: 549), VAPAVPSC, (SEQ ID NO: 550), EVPSSALS, (SEQ ID NO: 551), GTVVELRC, (SEQ ID NO: 552), QDKEGNPA, (SEQ ID NO: 553), PEYTWFKD, (SEQ ID NO: 554), GIRLLENP, (SEQ ID NO: 555), RLGSQSTN, (SEQ ID NO: 556), SSYTMNTK, (SEQ ID NO: 557), TGTLQFNT, (SEQ ID NO: 558), VSKLDTGE, (SEQ ID NO: 559), YSCEARNS, (SEQ ID NO: 560), VGYRRCPG, (SEQ ID NO: 561), KRMQVDDLN, (SEQ ID NO: 562).

Exemplary permeabilizing peptides of human JAM-3 further include GFSA, (SEQ ID NO: 563), PKDQQVVT, (SEQ ID NO: 564), AVEYQEAI, (SEQ ID NO: 565), LACKTPKK, (SEQ ID NO: 566), TVSSRLEW, (SEQ ID NO: 567), KKLGRSVS, (SEQ ID NO: 568), FVYYQQTL, (SEQ ID NO: 569), QGDFKNRA, (SEQ ID NO: 570), EMIDFNIR, (SEQ ID NO: 571), IKNVTRSD, (SEQ ID NO: 572), AGKYRCEV, (SEQ ID NO: 573), SAPSEQGQ, (SEQ ID NO: 574), NLEEDTVT, (SEQ ID NO: 575), LEVLVAPA, (SEQ ID NO: 576), VPSCEVPS, (SEQ ID NO: 577), SALSGTVV, (SEQ ID NO: 578), ELRCQDKE, (SEQ ID NO: 579), GNPAPEYT, (SEQ ID NO: 580), WFKDGIRL, (SEQ ID NO: 581), LENPRLGS, (SEQ ID NO: 582), QSTNSSYT, (SEQ ID NO: 583), MNTKTGTL, (SEQ ID NO: 584), QFNTVSKL, (SEQ ID NO: 585), DTGEYSCE, (SEQ ID NO: 586), ARNSVGYR, (SEQ ID NO: 587), RCPGKRMQ, (SEQ ID NO: 588), VDDLN, (SEQ ID NO: 589).

Exemplary permeabilizing peptides of human claudin 1 extracellular domain include, but are not limited to, RIYSYAGDNI, (SEQ ID NO: 590), VTAQAMYEGL, (SEQ ID NO: 591), WMSCVSQSTG, (SEQ ID NO: 592), QIQCKVFDSL, (SEQ ID NO: 593), LNLSSTLQATR, (SEQ ID NO: 594), RIYSY, (SEQ ID NO: 595), AGDNIVTAQA, (SEQ ID NO: 596), MYEGLWMSCV, (SEQ ID NO: 597), SQSTGQIQCK, (SEQ ID NO: 598), VFDSLLNLSS, (SEQ ID NO: 599), TLQATR, (SEQ ID NO: 600), QEFYDPMT, (SEQ ID NO: 601), PVNARYE, (SEQ ID NO: 602), QEFYDPMTPVN, (SEQ ID NO: 603), ARYE, (SEQ ID NO: 604).

Exemplary permeabilizing peptides of human claudin 2 extracellular domain include, but are not limited to, KTSSYVGASI, (SEQ ID NO: 605), VTAVGFSKGL, (SEQ ID NO: 606), WMECATHSTG, (SEQ ID NO: 607), ITQCDIYSTL, (SEQ ID NO: 608), LGLPADIQAAQ, (SEQ ID NO: 609), KTSSY, (SEQ ID NO: 610), VGASIVTAVG, (SEQ ID NO: 611), FSKGLWMECA, (SEQ ID NO: 612), THSTGITQCD, (SEQ ID NO: 613), IYSTLLGLPA, (SEQ ID NO: 614), DIQAAQ, (SEQ ID NO: 615), RDFYSPL, (SEQ ID NO: 616).

Exemplary permeabilizing peptides of human claudin 3 extracellular domain include, but are not limited to, RVSAFIGSNI, (SEQ ID NO: 617), ITSQNIWEGL, (SEQ ID NO: 618), WMNCVVQSTG, (SEQ ID NO: 619), QMQCKVYDSL, (SEQ ID NO: 620), LALPQDLQAAR, (SEQ ID NO: 621), RVSAF, (SEQ ID NO: 622), IGSNIITSQN, (SEQ ID NO: 623), IWEGLWMNCV, (SEQ ID NO: 624), VQSTGQMQCK, (SEQ ID NO: 625), VYDSLLALPQ, (SEQ ID NO: 626), DLQAAR, (SEQ ID NO: 627), RDFYNPVV, (SEQ ID NO: 628), PEAQKRE, (SEQ ID NO: 629).

Exemplary permeabilizing peptides of human claudin 4 extracellular domain include, but are not limited to, RVTAFIGSNI, (SEQ ID NO: 630), VTSQTIWEGL, (SEQ ID NO: 631), WMNCVVQSTG, (SEQ ID NO: 632), QMQCKVYDSL, (SEQ ID NO: 633), LALPQDLQAAR, (SEQ ID NO: 634), RVTAF, (SEQ ID NO: 635), IGSNIVTSQT, (SEQ ID NO: 636), IWEGLWMNCV, (SEQ ID NO: 637), VQSTGQMQCK, (SEQ ID NO: 638), VYDSLLALPQ, (SEQ ID NO: 639), DLQAAR, (SEQ ID NO: 640), QDFYNPLV, (SEQ ID NO: 641), ASGQKRE, (SEQ ID NO: 642).

Exemplary permeabilizing peptides of human claudin 5 extracellular domain include, but are not limited to, QVTAFLDHNI, (SEQ ID NO: 643), VTAQTTWKGL, (SEQ ID NO: 644), WMSCVVQSTG, (SEQ ID NO: 645), HMQCKVYDSV, (SEQ ID NO: 646), LALSTEVQAAR, (SEQ ID NO: 647), QVTAF, (SEQ ID NO: 648), LDHNIVTAQT, (SEQ ID NO: 649), TWKGLWMSCV, (SEQ ID NO: 650), VQSTGHMQCK, (SEQ ID NO: 651), VYDSVLALST, (SEQ ID NO: 652), EVQAAR, (SEQ ID NO: 653), REFYDPSV, (SEQ ID NO: 654).

Exemplary permeabilizing peptides of human claudin 6 extracellular domain include, but are not limited to, KVTAFIGNSI, (SEQ ID NO: 655), VVAQVVWEGL, (SEQ ID NO: 656), WMSCVVQSTG, (SEQ ID NO: 657), QMQCKVYDSL, (SEQ ID NO: 658), LALPQDLQAAR, (SEQ ID NO: 659), KVTAF, (SEQ ID NO: 660), IGNSIVVAQV, (SEQ ID NO: 661), VWEGLWMSCV, (SEQ ID NO: 662), VQSTGQMQCK, (SEQ ID NO: 663), VYDSLLALPQ, (SEQ ID NO: 664), DLQAAR, (SEQ ID NO: 665), RDFYNPLV, (SEQ ID NO: 666), AEAQKRE, (SEQ ID NO: 667).

Exemplary permeabilizing peptides of human claudin 7 extracellular domain include, but are not limited to, QMSSYAGDNI, (SEQ ID NO: 668), ITAQAMYKGL, (SEQ ID NO: 669), WMDCVTQSTG, (SEQ ID NO: 670), MMSCKMYDSV, (SEQ ID NO: 671), LALSAALQATR, (SEQ ID NO: 672), QMSSY, (SEQ ID NO: 673), AGDNIITAQA, (SEQ ID NO: 674), MYKGLWMDCV, (SEQ ID NO: 675), TQSTGMMSCK, (SEQ ID NO: 676), MYDSVLALSA, (SEQ ID NO: 677), ALQATR, (SEQ ID NO: 678), TDFYNPLI, (SEQ ID NO: 679), PTNIKYE, (SEQ ID NO: 680).

Exemplary permeabilizing peptides of human claudin 8 extracellular domain include, but are not limited to, RVSAFIENNI, (SEQ ID NO: 681), VVFENFWEGL, (SEQ ID NO: 682), WMNCVRQANI, (SEQ ID NO: 683), RMQCKIYDSL, (SEQ ID NO: 684), LALSPDLQAAR, (SEQ ID NO: 685), RVSAF, (SEQ ID NO: 686), IENNIVVFEN, (SEQ ID NO: 687), FWEGLWMNCV, (SEQ ID NO: 688), RQANIRMQCK, (SEQ ID NO: 689), IYDSLLALSP, (SEQ ID NO: 690), DLQAAR, (SEQ ID NO: 691), RDFYNSIV, (SEQ ID NO: 692), NVAQKRE, (SEQ ID NO: 693).

Exemplary permeabilizing peptides of human claudin 9 extracellular domain include, but are not limited to, KVTAFIGNSI, (SEQ ID NO: 694), VVAQVVWEGL, (SEQ ID NO: 695), WMSCVVQSTG, (SEQ ID NO: 696), QMQCKVYDSL, (SEQ ID NO: 697), LALPQDLQAAR, (SEQ ID NO: 698), KVTAF, (SEQ ID NO: 699), IGNSIVVAQV, (SEQ ID NO: 700), VWEGLWMSCV, (SEQ ID NO: 701), VQSTGQMQCK, (SEQ ID NO: 702), VYDSLLALPQ, (SEQ ID NO: 703), DLQAAR, (SEQ ID NO: 704), QDFYNPLV, (SEQ ID NO: 705), AEALKRE, (SEQ ID NO: 706).

Exemplary permeabilizing peptides of human claudin 10 extracellular domain include, but are not limited to, KVSTIDGTVI, (SEQ ID NO: 707), TTATYWANLW, (SEQ ID NO: 708), KACVTDSTGV, (SEQ ID NO: 709), SNCKDFPSML, (SEQ ID NO: 710), ALDGYIQACR, (SEQ ID NO: 711), KVSTI, (SEQ ID NO: 712), DGTVITTATY, (SEQ ID NO: 713), WANLWKACVT, (SEQ ID NO: 714), DSTGVSNCKD, (SEQ ID NO: 715), FPSMLALDGY, (SEQ ID NO: 716), IQACR, (SEQ ID NO: 717), EFFDPLF, (SEQ ID NO: 718), VEQKYE, (SEQ ID NO: 719).

Exemplary permeabilizing peptides of human occludin extracellular domain include, but are not limited to, DRGYGTSLLG, (SEQ ID NO: 720), GSVGYPYGGS, (SEQ ID NO: 721), GFGSYGSGYG, (SEQ ID NO: 722), YGYGYGYGYG, (SEQ ID NO: 723), GYTDPR, (SEQ ID NO: 724), DRGYG, (SEQ ID NO: 725), TSLLGGSVGY, (SEQ ID NO: 726), PYGGSGFGSY, (SEQ ID NO: 727), GSGYGYGYGY, (SEQ ID NO: 728), GYGYGGYTDPR, (SEQ ID NO: 729), GVNPTAQSSG, (SEQ ID NO: 730), SLYGSQIYAL, (SEQ ID NO: 731), CNQFYTPAAT, (SEQ ID NO: 732), GLYVDQYLYH, (SEQ ID NO: 733), YCVVDPQE, (SEQ ID NO: 734), GVNPT, (SEQ ID NO: 735), AQSSGSLYGS, (SEQ ID NO: 736), QIYALCNQFY, (SEQ ID NO: 737), TPAATGLYVD, (SEQ ID NO: 738), QYLYHYCVVD, (SEQ ID NO: 739), PQE, (SEQ ID NO: 740).

Further candidate permeabilizing peptides of human JAM-1 include, but are not limited to, VRIP, (SEQ ID NO: 4), VKLSCAY, (SEQ ID NO: 5), TGITFKSVT, (SEQ ID NO: 6), ITAS, (SEQ ID NO: 7), SVTR, (SEQ ID NO: 8), SVTVHSSEP, (SEQ ID NO: 741), KFDQGDTTR, (SEQ ID NO: 742), EDTGTYTCM, (SEQ ID NO: 9), GEVKVKLIV, (SEQ ID NO: 743), VSEEGGNSY, (SEQ ID NO: 744), LVCYNNKIT, (SEQ ID NO: 745), GFSSPRVEW, (SEQ ID NO: 10), VLPPS, (SEQ ID NO: 746), YEDRVTF, (SEQ ID NO: 747), PRVEW, (SEQ ID NO: 748).

Further candidate permeabilizing peptides of human claudin-1 include, but are not limited to, KVFDSLLNLS, (SEQ ID NO: 749), NRIVQEFYDP, (SEQ ID NO: 750), YAGDNIVTAQ, (SEQ ID NO: 751), VSQSTGQIQC, (SEQ ID NO: 752), MTPVNARYEF, (SEQ ID NO: 753), AMYEGLWMSC, (SEQ ID NO: 754), TTWLGLWMSC, (SEQ ID NO: 755).

Further candidate permeabilizing peptides of human claudin-2 include, but are not limited to, YVGASIVTAV, (SEQ ID NO: 756), GILRDFYSPL, (SEQ ID NO: 757), VPDSMKFEIG, (SEQ ID NO: 758), DIYSTLLGLP, (SEQ ID NO: 759), GFSLGLWMEC, (SEQ ID NO: 760), ATHSTGITQC, (SEQ ID NO: 761), GFSKGLWMEC, (SEQ ID NO: 762).

Further candidate permeabilizing peptides of human claudin-3 include, but are not limited to, KVYDSLLALP, (SEQ ID NO: 763), NTIIRDFYNP, (SEQ ID NO: 764), VVPEAQKREM, (SEQ ID NO: 765), NIWEGLWMNC, (SEQ ID NO: 766), VVQSTGQMQC, (SEQ ID NO: 767), FIGSNIITSQ, (SEQ ID NO: 768).

Further candidate permeabilizing peptides of human claudin-4 include, but are not limited to, VASGQKREMG, (SEQ ID NO: 769), NIIQDFYNPL, (SEQ ID NO: 770), FIGSNIVTSQ, (SEQ ID NO: 771), TIWEGLWMNC, (SEQ ID NO: 772).

Further candidate permeabilizing peptides of human claudin-5 include, but are not limited to, IVVREFYDPS, (SEQ ID NO: 773), VVQSTGHMQC, (SEQ ID NO: 774), FLDHNIVTAQ, (SEQ ID NO: 775), VPVSQKYELG, (SEQ ID NO: 776), KVYDSVLALS, (SEQ ID NO: 777), TTWKGLWMSC, (SEQ ID NO: 778).

Further candidate permeabilizing peptides of human occludin include, but are not limited to, DRGYGTSLL, (SEQ ID NO: 779), GYGYGYGYG, (SEQ ID NO: 780), GSGFGSYGS, (SEQ ID NO: 781), YGYGGYTDP, (SEQ ID NO: 782), GVNPTAQSS, (SEQ ID NO: 783), GSLYGSQIY, (SEQ ID NO: 784), AATGLYVDQ, (SEQ ID NO: 785), ALCNQFYTP, (SEQ ID NO: 786), YLYHYCVVD, (SEQ ID NO: 787), GGSVGYPYG, (SEQ ID NO: 788).

In addition to JAM, occludin and claudin peptides, proteins, analogs and mimetics, additional agents for modulating epithelial junctional physiology and/or structure are contemplated for use within the methods and formulations of the invention. Epithelial tight junctions are generally impermeable to molecules with radii of approximately 15 angstroms, unless treated with junctional physiological control agents that stimulate substantial junctional opening as provided within the instant invention. Among the “secondary” tight junctional regulatory components that will serve as useful targets for secondary physiological modulation within the methods and compositions of the invention, the ZO1-ZO2 heterodimeric complex has shown itself amenable to physiological regulation by exogenous agents that can readily and effectively alter paracellular permeability in mucosal epithelia. On such agent that has been extensively studied is the bacterial toxin from Vibrio cholerae known as the “zonula occludens toxin” (ZOT). This toxin mediates increased intestinal mucosal permeability and causes disease symptoms including diarrhea in infected subjects (Fasano et al, Proc. Nat. Acad. Sci., USA 8:5242-5246, 1991; Johnson et al, J. Clin. Microb. 31/3:732-733, 1993; and Karasawa et al, FEBS Let. 106:143-146, (1993). When tested on rabbit ileal mucosa, ZOT increased the intestinal permeability by modulating the structure of intercellular tight junctions. More recently, it has been found that ZOT is capable of reversibly opening tight junctions in the intestinal mucosa (see, e.g., WO 96/37196; U.S. Pat. Nos. 5,945,510; 5,948,629; 5,912,323; 5,864,014; 5,827,534; 5,665,389). It has also been reported that ZOT is capable of reversibly opening tight junctions in the nasal mucosa (U.S. Pat No. 5,908,825). Thus, ZOT and other agents that modulate the ZO1-ZO2 complex will be combinatorially formulated or coordinately administered with one or more JAM, occludin and claudin peptides, proteins, analogs and mimetics, and/or other biologically active agents disclosed herein.

Within the methods and compositions of the invention, ZOT, as well as various analogs and mimetics of ZOT that function as agonists or antagonists of ZOT activity, are useful for enhancing intranasal delivery of biologically active agents—by increasing paracellular absorption into and across the nasal mucosa. In this context, ZOT typically acts by causing a structural reorganization of tight junctions marked by altered localization of the junctional protein ZO1. Within these aspects of the invention, ZOT is coordinately administered or combinatorially formulated with the biologically active agent in an effective amount to yield significantly enhanced absorption of the active agent, by reversibly increasing nasal mucosal permeability without substantial adverse side effects

Suitable methods for determining ZOT biological activity may be selected from a variety of known assays, e.g., involving assaying for a decrease of tissue or cell culture resistance (Rt) using Ussing chambers (e.g., as described by Fasano et al, Proc. Natl. Acad. Sci., USA, 8:5242-5246, 1991), assaying for a decrease of tissue resistance (Rt) of intestinal epithelial cell monolayers in Ussing chambers; or directly assaying enhancement of absorption of a therapeutic agent across a mucosal surface in vivo.

In addition to ZOT, various other tight junction modulatory agents can be employed within the methods and compositions of the invention that mimic the activity of ZOT by reversibly increasing mucosal epithelial paracellular permeability. These include specific binding or blocking agents, such as antibodies, antibody fragments, peptides, peptide mimetics, bacterial toxins and other agents that serve as agonists or antagonists of ZOT activity, or which otherwise alter physiology of the ZO1-ZO2 complex (e.g., by blocking dimerization). Naturally, these additional regulatory agents include peptide analogs, including site-directed mutant variants, of the native ZOT protein, as well as truncated active forms of the protein and peptide mimetics that model functional domains or active sites of the native protein. In addition, these agents include a native mammalian protein “zonulin”, which has been proposed to be an endogenous regulator of tight junctional physiology similar in both structural and functional aspects to ZOT (see, e.g., WO 96/37196; WO 00/07609; U.S. Pat. Nos. 5,945,510; 5,948,629; 5,912,323; 5,864,014; 5,827,534; 5,665,389), which therefore suggests that ZOT is a convergent evolutionary development of Vibrio cholerae patterned after the endogenous mammalian zonulin regulatory mechanism to facilitate host entry. Both zonulin and ZOT are proposed to bind a specific membrane receptor, designated “ZOT receptor” (see, e.g., U.S. Pat. Nos. 5,864,014; 5,912,323; and 5,948,629), which can be used within the invention to screen for additional agonists and antagonists to ZOT and zonulin activity for regulation of tight junctional physiology. In this context, structure-function analysis of the ZOT receptor, and comparisons between ZOT and zonulin, will guide production and selection of specific binding or blocking agents, (e.g., antibodies, antibody fragments, peptides, peptide mimetics, additional bacterial toxins and other agents) to serve as ZOT or zonulin agonists or antagonists, for example with respect to ZOT or zonulin binding or activation of the ZOT receptor, to regulate tight junctional physiology within the methods and compositions of the invention.

Vasodilator Agents and Methods

Yet another class of absorption-promoting agents that shows beneficial utility within the coordinate administration and combinatorial formulation methods and compositions of the invention are vasoactive compounds, more specifically vasodilators. These compounds function within the invention to modulate the structure and physiology of the submucosal vasculature, increasing the transport rate of interferon-β peptides, proteins, analogs and mimetics, and other biologically active agents into or through the mucosal epithelium and/or to specific target tissues or compartments (e.g., the systemic circulation or central nervous system.).

Vasodilator agents for use within the invention typically cause submucosal blood vessel relaxation by either a decrease in cytoplasmic calcium, an increase in nitric oxide (NO) or by inhibiting myosin light chain kinase. They are generally divided into 9 classes: calcium antagonists, potassium channel openers, ACE inhibitors, angiotensin-II receptor antagonists, α-adrenergic and imidazole receptor antagonists, β1-adrenergic agonists, phosphodiesterase inhibitors, eicosanoids and NO donors.

Despite chemical differences, the pharmacokinetic properties of calcium antagonists are similar. Absorption into the systemic circulation is high, and these agents therefore undergo considerable first-pass metabolism by the liver, resulting in individual variation in pharmacokinetics. Except for the newer drugs of the dihydropyridine type (amlodipine, felodipine, isradipine, nilvadipine, nisoldipine and nitrendipine), the half-life of calcium antagonists is short. Therefore, to maintain an effective drug concentration for many of these may require delivery by multiple dosing, or controlled release formulations, as described elsewhere herein. Treatment with the potassium channel opener minoxidil may also be limited in manner and level of administration due to potential adverse side effects.

ACE inhibitors prevent conversion of angiotensin-I to angiotensin-II, and are most effective when renin production is increased. Since ACE is identical to kininase-II, which inactivates the potent endogenous vasodilator bradykinin, ACE inhibition causes a reduction in bradykinin degradation. ACE inhibitors provide the added advantage of cardioprotective and cardioreparative effects, by preventing and reversing cardiac fibrosis and ventricular hypertrophy in animal models. The predominant elimination pathway of most ACE inhibitors is via renal excretion. Therefore, renal impairment is associated with reduced elimination and a dosage reduction of 25 to 50% is recommended in patients with moderate to severe renal impairment.

With regard to NO donors, these compounds are particularly useful within the invention for their additional effects on mucosal permeability. In addition to the above-noted NO donors, complexes of NO with nucleophiles called NO/nucleophiles, or NONOates, spontaneously and nonenzymatically release NO when dissolved in aqueous solution at physiologic pH. In contrast, nitro vasodilators such as nitroglycerin require specific enzyme activity for NO release. NONOates release NO with a defined stoichiometry and at predictable rates ranging from <3 minutes for diethylamine/NO to approximately 20 hours for diethylenetriamine/NO (DETANO).

Within certain methods and compositions of the invention, a selected vasodilator agent is coordinately administered (e.g., systemically or intranasally, simultaneously or in combinatorially effective temporal association) or combinatorially formulated with one or more interferon-β peptides, proteins, analogs and mimetics, and other biologically active agent(s) in an amount effective to enhance the mucosal absorption of the active agent(s) to reach a target tissue or compartment in the subject (e.g., the systemic circulation or CNS).

Selective Transport-Enhancing Agents and Methods

Within certain aspects of the invention, mucosal delivery of biologically active agents is enhanced by methods and agents that target selective transport mechanisms and promote endo- or transcytocis of macromoloecular drugs. In this regard, the compositions and delivery methods of the invention optionally incorporate a selective transport-enhancing agent that facilitates transport of one or more biologically active agents. These transport-enhancing agents may be employed in a combinatorial formulation or coordinate administration protocol with one or more of the interferon-β peptides, proteins, analogs and mimetics disclosed herein, to coordinately enhance delivery of one or more additional biologically active agent(s) across mucosal transport barriers, to enhance mucosal delivery of the active agent(s) to reach a target tissue or compartment in the subject (e.g., the mucosal epithelium, the systemic circulation or the CNS). Alternatively, the transport-enhancing agents may be employed in a combinatorial formulation or coordinate administration protocol to directly enhance mucosal delivery of one or more of the interferon-β peptides, proteins, analogs and mimetics, with or without enhanced delivery of an additional biologically active agent.

Exemplary selective transport-enhancing agents for use within this aspect of the invention include, but are not limited to, glycosides, sugar-containing molecules, and binding agents such as lectin binding agents, which are known to interact specifically with epithelial transport barrier components (see, e.g., Goldstein et al., Annu. Rev. Cell. Biol. 1:1-39, 1985). For example, specific “bioadhesive” ligands, including various plant and bacterial lectins, which bind to cell surface sugar moieties by receptor-mediated interactions can be employed as carriers or conjugated transport mediators for enhancing mucosal, e.g., nasal delivery of biologically active agents within the invention. Certain bioadhesive ligands for use within the invention will mediate transmission of biological signals to epithelial target cells that trigger selective uptake of the adhesive ligand by specialized cellular transport processes (endocytosis or transcytosis). These transport mediators can therefore be employed as a “carrier system” to stimulate or direct selective uptake of one or more interferon-β peptides, proteins, analogs and mimetics, and other biologically active agent(s) into and/or through mucosal epithelia. These and other selective transport-enhancing agents significantly enhance mucosal delivery of macromolecular biopharmaceuticals (particularly peptides, proteins, oligonucleotides and polynucleotide vectors) within the invention. To utilize these transport-enhancing agents, general carrier formulation and/or conjugation methods as described elsewhere herein are used to coordinately administer a selective transport enhancer (e.g., a receptor-specific ligand) and a biologically active agent to a mucosal surface, whereby the transport-enhancing agent is effective to trigger or mediate enhanced endo- or transcytosis of the active agent into or across the mucosal epithelium and/or to additional target cell(s), tissue(s) or compartment(s).

Lectins are plant proteins that bind to specific sugars found on the surface of glycoproteins and glycolipids of eukaryotic cells. Concentrated solutions of lectins have a ‘mucotractive’ effect, and various studies have demonstrated rapid receptor mediated endocytocis (RME) of lectins and lectin conjugates (e.g., concanavalin A conjugated with colloidal gold particles) across mucosal surfaces. Additional studies have reported that the uptake mechanisms for lectins can be utilized for intestinal drug targeting in vivo. In certain of these studies, polystyrene nanoparticles (500 nm) were covalently coupled to tomato lectin and reported yielded improved systemic uptake after oral administration to rats.

In addition to plant lectins, microbial adhesion and invasion factors provide a rich source of candidates for use as adhesive/selective transport carriers within the mucosal delivery methods and compositions of the invention (see, e.g., Lehr, Crit. Rev. Therap. Drug Carrier Syst. 11:177-218, 1995; Swann, P A, Pharmaceutical Research 15:826-832, 1998). Two components are necessary for bacterial adherence processes, a bacterial ‘adhesin’ (adherence or colonization factor) and a receptor on the host cell surface. Bacteria causing mucosal infections need to penetrate the mucus layer before attaching themselves to the epithelial surface. This attachment is usually mediated by bacterial fimbriae or pilus structures, although other cell surface components may also take part in the process. Adherent bacteria colonize mucosal epithelia by multiplication and initiation of a series of biochemical reactions inside the target cell through signal transduction mechanisms (with or without the help of toxins). Associated with these invasive mechanisms, a wide diversity of bioadhesive proteins (e.g., invasin, internalin) originally produced by various bacteria and viruses are known. These allow for extracellular attachment of such microorganisms with an impressive selectivity for host species and even particular target tissues. Signals transmitted by such receptor-ligand interactions trigger the transport of intact, living microorganisms into, and eventually through, epithelial cells by endo- and transcytotic processes. Such naturally occurring phenomena may be harnessed (e.g., by complexing biologically active agents such as a interferon-β peptide with an adhesin) according to the teachings herein for enhanced delivery of biologically active compounds into or across mucosal epithelia and/or to other designated target sites of drug action. One advantage of this strategy is that the selective carrier partners thus employed are substrate-specific, leaving the natural barrier function of epithelial tissues intact against other solutes.

Various bacterial and plant toxins that bind epithelial surfaces in a specific, lectin-like manner are also useful within the methods and compositions of the invention. For example, diptheria toxin (DT) enters host cells rapidly by RME. Likewise, the B subunit of the E. coli heat labile toxin binds to the brush border of intestinal epithelial cells in a highly specific, lectin-like manner. Uptake of this toxin and transcytosis to the basolateral side of the enterocytes has been reported in vivo and in vitro. Other researches have expressed the transmembrane domain of diphtheria toxin in E. coli as a maltose-binding fusion protein and coupled it chemically to high-Mw poly-L-lysine. The resulting complex was successfully used to mediate internalization of a reporter gene in vitro. In addition to these examples, Staphylococcus aureus produces a set of proteins (e.g., staphylococcal enterotoxin A (SEA), SEB, toxic shock syndrome toxin 1 (TSST-1) which act both as superantigens and toxins. Studies relating to these proteins have reported dose-dependent, facilitated transcytosis of SEB and TSST-I in Caco-2 cells.

Various plant toxins, mostly ribosome-inactivating proteins (RIPs), have been identified that bind to any mammalian cell surface expressing galactose units and are subsequently internalized by RME. Toxins such as nigrin b, α-sarcin, ricin and saporin, viscumin, and modeccin are highly toxic upon oral administration (i.e., are rapidly internalized). Therefore, modified, less toxic subunits of these compound will be useful within the invention to facilitate the uptake of biologically active agents, including interferon-β peptides, proteins, analogs and mimetics.

Viral haemagglutinins comprise another type of transport agent to facilitate mucosal delivery of biologically active agents within the methods and compositions of the invention. The initial step in many viral infections is the binding of surface proteins (haemagglutinins) to mucosal cells. These binding proteins have been identified for most viruses, including rotaviruses, varicella zoster virus, semliki forest virus, adenoviruses, potato leafroll virus, and reovirus. These and other exemplary viral hemagglutinins can be employed in a combinatorial formulation (e.g., a mixture or conjugate formulation) or coordinate administration protocol with one or more of the interferon-β peptides, proteins, analogs and mimetics disclosed herein, to coordinately enhance mucosal delivery of one or more additional biologically active agent(s). Alternatively, viral hemagglutinins can be employed in a combinatorial formulation or coordinate administration protocol to directly enhance mucosal delivery of one or more of the interferon-β peptides, proteins, analogs and mimetics, with or without enhanced delivery of an additional biologically active agent.

A variety of endogenous, selective transport-mediating factors are also available for use within the invention. Mammalian cells have developed an assortment of mechanisms to facilitate the internalization of specific substrates and target these to defined compartments. Collectively, these processes of membrane deformations are termed ‘endocytosis’ and comprise phagocytosis, pinocytosis, receptor-mediated endocytosis (clathrin-mediated RME), and potocytosis (non-clathrin-mediated RME). RME is a highly specific cellular biologic process by which, as its name implies, various ligands bind to cell surface receptors and are subsequently internalized and trafficked within the cell. In many cells the process of endocytosis is so active that the entire membrane surface is internalized and replaced in less than a half hour.

RME is initiated when specific ligands bind externally oriented membrane receptors. Binding occurs quickly and is followed by membrane invagination until an internal vesicle forms within the cell (the early endosome, “receptosome”, or CURL (compartment of uncoupling receptor and ligand). Localized membrane proteins, lipids and extracellular solutes are also internalized during this process. When the ligand binds to its specific receptor, the ligand-receptor complex accumulates in coated pits. Coated pits are areas of the membrane with high concentration of endocellular clathrin subunits. The assembly of clathrin molecules on the coated pit is believed to aid the invagination process. Specialized coat proteins called adaptins, trap specific membrane receptors that move laterally through the membrane in the coated pit area by binding to a signal sequence (Tyr-X-Arg-Phe, where X=any amino acid) at the endocellular carboxy terminus of the receptor. This process ensures that the correct receptors are concentrated in the coated pit areas and minimizes the amount of extracellular fluid that is taken up in the cell.

Following the internalization process, the clathrin coat is lost through the help of chaperone proteins, and proton pumps lower the endosomal pH to approximately 5.5, which causes dissociation of the receptor-ligand complex. CURL serves as a compartment to segregate the recycling receptor (e.g. transferrin) from receptor involved in transcytosis (e.g. transcoba-lamin). Endosomes may then move randomly or by saltatory motion along the microtubules until they reach the trans-Golgi reticulum where they are believed to fuse with Golgi components or other membranous compartments and convert into tubulovesicular complexes and late endosomes or multivesicular bodies. The fate of the receptor and ligand are determined in these sorting vesicles. Some ligands and receptors are returned to the cell surface where the ligand is released into the extracellular milieu and the receptor is recycled. Alternatively, the ligand is directed to lysosomes for destruction while the receptor is recycled to the cell membrane. The endocytotic recycling pathways of polarized epithelial cells are generally more complex than in non-polarized cells. In these enterocytes a common recycling compartment exists that receives molecules from both apical and basolateral membranes and is able to correctly return them to the appropriate membrane or membrane recycling compartment.

Current understanding of RME receptor structure and related structure-function relationships has been significantly enhanced by the cloning of mRNA sequences coding for endocytotic receptors. Most RME receptors share principal structural features, such as an extracellular ligand binding site, a single hydrophobic transmembrane domain (unless the receptor is expressed as a dimer), and a cytoplasmic tail encoding endocytosis and other functional signals. Two classes of receptors are proposed based on their orientation in the cell membrane; the amino terminus of Type I receptors is located on the extracellular side of the membrane, whereas Type II receptors have this same protein tail in the intracellular milieu.

As noted above, potocytosis, or non-clathrin coated endocytosis, takes place through caveolae, which are uniform omega- or flask-shaped membrane invaginations 50-80 nm in diameter. This process was first described as the internalization mechanism of the vitamin folic acid. Morphological studies have implicated caveolae in i) the transcytosis of macromolecules across endothelial cells; (ii) the uptake of small molecules via potocytosis involving GPI-linked receptor molecules and an unknown anion transport protein; iii) interactions with the actin-based cytoskeleton; and (iv) the compartmentalization of certain signaling molecules involved in signal transduction, including G-protein coupled receptors. Caveolae are characterized by the presence of an integral 22-kDa membrane protein termed VIP21-caveolin, which coats the cytoplasmic surface of the membrane. From a drug delivery standpoint, the advantage of potocytosis pathways over clathrin-coated RME pathways lies in the absence of the pH lowering step, which circumvents the endosomal/lysosomal pathway. This pathway for selective transporter-mediated delivery of biologically active agents is therefore particularly effective for enhanced delivery of pH-sensitive macromolecules.

Exemplary among potocytotic transport carriers mechanisms for use within the invention is the folate carrier system, which mediates transport of the vitamin folic acid (FA) into target cells via specific binding to the folate receptor (FR) (see, e.g., Reddy et al., Crit. Rev. Ther. Drug Car. Syst. 15:587-627, 1998). The cellular uptake of free folic acid is mediated by the folate receptor and/or the reduced folate carrier. The folate receptor is a glycosylphosphatidylinositol (GPI)-anchored 38 kDa glycoprotein clustered in caveolae mediating cell transport by potocytosis. While the expression of the reduced folate carrier is ubiquitously distributed in eukaryotic cells, the folate receptor is principally overexpressed in human tumors. Two homologous isoforms (α and β) of the receptor have been identified in humans. The α-isoform is found to be frequently overexprssed in epithelial tumors, whereas the β-form is often found in non-epithelial lineage tumors. Consequently, this receptor system has been used in drug-targeting approaches to cancer cells, but also in protein delivery, gene delivery, and targeting of antisense oligonucleotides to a variety of cell types.

Folate-drug conjugates are well suited for use within the mucosal delivery methods of the invention, because they allow penetration of target cells exclusively via FR-mediated endocytosis. When FA is covalently linked, for example, via its γ-carboxyl to a biologically active agent, FR binding affinity (KD˜10⁻¹⁰M) is not significantly compromised, and endocytosis proceeds relatively unhindered, promoting uptake of the attached active agent by the FR-expressing cell. Because FRs are significantly overexpressed on a large fraction of human cancer cells (e.g., ovarian, lung, breast, endometrial, renal, colon, and cancers of myeloid hematopoietic cells), this methodology allows for selective delivery of a wide range of therapeutic as well as diagnostic agents to tumors. Folate-mediated tumor targeting has been exploited to date for delivery of the following classes of molecules and molecular complexes that find use within the invention: (i) protein toxins, (ii) low-molecular-weight chemotherapeutic agents, (iii) radioimaging agents, (iv) MRI contrast agents, (v) radio-therapeutic agents, (vi) liposomes with entrapped drugs, (vii) genes, (viii) antisense oligonucleotides, (ix) ribozymes, and (x) immunotherapeutic agents (see, e.g., Swann, P A, Pharmaceutical Research 15:826-832, 1998). In virtually all cases, in vitro studies demonstrate a significant improvement in potency and/or cancer-cell specificity over the nontargeted form of the same pharmaceutical agent.

In addition to the folate receptor pathway, a variety of additional methods to stimulate transcytosis within the invention are directed to the transferrin receptor pathway, and the riboflavin receptor pathway. In one aspect, conjugation of a biologically active agent to riboflavin can effectuate RME-mediated uptake. Yet additional embodiments of the invention utilize vitamin B12 (cobalamin) as a specialized transport protein (e.g., conjugation partner) to facilitate entry of biologically active agents into target cells. Certain studies suggest that this particular system can be employed for the intestinal uptake of luteinizing hormone releasing factor (LHRH)-analogs, granulocyte colony stimulating factor (G-CSF, 18.8 kDa), erythropoietin (29.5 kDa), α-interferon, and the LHRH-antagonist ANTIDE.

Still other embodiments of the invention utilize transferrin as a carrier or stimulant of RME of mucosally delivered biologically active agents. Transferrin, an 80 kDa iron-transporting glycoprotein, is efficiently taken up into cells by RME. Transferrin receptors are found on the surface of most proliferating cells, in elevated numbers on erythroblasts and on many kinds of tumors. According to current knowledge of intestinal iron absorption, transferrin is excreted into the intestinal lumen in the form of apotransferrin and is highly stable to attacks from intestinal peptidases. In most cells, diferric transferrin binds to transferrin receptor (TfR), a dimeric transmembrane glycoprotein of 180 kDa, and the ligand-receptor complex is endocytosed within clathrin-coated vesicles. After acidification of these vesicles, iron dissociates from the transferrin/TfR complex and enters the cytoplasm, where it is bound by ferritin (Fn). Recent reports suggest that insulin covalently coupled to transferrin, is transported across Caco-2 cell monolayers by RME. Other studies suggest that oral administration of this complex to streptozotocin-induced diabetic mice significantly reduces plasma glucose levels (˜28%), which is further potentiated by BFA pretreatment. The transcytosis of transferrin (Tf) and transferrin conjugates is reportedly enhanced in the presence of Brefeldin A (BFA), a fungal metabolite. In other studies, BFA treatment has been reported to rapidly increase apical endocytosis of both ricin and HRP in MDCK cells. Thus, BFA and other agents that stimulate receptor-mediated transport can be employed within the methods of the invention as combinatorially formulated (e.g., conjugated) and/or coordinately administered agents to enhance receptor-mediated transport of biologically active agents, including interferon-β peptides, proteins, analogs and mimetics.

Immunoglobulin transport mechanisms provide yet additional endogenous pathways and reagents for incorporation within the mucosal delivery methods and compositions of the invention. Receptor-mediated transcytosis of immunoglobulin G (IgG) across the neonatal small intestine serves to convey passive immunity to many newborn mammals. In rats, IgG in milk selectively binds to neonatal Fc receptors (FcRn) expressed on the surface of the proximal small intestinal enterocytes during the first three weeks after birth. FcRn binds IgG in a pH-dependent manner, with binding occurring at the luminal pH (approx. 6-6.5) of the jejunum and release at the pH of plasma (approx. 7.4). The Fc receptor resembles the major histocompatibility complex (MHC) class I antigens in that it consists of two subunits, a transmembrane glycoprotein (gp50) in association with β₂-microglobulin. In mature absorptive cells both subunits are colocalized in each of the membrane compartments that mediate transcytosis of IgG. IgG administered in situ apparently causes both subunits to concentrate within endocytic pits of the apical plasma membrane, suggesting that ligand causes redistribution of receptors at this site. These results support a model for transport in which IgG is transferred across the cell as a complex with both subunits.

Within the methods and compositions of the present invention, IgG and other immune system-related carriers (including polyclonal and monoclonal antibodies and various fragments thereof) can be coordinate administered with biologically active agents to provide for targeted delivery, typically by receptor-mediated transport, of the biologically active agent. For example, the biologically active agent (including interferon-β peptides, proteins, analogs and mimetics) may be covalently linked to the IgG or other immunological active agent or, alternatively, formulated in liposomes or other carrier vehicle which is in turn modified (e.g., coated or covalently linked) to incorporate IgG or other immunological transport enhancer. In certain embodiments, polymeric IgA and/or IgM transport agents are employed, which bind to the polymeric immunoglobulin receptors (pIgRs) of target epithelial cells. Within these methods, expression of pIgR can be enhanced by cytokines.

Within more detailed aspects of the invention, antibodies and other immunological transport agents may be themselves modified for enhanced mucosal delivery, for example, as described in detail elsewhere herein, antibodies may be more effectively administered within the methods and compositions of the invention by charge modifying techniques. In one such aspect, an antibody drug delivery strategy involving antibody cationization is utilized that facilitates both trans-endothelial migration and target cell endocytosis (see, e.g., Pardridge, et al., JPET 286:548-544, 1998). In one such strategy, the pI of the antibody is increased by converting surface carboxyl groups of the protein to extended primary amino groups. These cationized homologous proteins have no measurable tissue toxicity and have minimal immunogenicity. In addition, monoclonal antibodies may be cationized with retention of affinity for the target protein.

Additional selective transport-enhancing agents for use within the invention comprise whole bacteria and viruses, including genetically engineered bacteria and viruses, as well as components of such bacteria and viruses. Aside from conventional gene delivery vectors (e.g., adenovirus), this aspect of the invention includes the use of bacterial ghosts and subunit constructs, e.g., as described by Huter et al., Journal of Controlled Release 61:51-63, 1999. Bacterial ghosts are non-denatured bacterial cell envelopes, for example as produced by the controlled expression of the plasmid-encoded lysis gene E of bacteriophage PhiX174 in gram-negative bacteria. Protein E-specific lysis does not cause any physical or chemical denaturation to bacterial surface structures, and bacterial ghosts are therefore useful in development of inactivated whole-cell vaccines. Ghosts produced from Actinobacillus pleuropneumoniae, Pasteurella haemolytica and Salmonella sp. have proved successful in vaccination experiments. Recombinant bacterial ghosts can be created by the expression of foreign genes fused to a membrane-targeting sequence, and thus can carry foreign therapeutic peptides and proteins anchored in their envelope. The fact that bacterial ghosts preserve a native cell wall, including bioadhesive structures like fimbriae of their living counterparts, makes them suitable for the attachment to specific target tissues such as mucosal surfaces. Bacterial ghosts have been shown to be readily taken up by macrophages, thus adhesion of ghosts to specific tissues can be followed by uptake through phagocytes.

In view of the foregoing, a wide variety of ligands involved in receptor-mediated transport mechanisms are known in the art and can be variously employed within the methods and compositions of the invention (e.g., as conjugate partners or coordinately administered mediators) to enhance receptor-mediated transport of biologically active agents, including interferon-β peptides, proteins, analogs and mimetics, and other biologically active agents disclosed herein. Generally, these ligands include hormones and growth factors, bacterial adhesins and toxins, lectins, metal ions and their carriers, vitamins, immunoglobulins, whole viruses and bacteria or selected components thereof. Exemplary ligands among these classes include, for example, calcitonin, prolactin, epidermal growth factor, glucagon, growth hormone, interferon-β, estrogen, lutenizing hormone, platelet derived growth factor, thyroid stimulating hormone, thyroid hormone, cholera toxin, diptheria toxin, E. coli heat labile toxin, Staphylococcal enterotoxins A and B, ricin, saporin, modeccin, nigrin, sarcin, concanavalin A, transcobalantin, catecholamines, transferrin, folate, riboflavin, vitamin B1, low density lipoprotein, maternal IgO, polymeric IgA, adenovirus, vesicular stomatitis virus, Rous sarcoma virus, V. cholerae, Kiebsiella strains, Serratia strains, parainfluenza virus, respiratory syncytial virus, Varicella zoster, and Enterobacter strains (see, e.g., Swann, P A, Pharmaceutical Research 15:826-832, 1998).

In certain additional embodiments of the invention, membrane-permeable peptides (e.g., “arginine rich peptides”) are employed to facilitate delivery of biologically active agents. While the mechanism of action of these peptides remains to be fully elucidated, they provide useful delivery enhancing adjuncts for use within the mucosal delivery compositions and methods herein. In one example, a basic peptide derived from human immunodeficiency virus (HIV)-1 Tat protein (e.g., residues 48-60) has been reported to translocate effectively through cell membranes and accumulate in the nucleus, a characteristic which can be utilized for the delivery of exogenous proteins into cells. The sequence of Tat (GRKKRRQRRRPPQ) (SEQ ID NO: 789) comprises a highly basic and hydrophilic peptide, which contains 6 arginine and 2 lysine residues in its 13 amino acid residues. Various other arginine-rich peptides have been identified which have a translocation activity very similar to Tat-(48-60). These include such peptides as the D-amino acid- and arginine-substituted Tat-(48-60), the RNA-binding peptides derived from virus proteins, such as HIV-1 Rev, and flock house virus coat proteins, and the DNA binding segments of leucine zipper proteins, such as cancer-related proteins c-Fos and c-Jun, and the yeast transcription factor GCN4 (see, e.g., Futaki et al., Journal Biological Chemistry 276:5836-5840, 2000). These peptides reportedly have several arginine residues marking their only identified common structural characteristic, suggesting a common internalization mechanism ubiquitous to arginine-rich peptides, which is not explained by typical endocytosis. Using (Arg)n (n=4-16) peptides, Futaki et al. teach optimization of arginine residues (n˜8) for efficient translocation. Recently, methods have been developed for the delivery of exogenous proteins into living cells with the help of arginine rich membrane-permeable carrier peptides such as HIV-1 Tat- and Antennapedia-(see, Futaki et al., supra, and references cited therein). By genetically or chemically hybridizing these carrier peptides with biologically active agents as described herein, additional methods and compositions are thus provided within the invention to enhance mucosal delivery.

Polymeric Delivery Vehicles and Methods

Within certain aspects of the invention, interferon-β peptides, proteins, analogs and mimetics, other biologically active agents disclosed herein, and delivery-enhancing agents as described above, are, individually or combinatorially, incorporated within a mucosally (e.g., nasally) administered formulation that includes a biocompatible polymer functioning as a carrier or base. Such polymer carriers include polymeric powders, matrices or microparticulate delivery vehicles, among other polymer forms. The polymer can be of plant, animal, or synthetic origin. Often the polymer is crosslinked. Additionally, in these delivery systems the biologically active agent (e.g., a interferon-β peptide, protein, analog or mimetic), can be functionalized in a manner where it can be covalently bound to the polymer and rendered inseparable from the polymer by simple washing. In other embodiments, the polymer is chemically modified with an inhibitor of enzymes or other agents which may degrade or inactivate the biologically active agent(s) and/or delivery enhancing agent(s). In certain formulations, the polymer is a partially or completely water insoluble but water swellable polymer, e.g., a hydrogel. Polymers useful in this aspect of the invention are desirably water interactive and/or hydrophilic in nature to absorb significant quantities of water, and they often form hydrogels when placed in contact with water or aqueous media for a period of time sufficient to reach equilibrium with water. In more detailed embodiments, the polymer is a hydrogel which, when placed in contact with excess water, absorbs at least two times its weight of water at equilibrium when exposed to water at room temperature (see, e.g., U.S. Pat. No. 6,004,583).

Drug delivery systems based on biodegradable polymers are preferred in many biomedical applications because such systems are broken down either by hydrolysis or by enzymatic reaction into non-toxic molecules. The rate of degradation is controlled by manipulating the composition of the biodegradable polymer matrix. These types of systems can therefore be employed in certain settings for long-term release of biologically active agents. Biodegradable polymers such as poly(glycolic acid) (PGA), poly-(lactic acid) (PLA), and poly(D,L-lactic-co-glycolic acid) (PLGA), have received considerable attention as possible drug delivery carriers, since the degradation products of these polymers have been found to have low toxicity. During the normal metabolic function of the body these polymers degrade into carbon dioxide and water. These polymers have also exhibited excellent biocompatibility.

For prolonging the biological activity of interferon-β peptides, proteins, analogs and mimetics, and other biologically active agents disclosed herein, as well as optional delivery-enhancing agents, these agents may be incorporated into polymeric matrices, e.g., polyorthoesters, polyanhydrides, or polyesters. This yields sustained activity and release of the active agent(s), e.g., as determined by the degradation of the polymer matrix Although the encapsulation of biotherapeutic molecules inside synthetic polymers may stabilize them during storage and delivery, the largest obstacle of polymer-based release technology is the activity loss of the therapeutic molecules during the formulation processes that often involve heat, sonication or organic solvents.

Absorption-promoting polymers contemplated for use within the invention may include derivatives and chemically or physically modified versions of the foregoing types of polymers, in addition to other naturally occurring or synthetic polymers, gums, resins, and other agents, as well as blends of these materials with each other or other polymers, so long as the alterations, modifications or blending do not adversely affect the desired properties, such as water absorption, hydrogel formation, and/or chemical stability for useful application. In more detailed aspects of the invention, polymers such as nylon, acrylan and other normally hydrophobic synthetic polymers may be sufficiently modified by reaction to become water swellable and/or form stable gels in aqueous media.

Suitable polymers for use within the invention should generally be stable alone and in combination with the selected biologically active agent(s) and additional components of a mucosal formulation, and form stable hydrogels in a range of pH conditions from about pH 1 to pH 10. More typically, they should be stable and form polymers under pH conditions ranging from about 3 to 9, without additional protective coatings. However, desired stability properties may be adapted to physiological parameters characteristic of the targeted site of delivery (e.g., nasal mucosa or secondary site of delivery such as the systemic circulation). Therefore, in certain formulations higher or lower stabilities at a particular pH and in a selected chemical or biological environment will be more desirable.

Absorption-promoting polymers of the invention may include polymers from the group of homo- and copolymers based on various combinations of the following vinyl monomers: acrylic and methacrylic acids, acrylamide, methacrylamide, hydroxyethylacrylate or methacrylate, vinylpyrrolidones, as well as polyvinylalcohol and its co- and terpolymers, polyvinylacetate, its co- and terpolymers with the above listed monomers and 2-acrylamido-2-methyl-propanesulfonic acid (AMPS®). Very useful are copolymers of the above listed monomers with copolymerizable functional monomers such as acryl or methacryl amide acrylate or methacrylate esters where the ester groups are derived from straight or branched chain alkyl, aryl having up to four aromatic rings which may contain alkyl substituents of 1 to 6 carbons; steroidal, sulfates, phosphates or cationic monomers such as N,N-dimethylaminoalkyl(meth)acrylamide, dimethylaminoalkyl(meth)acrylate, (meth)acryloxyalkyltrimethylammonium chloride, (meth)acryloxyalkyldimethylbenzyl ammonium chloride.

Additional absorption-promoting polymers for use within the invention are those classified as dextrans, dextrins, and from the class of materials classified as natural gums and resins, or from the class of natural polymers such as processed collagen, chitin, chitosan, pullalan, zooglan, alginates and modified alginates such as “Kelcoloid” (a polypropylene glycol modified alginate) gellan gums such as “Kelocogel”, Xanathan gums such as “Keltrol”, estastin, alpha hydroxy butyrate and its copolymers, hyaluronic acid and its derivatives, polylactic and glycolic acids.

A very useful class of polymers applicable within the instant invention are olefinically-unsaturated carboxylic acids containing at least one activated carbon-to-carbon olefinic double bond, and at least one carboxyl group; that is, an acid or functional group readily converted to an acid containing an olefinic double bond which readily functions in polymerization because of its presence in the monomer molecule, either in the alpha-beta position with respect to a carboxyl group, or as part of a terminal methylene grouping. Olefinically-unsaturated acids of this class include such materials as the acrylic acids typified by the acrylic acid itself, alpha-cyano acrylic acid, beta methylacrylic acid (crotonic acid), alpha-phenyl acrylic acid, beta-acryloxy propionic acid, cinnamic acid, p-chloro cinnamic acid, 1-carboxy-4-phenyl butadiene-1,3, itaconic acid, citraconic acid, mesaconic acid, glutaconic acid, aconitic acid, maleic acid, fumaric acid, and tricarboxy ethylene. As used herein, the term “carboxylic acid” includes the polycarboxylic acids and those acid anhydrides, such as maleic anhydride, wherein the anhydride group is formed by the elimination of one molecule of water from two carboxyl groups located on the same carboxylic acid molecule.

Representative acrylates useful as absorption-promoting agents within the invention include methyl acrylate, ethyl acrylate, propyl acrylate, isopropyl acrylate, butyl acrylate, isobutyl acrylate, methyl methacrylate, methyl ethacrylate, ethyl methacrylate, octyl acrylate, heptyl acrylate, octyl methacrylate, isopropyl methacrylate, 2-ethylhexyl methacrylate, nonyl acrylate, hexyl acrylate, n-hexyl methacrylate, and the like. Higher alkyl acrylic esters are decyl acrylate, isodecyl methacrylate, lauryl acrylate, stearyl acrylate, behenyl acrylate and melissyl acrylate and methacrylate versions thereof. Mixtures of two or three or more long chain acrylic esters may be successfully polymerized with one of the carboxylic monomers. Other comonomers include olefins, including alpha olefins, vinyl ethers, vinyl esters, and mixtures thereof.

Other vinylidene monomers, including the acrylic nitriles, may also be used as absorption-promoting agents within the methods and compositions of the invention to enhance delivery and absorption of one or more interferon-β peptides, proteins, analogs and mimetics, and other biologically active agent(s), including to enhance delivery of the active agent(s) to a target tissue or compartment in the subject (e.g., the systemic circulation or CNS). Useful alpha, beta-olefinically unsaturated nitriles are preferably monoolefinically unsaturated nitriles having from 3 to 10 carbon atoms such as acrylonitrile, methacrylonitrile, and the like. Most preferred are acrylonitrile and methacrylonitrile. Acrylic amides containing from 3 to 35 carbon atoms including monoolefinically unsaturated amides also may be used. Representative amides include acrylamide, methacrylamide, N-t-butyl acrylamide, N-cyclohexyl acrylamide, higher alkyl amides, where the alkyl group on the nitrogen contains from 8 to 32 carbon atoms, acrylic amides including N-alkylol amides of alpha, beta-olefinically unsaturated carboxylic acids including those having from 4 to 10 carbon atoms such as N-methylol acrylamide, N-propanol acrylamide, N-methylol methacrylamide, N-methylol maleimide, N-methylol maleamic acid esters, N-methylol-p-vinyl benzamide, and the like.

Yet additional useful absorption promoting materials are alpha-olefins containing from 2 to 18 carbon atoms, more preferably from 2 to 8 carbon atoms; dienes containing from 4 to 10 carbon atoms; vinyl esters and allyl esters such as vinyl acetate; vinyl aromatics such as styrene, methyl styrene and chloro-styrene; vinyl and allyl ethers and ketones such as vinyl methyl ether and methyl vinyl ketone; chloroacrylates; cyanoalkyl acrylates such as alpha-cyanomethyl acrylate, and the alpha-, beta-, and gamma-cyanopropyl acrylates; alkoxyacrylates such as methoxy ethyl acrylate; haloacrylates as chloroethyl acrylate; vinyl halides and vinyl chloride, vinylidene chloride and the like; divinyls, diacrylates and other polyfunctional monomers such as divinyl ether, diethylene glycol diacrylate, ethylene glycol dimethacrylate, methylene-bis-acrylamide, allylpentaerythritol, and the like; and bis(beta-haloalkyl) alkenyl phosphonates such as bis(beta-chloroethyl) vinyl phosphonate and the like as are known to those skilled in the art. Copolymers wherein the carboxy containing monomer is a minor constituent, and the other vinylidene monomers present as major components are readily prepared in accordance with the methods disclosed herein.

When hydrogels are employed as absorption promoting agents within the invention, these may be composed of synthetic copolymers from the group of acrylic and methacrylic acids, acrylamide, methacrylamide, hydroxyethylacrylate (HEA) or methacrylate (HEMA), and vinylpyrrolidones which are water interactive and swellable. Specific illustrative examples of useful polymers, especially for the delivery of peptides or proteins, are the following types of polymers: (meth)acrylamide and 0.1 to 99 wt. % (meth)acrylic acid; (meth)acrylamides and 0.1-75 wt % (meth)acryloxyethyl trimethyammonium chloride; (meth)acrylamide and 0.1-75 wt % (meth)acrylamide; acrylic acid and 0.1-75 wt % alkyl(meth)acrylates; (meth)acrylamide and 0.1-75 wt % AMPS.RTM. (trademark of Lubrizol Corp.); (meth)acrylamide and 0 to 30 wt % alkyl(meth)acrylamides and 0.1-75 wt % AMPS.RTM.; (meth)acrylamide and 0.1-99 wt. % HEMA; (metb)acrylamide and 0.1 to 75 wt % HEMA and 0.1 to 99%(meth)acrylic acid; (meth)acrylic acid and 0.1-99 wt % HEMA; 50 mole % vinyl ether and 50 mole % maleic anhydride; (meth)acrylamide and 0.1 to 75 wt % (meth)acryloxyalky dimethyl benzylammonium chloride; (meth)acrylamide and 0.1 to 99 wt % vinyl pyrrolidone; (meth)acrylamide and 50 wt % vinyl pyrrolidone and 0.1-99.9 wt % (meth)acrylic acid; (meth)acrylic acid and 0.1 to 75 wt % AMPS.RTM. and 0.1-75 wt % alkyl(meth)acrylamide. In the above examples, alkyl means C₁ to C₃₀, preferably C₁ to C₂₂, linear and branched and C₄ to C₁₆ cyclic; where (meth) is used, it means that the monomers with and without the methyl group are included. Other very useful hydrogel polymers are swellable, but insoluble versions of poly(vinyl pyrrolidone) starch, carboxymethyl cellulose and polyvinyl alcohol.

Additional polymeric hydrogel materials useful within the invention include (poly) hydroxyalkyl (meth)acrylate: anionic and cationic hydrogels: poly(electrolyte) complexes; poly(vinyl alcohols) having a low acetate residual: a swellable mixture of crosslinked agar and crosslinked carboxymethyl cellulose: a swellable composition comprising methyl cellulose mixed with a sparingly crosslinked agar; a water swellable copolymer produced by a dispersion of finely divided copolymer of maleic anhydride with styrene, ethylene, propylene, or isobutylene; a water swellable polymer of N-vinyl lactams; swellable sodium salts of carboxymethyl cellulose; and the like.

Other gelable, fluid imbibing and retaining polymers useful for forming the hydrophilic hydrogel for mucosal delivery of biologically active agents within the invention include pectin; polysaccharides such as agar, acacia, karaya, tragacenth, algins and guar and their crosslinked versions; acrylic acid polymers, copolymers and salt derivatives, polyacrylamides; water swellable indene maleic anhydride polymers; starch graft copolymers; acrylate type polymers and copolymers with water absorbability of about 2 to 400 times its original weight; diesters of polyglucan; a mixture of crosslinked poly(vinyl alcohol) and poly(N-vinyl-2-pyrrolidone); polyoxybutylene-polyethylene block copolymer gels; carob gum; polyester gels; poly urea gels; polyether gels; polyamide gels; polyimide gels; polypeptide gels; polyamino acid gels; poly cellulosic gels; crosslinked indene-maleic anhydride acrylate polymers; and polysaccharides.

Synthetic hydrogel polymers for use within the invention may be made by an infinite combination of several monomers in several ratios. The hydrogel can be crosslinked and generally possesses the ability to imbibe and absorb fluid and swell or expand to an enlarged equilibrium state. The hydrogel typically swells or expands upon delivery to the nasal mucosal surface, absorbing about 2-5, 5-10, 10-50, up to 50-100 or more times fold its weight of water. The optimum degree of swellability for a given hydrogel will be determined for different biologically active agents depending upon such factors as molecular weight, size, solubility and diffusion characteristics of the active agent carried by or entrapped or encapsulated within the polymer, and the specific spacing and cooperative chain motion associated with each individual polymer.

Hydrophilic polymers useful within the invention are water insoluble but water swellable. Such water swollen polymers as typically referred to as hydrogels or gels. Such gels may be conveniently produced from water soluble polymer by the process of crosslinking the polymers by a suitable crosslinking agent. However, stable hydrogels may also be formed from specific polymers under defined conditions of pH, temperature and/or ionic concentration, according to know methods in the art. Typically the polymers are cross-linked, that is, cross-linked to the extent that the polymers possess good hydrophilic properties, have improved physical integrity (as compared to non cross-linked polymers of the same or similar type) and exhibit improved ability to retain within the gel network both the biologically active agent of interest and additional compounds for coadministration therewith such as a cytokine or enzyme inhibitor, while retaining the ability to release the active agent(s) at the appropriate location and time.

Generally hydrogel polymers for use within the invention are crosslinked with a difunctional cross-linking in the amount of from 0.01 to 25 weight percent, based on the weight of the monomers forming the copolymer, and more preferably from 0.1 to 20 weight percent and more often from 0.1 to 15 weight percent of the crosslinking agent. Another useful amount of a crosslinking agent is 0.1 to 10 weight percent. Tri, tetra or higher multifunctional crosslinking agents may also be employed. When such reagents are utilized, lower amounts may be required to attain equivalent crosslinking density, i.e., the degree of crosslinking, or network properties that are sufficient to contain effectively the biologically active agent(s).

The crosslinks can be covalent, ionic or hydrogen bonds with the polymer possessing the ability to swell in the presence of water containing fluids. Such crosslinkers and crosslinking reactions are known to those skilled in the art and in many cases are dependent upon the polymer system. Thus a crosslinked network may be formed by free radical copolymerization of unsaturated monomers. Polymeric hydrogels may also be formed by crosslinking preformed polymers by reacting functional groups found on the polymers such as alcohols, acids, amines with such groups as glyoxal, formaldehyde or glutaraldehyde, bis anhydrides and the like.

The polymers also may be cross-linked with any polyene, e.g. decadiene or trivinyl cyclohexane; acrylamides, such as N,N-methylene-bis(acrylamide); polyfunctional acrylates, such as trimethylol propane triacrylate; or polyfunctional vinylidene monomer containing at least 2 terminal CH.sub.2<groups, including, for example, divinyl benzene, divinyl naphthlene, allyl acrylates and the like. In certain embodiments, cross-linking monomers for use in preparing the copolymers are polyalkenyl polyethers having more than one alkenyl ether grouping per molecule, which may optionally possess alkenyl groups in which an olefinic double bond is present attached to a terminal methylene grouping (e.g., made by the etherification of a polyhydric alcohol containing at least 2 carbon atoms and at least 2 hydroxyl groups). Compounds of this class may be produced by reacting an alkenyl halide, such as allyl chloride or allyl bromide, with a strongly alkaline aqueous solution of one or more polyhydric alcohols. The product may be a complex mixture of polyethers with varying numbers of ether groups. Efficiency of the polyether cross-linking agent increases with the number of potentially polymerizable groups on the molecule. Typically, polyethers containing an average of two or more alkenyl ether groupings per molecule are used. Other cross-linking monomers include for example, diallyl esters, dimethallyl ethers, allyl or methallyl acrylates and acrylamides, tetravinyl silane, polyalkenyl methanes, diacrylates, and dimethacrylates, divinyl compounds such as divinyl benzene, polyallyl phosphate, diallyloxy compounds and phosphite esters and the like. Typical agents are allyl pentaerythritol, allyl sucrose, trimethylolpropane triacrylate, 1,6-hexanediol diacrylate, trimethylolpropane diallyl ether, pentaerythritol triacrylate, tetramethylene dimethacrylate, ethylene diacrylate, ethylene dimethacrylate, triethylene glycol dimethacrylate, and the like. Allyl pentaerythritol, trimethylolpropane diallylether and allyl sucrose provide suitable polymers. When the cross-linking agent is present, the polymeric mixtures usually contain between about 0.01 to 20 weight percent, e.g., 1%, 5%, or 10% or more by weight of cross-linking monomer based on the total of carboxylic acid monomer, plus other monomers.

In more detailed aspects of the invention, mucosal delivery of interferon-β peptides, proteins, analogs and mimetics, and other biologically active agents disclosed herein, is enhanced by retaining the active agent(s) in a slow-release or enzymatically or physiologically protective carrier or vehicle, for example a hydrogel that shields the active agent from the action of the degradative enzymes. In certain embodiments, the active agent is bound by chemical means to the carrier or vehicle, to which may also be admixed or bound additional agents such as enzyme inhibitors, cytokines, etc. The active agent may alternately be immobilized through sufficient physical entrapment within the carrier or vehicle, e.g., a polymer matrix.

Polymers such as hydrogels useful within the invention may incorporate functional linked agents such as glycosides chemically incorporated into the polymer for enhancing intranasal bioavailability of active agents formulated therewith. Examples of such glycosides are glucosides, fructosides, galactosides, arabinosides, mannosides and their alkyl substituted derivatives and natural glycosides such as arbutin, phlorizin, amygdalin, digitonin, saponin, and indican. There are several ways in which a typical glycoside may be bound to a polymer. For example, the hydrogen of the hydroxyl groups of a glycoside or other similar carbohydrate may be replaced by the alkyl group from a hydrogel polymer to form an ether. Also, the hydroxyl groups of the glycosides may be reacted to esterify the carboxyl groups of a polymeric hydrogel to form polymeric esters in situ. Another approach is to employ condensation of acetobromoglucose with cholest-5-en-3beta-ol on a copolymer of maleic acid. N-substituted polyacrylamides can be synthesized by the reaction of activated polymers with omega-aminoalkylglycosides: (1) (carbohydrate-spacer)(n)-polyacrylamide, ‘pseudopolysaccharides’; (2) (carbohydrate spacer)(n)-phosphatidylethanolamine(m)-polyacrylamide, neoglycolipids, derivatives of phosphatidylethanolamine; (3) (carbohydrate-spacer)(n)-biotin(m)-polyacrylamide. These biotinylated derivatives may attach to lectins on the mucosal surface to facilitate absorption of the biologically active agent(s), e.g., a polymer-encapsulated interferon-βprotein or peptide.

Within more detailed aspects of the invention, one or more interferon-β peptides, proteins, analogs and mimetics, and/or other biologically active agents, disclosed herein, optionally including secondary active agents such as protease inhibitor(s), cytokine(s), additional modulator(s) of intercellular junctional physiology, etc., are modified and bound to a polymeric carrier or matrix. For example, this may be accomplished by chemically binding a peptide or protein active agent and other optional agent(s) within a crosslinked polymer network. It is also possible to chemically modify the polymer separately with an interactive agent such as a glycosidal containing molecule. In certain aspects, the biologically active agent(s), and optional secondary active agent(s), may be functionalized, i.e., wherein an appropriate reactive group is identified or is chemically added to the active agent(s). Most often an ethylenic polymerizable group is added, and the functionalized active agent is then copolymerized with monomers and a crosslinking agent using a standard polymerization method such as solution polymerization (usually in water), emulsion, suspension or dispersion polymerization. Often, the functionalizing agent is provided with a high enough concentration of functional or polymerizable groups to insure that several sites on the active agent(s) are functionalized. For example, in a polypeptide comprising 16 amine sites, it is generally desired to functionalize at least 2, 4, 5, 7, and up to 8 or more of said sites.

After functionalization, the functionalized active agent(s) is/are mixed with monomers and a crosslinking agent that comprise the reagents from which the polymer of interest is formed. Polymerization is then induced in this medium to create a polymer containing the bound active agent(s). The polymer is then washed with water or other appropriate solvents and otherwise purified to remove trace unreacted impurities and, if necessary, ground or broken up by physical means such as by stirring, forcing it through a mesh, ultrasonication or other suitable means to a desired particle size. The solvent, usually water, is then removed in such a manner as to not denature or otherwise degrade the active agent(s). One desired method is lyophilization (freeze drying) but other methods are available and may be used (e.g., vacuum drying, air drying, spray drying, etc.).

To introduce polymerizable groups in peptides, proteins and other active agents within the invention, it is possible to react available amino, hydroxyl, thiol and other reactive groups with electrophiles containing unsaturated groups. For example, unsaturated monomers containing N-hydroxy succinimidyl groups, active carbonates such as p-nitrophenyl carbonate, trichlorophenyl carbonates, tresylate, oxycarbonylimidazoles, epoxide, isocyanates and aldehyde, and unsaturated carboxymethyl azides and unsaturated orthopyridyl-disulfide belong to this category of reagents. Illustrative examples of unsaturated reagents are allyl glycidyl ether, allyl chloride, allylbromide, allyl iodide, acryloyl chloride, allyl isocyanate, allylsulfonyl chloride, maleic anhydride, copolymers of maleic anhydride and allyl ether, and the like.

All of the lysine active derivatives, except aldehyde, can generally react with other amino acids such as imidazole groups of histidine and hydroxyl groups of tyrosine and the thiol groups of cystine if the local environment enhances nucleophilicity of these groups. Aldehyde containing functionalizing reagents are specific to lysine. These types of reactions with available groups from lysines, cysteines, tyrosine have been extensively documented in the literature and are known to those skilled in the art.

In the case of biologically active agents that contain amine groups, it is convenient to react such groups with an acyloyl chloride, such as acryloyl chloride, and introduce the polymerizable acrylic group onto the reacted agent. Then during preparation of the polymer, such as during the crosslinking of the copolymer of acrylamide and acrylic acid, the functionalized active agent, through the acrylic groups, is attached to the polymer and becomes bound thereto.

In additional aspects of the invention, biologically active agents, including peptides, proteins, nucleosides, and other molecules which are bioactive in vivo, are conjugation-stabilized by covalently bonding one or more active agent(s) to a polymer incorporating as an integral part thereof both a hydrophilic moiety, e.g., a linear polyalkylene glycol, a lipophilic moiety (see, e.g., U.S. Pat. No. 5,681,811). In one aspect, a biologically active agent is covalently coupled with a polymer comprising (i) a linear polyalkylene glycol moiety and (ii) a lipophilic moiety, wherein the active agent, linear polyalkylene glycol moiety, and the lipophilic moiety are conformationally arranged in relation to one another such that the active therapeutic agent has an enhanced in vivo resistance to enzymatic degradation (i.e., relative to its stability under similar conditions in an unconjugated form devoid of the polymer coupled thereto). In another aspect, the conjugation-stabilized formulation has a three-dimensional conformation comprising the biologically active agent covalently coupled with a polysorbate complex comprising (i) a linear polyalkylene glycol moiety and (ii) a lipophilic moiety, wherein the active agent, the linear polyalkylene glycol moiety and the lipophilic moiety are conformationally arranged in relation to one another such that (a) the lipophilic moiety is exteriorly available in the three-dimensional conformation, and (b) the active agent in the composition has an enhanced in vivo resistance to enzymatic degradation.

In a further related aspect, a multiligand conjugated complex is provided which comprises a biologically active agent covalently coupled with a triglyceride backbone moiety through a polyalkylene glycol spacer group bonded at a carbon atom of the triglyceride backbone moiety, and at least one fatty acid moiety covalently attached either directly to a carbon atom of the triglyceride backbone moiety or covalently joined through a polyalkylene glycol spacer moiety (see, e.g., U.S. Pat. No. 5,681,811). In such a multiligand conjugated therapeutic agent complex, the alpha′ and beta carbon atoms of the triglyceride bioactive moiety may have fatty acid moieties attached by covalently bonding either directly thereto, or indirectly covalently bonded thereto through polyalkylene glycol spacer moieties. Alternatively, a fatty acid moiety may be covalently attached either directly or through a polyalkylene glycol spacer moiety to the alpha and alpha′ carbons of the triglyceride backbone moiety, with the bioactive therapeutic agent being covalently coupled with the gamma-carbon of the triglyceride backbone moiety, either being directly covalently bonded thereto or indirectly bonded thereto through a polyalkylene spacer moiety. It will be recognized that a wide variety of structural, compositional, and conformational forms are possible for the multiligand conjugated therapeutic agent complex comprising the triglyceride backbone moiety, within the scope of the invention. It is further noted that in such a multiligand conjugated therapeutic agent complex, the biologically active agent(s) may advantageously be covalently coupled with the triglyceride modified backbone moiety through alkyl spacer groups, or alternatively other acceptable spacer groups, within the scope of the invention. As used in such context, acceptability of the spacer group refers to steric, compositional, and end use application specific acceptability characteristics.

In yet additional aspects of the invention, a conjugation-stabilized complex is provided which comprises a polysorbate complex comprising a polysorbate moiety including a triglyceride backbone having covalently coupled to alpha, alpha′ and beta carbon atoms thereof functionalizing groups including (i) a fatty acid group; and (ii) a polyethylene glycol group having a biologically active agent or moiety covalently bonded thereto, e.g., bonded to an appropriate functionality of the polyethylene glycol group (see, e.g., U.S. Pat. No. 5,681,811). Such covalent bonding may be either direct, e.g., to a hydroxy terminal functionality of the polyethylene glycol group, or alternatively, the covalent bonding may be indirect, e.g., by reactively capping the hydroxy terminus of the polyethylene glycol group with a terminal carboxy functionality spacer group, so that the resulting capped polyethylene glycol group has a terminal carboxy functionality to which the biologically active agent or moiety may be covalently bonded.

In yet additional aspects of the invention, a stable, aqueously soluble, conjugation-stabilized complex is provided which comprises one or more interferon-β peptides, proteins, analogs and mimetics, and/or other biologically active agent(s)+ disclosed herein covalently coupled to a physiologically compatible polyethylene glycol (PEG) modified glycolipid moiety. In such complex, the biologically active agent(s) may be covalently coupled to the physiologically compatible PEG modified glycolipid moiety by a labile covalent bond at a free amino acid group of the active agent, wherein the labile covalent bond is scissionable in vivo by biochemical hydrolysis and/or proteolysis. The physiologically compatible PEG modified glycolipid moiety may advantageously comprise a polysorbate polymer, e.g., a polysorbate polymer comprising fatty acid ester groups selected from the group consisting of monopalmitate, dipalmitate, monolaurate, dilaurate, trilaurate, monoleate, dioleate, trioleate, monostearate, distearate, and tristearate. In such complex, the physiologically compatible PEG modified glycolipid moiety may suitably comprise a polymer selected from the group consisting of polyethylene glycol ethers of fatty acids, and polyethylene glycol esters of fatty acids, wherein the fatty acids for example comprise a fatty acid selected from the group consisting of lauric, palmitic, oleic, and stearic acids.

Bioadhesive Delivery Vehicles and Methods

In certain aspects of the invention, the combinatorial formulations and/or coordinate administration methods herein incorporate an effective amount of a nontoxic bioadhesive as an adjunct compound or carrier to enhance mucosal delivery of one or more biologically active agent(s). Bioadhesive agents in this context exhibit general or specific adhesion to one or more components or surfaces of the targeted mucosa. The bioadhesive maintains a desired concentration gradient of the biologically active agent into or across the mucosa to ensure penetration of even large molecules (e.g., peptides and proteins) into or through the mucosal epithelium. Typically, employment of a bioadhesive within the methods and compositions of the invention yields a two- to five-fold, often a five- to ten-fold increase in permeability for peptides and proteins into or through the mucosal epithelium. This enhancement of epithelial permeation often permits effective transmucosal delivery of large macromolecules, for example to the basal portion of the nasal epithelium or into the adjacent extracellular compartments or the systemic circulation or CNS.

This enhanced delivery provides for greatly improved effectiveness of delivery of bioactive peptides, proteins and other macromolecular therapeutic species. These results will depend in part on the hydrophilicity of the compound, whereby greater penetration will be achieved with hydrophilic species compared to water insoluble compounds. In addition to these effects, employment of bioadhesives to enhance drug persistence at the mucosal surface can elicit a reservoir mechanism for protracted drug delivery, whereby compounds not only penetrate across the mucosal tissue but also back-diffuse toward the mucosal surface once the material at the surface is depleted.

A variety of suitable bioadhesives are disclosed in the art for oral administration (see, e.g., U.S. Pat. Nos. 3,972,995; 4,259,314; 4,680,323; 4,740,365; 4,573,996; 4,292,299; 4,715,369; 4,876,092; 4,855,142; 4,250,163; 4,226,848; 4,948,580; U.S. Pat. No. Reissue 33,093; and Robinson, 18 Proc. Intern. Symp. Control. Rel. Bioact. Mater. 75 (1991), which find use within the novel methods and compositions of the invention. The potential of various bioadhesive polymers as a mucosal, e.g., nasal, delivery platform within the methods and compositions of the invention can be readily assessed by determining their ability to retain and release a specific biologically active agent, e.g., a interferon-β peptide or protein, as well as by their capacity to interact with the mucosal surfaces following incorporation of the active agent therein. In addition, well known methods will be applied to determine the biocompatibility of selected polymers with the tissue at the site of mucosal administration. One aspect of polymer biocompatibility is the potential effect for the polymer to induce a cytokine response. In certain circumstances, implanted polymers have been shown to induce the release of inflammatory cytokines from adhering cells, such as monocytes and macrophages. Similar potential adverse reactions of mucosal epithelial cells in contact with candidate bioadhesive polymers will be determined using routine in vitro and in vivo assays. Since epithelial cells have the ability to secrete a number of cytokines, the induction of cytokine responses in epithelial cells will often provide an adequate measure of biocompatibility of a selected polymer delivery platform.

When the target mucosa is covered by mucus (i.e., in the absence of mucolytic or mucus-clearing treatment), it can serve as a connecting link to the underlying mucosal epithelium. Therefore, the term “bioadhesive” as used herein also covers mucoadhesive compounds useful for enhancing mucosal delivery of biologically active agents within the invention. However, adhesive contact to mucosal tissue mediated through adhesion to a mucus gel layer may be limited by incomplete or transient attachment between the mucus layer and the underlying tissue, particularly at nasal surfaces where rapid mucus clearance occurs. In this regard, mucin glycoproteins are continuously secreted and, immediately after their release from cells or glands, form a viscoelastic gel. The luminal surface of the adherent gel layer, however, is continuously eroded by mechanical, enzymatic and/or ciliary action. Where such activities are more prominent, or where longer adhesion times are desired, the coordinate administration methods and combinatorial formulation methods of the invention may further incorporate mucolytic and/or ciliostatic methods or agents as disclosed herein above.

Bioadhesive and other delivery enhancing agents within the methods and compositions of the invention can improve the effectiveness of a treatment by helping maintain the drug concentration between effective and toxic levels, by inhibiting dilution of the drug away from the delivery point, and improving targeting and localization of the drug. In this context, bioadhesion increases the intimacy and duration of contact between a drug-containing polymer and the mucosal surface. The combined effects of this enhanced, direct drug absorption, and the decrease in excretion rate that results from reduced diffusion and improved localization, significantly enhances bioavailability of the drug and allows for a smaller dosage and less frequent administration.

Typically, mucoadhesive polymers for use within the invention are natural or synthetic macromolecules which adhere to wet mucosal tissue surfaces by complex, but non-specific, mechanisms. In addition to these mucoadhesive polymers, the invention also provides methods and compositions incorporating bioadhesives that adhere directly to a cell surface, rather than to mucus, by means of specific, including receptor-mediated, interactions. One example of bioadhesives that function in this specific manner is the group of compounds known as lectins. These are glycoproteins with an ability to specifically recognize and bind to sugar molecules, e.g. glycoproteins or glycolipids, which form part of intranasal epithelial cell membranes and can be considered as “lectin receptors”.

In various embodiments, the coordinate administration methods of the invention optionally incorporate bioadhesive materials that yield prolonged residence time at the mucosal surface. Alternatively, the bioadhesive material may otherwise facilitate mucosal absorption of the biologically active agent, e.g., by facilitating localization of the active agent to a selected target site of activity (e.g., bloodstream or CNS). In additional aspects, adjunct delivery or combinatorial formulation of bioadhesive agents within the methods and compositions of the invention intensify contact of the biologically active agent with the target mucosa, including by increasing epithelial permeability, (e.g., to effectively increase the drug concentration gradient). In further alternate embodiments, bioadhesives and other polymers disclosed herein serve to inhibit proteolytic or other enzymes that might degrade the biologically active agent. For a review of different approaches to bioadhesion that are useful within the coordinate administration, multi-processing and/or combinatorial formulation methods and compositions of the invention, see, e.g., Lehr C. M., Eur J. Drug Metab. Pharmacokinetics 21(2):139-148, 1996.

In certain aspects of the invention, bioadhesive materials for enhancing intranasal delivery of biologically active agents comprise a matrix of a hydrophilic, e.g., water soluble or swellable, polymer or a mixture of polymers that can adhere to a wet mucous surface. These adhesives may be formulated as ointments, hydrogels (see above) thin films, and other application forms. Often, these adhesives have the biologically active agent mixed therewith to effectuate slow release or local delivery of the active agent. Some are formulated with additional ingredients to facilitate penetration of the active agent through the nasal mucosa, e.g., into the circulatory system of the individual.

Various polymers, both natural and synthetic ones, show significant binding to mucus and/or mucosal epithelial surfaces under physiological conditions. The strength of this interaction can readily be measured by mechanical peel or shear tests. A variety of suitable test methods and instruments to serve such purposes are known in the art (see, e.g., Gu et al., Crit. Rev. Ther. Drug Carrier Syst. 5:21-67, 1988; Duchene et al., Drug Dev. Ind. Pharm. 14:283-318, 1988). When applied to a humid mucosal surface, many dry materials will spontaneously adhere, at least slightly. After such an initial contact, some hydrophilic materials start to attract water by adsorption, swelling or capillary forces, and if this water is absorbed from the underlying substrate or from the polymer-tissue interface, the adhesion may be sufficient to achieve the goal of enhancing mucosal absorption of biologically active agents (see, e.g., Al-Dujaili et al., Int. J. Pharm. 34:75-79, 1986; Marvola et al., J. Pharm. Sci. 72:1034-1036, 1983; Marvola et al., J. Pharm. Sci. 71:975-977, 1982; and Swisher et al., Int. J. Pharm. 22:219, 1984; Chen, et al., Adhesion in Biological Systems, p. 172, Manly, Ed., Academic Press, London, 1970). Such ‘adhesion by hydration’ can be quite strong, but formulations adapted to employ this mechanism must account for swelling which continues as the dosage transforms into a hydrated mucilage. This is projected for many hydrocolloids useful within the invention, especially some cellulose-derivatives, which are generally non-adhesive when applied in pre-hydrated state. Nevertheless, bioadhesive drug delivery systems for mucosal administration are effective within the invention when such materials are applied in the form of a dry polymeric powder, microsphere, or film-type delivery form.

Other polymers adhere to mucosal surfaces not only when applied in dry, but also in fully hydrated state, and in the presence of excess amounts of water. The selection of a mucoadhesive thus requires due consideration of the conditions, physiological as well as physico-chemical, under which the contact to the tissue will be formed and maintained. In particular, the amount of water or humidity usually present at the intended site of adhesion, and the prevailing pH, are known to largely affect the mucoadhesive binding strength of different polymers.

Several polymeric bioadhesive drug delivery systems have been fabricated and studied in the past 20 years, not always with success. A variety of such carriers are, however, currently used in clinical applications involving dental, orthopedic, ophthalmological, and surgical uses. For example, acrylic-based hydrogels have been used extensively for bioadhesive devices. Acrylic-based hydrogels are well-suited for bioadhesion due to their flexibility and nonabrasive characteristics in the partially swollen state which reduce damage-causing attrition to the tissues in contact. Furthermore, their high permeability in the swollen state allows unreacted monomer, un-crosslinked polymer chains, and the initiator to be washed out of the matrix after polymerization, which is an important feature for selection of bioadhesive materials for use within the invention. Acrylic-based polymer devices exhibit very high adhesive bond strength, as determined by various known methods (Park et al., J. Control. Release 2:47-57, 1985; Park et al., Pharm. Res. 4:457-464, 1987; and Ch'ng et al., J. Pharm. Sci. 74:399-405, 1985).

For controlled mucosal delivery of peptide and protein drugs, the methods and compositions of the invention optionally include the use of carriers, e.g., polymeric delivery vehicles, that function in part to shield the biologically active agent from proteolytic breakdown, while at the same time providing for enhanced penetration of the peptide or protein into or through the nasal mucosa. In this context, bioadhesive polymers have demonstrated considerable potential for enhancing oral drug delivery.

In addition to protecting against enzymatic degradation, bioadhesives and other polymeric or non-polymeric absorption-promoting agents for use within the invention may directly increase mucosal permeability to biologically active agents. To facilitate the transport of large and hydrophilic molecules, such as peptides and proteins, across the nasal epithelial barrier, mucoadhesive polymers and other agents have been postulated to yield enhanced permeation effects beyond what is accounted for by prolonged premucosal residence time of the delivery system. In other studies using in vitro cultured epithelial cell monolayers, it was reported that dry, swellable materials such as starch microspheres induce reversible focal dilations of the tight junctions, allowing for enhanced drug transport along the paracellular route. According to this adhesion-dehydration theory, the hydrophilic polymer, applied as a dry powder, absorbs water from the mucosal tissue in such a way that the epithelial cells are dehydrated and shrink until the normally tight intercellular junctions between the cells become physically separated. Because this effect is of relatively short duration and appears to be completely reversible, it provides yet another useful tool for incorporation within the coordinate administration, multi-processing and/or combinatorial formulation methods and compositions of the invention.

Other mucoadhesive polymers for use within the invention, for example chitosan, reportedly enhance the permeability of certain mucosal epithelia even when they are applied as an aqueous solution or gel. In one study, absorption of the peptide drugs insulin and calcitonin, and the hydrophilic compound phenol red, from an aqueous gel base of poly(acrylic acid) was reported after rectal, vaginal and nasal administration. Another mucoadhesive polymer reported to directly affect epithelial permeability is hyaluronic acid. In particular, hyaluronic acid gel formulation reportedly enhanced nasal absorption of vasopressin and some of its analogues. Ester derivatives of hyaluronic acid in the form of lyophilized microspheres were described as a nasal delivery system for insulin (Illum et al., J. Contr. Rel. 29:133-141, 1994).

A particularly useful bioadhesive agent within the coordinate administration, and/or combinatorial formulation methods and compositions of the invention is chitosan, as well as its analogs and derivatives. Chitosan is a non-toxic, biocompatible and biodegradable polymer that is widely used for pharmaceutical and medical applications because of its favorable properties of low toxicity and good biocompatibility (Yomota, Pharm. Tech. Japan 10:557-564, 1994). It is a natural polyaminosaccharide prepared from chitin by N-deacetylation with alkali. Furthermore, chitosan has been reported to promote absorption of small polar molecules and peptide and protein drugs through nasal mucosa in animal models and human volunteers

As used within the methods and compositions of the invention, chitosan increases the retention of interferon-β peptides, proteins, analogs and mimetics, and other biologically active agents disclosed herein at a mucosal site of application. This is may be mediated in part by a positive charge characteristic of chitosan, which may influence epithelial permeability even after physical removal of chitosan from the surface. As with other bioadhesive gels provided herein, the use of chitosan can reduce the frequency of application and the amount of biologically active agent administered while yielding an effective delivery amount or dose. This mode of administration can also improve patient compliance and acceptance. The occlusion and lubrication of chitosan and other bioadhesive gels is expected to reduce the discomfort of inflammatory, allergic and ulcerative conditions of the nasal mucosa.

As further provided herein, the methods and compositions of the invention will optionally include a novel chitosan derivative or chemically modified form of chitosan. One such novel derivative for use within the invention is denoted as a β-[1→4]-2-guanidino-2-deoxy-D-glucose polymer (poly-GuD). Chitosan is the N-deacetylated product of chitin, a naturally occurring polymer that has been used extensively to prepare microspheres for oral and intra-nasal formulations. The chitosan polymer has also been proposed as a soluble carrier for parenteral drug delivery. Within one aspect of the invention, o-methylisourea is used to convert a chitosan amine to its guanidinium moiety. The guanidinium compound is prepared, for example, by the reaction between equi-normal solutions of chitosan and o-methylisourea at pH above 8.0.

The guanidinium product is -[14]-guanidino-2-deoxy-D-glucose polymer. It is abbreviated as Poly-GuD in this context (Monomer F.W. of Amine in Chitosan=161; Monomer F.W. of Guanidinium in Poly-GuD=203).

One exemplary Poly-GuD preparation method for use within the invention involves the following protocol.

Solutions:

Preparation of 0.5% Acetic Acid Solution (0.088N):

Pipette 2.5 mL glacial acetic acid into a 500 mL volumetric flask, dilute to volume with purified water.

Preparation of 2N NaOH Solution:

Transfer about 20 g NaOH pellets into a beaker with about 150 mL of purified water. Dissolve and cool to room temperature. Transfer the solution into a 250-mL volumetric flask, dilute to volume with purified water.

Preparation of O-methylisourea Sulfate (0.4N urea group equivalent):

Transfer about 493 mg of O-methylisourea sulfate into a 10-mL volumetric flask, dissolve and dilute to volume with purified water.

The pH of the solution is 4.2

Preparation of Barium Chloride Solution (0.2M):

Transfer about 2.086 g of Barium chloride into a 50-mL volumetric flask, dissolve and dilute to volume with purified water.

Preparation of Chitosan Solution (0.06N amine equivalent):

Transfer about 100 mg Chitosan into a 50 mL beaker, add 10 mL 0.5% Acetic Acid (0.088 N). Stir to dissolve completely.

The pH of the solution is about 4.5

Preparation of O-methylisourea Chloride Solution (0.2N urea group equivalent):

Pipette 5.0 mL of O-methylisourea sulfate solution (0.4 N urea group equivalent) and 5 mL of 0.2M Barium chloride solution into a beaker. A precipitate is formed. Continue to mix the solution for additional 5 minutes. Filter the solution through 0.45 m filter and discard the precipitate. The concentration of O-methylisourea chloride in the supernatant solution is 0.2 N urea group equivalent.

The pH of the solution is 4.2.

Procedure:

Add 1.5 mL of 2 N NaOH to 10 mL of the chitosan solution (0.06N amine equivalent) prepared as described in Section 2.5. Adjust the pH of the solution with 2N NaOH to about 8.2 to 8.4. Stir the solution for additional 10 minutes. Add 3.0 mL O-methylisourea chloride solution (0.2N urea group equivalent) prepared as described above. Stir the solution overnight.

Adjust the pH of solution to 5.5 with 0.5% Acetic Acid (0.088N).

Dilute the solution to a final volume of 25 mL using purified water.

The Poly-GuD concentration in the solution is 5 mg/mL, equivalent to 0.025 N (guanidium group).

Additional compounds classified as bioadhesive agents for use within the present invention act by mediating specific interactions, typically classified as “receptor-ligand interactions” between complementary structures of the bioadhesive compound and a component of the mucosal epithelial surface. Many natural examples illustrate this form of specific binding bioadhesion, as exemplified by lectin-sugar interactions. Lectins are (glyco)proteins of non-immune origin which bind to polysaccharides or glycoconjugates. Several plant lectins have been investigated as possible pharmaceutical absorption-promoting agents. One plant lectin, Phaseolus vulgaris hemagglutinin (PHA), exhibits high oral bioavailability of more than 10% after feeding to rats. In contrast, tomato (Lycopersicon esculeutum) lectin (TL) appears safe for various modes of administration. This glycoprotein (approximately 70 kDa) resists digestion and binds to rat intestinal villi without inducing any deleterious effects.

Therefore, the invention provides for coordinate administration or combinatorial formulation of non-toxic lectins identified or obtained by modification of existing lectins which have a high specific affinity for mucosal, e.g., nasal epithelial, cells, but low cross reactivity with mucus. In this regard, detailed teachings regarding lectin structure-activity relationships will allow selection of non-toxic, strongly bioadhesive candidates to produce optimized lectins for therapeutic purposes (see, e.g., Lehr et al., Lectins: Biomedical Perspectives, pp. 117-140, Pustai et al., Eds., Taylor and Francis, London, 1995). In additional embodiments of the invention, mucolytic agents and/or ciliostatic agents are coordinately administered or combinatorially formulated with a biologically active agent and a lectin or other specific binding bioadhesive—in order to counter the effects of non-specific binding of the bioadhesive to mucosal mucus.

In addition to the use of lectins, certain antibodies or amino acid sequences exhibit high affinity binding to complementary elements on cell and mucosal surfaces. Thus, for example, various adhesive amino acids sequences such as Arg-Gly-Asp and others, if attached to a carrier matrix, will promote adhesion by binding with specific cell surface glycoproteins. In other embodiments, adhesive ligand components are integrated in a carrier or delivery vehicle that selectively adheres to a particular cell type, or diseased target tissue.

In summary, the foregoing bioadhesive agents are useful in the combinatorial formulations and coordinate administration methods of the instant invention, which optionally incorporate an effective amount and form of a bioadhesive agent to prolong persistence or otherwise increase mucosal absorption of one or more interferon-β peptides, proteins, analogs and mimetics, and other biologically active agents. The bioadhesive agents may be coordinately administered as adjunct compounds or as additives within the combinatorial formulations of the invention. In certain embodiments, the bioadhesive agent acts as a ‘pharmaceutical glue’, whereas in other embodiments adjunct delivery or combinatorial formulation of the bioadhesive agent serves to intensify contact of the biologically active agent with the nasal mucosa, in some cases by promoting specific receptor-ligand interactions with epithelial cell “receptors”, and in others by increasing epithelial permeability to significantly increase the drug concentration gradient measured at a target site of delivery (e.g., the CNS or in the systemic circulation). Yet additional bioadhesive agents for use within the invention act as enzyme (e.g., protease) inhibitors to enhance the stability of mucosally administered biotherapeutic agents delivered coordinately or in a combinatorial formulation with the bioadhesive agent.

Liposomes and Micellar Delivery Vehicles

The coordinate administration methods and combinatorial formulations of the instant invention optionally incorporate effective lipid or fatty acid based carriers, processing agents, or delivery vehicles, to provide improved formulations for mucosal delivery of interferon-β peptides, proteins, analogs and mimetics, and other biologically active agents. For example, a variety of formulations and methods are provided for mucosal delivery which comprise one or more of these active agents, such as a peptide or protein, admixed or encapsulated by, or coordinately administered with, a liposome, mixed micellar carrier, or emulsion, to enhance chemical and physical stability and increase the half life of the biologically active agents (e.g., by reducing susceptibility to proteolysis, chemical modification and/or denaturation) upon mucosal delivery.

Within certain aspects of the invention, specialized delivery systems for biologically active agents comprise small lipid vesicles known as liposomes (see, e.g., Chonn et al., Curr. Opin. Biotechnol. 6:698-708, 1995; Lasic, Trends Biotechnol. 16:307-321, 1998; and Gregoriadis, Trends Biotechnol. 13:527-537, 1995). These are typically made from natural, biodegradable, non-toxic, and non-immunogenic lipid molecules, and can efficiently entrap or bind drug molecules, including peptides and proteins, into, or onto, their membranes. The attractiveness of liposomes as a peptide and protein delivery system within the invention is increased by the fact that the encapsulated proteins can remain in their preferred aqueous environment within the vesicles, while the liposomal membrane protects them against proteolysis and other destabilizing factors. Even though not all liposome preparation methods known are feasible in the encapsulation of peptides and proteins due to their unique physical and chemical properties, several methods allow the encapsulation of these macromolecules without substantial deactivation (see, e.g., Weiner, Immunomethods 4:201-209, 1994).

A variety of methods are available for preparing liposomes for use within the invention (e.g., as described in Szoka et al., Ann. Rev. Biophys. Bioeng. 9:467, 1980; and U.S. Pat. Nos. 4,235,871, 4,501,728, and 4,837,028). For use with liposome delivery, the biologically active agent is typically entrapped within the liposome, or lipid vesicle, or is bound to the outside of the vesicle. Several strategies have been devised to increase the effectiveness of liposome-mediated delivery by targeting liposomes to specific tissues and specific cell types. Liposome formulations, including those containing a cationic lipid, have been shown to be safe and well tolerated in human patients (Treat et al., J. Natl. Cancer Instit. 82:1706-1710, 1990).

Like liposomes, unsaturated long chain fatty acids, which also have enhancing activity for mucosal absorption, can form closed vesicles with bilayer-like structures (so called “ufasomes”). These can be formed, for example, using oleic acid to entrap biologically active peptides and proteins for mucosal, e.g., intranasal, delivery within the invention.

More simplified delivery systems for use within the invention include the use of cationic lipids as delivery vehicles or carriers, which can be effectively employed to provide an electrostatic interaction between the lipid carrier and such charged biologically active agents as proteins and polyanionic nucleic acids (see, e.g., Hope et al., Molecular Membrane Biology 15:1-14, 1998). This allows efficient packaging of the drugs into a form suitable for mucosal administration and/or subsequent delivery to systemic compartments. These and related systems are particularly well suited for delivery of polymeric nucleic acids, e.g., in the form of gene constructs, antisense oligonucleotides and ribozymes. These drugs are large, usually negatively charged molecules with molecular weights on the order of 106 for a gene to 103 for an oligonucleotide. The targets for these drugs are intracellular, but their physical properties prevent them from crossing cell membranes by passive diffusion as with conventional drugs. Furthermore, unprotected DNA is degraded within minutes by nucleases present in normal plasma. To avoid inactivation by endogenous nucleases, antisense oligonucleotides and ribozymes can be chemically modified to be enzyme resistant by a variety of known methods, but plasmid DNA must ordinarily be protected by encapsulation in viral or non-viral envelopes, or condensation into a tightly packed particulate form by polycations such as proteins or cationic lipid vesicles. More recently, small unilamellar vesicles (SUVs) composed of a cationic lipid and dioleoylphosphatidylethanolamine (DOPE) have been successfully employed as vehicles for polynucleic acids, such as plasmid DNA, to form particles capable of transportation of the active polynucleotide across plasma membranes into the cytoplasm of a broad spectrum of cells. This process (referred to as lipofection or cytofection) is now widely employed as a means of introducing plasmid constructs into cells to study the effects of transient gene expression. Exemplary delivery vehicles of this type for use within the invention include cationic lipids (e.g., N-(2,3-(dioleyloxy)propyl)-N,N,N-trimethyl am-monium chloride (DOTMA)), quarternary ammonium salts (e.g., N,N-dioleyl-N,N-dimethylammonium chloride (DODAC)), cationic derivatives of cholesterol (e.g., 3β(N-(N′,N-dimethylaminoethane-carbamoyl-cholesterol (DC-chol)), and lipids characterized by multivalent headgroups (e.g., dioctadecyldimethylammonium chloride (DOGS), commercially available as Transfectam®).

Additional delivery vehicles for use within the invention include long and medium chain fatty acids, as well as surfactant mixed micelles with fatty acids (see, e.g., Muranishi, Crit. Rev. Ther. Drug Carrier Syst. 7:1-33, 1990). Most naturally occurring lipids in the form of esters have important implications with regard to their own transport across mucosal surfaces. Free fatty acids and their monoglycerides which have polar groups attached have been demonstrated in the form of mixed micelles to act on the intestinal barrier as penetration enhancers. This discovery of barrier modifying function of free fatty acids (carboxylic acids with a chain length varying from 12 to 20 carbon atoms) and their polar derivatives has stimulated extensive research on the application of these agents as mucosal absorption enhancers.

For use within the methods of the invention, long chain fatty acids, especially fusogenic lipids (unsaturated fatty acids and monoglycerides such as oleic acid, linoleic acid, linoleic acid, monoolein, etc.) provide useful carriers to enhance mucosal delivery of interferon-β peptides, proteins, analogs and mimetics, and other biologically active agents disclosed herein. Medium chain fatty acids (C6 to C12) and monoglycerides have also been shown to have enhancing activity in intestinal drug absorption and can be adapted for use within the mocosal delivery formulations and methods of the invention. In addition, sodium salts of medium and long chain fatty acids are effective delivery vehicles and absorption-enhancing agents for mucosal delivery of biologically active agents within the invention. Thus, fatty acids can be employed in soluble forms of sodium salts or by the addition of non-toxic surfactants, e.g., polyoxyethylated hydrogenated castor oil, sodium taurocholate, etc. Mixed micelles of naturally occurring unsaturated long chain fatty acids (oleic acid or linoleic acid) and their monoglycerides with bile salts have been shown to exhibit absorption-enhancing abilities which are basically harmless to the intestinal mucosa (see, e.g., Muranishi, Pharm. Res. 2:108-118, 1985; and Crit. Rev. Ther. drug carrier Syst. 7:1-33, 1990). Other fatty acid and mixed micellar preparations that are useful within the invention include, but are not limited to, Na caprylate (C8), Na caprate (C10), Na laurate (C12) or Na oleate (C18), optionally combined with bile salts, such as glycocholate and taurocholate.

Pegylation

Additional methods and compositions provided within the invention involve chemical modification of biologically active peptides and proteins by covalent attachment of polymeric materials, for example dextrans, polyvinyl pyrrolidones, glycopeptides, polyethylene glycol and polyamino acids. The resulting conjugated peptides and proteins retain their biological activities and solubility for mucosal administration. In alternate embodiments, interferon-β peptides, proteins, analogs and mimetics, and other biologically active peptides and proteins, are conjugated to polyalkylene oxide polymers, particularly polyethylene glycols (PEG) (see, e.g., U.S. Pat. No. 4,179,337). Numerous reports in the literature describe the potential advantages of pegylated peptides and proteins, which often exhibit increased resistance to proteolytic degradation, increased plasma half-life, increased solubility and decreased antigenicity and immunogenicity (Nucci, et al., Advanced Drug Deliver Reviews 6:133-155, 1991; Lu et al., Int. J. Peptide Protein Res. 43:127-138, 1994).

Several procedures have been reported for the attachment of PEG to proteins and peptides and their subsequent purification (Abuchowski et al., J. Biol. Chem. 252:3582-3586,1977; Beauchamp et al., Anal. Biochem. 131:25-33, 1983). In addition, Lu et al., Int. J. Peptide Protein Res. 43:127-138, 1994, describe various technical considerations and compare PEGylation procedures for proteins versus peptides (see also, Katre et al., Proc. Natl. Acad. Sci. USA 84:1487-1491, 1987; Becker et al., Makromol. Chem. Rapid Commun. 3:217-223, 1982; Mutter et al., Makromol. Chem. Rapid Commun. 13:151-157, 1992; Merrifield, R. B., J. Am. Chem. Soc. 85:2149-2154, 1993; Lu et al., Peptide Res. 6:142-146, 1993; Lee et al., Bioconjugate Chem. 10:973-981, 1999, Nucci et al., Adv. Drug Deliv. Rev. 6:133-151, 1991; Francis et al., J. Drug Targeting 3:321-340, 1996; Zalipsky, S., Bioconjugate Chem. 6:150-165, 1995; Clark et al., J. Biol. Chem. 271:21969-21977, 1996; Pettit et al., J. Biol. Chem. 272:2312-2318, 1997; Delgado et al., Br. J. Cancer 73:175-182, 1996; Benhar et al., Bioconjugate Chem. 5:321-326, 1994; Benhar et al., J. Biol. Chem. 269:13398-13404, 1994; Wang et al., Cancer Res. 53:4588-4594, 1993; Kinstler et al., Pharm. Res. 13:996-1002, 1996, Filpula et al., Exp. Opin. Ther. Patents 9:231-245, 1999; Pelegrin et al., Hum. Gene Ther. 9:2165-2175, 1998).

Following these and other teachings in the art, the conjugation of biologically active peptides and proteins for with polyethyleneglycol polymers, is readily undertaken, with the expected result of prolonging circulating life and/or reducing immunogenicity while maintaining an acceptable level of activity of the PEGylated active agent. Amine-reactive PEG polymers for use within the invention include SC-PEG with molecular masses of 2000, 5000, 10000, 12000, and 20 000; U-PEG-10000; NHS-PEG-3400-biotin; T-PEG-5000; T-PEG-12000; and TPC-PEG-5000. Chemical conjugation chemistries for these polymers have been published (see, e.g., Zalipsky, S., Bioconjugate Chem. 6:150-165, 1995; Greenwald et al., Bioconjugate Chem. 7:638-641, 1996; Martinez et al., Macromol. Chem. Phys. 198:2489-2498, 1997; Hermanson, G. T. , Bioconjugate Techniques, pp. 605-618, 1996; Whitlow et al., Protein Eng. 6:989-995, 1993; Habeeb, A. F. S. A., Anal. Biochem. 14:328-336, 1966; Zalipsky et al., Poly(ethyleneglycol) Chemistry and Biological Applications, pp. 318-341, 1997; Harlow et al., Antibodies: a Laboratory Manual, pp. 553-612, Cold Spring Harbor Laboratory, Plainview, N.Y., 1988; Milenic et al, Cancer Res. 51:6363-6371, 1991; Friguet et al., J. Immunol. Methods 77:305-319, 1985). While phosphate buffers are commonly employed in these protocols, the choice of borate buffers may beneficially influence the PEGylation reaction rates and resulting products.

PEGylation of biologically active peptides and proteins may be achieved by modification of carboxyl sites (e.g., aspartic acid or glutamic acid groups in addition to the carboxyl terminus). The utility of PEG-hydrazide in selective modification of carbodiimide-activated protein carboxyl groups under acidic conditions has been described (Zalipsky, S., Bioconjugate Chem. 6:150-165, 1995; Zalipsky et al., Poly(ethyleneglycol) Chemistry and Biological Applications, pp. 318-341, American Chemical Society, Washington, D.C., 1997). Alternatively, bifunctional PEG modification of biologically active peptides and proteins can be employed. In some procedures, charged amino acid residues, including lysine, aspartic acid, and glutamic acid, have a marked tendency to be solvent accessible on protein surfaces. Conjugation to carboxylic acid groups of proteins is a less frequently explored approach for production of protein bioconjugates. However, the hydrazide/EDC chemistry described by Zalipsky and colleagues (Zalipsky, S., Bioconjugate Chem. 6:150-165, 1995; Zalipsky et al., Poly(ethyleneglycol) Chemistry and Biological Applications, pp. 318-341, American Chemical Society, Washington, D.C., 1997) offers a practical method of linking PEG polymers to protein carboxylic sites.

Often, PEGylation of peptides and proteins for use within the invention involves activating PEG with a functional group that will react with lysine residues on the surface of the peptide or protein. Within certain alternate aspects of the invention, biologically active peptides and proteins are modified by PEGylation of other residues such as His, Trp, Cys, Asp, Glu, etc., without substantial loss of activity. If PEG modification of a selected peptide or protein proceeds to completion, the activity of the peptide or protein is often diminished. Therefore, PEG modification procedures herein are generally limited to partial PEGylation of the peptide or protein, resulting in less than about 50%, more commonly less than about 25%, loss of activity, while providing for substantially increased half-life (e.g., serum half life) and a substantially decreased effective dose requirement of the PEGylated active agent.

An unavoidable result of partial PEG modification is the production of a heterogenous mixture of PEGylated peptide or protein having a statistical distribution of the number of PEG groups bound per molecule. In addition, the usage of lysine residues within the peptide or protein is random. These two factors result in the production of a heterogeneous mixture of PEGylated proteins which differ in both the number and position of the PEG groups attached. For instance, when adenosine deaminase is optimally modified there is a loss of 50% activity when the protein has about 14 PEG per protein, with a broad distribution of the actual number of PEG moieties per individual protein and a broad distribution of the position of the actual lysine residues used. Such mixtures of diversely modified proteins are not optimally suited for pharmaceutical use. At the same time, purification and isolation of a class of PEGylated proteins (e.g., proteins containing the same number of PEG moieties) or a single type of PEGylated protein (e.g., proteins containing both the same number of moieties and having the PEG moieties at the same position) involves time-consuming and expensive procedures which result in an overall reduction in the yield of the specific PEGylated peptide or protein of interest.

Within certain alternate aspects of the invention, biologically active peptides and proteins are modified by PEGylation methods that employ activated PEG reagents that react with thio groups of the protein, resulting in covalent attachment of PEG to a cysteine residue, which residue may be inserted in place of a naturally-occurring lysine residue of the protein. Yet additional methods employed within the invention for generating PEGylated peptides and proteins do not require extensive knowledge of protein structure-function (e.g., mapping amino acid residues essential for biological activity). Exemplifying these methods, U.S. Pat. No. 5,766,897 describes methods for production and characterization of cysteine-PEGylated proteins suitable for therapeutic applications. These are produced by attaching a polyethylene glycol to a cysteine residue within the protein. To obtain the desired result of a stable, biologically active compound the PEG is attached in a specific manner, often to a cysteine residue present at or near a site that is normally glycosylated. Typically, the specific amino acid modified by glycosylation (e.g., asparagine in N-linked glycosylation or serine or threonine in O-linked glycosylation) is replaced by a cysteine residue, which is subsequently chemically modified by attachment of PEG. It may be useful for employment of this method to generation cysteine-containing mutants of selected biologically active peptides and proteins, which can be readily accomplished by, for example, site-directed mutagenesis using methods well known in the art. In addition, if the active peptide or protein is one member of a family of structurally related proteins, glycosylation sites for any other member can be matched to an amino acid on the protein of interest, and that amino acid changed to cysteine for attachment of the polyethylene glycol. Alternatively, if a crystal structure has been determined for the protein of interest or a related protein, surface residues away from the active site or binding site can be changed to cysteine for the attachment of polyethylene glycol.

These strategies for identifying useful PEG attachment sites for use within the invention are advantageous in that they are readily implemented without extensive knowledge of protein structure-function details. Moreover, these strategies also take advantage of the fact that the presence and location of glycosylation residues are often related, as a natural evolutionary consequence, to increased stability and serum half-life of the subject peptide or protein. Replacement of these glycosylation residues by cysteine, followed by cysteine-specific PEGylation, commonly yields modified peptides and proteins that retain substantial biological activity while exhibiting significantly increased stability.

If a higher degree of PEG modification is required, and/or if the peptide or protein to be chemically modified is not normally glycosylated, other solvent accessible residues can be changed to cysteine, and the resultant protein subjected to PEGylation. Appropriate residues can easily be determined by those skilled in the art. For instance, if a three-dimensional structure is available for the protein of interest, or a related protein, solvent accessible amino acids are easily identified. Also, charged amino acids such as Lys, Arg, Asp and Glu are almost exclusively found on the surface of proteins. Substitution of one, two or many of these residues with cysteine will provide additional sites for PEG attachment. In addition, amino acid sequences in the native protein that are recognized by antibodies are usually on the surface of the protein. These and other methods for determining solvent accessible amino acids are well known to those skilled in the art.

Modification of peptides and proteins with PEG can also be used to generate multimeric complexes of proteins, fragments, and/or peptides that have increased biological stability and/or potency. These multimeric peptides and proteins of the invention, e.g., dimers or tetramers of a interferon-β peptide or protein, may be produced synthetically according to well known methods. Alternatively, other biologically active peptides and proteins may be produced in this manner that are naturally occurring dimeric or multimeric proteins. For example, dimeric peptides and proteins useful within the invention may be produced by reacting the peptide or protein with (Maleimido)₂-PEG, a reagent composed of PEG having two protein-reactive moieties. In the case of cysteine-pegylated peptides and proteins, the degree of multimeric cross-linking can be controlled by the number of cysteines either present and/or engineered into the peptide or protein, and by the concentration of reagents, e.g., (Maleimido)₂ PEG, used in the reaction mixture.

It is further contemplated to attach other groups to thio groups of cysteines present in biologically active peptides and proteins for use within the invention. For example, the peptide or protein may be biotinylated by attaching biotin to a thio group of a cysteine residue. Examples of cysteine-PEGylated proteins of the invention, as well as proteins having a group other than PEG covalently attached via a cysteine residue according to the invention, are as follows:

Other Stabilizing Modifications of Active Agents

In addition to PEGylation, biologically active agents such as peptides and proteins for use within the invention can be modified to enhance circulating half-life by shielding the active agent via conjugation to other known protecting or stabilizing compounds, for example by the creation of fusion proteins with an active peptide, protein, analog or mimetic linked to one or more carrier proteins, such as one or more immunoglobulin chains (see, e.g., U.S. Pat. Nos. 5,750,375; 5,843,725; 5,567,584 and 6,018,026). These modifications will decrease the degradation, sequestration or clearance of the active agent and result in a longer half-life in a physiological environment (e.g., in the circulatory system, or at a mucosal surface). The active agents modified by these and other stabilizing conjugations methods are therefore useful with enhanced efficacy within the methods of the invention. In particular, the active agents thus modified maintain activity for greater periods at a target site of delivery or action compared to the unmodified active agent. Even when the active agent is thus modified, it retains substantial biological activity in comparison to a biological activity of the unmodified compound.

Thus, in certain aspects of the invention, interferon-β peptides, proteins, analogs and mimetics, and other biologically active agents, including other active peptides and proteins, for mucosal administration according to the methods of the invention are modified for enhanced activity, e.g., to increase circulating half-life, by shielding the active agent through conjugation to other known protecting or stabilizing compounds, or by the creation of fusion proteins with the peptide, protein, analog or mimetic linked to one or more carrier proteins, such as one or more immunoglobulin chains (see, e.g., U.S. Pat. Nos. 5,750,375; 5,843,725; 5,567,584; and 6,018,026). These modifications will decrease the degradation, sequestration or clearance of the active peptide or protein and result in a longer half-life in a physiological environment (e.g., at the nasal mucosal surface or in the systemic circulation). The active peptides and proteins thus modified exhibit enhanced efficacy within the compositions and methods of the invention, for example by increased or temporally extended activity at a target site of delivery or action compared to the unmodified peptide, protein, analog or mimetic.

In other aspects of the invention, peptide and protein therapeutic compounds are conjugated for enhanced stability with relatively low molecular weight compounds, such as aminolethicin, fatty acids, vitamin B₁₂, and glycosides (see, e.g., Igarishi et al., Proc. Int. Symp. Control. Rel. Bioact. Materials, 17, 366, (1990). Additional exemplary modified peptides and proteins for use within the compositions and methods of the invention will be beneficially modified for in vivo use by:

(a) chemical or recombinant DNA methods to link mammalian signal peptides (see, e.g., Lin et al., J. Biol. Chem. 270:14255, 1995) or bacterial peptides (see, e.g., Joliot et al., Proc. Natl. Acad. Sci. USA 88:1864, 1991) to the active peptide or protein, which serves to direct the active peptide or protein across cytoplasmic and organellar membranes and/or traffic the active peptide or protein to the a desired intracellular compartment (e.g., the endoplasmic reticulum (ER) of antigen presenting cells (APCs), such as dendritic cells for enhanced CTL induction);

(b) addition of a biotin residue to the active peptide or protein which serves to direct the active conjugate across cell membranes by virtue of its ability to bind specifically (i.e., with a binding affinity greater than about 10⁶, 10⁷, 10⁸, 10⁹, or 10¹⁰ M⁻¹) to a translocator present on the surface of cells (Chen et al., Analytical Biochem. 227:168, 1995);

(c) addition at either or both the amino- and carboxy-terminal ends of the active peptide or protein of a blocking agent in order to increase stability in vivo. This can be useful in situations in which the termini of the active peptide or protein tend to be degraded by proteases prior to cellular uptake or during intracellular trafficking. Such blocking agents can include, without limitation, additional related or unrelated peptide sequences that can be attached to the amino and/or carboxy terminal residues of the therapeutic polypeptide or peptide to be administered. This can be done either chemically during the synthesis of the peptide or by recombinant DNA technology. Blocking agents such as pyroglutamic acid or other molecules known to those skilled in the art can also be attached to the amino and/or carboxy terminal residues, or the amino group at the amino terminus or carboxyl group at the carboxy terminus can be replaced with a different moiety.

Biologically active agents modified by PEGylation and other stabilizing methods for use within the methods and compositions of the invention will preferably retain at least 25%, more preferably at least 50%, even more preferably between about 50% to 75%, most preferably 100% of the biological activity associated with the unmodified active agent, e.g., a native peptide or protein. Typically, the modified active agent, e.g., a conjugated peptide or protein, has a half-life (t_{1/2}), for example in serum following mucosal delivery, which is enhanced relative to the half-life of the unmodified active agent from which it was derived. In certain aspects, the half-life of a modified active agent (e.g., interferon-β peptides, proteins, analogs and mimetics, and other biologically active peptides and proteins disclosed herein) for use within the invention is enhanced by at least 1.5-fold to 2-fold, often by about 2-fold to 3-fold, in other cases by about 5-fold to 10-fold, and up to 100-fold or more relative to the half-life of the unmodified active agent.

Prodrug Modifications

Yet another processing and formulation strategy useful within the invention is that of prodrug modification. By transiently (i.e., bioreversibly) derivatizing such groups as carboxyl, hydroxyl, and amino groups in small organic molecules, the undesirable physicochemical characteristics (e.g., charge, hydrogen bonding potential, etc. that diminish mucosal penetration) of these molecules can be “masked” without permanently altering the pharmacological properties of the molecule. Bioreversible prodrug derivatives of therapeutic small molecule drugs have been shown to improve the physicochemical (e.g., solubility, lipophilicity) properties of numerous exemplary therapeutics, particularly those that contain hydroxyl and carboxylic acid groups.

One approach to making prodrugs of amine-containing active agents, such as the peptides and proteins of the invention, is through the acylation of the amino group. Optionally, the use of acyloxyalkoxycarbamate derivatives of amines as prodrugs has been discussed. 3-(2′-hydroxy-4′,6′-dimethylphenyl)-3,3-dimethylpropionic acid has been employed to prepare linear, esterase-, phosphatase-, and dehydrogenase-sensitive prodrugs of amines (Amsberry et al., Pharm. Res. 8:455-461, 1991; Wolfe et al., J. Org. Chem. 57:6138, 1992). These systems have been shown to degrade through a two-step mechanism, with the first step being the slow, rate-determining enzyme-catalyzed (esterase, phosphatase, or dehydrogenase) step, and the second step being a rapid (t_{1/2}=100 sec., pH 7.4, 37° C.) chemical step (Amsberry et al., J. Org. Chem. 55:5867-5877, 1990, incorporated herein by reference). Interestingly, the phosphatase-sensitive system has recently been employed to prepare a very water-soluble (greater than 10 mg/ml) prodrug of TAXOL which shows significant antitumor activity in vivo. These and other prodrug modification systems and resultant therapeutic agents are useful within the methods and compositions of the invention.

For the purpose of preparing prodrugs of peptides that are useful within the invention, U.S. Pat. No. 5,672,584 further describes the preparation and use of cyclic prodrugs of biologically active peptides and peptide nucleic acids (PNAs). To produce these cyclic prodrugs, the N-terminal amino group and the C-terminal carboxyl group of a biologically active peptide or PNA is linked via a linker, or the C-terminal carboxyl group of the peptide is linked to a side chain amino group or a side chain hydroxyl group via a linker, or the N-terminal amino group of said peptide is linked to a side chain carboxyl group via a linker, or a side chain carboxyl group of said peptide is linked to a side chain amino group or a side chain hydroxyl group via a linker. Useful linkers in this context include 3-(2′-hydroxy-4′,6′-dimethyl phenyl)-3,3-dimethyl propionic acid linkers and its derivatives, and acyloxyalkoxy derivatives. The incorporated disclosure provides methods useful for the production and characterization of cyclic prodrugs synthesized from linear peptides, e.g., opioid peptides that exhibit advantageous physicochemical features (e.g., reduced size, intramolecular hydrogen bond, and amphophilic characteristics) for enhanced cell membrane permeability and metabolic stability. These methods for peptide prodrug modification are also useful to prepare modified peptide therapeutic derivatives for use within the methods and compositions of the invention.

Formulation and Administration

Mucosal delivery formulations of the present invention comprise the biologically active agent to be administered (e.g., one or more of the interferon-β peptides, proteins, analogs and mimetics, and other biologically active agents disclosed herein), typically combined together with one or more pharmaceutically acceptable carriers and, optionally, other therapeutic ingredients. The carrier(s) must be “pharmaceutically acceptable” in the sense of being compatible with the other ingredients of the formulation and not eliciting an unacceptable deleterious effect in the subject. Such carriers are described herein above or are otherwise well known to those skilled in the art of pharmacology. Desirably, the formulation should not include substances such as enzymes or oxidizing agents with which the biologically active agent to be administered is known to be incompatible. The formulations may be prepared by any of the methods well known in the art of pharmacy.

Within the compositions and methods of the invention, the interferon-β peptides, proteins, analogs and mimetics, and other biologically active agents disclosed herein may be administered to subjects by a variety of mucosal administration modes, including by oral, rectal, vaginal, intranasal, intrapulmonary, or transdermal delivery, or by topical delivery to the eyes, ears, skin or other mucosal surfaces. Optionally, interferon-β peptides, proteins, analogs and mimetics, and other biologically active agents disclosed herein can be coordinately or adjunctively administered by non-mucosal routes, including by intramuscular, subcutaneous, intravenous, intra-atrial, intra-articular, intraperitoneal, or parenteral routes. In other alternative embodiments, the biologically active agent(s) can be administered ex vivo by direct exposure to cells, tissues or organs originating from a mammalian subject, for example as a component of an ex vivo tissue or organ treatment formulation that contains the biologically active agent in a suitable, liquid or solid carrier.

Compositions according to the present invention are often administered in an aqueous solution as a nasal or pulmonary spray and may be dispensed in spray form by a variety of methods known to those skilled in the art. Preferred systems for dispensing liquids as a nasal spray are disclosed in U.S. Pat. No. 4,511,069. Such formulations may be conveniently prepared by dissolving compositions according to the present invention in water to produce an aqueous solution, and rendering said solution sterile. The formulations may be presented in multi-dose containers, for example in the sealed dispensing system disclosed in U.S. Pat. No. 4,511,069. Other suitable nasal spray delivery systems have been described in Transdermal Systemic Medication, Y. W. Chien Ed., Elsevier Publishers, New York, 1985; and in U.S. Pat. No. 4,778,810. Additional aerosol delivery forms may include, e.g., compressed air-, jet-, ultrasonic-, and piezoelectric nebulizers, which deliver the biologically active agent dissolved or suspended in a pharmaceutical solvent, e.g., water, ethanol, or a mixture thereof.

Nasal and pulmonary spray solutions of the present invention typically comprise the drug or drug to be delivered, optionally formulated with a surface active agent, such as a nonionic surfactant (e.g., polysorbate-80), and one or more buffers. In some embodiments of the present invention, the nasal spray solution further comprises a propellant. The pH of the nasal spray solution is optionally between about pH 6.8 and 7.2, but when desired the pH is adjusted to optimize delivery of a charged macromolecular species (e.g., a therapeutic protein or peptide) in a substantially unionized state. The pharmaceutical solvents employed can also be a slightly acidic aqueous buffer (pH 4-6). Suitable buffers for use within these compositions are as described above or as otherwise known in the art. Other components may be added to enhance or maintain chemical stability, including preservatives, surfactants, dispersants, or gases. Suitable preservatives include, but are not limited to, phenol, methyl paraben, paraben, m-cresol, thiomersal, benzylalkonimum chloride, and the like. Suitable surfactants include, but are not limited to, oleic acid, sorbitan trioleate, polysorbates, lecithin, phosphotidyl cholines, and various long chain diglycerides and phospholipids. Suitable dispersants include, but are not limited to, ethylenediaminetetraacetic acid, and the like. Suitable gases include, but are not limited to, nitrogen, helium, chlorofluorocarbons (CFCs), hydrofluorocarbons (HFCs), carbon dioxide, air, and the like.

Within alternate embodiments, mucosal formulations are administered as dry powder formulations comprising the biologically active agent in a dry, usually lyophilized, form of an appropriate particle size, or within an appropriate particle size range, for intranasal delivery. Minimum particle size appropriate for deposition within the nasal or pulmonary passages is often about 0.5μ mass median equivalent aerodynamic diameter (MMEAD), commonly about 1μ MMEAD, and more typically about 2μ MMEAD. Maximum particle size appropriate for deposition within the nasal passages is often about 10μ MMEAD, commonly about 8μ MMEAD, and more typically about 4μ MMEAD. Intranasally respirable powders within these size ranges can be produced by a variety of conventional techniques, such as jet milling, spray drying, solvent precipitation, supercritical fluid condensation, and the like. These dry powders of appropriate MMEAD can be administered to a patient via a conventional dry powder inhaler (DPI) which rely on the patient's breath, upon pulmonary or nasal inhalation, to disperse the power into an aerosolized amount. Alternatively, the dry powder may be administered via air assisted devices that use an external power source to disperse the powder into an aerosolized amount, e.g., a piston pump.

Dry powder devices typically require a powder mass in the range from about 1 mg to 20 mg to produce a single aerosolized dose (“puff”). If the required or desired dose of the biologically active agent is lower than this amount, the powdered active agent will typically be combined with a pharmaceutical dry bulking powder to provide the required total powder mass. Preferred dry bulking powders include sucrose, lactose, dextrose, mannitol, glycine, trehalose, human serum albumin (HSA), and starch. Other suitable dry bulking powders include cellobiose, dextrans, maltotriose, pectin, sodium citrate, sodium ascorbate, and the like.

To formulate compositions for mucosal delivery within the present invention, the biologically active agent can be combined with various pharmaceutically acceptable additives, as well as a base or carrier for dispersion of the active agent(s). Desired additives include, but are not limited to, pH control agents, such as arginine, sodium hydroxide, glycine, hydrochloric acid, citric acid, etc. In addition, local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione) can be included. When the composition for mucosal delivery is a liquid, the tonicity of the formulation, as measured with reference to the tonicity of 0.9% (w/v) physiological saline solution taken as unity, is typically adjusted to a value at which no substantial, irreversible tissue damage will be induced in the nasal mucosa at the site of administration. Generally, the tonicity of the solution is adjusted to a value of about ⅓ to 3, more typically ½ to 2, and most often ¾ to 1.7.

The biologically active agent may be dispersed in a base or vehicle, which may comprise a hydrophilic compound having a capacity to disperse the active agent and any desired additives. The base may be selected from a wide range of suitable carriers, including but not limited to, copolymers of polycarboxylic acids or salts thereof, carboxylic anhydrides (e.g. maleic anhydride) with other monomers (e.g. methyl (meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters, etc. can be employed as carriers. Hydrophilic polymers and other carriers can be used alone or in combination, and enhanced structural integrity can be imparted to the carrier by partial crystallization, ionic bonding, crosslinking and the like. The carrier can be provided in a variety of forms, including, fluid or viscous solutions, gels, pastes, powders, microspheres and films for direct application to the nasal mucosa. The use of a selected carrier in this context may result in promotion of absorption of the biologically active agent.

The biologically active agent can be combined with the base or carrier according to a variety of methods, and release of the active agent may be by diffusion, disintegration of the carrier, or associated formulation of water channels. In some circumstances, the active agent is dispersed in microcapsules (microspheres) or nanocapsules (nanospheres) prepared from a suitable polymer, e.g., isobutyl 2-cyanoacrylate (see, e.g., Michael et al., J. Pharmacy Pharmacol. 43: 1-5, 1991), and dispersed in a biocompatible dispersing medium applied to the nasal mucosa, which yields sustained delivery and biological activity over a protracted time.

To further enhance mucosal delivery of pharmaceutical agents within the invention, formulations comprising the active agent may also contain a hydrophilic low molecular weight compound as a base or excipient. Such hydrophilic low molecular weight compounds provide a passage medium through which a water-soluble active agent, such as a physiologically active peptide or protein, may diffuse through the base to the body surface where the active agent is absorbed. The hydrophilic low molecular weight compound optionally absorbs moisture from the mucosa or the administration atmosphere and dissolves the water-soluble active peptide. The molecular weight of the hydrophilic low molecular weight compound is generally not more than 10000 and preferably not more than 3000. Exemplary hydrophilic low molecular weight compound include polyol compounds, such as oligo-, di- and monosaccarides such as sucrose, mannitol, lactose, L-arabinose, D-erythrose, D-ribose, D-xylose, D-mannose, D-galactose, lactulose, cellobiose, gentibiose, glycerin and polyethylene glycol. Other examples of hydrophilic low molecular weight compounds useful as carriers within the invention include N-methylpyrrolidone, and alcohols (e.g. oligovinyl alcohol, ethanol, ethylene glycol, propylene glycol, etc.) These hydrophilic low molecular weight compounds can be used alone or in combination with one another or with other active or inactive components of the intranasal formulation.

The compositions of the invention may alternatively contain as pharmaceutically acceptable carriers substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

Therapeutic compositions for administering the biologically active agent can also be formulated as a solution, microemulsion, or other ordered structure suitable for high concentration of active ingredients. The carrier can be a solvent or dispersion medium containing, for example, water, ethanol, polyol (for example, glycerol, propylene glycol, and liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols such as mannitol, sorbitol, or sodium chloride in the composition. Prolonged absorption of the biologically active agent can be brought about by including in the composition an agent which delays absorption, for example, monostearate salts and gelatin.

In certain embodiments of the invention, the biologically active agent is administered in a time release formulation, for example in a composition which includes a slow release polymer. The active agent can be prepared with carriers that will protect against rapid release, for example a controlled release vehicle such as a polymer, microencapsulated delivery system or bioadhesive gel. Prolonged delivery of the active agent, in various compositions of the invention can be brought about by including in the composition agents that delay absorption, for example, aluminum monosterate hydrogels and gelatin. When controlled release formulations of the biologically active agent is desired, controlled release binders suitable for use in accordance with the invention include any biocompatible controlled-release material which is inert to the active agent and which is capable of incorporating the biologically active agent. Numerous such materials are known in the art. Useful controlled-release binders are materials that are metabolized slowly under physiological conditions following their intranasal delivery (e.g., at the nasal mucosal surface, or in the presence of bodily fluids following transmucosal delivery). Appropriate binders include but are not limited to biocompatible polymers and copolymers previously used in the art in sustained release formulations. Such biocompatible compounds are non-toxic and inert to surrounding tissues, and do not trigger significant adverse side effects such as nasal irritation, immune response, inflammation, or the like. They are metabolized into metabolic products that are also biocompatible and easily eliminated from the body.

Exemplary polymeric materials for use in this context include, but are not limited to, polymeric matrices derived from copolymeric and homopolymeric polyesters having hydrolysable ester linkages. A number of these are known in the art to be biodegradable and to lead to degradation products having no or low toxicity. Exemplary polymers include polyglycolic acids (PGA) and polylactic acids (PLA), poly(DL-lactic acid-co-glycolic acid)(DL PLGA), poly(D-lactic acid-coglycolic acid)(D PLGA) and poly(L-lactic acid-co-glycolic acid)(L PLGA). Other useful biodegradable or bioerodable polymers include but are not limited to such polymers as poly(epsilon-caprolactone), poly(epsilon-aprolactone-CO-lactic acid), poly(ε-aprolactone-CO-glycolic acid), poly(beta-hydroxy butyric acid), poly(alkyl-2-cyanoacrilate), hydrogels such as poly(hydroxyethyl methacrylate), polyamides, poly(amino acids) (i.e., L-leucine, glutamic acid, L-aspartic acid and the like), poly(ester urea), poly(2-hydroxyethyl DL-aspartamide), polyacetal polymers, polyorthoesters, polycarbonate, polymaleamides, polysaccharides and copolymers thereof. Other useful formulations include controlled-release compositions such as lactic acid-glycolic acid copolymers useful in making microcapsules and other formulations (U.S. Pat. Nos. 4,677,191 and 4,728,721), and sustained-release compositions for water-soluble peptides (U.S. Pat. No. 4,675,189).

The mucosal formulations of the invention typically must be sterile and stable under all conditions of manufacture, storage and use. Sterile solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders, methods of preparation include vacuum drying and freeze-drying which yields a powder of the active ingredient plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

In more detailed aspects of the invention, the biologically active agent is stabilized to extend its effective half-life following delivery to the subject, particularly for extending metabolic persistence in an active state within the physiological environment (e.g., at the nasal mucosal surface, in the bloodstream, or within a connective tissue compartment or fluid-filled body cavity). For this purpose, the biologically active agent may be modified by chemical means, e.g., chemical conjugation, N-terminal capping, PEGylation, or recombinant means, e.g., site-directed mutagenesis or construction of fusion proteins, or formulated with various stabilizing agents or carriers. Thus stabilized, the active agent administered as above retains biological activity for an extended period (e.g., 2-3, up to 5-10 fold greater stability) under physiological conditions compared to its non-stabilized form.

In accordance with the various treatment methods of the invention, the biologically active agent is delivered to a mammalian subject in a manner consistent with conventional methodologies associated with management of the disorder for which treatment or prevention is sought. In accordance with the disclosure herein, a prophylactically or therapeutically effective amount of the biologically active agent is administered to a subject in need of such treatment for a time and under conditions sufficient to prevent, inhibit, and/or ameliorate a selected disease or condition or one or more symptom(s) thereof.

The term “subject” as used herein means any mammalian patient to which the compositions of the invention may be administered. Typical subjects intended for treatment with the compositions and methods of the present invention include humans, as well as non-human primates and other animals. To identify subject patients for prophylaxis or treatment according to the methods of the invention, accepted screening methods are employed to determine risk factors associated with a targeted or suspected disease of condition as discussed above, or to determine the status of an existing disease or condition in a subject. These screening methods include, for example, conventional work-ups to determine familial, sexual, drug-use and other such risk factors that may be associated with the targeted or suspected disease or condition, as well as diagnostic methods such as various ELISA immunoassay methods, which are available and well known in the art to detect and/or characterize disease-associated markers. These and other routine methods allow the clinician to select patients in need of therapy using the mucosal methods and formulations of the invention. In accordance with these methods and principles, biologically active agents may be mucosally administered according to the teachings herein as an independent prophylaxis or treatment program, or as a follow-up, adjunct or coordinate treatment regimen to other treatments, including surgery, vaccination, immunotherapy, hormone treatment, cell, tissue, or organ transplants, and the like.

Mucosal administration according to the invention allows effective self-administration of treatment by patients, provided that sufficient safeguards are in place to control and monitor dosing and side effects. Mucosal administration also overcomes certain drawbacks of other administration forms, such as injections, that are painful and expose the patient to possible infections and may present drug bioavailability problems. For nasal and pulmonary delivery, systems for controlled aerosol dispensing of therapeutic liquids as a spray are well known. In one embodiment, metered doses of active agent are delivered by means of a specially constructed mechanical pump valve (U.S. Pat. No. 4,511,069). This hand-held delivery device is uniquely nonvented so that sterility of the solution in the aerosol container is maintained indefinitely.

Dosage

For prophylactic and treatment purposes, the biologically active agent(s) disclosed herein may be administered to the subject in a single bolus delivery, via continuous delivery (e.g., continuous transdermal, mucosal, or intravenous delivery) over an extended time period, or in a repeated administration protocol (e.g., by an hourly, daily or weekly, repeated administration protocol). In this context, a therapeutically effective dosage of the biologically active agent(s) may include repeated doses within a prolonged prophylaxis or treatment regimen, that will yield clinically significant results to alleviate one or more symptoms or detectable conditions associated with a targeted disease or condition as set forth above. Determination of effective dosages in this context is typically based on animal model studies followed up by human clinical trials and is guided by determining effective dosages and administration protocols that significantly reduce the occurrence or severity of targeted disease symptoms or conditions in the subject. Suitable models in this regard include, for example, murine, rat, porcine, feline, non-human primate, and other accepted animal model subjects known in the art. Alternatively, effective dosages can be determined using in vitro models (e.g., immunologic and histopathologic assays). Using such models, only ordinary calculations and adjustments are typically required to determine an appropriate concentration and dose to administer a therapeutically effective amount of the biologically active agent(s) (e.g., amounts that are intranasally effective, transdermally effective, intravenously effective, or intramuscularly effective to elicit a desired response). In alternative embodiments, an “effective amount” or “effective dose” of the biologically active agent(s) may simply inhibit or enhance one or more selected biological activity(ies) correlated with a disease or condition, as set forth above, for either therapeutic or diagnostic purposes.

The actual dosage of biologically active agents will of course vary according to factors such as the disease indication and particular status of the subject (e.g., the subject's age, size, fitness, extent of symptoms, susceptibility factors, etc), time and route of administration, other drugs or treatments being administered concurrently, as well as the specific pharmacology of the biologically active agent(s) for eliciting the desired activity or biological response in the subject. Dosage regimens may be adjusted to provide an optimum prophylactic or therapeutic response. A therapeutically effective amount is also one in which any toxic or detrimental side effects of the biologically active agent is outweighed in clinical terms by therapeutically beneficial effects. A non-limiting range for a therapeutically effective amount of a biologically active agent within the methods and formulations of the invention is 0.01 μg/kg-10 mg/kg, more typically between about 0.05 and 5 mg/kg, and in certain embodiments between about 0.2 and 2 mg/kg. Dosages within this range can be achieved by single or multiple administrations, including, e.g., multiple administrations per day, daily or weekly administrations. Per administration, it is desirable to administer at least one microgram of the biologically active agent (e.g., one or more interferon-β peptides, proteins, analogs and mimetics, and other biologically active agents), more typically between about 10 μg and 5.0 mg, and in certain embodiments between about 100 μg and 1.0 or 2.0 mg to an average human subject. It is to be further noted that for each particular subject, specific dosage regimens should be evaluated and adjusted over time according to the individual need and professional judgment of the person administering or supervising the administration of the permeabilizing peptide(s) and other biologically active agent(s).

Dosage of biologically active agents may be varied by the attending clinician to maintain a desired concentration at the target site. For example, a selected local concentration of the biologically active agent in the bloodstream or CNS may be about 1-50 nanomoles per liter, sometimes between about 1.0 nanomole per liter and 10, 15 or 25 nanomoles per liter, depending on the subject's status and projected or measured response. Higher or lower concentrations may be selected based on the mode of delivery, e.g., trans-epidermal, rectal, oral, or intranasal delivery versus intravenous or subcutaneous delivery. Dosage should also be adjusted based on the release rate of the administered formulation, e.g., of a nasal spray versus powder, sustained release oral versus injected particulate or transdermal delivery formulations, etc. To achieve the same serum concentration level, for example, slow-release particles with a release rate of 5 nanomolar (under standard conditions) would be administered at about twice the dosage of particles with a release rate of 10 nanomolar.

Additional guidance as to particular dosages for selected biologically active agents for use within the invention may be found widely disseminated in the literature. This is true for many of the therapeutic peptide and protein agents disclosed herein.

Kits

The instant invention also includes kits, packages and multicontainer units containing the above described pharmaceutical compositions, active ingredients, and/or means for administering the same for use in the prevention and treatment of diseases and other conditions in mammalian subjects. Briefly, these kits include a container or formulation that contains one or more interferon-β peptides, proteins, analogs and mimetics, and other biologically active agents disclosed herein formulated in a pharmaceutical preparation for mucosal delivery. The biologically active agent(s) is/are optionally contained in a bulk dispensing container or unit or multi-unit dosage form. Optional dispensing means may be provided, for example a pulmonary or intranasal spray applicator. Packaging materials optionally include a label or instruction indicating that the pharmaceutical agent packaged therewith can be used mucosally, e.g., intranasally, for treating or preventing a specific disease or condition.

The following examples are provided by way of illustration, not limitation.

### EXAMPLE 1

### Dosages for Parenteral and Nasal Mucosal Delivery of Interferon-β

Table 1 indicates dosages for parenteral and nasal mucosal delivery of interferon-β and interferon-γ for treatment of disease including multiple sclerosis and tumors.

TABLE 1
Mucosal delivery of IFN-β as an adjunct with
parenteral (subcutaneous or intramuscular)
interferon to increase efficacy and safety.

| | | | | Nasal |
|---|---|---|---|---|
| | | | Dosages and | Adjunct |
| Drug Name | Route | Indication | Administration | Therapy |
| Interferon β-1a | Parenteral | Multiple | 30 μg once | Yes |
| | | Sclerosis | per week or | |
| | | | 44 μg thrice | |
| | | | a week | |
| Interferon β-1b | Parenteral | Multiple | 0.25 mg | Yes |
| | | Sclerosis | every other day | |
| Inteferon γ-1b | Parenteral | Chronic | 1.5 MIU | Yes |
| | | Granulomatosis | 3 times | |
| | | and | per week | |
| | | Osteoporosis | | |

### EXAMPLE 2

### Exemplary Pharmaceutical Formulations Comprising Interferon-β-1a and Intranasal Delivery-Enhancing Agents

The formulations in Table 2 comprise interferon-β-1a (Avonex®; Biogen, Inc.) in combination with intranasal delivery-enhancing agents of the present invention. The freeze dried powder component of one vial of Avonex® contained 6.6 MIU interferon-β-1a/33 μg, 16.5 mg human serum albumin, USP, 6.38 mg sodium chloride, USP, 6.27 mg dibasic sodium phosphate USP, and 1.32 mg monobasic sodium phosphate, USP. Solutions containing intranasal delivery-enhancing agents were reconstituted by adding 0.55 mL of solution containing intranasal delivery-enhancing agents to powder content of Avonex® 6.6 MIU/vial.

TABLE 2
Formulations comprising interferon-β-1a
and intranasal delivery-enhancing agents.

| Intranasal | | | |
|---|---|---|---|
| Delivery- | | | |
| Enhancing Agent | Component | Quantity | |
| N-Caproic | Interferon-β-1a | 6.6 | MIU |
| Acid Sodium | Albumin human USP | 16.5 | mg |
| | Sodium Chloride USP | 6.38 | mg |
| | Dibasic Sodium Phosphate USP | 6.27 | mg |
| | Monobasic Sodium Phosphate USP | 1.32 | mg |
| | N-Caproic Acid Sodium | 1.38 | mg |
| | Purified Water, USP q.s. to | 1 | mL |
| Pluronic-127 | Interferon-β-1a | 6.6 | MIU |
| | Albumin human USP | 16.5 | mg |
| | Sodium Chloride USP | 6.38 | mg |
| | Dibasic Sodium Phosphate USP | 6.27 | mg |
| | Monobasic Sodium Phosphate USP | 1.32 | mg |
| | Pluronic-127 | 3 | mg |
| | Purified Water, USP q.s. to | 1 | mL |
| Alpha- | Interferon-β-1a | 6.6 | MIU |
| Cyclodextrin | Albumin human USP | 16.5 | mg |
| | Sodium Chloride USP | 6.38 | mg |
| | Dibasic Sodium Phosphate USP | 6.27 | mg |
| | Monobasic Sodium Phosphate USP | 1.32 | mg |
| | Alpha-Cyclodextrin | 50 | mg |
| | Purified Water, USP q.s. to | 1 mL | mL |

### EXAMPLE 3

### Exemplary Pharmaceutical Formulations Comprising Interferon-β-1a and Intranasal Delivery-Enhancing Agents

Pharmaceutical formulations in Table 3 comprise interferon-β-1a (Avonex®; Biogen, Inc.) in combination with intranasal delivery-enhancing agents of the present invention. The freeze dried powder component of one vial of Avonex® contained 6.6 MIU interferon-β-1a/33 μg, 16.5 mg human serum albumin, USP, 6.38 mg sodium chloride, USP, 6.27 mg dibasic sodium phosphate USP, and 1.32 mg monobasic sodium phosphate, USP. Solutions containing intranasal delivery-enhancing agents were reconstituted by adding 0.55 mL of aqueous solution containing intranasal delivery-enhancing agents to content of the vial of Avonex® (6.6 MIU/vial).

TABLE 3
Formulations comprising interferon-β-1a
and intranasal delivery-enhancing agents.

| Formulation | Composition | Quantity | |
|---|---|---|---|
| Avonex ® | Avonex ® (Interferon-β-1a) | 12 | MIU |
| | Albumin human USP | 30 | mg |
| | Sodium Chloride USP | 11.6 | mg |
| | Dibasic Sodium Phosphate USP | 11.4 | mg |
| | Monobasic Sodium Phosphate USP | 2.4 | mg |
| | Purified Water, USP q.s. to | 1 | mL |
| F2 | Interferon-β-1a | 12 | MIU |
| | Albumin human USP | 30 | mg |
| | Sodium Chloride USP | 11.6 | mg |
| | Dibasic Sodium Phosphate USP | 11.4 | mg |
| | Monobasic Sodium Phosphate USP | 2.4 | mg |
| | Benzalkonium Chloride 50% | 2 | mg |
| | Sodium Taurocholate | 15 | mg |
| | EDTA | 1 | mg |
| | Arginine | 150 | mg |
| | Alpha Cyclodextrin | 50 | mg |
| | HPC (4000-6500 cps) | 5 | mg |
| | Purified Water, q.s. to | 1 | mL |
| F3 | Interferon-β-1a | 12 | MIU |
| | Albumin human USP | 30 | mg |
| | Sodium Chloride USP | 11.6 | mg |
| | Dibasic Sodium Phosphate USP | 11.4 | mg |
| | Monobasic Sodium Phosphate USP | 2.4 | mg |
| | Benzalkonium Chloride 50% | 2 | mg |
| | Sodium Taurocholate | 15 | mg |
| | EDTA | 1 | mg |
| | Arginine Hydrochloride | 150 | mg |
| | Gamma Cyclodextrin | 20 | mg |
| | HPC (4000-6500 cps) | 5 | mg |
| | Purified Water, q.s. to | 1 | mL |
| F4 | Interferon-β-1a | 12 | MIU |
| | Albumin human USP | 30 | mg |
| | Sodium Chloride USP | 11.6 | mg |
| | Dibasic Sodium Phosphate USP | 11.4 | mg |
| | Monobasic Sodium Phosphate USP | 2.4 | mg |
| | Chitosan (Chitoclear, 95%) | 5 | mg |
| | Acetic Acid 1N | 5 | mg |
| | Benzalkonium Chloride | 0.01 | mg |
| | Sodium Deoxycholate | 1 | mg |
| | Methyl-b-Cyclodextrin | 50 | mg |
| | EDTA | 0.1 | mg |
| | Sodium Hydroxide | QS | mg |
| | Purified Water, USP q.s. to | 1 | mL |
| F5 | Interferon-β-1a | 12 | MIU |
| | Albumin human USP | 30 | mg |
| | Sodium Chloride USP | 11.6 | mg |
| | Dibasic Sodium Phosphate USP | 11.4 | mg |
| | Monobasic Sodium Phosphate USP | 2.4 | mg |
| | L-α-phosphatidylcholine didecanoyl | 5 | mg |
| | Methyl Beta Cyclodextrin | 30 | mg |
| | EDTA | 1 | mg |
| | Gelatin | 5 | mg |
| | Purified Water, USP q.s. to | 1 | mL |
| F6 | Interferon-β-1a | 12 | MIU |
| | Albumin human USP | 30 | mg |
| | Sodium Chloride USP | 11.6 | mg |
| | Dibasic Sodium Phosphate USP | 11.4 | mg |
| | Monobasic Sodium Phosphate USP | 2.4 | mg |
| | Benzalkonium Chloride 50% | 2 | mg |
| | Sodium Taurocholate | 15 | mg |
| | EDTA | 1 | mg |
| | Arginine Hydrochloride | 150 | mg |
| | HPC (4000-6500 cps) | 5 | mg |
| | Purified Water, q.s. to | 1 | mL |
| F7 | Interferon-β-1a | 12 | MIU |
| | Albumin human USP | 30 | mg |
| | Sodium Chloride USP | 11.6 | mg |
| | Dibasic Sodium Phosphate USP | 11.4 | mg |
| | Monobasic Sodium Phosphate USP | 2.4 | mg |
| | Benzalkonium Chloride 50% | 4 | mg |
| | Sodium Glycocolate | 10 | mg |
| | Methyl Beta Cyclodextrin | 25 | mg |
| | EDTA | 1 | mg |
| | Chitosan | 5 | mg |
| | Purified Water q.s. to | 1 | mL |
| F8 | Interferon-β-1a | 6 | MIU |
| | Albumin human USP | 5.18 | mg |
| | Sodium Chloride USP | 1.09 | mg |
| | Dibasic Sodium Phosphate USP | 13.64 | mg |
| | Monobasic Sodium Phosphate USP | 5.8 | mg |
| | Benzalkonium Chloride 50% | 0.4 | mg |
| | Sodium Taurocholate | 2.5 | mg |
| | Hydroxy propyl Cyclodextrin | 50 | mg |
| | HPMC | 1 | mg |
| | Purified Water, USP q.s. to | 1 | mL |
| F9 | Interferon-β-1a (300 μg) | 60 | MIU |
| | albumin human | 15 | mg |
| | dibasic sodium phosphate | 5.7 | mg |
| | monobasic sodium phosphate | 1.2 | mg |
| | sodium chloride | 5.8 | mg |
| | benzalkonium chloride (50%) | 1.0 | mg |
| | L-α-phosphatidylcholine didecanoyl | 0.5 | mg |
| | methyl-β-cyclodextrin | 30 | mg |
| | EDTA disodium | 1.0 | mg |
| | gelatin | 5.0 | mg |
| | purified water USP q.s. to | 1.0 | mL |

### EXAMPLE 4

### Mucosal Delivery—Permeation Kinetics and Cytotoxicity

1. Organotypic Model

The following methods are generally useful for evaluating mucosal delivery parameters, kinetics and side effects for IFN-β within the formulations and method of the invention, as well as for determining the efficacy and characteristics of the various mucosal delivery-enhancing agents disclosed herein for combinatorial formulation or coordinate administration with IFN-β.

Permeation kinetics and cytotoxicity are also useful for determining the efficacy and characteristics of the various mucosal delivery-enhancing agents disclosed herein for combinatorial formulation or coordinate administration with mucosal delivery-enhancing agents. In one exemplary protocol, permeation kinetics and lack of unacceptable cytotoxicity are demonstrated for an intranasal delivery-enhancing agents as disclosed above in combination with a biologically active therapeutic agent, exemplified by interferon-β.

The EpiAirway system was developed by MatTek Corp (Ashland, Mass.) as a model of the pseudostratified epithelium lining the respiratory tract. The epithelial cells are grown on porous membrane-bottomed cell culture inserts at an air-liquid interface, which results in differentiation of the cells to a highly polarized morphology. The apical surface is ciliated with a microvillous ultrastructure and the epithelium produces mucus (the presence of mucin has been confirmed by immunoblotting). The inserts have a diameter of 0.875 cm, providing a surface area of 0.6 cm². The cells are plated onto the inserts at the factory approximately three weeks before shipping. One “kit” consists of 24 units.

A. On arrival, the units are placed onto sterile supports in 6-well microplates. Each well receives 5 mL of proprietary culture medium. This DMEM-based medium is serum free but is supplemented with epidermal growth factor and other factors. The medium is always tested for endogenous levels of any cytokine or growth factor which is being considered for intranasal delivery, but has been free of all cytokines and factors studied to date except insulin. The 5 mL volume is just sufficient to provide contact to the bottoms of the units on their stands, but the apical surface of the epithelium is allowed to remain in direct contact with air. Sterile tweezers are used in this step and in all subsequent steps involving transfer of units to liquid-containing wells to ensure that no air is trapped between the bottoms of the units and the medium.

B. The units in their plates are maintained at 37° C. in an incubator in an atmosphere of 5% CO₂ in air for 24 hours. At the end of this time the medium is replaced with fresh medium and the units are returned to the incubator for another 24 hours.

2. Experimental Protocol—Permeation Kinetics

A. A “kit” of 24 EpiAirway units can routinely be employed for evaluating five different formulations, each of which is applied to quadruplicate wells. Each well is employed for determination of permeation kinetics (4 time points), transepithelial resistance, mitochondrial reductase activity as measured by MTT reduction, and cytolysis as measured by release of LDH. An additional set of wells is employed as controls, which are sham treated during determination of permeation kinetics, but are otherwise handled identically to the test sample-containing units for determinations of transepithelial resistance and viability. The determinations on the controls are routinely also made on quadruplicate units, but occasionally we have employed triplicate units for the controls and have dedicated the remaining four units in the kit to measurements of transepithelial resistance and viability on untreated units or we have frozen and thawed the units for determinations of total LDH levels to serve as a reference for 100% cytolysis.

B. In all experiments, the mucosal delivery formulation to be studied is applied to the apical surface of each unit in a volume of 100 μL, which is sufficient to cover the entire apical surface. An appropriate volume of the test formulation at the concentration applied to the apical surface (no more than 100 μL is generally needed) is set aside for subsequent determination of concentration of the active material by ELISA or other designated assay.

C. The units are placed in 6 well plates without stands for the experiment: each well contains 0.9 mL of medium which is sufficient to contact the porous membrane bottom of the unit but does not generate any significant upward hydrostatic pressure on the unit.

D. In order to minimize potential sources of error and avoid any formation of concentration gradients, the units are transferred from one 0.9 mL-containing well to another at each time point in the study. These transfers are made at the following time points, based on a zero time at which the 100 μL volume of test material was applied to the apical surface: 15 minutes, 30 minutes, 60 minutes, and 120 minutes.

E. In between time points the units in their plates are kept in the 37° C. incubator. Plates containing 0.9 mL medium per well are also maintained in the incubator so that minimal change in temperature occurs during the brief periods when the plates are removed and the units are transferred from one well to another using sterile forceps.

F. At the completion of each time point, the medium is removed from the well from which each unit was transferred, and aliquotted into two tubes (one tube receives 700 μL and the other 200 μL) for determination of the concentration of permeated test material and, in the event that the test material is cytotoxic, for release of the cytosolic enzyme, lactic dehydrogenase, from the epithelium. These samples are kept in the refrigerator if the assays are to be conducted within 24 hours, or the samples are subaliquotted and kept frozen at −80° C. until thawed once for assays. Repeated freeze-thaw cycles are to be avoided.

G. In order to minimize errors, all tubes, plates, and wells are prelabeled before initiating an experiment.

H. At the end of the 120 minute time point, the units are transferred from the last of the 0.9 mL containing wells to 24-well microplates, containing 0.3 mL medium per well. This volume is again sufficient to contact the bottoms of the units, but not to exert upward hydrostatic pressure on the units. The units are returned to the incubator prior to measurement of transepithelial resistance.

3. Experimental Protocol—Transepithelial Resistance

A. Respiratory airway epithelial cells form tight junctions in vivo as well as in vitro, restricting the flow of solutes across the tissue. These junctions confer a transepithelial resistance of several hundred ohms×cm² in excised airway tissues; in the MatTek EpiAirway units, the transepithelial resistance (TER) is claimed by the manufacturer to be routinely around 1000 ohms×cm². We have found that the TER of control EpiAirway units which have been sham-exposed during the sequence of steps in the permeation study is somewhat lower (700-800 ohms×cm²), but, since permeation of small molecules is proportional to the inverse of the TER, this value is still sufficiently high to provide a major barrier to permeation. The porous membrane-bottomed units without cells, conversely, provide only minimal transmembrane resistance (5-20 ohms×cm²).

B. Accurate determinations of TER require that the electrodes of the ohmmeter be positioned over a significant surface area above and below the membrane, and that the distance of the electrodes from the membrane be reproducibly controlled. The method for TER determination recommended by MatTek and employed for all experiments here employs an “EVOM”™ epithelial voltohmmeter and an “ENDOHM”™ tissue resistance measurement chamber from World Precision Instruments, Inc. Sarasota, Fla.

C. The chamber is initially filled with Dulbecco's phosphate buffered saline (PBS) for at least 20 minutes prior to TER determinations in order to equilibrate the electrodes.

D. Determinations of TER are made with 1.5 mL of PBS in the chamber and 350 μL of PBS in the membrane-bottomed unit being measured. The top electrode is adjusted to a position just above the membrane of a unit containing no cells (but containing 350 μL of PBS) and then fixed to ensure reproducible positioning. The resistance of a cell-free unit is typically 5-20 ohms×cm² (“background resistance”).

E. Once the chamber is prepared and the background resistance is recorded, units in a 24-well plate which had just been employed in permeation determinations are removed from the incubator and individually placed in the chamber for TER determinations.

F. Each unit is first transferred to a petri dish containing PBS to ensure that the membrane bottom is moistened. An aliquot of 350 μL PBS is added to the unit and then carefully aspirated into a labeled tube to rinse the apical surface. A second wash of 350 μL PBS is then applied to the unit and aspirated into the same collection tube.

G. The unit is gently blotted free of excess PBS on its exterior surface only before being placed into the chamber (containing a fresh 1.5 mL aliquot of PBS). An aliquot of 350 μL PBS is added to the unit before the top electrode is placed on the chamber and the TER is read on the EVOM meter.

H. After the TER of the unit is read in the ENDOHM chamber, the unit is removed, the PBS is aspirated and saved, and the unit is returned with an air interface on the apical surface to a 24-well plate containing 0.3 mL medium per well.

I. The units are read in the following sequence: all sham-treated controls, followed by all formulation-treated samples, followed by a second TER reading of each of the sham-treated controls. After all the TER determinations are complete, the units in the 24-well microplate are returned to the incubator for determination of viability by MTT reduction.

4. Experimental Protocol—Viability by MTT Reduction

MTT is a cell-permeable tetrazolium salt which is reduced by mitochondrial dehydrogenase activity to an insoluble colored formazan by viable cells with intact mitochondrial function or by nonmitochondrial NAD(P)H dehydrogenase activity from cells capable of generating a respiratory burst. Formation of formazan is a good indicator of viability of epithelial cells since these cells do not generate a significant respiratory burst. We have employed a MTT reagent kit prepared by MatTek Corp for their units in order to assess viability.

A. The MTT reagent is supplied as a concentrate and is diluted into a proprietary DMEM-based diluent on the day viability is to be assayed (typically the afternoon of the day in which permeation kinetics and TER were determined in the morning). Insoluble reagent is removed by a brief centrifugation before use. The final MTT concentration is 1 mg/mL

B. The final MTT solution is added to wells of a 24-well microplate at a volume of 300 μL per well. As has been noted above, this volume is sufficient to contact the membranes of the EpiAirway units but imposes no significant positive hydrostatic pressure on the cells.

C. The units are removed from the 24-well plate in which they were placed after TER measurements, and after removing any excess liquid from the exterior surface of the units, they are transferred to the plate containing MTT reagent. The units in the plate are then placed in an incubator at 37° C. in an atmosphere of 5% CO₂ in air for 3 hours.

D. At the end of the 3-hour incubation, the units containing viable cells will have turned visibly purple. The insoluble formazan must be extracted from the cells in their units to quantitate the extent of MTT reduction. Extraction of the formazan is accomplished by transferring the units to a 24-well microplate containing 2 mL extractant solution per well, after removing excess liquid from the exterior surface of the units as before. This volume is sufficient to completely cover both the membrane and the apical surface of the units. Extraction is allowed to proceed overnight at room temperature in a light-tight chamber. MTT extractants traditionally contain high concentrations of detergent, and destroy the cells.

E. At the end of the extraction, the fluid from within each unit and the fluid in its surrounding well are combined and transferred to a tube for subsequent aliquotting into a 96-well microplate (200 μL aliquots are optimal) and determination of absorbance at 570 nm on a VMax multiwell microplate spectrophotometer. To ensure that turbidity from debris coming from the extracted units does not contribute to the absorbance, the absorbance at 650 nm is also determined for each well in the VMax and is automatically subtracted from the absorbance at 570 nm. The “blank” for the determination of formazan absorbance is a 200 μL aliquot of extractant to which no unit had been exposed. This absorbance value is assumed to constitute zero viability.

F. Two units from each kit of 24 EpiAirway units are left untreated during determination of permeation kinetics and TER. These units are employed as the positive control for 100% cell viability. In all the studies we have conducted, there has been no statistically significant difference in the viability of the cells in these untreated units vs cells in control units which had been sham treated for permeation kinetics and on which TER determinations had been performed. The absorbance of all units treated with test formulations is assumed to be linearly proportional to the percent viability of the cells in the units at the time of the incubation with MTT. It should be noted that this assay is carried out typically no sooner than four hours after introduction of the test material to the apical surface, and subsequent to rinsing of the apical surface of the units during TER determination.

5. Determination of Viability by LDH Release

While measurement of mitochondrial reductase activity by MTT reduction is a sensitive probe of cell viability, the assay necessarily destroys the cells and therefore can be carried out only at the end of each study. When cells undergo necrotic lysis, their cytotosolic contents are spilled into the surrounding medium, and cytosolic enzymes such as lactic dehydrogenase (LDH) can be detected in this medium. An assay for LDH in the medium can be performed on samples of medium removed at each time point of the two-hour determination of permeation kinetics. Thus, cytotoxic effects of formulations which do not develop until significant time has passed can be detected as well as effects of formulations which induce cytolysis with the first few minutes of exposure to airway epithelium.

A. The recommended LDH assay for evaluating cytolysis of the EpiAirway units is based on conversion of lactate to pyruvate with generation of NADH from NAD. The NADH is then reoxidized along with simultaneous reduction of the tetrazolium salt INT, catalyzed by a crude “diaphorase” preparation. The formazan formed from reduction of INT is soluble, so that the entire assay for LDH activity can be carried out in a homogenous aqueous medium containing lactate, NAD, diaphorase, and INT.

B. The assay for LDH activity is carried out on 50 μL aliquots from samples of “supernatant” medium surrounding an EpiAirway unit and collected at each time point. These samples were either stored for no longer than 24 h in the refrigerator or were thawed after being frozen within a few hours after collection. Each EpiAirway unit generates samples of supernatant medium collected at 15 min, 30 min, 1 h, and 2 h after application of the test material. The aliquots are all transferred to a 96 well microplate.

C. A 50 μL aliquot of medium which had not been exposed to a unit serves as a “blank” or negative control of 0% cytotoxicity. We have found that the apparent level of “endogenous” LDH present after reaction of the assay reagent mixture with the unexposed medium is the same within experimental error as the apparent level of LDH released by all the sham-treated control units over the entire time course of 2 hours required to conduct a permeation kinetics study. Thus, within experimental error, these sham-treated units show no cytolysis of the epithelial cells over the time course of the permeation kinetics measurements.

D. To prepare a sample of supernatant medium reflecting the level of LDH released after 100% of the cells in a unit have lysed, a unit which had not been subjected to any prior manipulations is added to a well of a 6-well microplate containing 0.9 mL of medium as in the protocol for determination of permeation kinetics, the plate containing the unit is frozen at −80° C., and the contents of the well are then allowed to thaw. This freeze-thaw cycle effectively lyses the cells and releases their cytosolic contents, including LDH, into the supernatant medium. A 50 μL aliquot of the medium from the frozen and thawed cells is added to the 96-well plate as a positive control reflecting 100% cytotoxicity.

E. To each well containing an aliquot of supernatant medium, a 50 μL aliquot of the LDH assay reagent is added. The plate is then incubated for 30 minutes in the dark.

F. The reactions are terminated by addition of a “stop” solution of 1 M acetic acid, and within one hour of addition of the stop solution, the absorbance of the plate is determined at 490 nm.

G. Computation of percent cytolysis is based on the assumption of a linear relationship between absorbance and cytolysis, with the absorbance obtained from the medium alone serving as a reference for 0% cytolysis and the absorbance obtained from the medium surrounding a frozen and thawed unit serving as a reference for 100% cytolysis.

6. ELISA Determinations

The procedures for determining the concentrations of biologically active agents as test materials for evaluating enhanced permeation of active agents in conjunction with coordinate administration of mucosal delivery-enhancing agents or combinatorial formulation of the invention are generally as described above and in accordance with known methods and specific manufacturer instructions of ELISA kits employed for each particular assay. Permeation kinetics of the biologically active agent is generally determined by taking measurements at multiple time points (for example 15 min., 30 min., 60 min. and 120 min) after the biologically active agent is contacted with the apical epithelial cell surface (which may be simultaneous with, or subsequent to, exposure of the apical cell surface to the mucosal delivery-enhancing agent(s)).

EpiAirway™ tissue membranes are cultured in phenol red and hydrocortisone free medium (MatTek Corp., Ashland, Mass.). The tissue membranes are cultured at 37° C. for 48 hours to allow the tissues to equilibrate. Each tissue membrane is placed in an individual well of a 6-well plate containing 0.9 mL of serum free medium. 100 μL of the formulation (test sample or control) is applied to the apical surface of the membrane. Triplicate or quadruplicate samples of each test sample (mucosal delivery-enhancing agent in combination with a biologically active agent, interferon-β) and control (biologically active agent, interferon-β, alone) are evaluated in each assay. At each time point (15, 30, 60 and 120 minutes) the tissue membranes are moved to new wells containing fresh medium. The underlying 0.9 mL medium samples is harvested at each time point and stored at 4° C. for use in ELISA and lactate dehydrogenase (LDH) assays.

The ELISA kits are typically two-step sandwich ELISAs: the immunoreactive form of the agent being studied is first “captured” by an antibody immobilized on a 96-well microplate and after washing unbound material out of the wells, a “detection” antibody is allowed to react with the bound immunoreactive agent. This detection antibody is typically conjugated to an enzyme (most often horseradish peroxidase) and the amount of enzyme bound to the plate in immune complexes is then measured by assaying its activity with a chromogenic reagent. In addition to samples of supernatant medium collected at each of the time points in the permeation kinetics studies, appropriately diluted samples of the formulation (i.e., containing the subject biologically active test agent) that was applied to the apical surface of the units at the start of the kinetics study are also assayed in the ELISA plate, along with a set of manufacturer-provided standards. Each supernatant medium sample is generally assayed in duplicate wells by ELISA (it will be recalled that quadruplicate units are employed for each formulation in a permeation kinetics determination, generating a total of sixteen samples of supernatant medium collected over all four time points).

A. It is not uncommon for the apparent concentrations of active test agent in samples of supernatant medium or in diluted samples of material applied to the apical surface of the units to lie outside the range of concentrations of the standards after completion of an ELISA. No concentrations of material present in experimental samples are determined by extrapolation beyond the concentrations of the standards; rather, samples are rediluted appropriately to generate concentrations of the test material which can be more accurately determined by interpolation between the standards in a repeat ELISA.

B. The ELISA for a biologically active test agent, for example, interferon-β, is unique in its design and recommended protocol. Unlike most kits, the ELISA employs two monoclonal antibodies, one for capture and another, directed towards a nonoverlapping determinant for the biologically active test agent, e.g., interferon-β, as the detection antibody (this antibody is conjugated to horseradish peroxidase). As long as concentrations of IFN-β that lie below the upper limit of the assay are present in experimental samples, the assay protocol can be employed as per the manufacturer's instructions, which allow for incubation of the samples on the ELISA plate with both antibodies present simultanously. When the IFN-β levels in a sample are significantly higher than this upper limit, the levels of immunoreactive IFN-β may exceed the amounts of the antibodies in the incubation mixture, and some IFN-β which has no detection antibody bound will be captured on the plate, while some IFN-β which has detection antibody bound may not be captured. This leads to serious underestimation of the IFN-β levels in the sample (it will appear that the IFN-β levels in such a sample lie significantly below the upper limit of the assay). To eliminate this possibility, the assay protocol has been modified:

B.1. The diluted samples are first incubated on the ELISA plate containing the immobilized capture antibody for one hour in the absence of any detection antibody. After the one hour incubation, the wells are washed free of unbound material.

B.2. The detection antibody is incubated with the plate for one hour to permit formation of immune complexes with all captured antigen. The concentration of detection antibody is sufficient to react with the maximum level of IFN-β which has been bound by the capture antibody. The plate is then washed again to remove any unbound detection antibody.

B.3. The peroxidase substrate is added to the plate and incubated for fifteen minutes to allow color development to take place.

B.4. The “stop” solution is added to the plate, and the absorbance is read at 450 nm as well as 490 nm in the VMax microplate spectrophotometer. The absorbance of the colored product at 490 nm is much lower than that at 450 nm, but the absorbance at each wavelength is still proportional to concentration of product. The two readings ensure that the absorbance is linearly related to the amount of bound IFN-β over the working range of the VMax instrument (we routinely restrict the range from 0 to 2.5 OD, although the instrument is reported to be accurate over a range from 0 to 3.0 OD). The amount of IFN-β in the samples is determined by interpolation between the OD values obtained for the different standards included in the ELISA. Samples with OD readings outside the range obtained for the standards are rediluted and run in a repeat ELISA.

### Results

Measurement of transepithelial resistance by TER Assay: After the final assay time points, membranes were placed in individual wells of a 24 well culture plate in 0.3 mL of fresh medium and the transepithelial electrical resistance (TER) was measured using the EVOM Epithelial Voltohmmeter and an Endohm chamber (World Precision Instruments, Sarasota, Fla.). The top electrode was adjusted to be close to, but not in contact with, the top surface of the membrane. Tissues were removed, one at a time, from their respective wells and basal surfaces were rinsed by dipping in clean PBS. Apical surfaces were gently rinsed twice with PBS. The tissue unit was placed in the Endohm chamber, 250 μL of PBS added to the insert, the top electrode replaced and the resistance measured and recorded. Following measurement, the PBS was decanted and the tissue insert was returned to the culture plate. All TER values are reported as a function of the surface area of the tissue.

The final numbers were calculated as:

TER of cell membrane=(Resistance (R) of Insert with membrane−R of blank Insert)×Area of membrane (0.6 cm²).

The effect of pharmaceutical formulations comprising interferon-β-1a and intranasal delivery-enhancing agents on TER measurements across the EpiAirway™ Cell Membrane (mucosal epithelial cell layer) is shown in Tables 6 and 7. A decrease in TER value relative to the control value (control=approximately 1000 ohms-cm²; normalized to 100.) indicates a decrease in cell membrane resistance and an increase in mucosal epithelial cell permeability.

Exemplary formulations F2, F3, F4, F5, and F6 showed the greatest decrease in cell membrane resistance. Formulation F7 showed a significant decrease in cell membrane resistance. (Table 5) The results indicate that these exemplary formulations provide significant increases in mucosal epithelial cell permeability. The exemplary formulations will enhance intranasal delivery of interferon-β to the blood serum or central nervous system. The results indicate that these exemplary formulations when contacted with a mucosal epithelium yield significant increases in mucosal epithelial cell permeability to interferon-β.

TABLE 4
Influence of Pharmaceutical Formulations
Comprising Interferon-β-1a and Intranasal
Delivery-Enhancing Agents on TER of
EpiAirway ™ Cell Membrane

| Formulation with Interferon-β-1a | % TER |
|---|---|
| No Treatment Control-1 | 100 |
| N-Caproic Acid Sodium (0.138% w/v) | 100.00 |
| Pluronic 127 (0.3% w/v) | 100.00 |
| α-Cyclodextrin (5% w/v, Inf. Conc. 33 ug/mL) | 57.38 |
| Chenodeoxycholic Acid, sodium salt (0.5% w/v, Inf. | 2.52 |
| Conc. 165 ug/mL) | |
| Na-Nitroprusside (0.3% w/v, Inf. Conc. 165 ug/mL) | 13.68 |
| No TreatmentControl-2 | 100 |
| Chitosan 0.5% w/v | 13.44 |
| Arginine 10% w/v | 44.34 |
| Gamma-CD | 100.00 |
| SNAP | 100.00 |
| Avonex ® (Interferon-β-1a) | 100.00 |
| No TreatmentControl-3 | 100 |
| Diedecanoyl-1-alpha-phosphatidylcholine | 1.78 |
| palmotoyl-Dl-Carnitine | 89.83 |
| Poly-Arginine | 30.65 |
| No TreatmentControl-4 | 100.00 |
| Avonex ® (Interferon-β-1a)-2 | 90.00 |
| α-Cyclodextrin (5% w/v)-2 | 28.96 |
| Chitosan 0.5% w/v-2 | 20.32 |
| Poly (Gud) 0.5% w/v | 16.29 |
| Na-Nitroprusside (0.3% w/v)-2 | 76.64 |

TABLE 5
Influence of Pharmaceutical Formulations
Comprising Interferon-β-1a and
Intranasal Delivery-Enhancing Agents
TER of EpiAirway ™ Cell Membrane

| Formulations | |
|---|---|
| with Interferon-β-1a | % TER |
| No Treatment-1 | 100 |
| Avonex ® (Control-1) | 63 |
| F2 (HPC, Alpha-CD, Arginine, Na-TC, EDTA, BC) | 3 |
| F7 (Chitosan, EDTA, M-beta-CD, SGC, BC) | 22 |
| F8 (HPMC, HPCD, Na TC, BC) | 80 |
| No Treatment-2 | 100 |
| Avonex ® (Control-2) | 100 |
| F3 (HPC, Gamma-CD, Arginine, STC, EDTA, BC) | 2 |
| F6 (HPC, EDTA, Arginine, STC, BC) | 0 |
| F4 (Chitosan, BC, SDC, EDTA, Methyl-B-CD) | 1 |
| F5 (DDPC, MBCD, EDTA, Gelatin) | 2 |

Permeation Kinetics as Measured by ELISA Assay:

The effect of pharmaceutical formulations comprising interferon-β-1a and intranasal delivery-enhancing agents on the permeation of interferon-β-1a across the EpiAirway™ Cell Membrane (mucosal epithelial cell layer) is measured as described above. The results are shown in Tables 4 and 5. Permeation of interferon-β-1a across the EpiAirway™ Cell Membrane is measured by ELISA assay.

For the exemplary intranasal formulations of the present invention, the greatest increase in interferon-β-1a permeation occurred in Formulation F5, (about 316 fold increase in permeation), Formulation F4 (85 fold increase in permeation), Formulation F6 (69 fold increase in permeation), Formulation F3 (25 fold increase in permeation) compared to the Avonex® (interferon-β-1a) control. The results indicate that these exemplary formulations provide significant increases in mucosal epithelial cell permeability. The exemplary formulations will provide enhanced intranasal delivery of interferon-β-1a.

TABLE 6
Influence of Pharmaceutical Formulations Comprising Interferon-β-
1a and Intranasal Delivery-Enhancing Agents on Permeation of
Interferon-β-1a through EpiAirway ™ Cell Membrane.

| Formulations: | | | | | |
|---|---|---|---|---|---|
| Interferon-β-1a | % Permeation at Time points (min) | | | | |
| +/− Enhancer Solutions | 0 | 15 | 30 | 60 | 120 |
| Avonex ® Control-1 | 0.0 | 0.00059 | 0.00116 | 0.00173 | 0.00234 |
| (Interferon-β-1a) | | | | | |
| N-Caproic Acid Sodium | 0.0 | 0.00039 | 0.00078 | 0.00121 | 0.00166 |
| (0.138% w/v, Inf. Conc. | | | | | |
| 33 ug/mL) | | | | | |
| Pluronic −127 | 0.0 | 0.00037 | 0.00078 | 0.00118 | 0.00159 |
| (0.3% w/v, Inf. Conc. | | | | | |
| 33 ug/mL) | | | | | |
| Alpha-Cyclodextrin | 0.0 | 0.00059 | 0.00275 | 0.01351 | 0.03338 |
| (5% w/v, Inf. Conc. | | | | | |
| 33 ug/mL) | | | | | |
| Sodium-salt of | 0.0 | 0.00015 | 0.00178 | 0.00741 | 0.01492 |
| Chenodeoxycholic Acid | | | | | |
| (0.5% w/v, Inf. Conc. 165 | | | | | |
| ug/mL) | | | | | |
| Sodium-Nitroprusside | 0.0 | 0.00120 | 0.00541 | 0.01248 | 0.01978 |
| (0.3% w/v, Inf. Conc. 165 | | | | | |
| ug/mL) | | | | | |
| Chitosan 0.5% w/v | 0.0 | 0.00055 | 0.00128 | 0.00618 | 0.13256 |
| Arginine 10% w/v | 0.0 | 0.00317 | 0.01293 | 0.03194 | 0.06569 |
| Gamma Cyclodextrin | 0.0 | 0.00062 | 0.00115 | 0.00175 | 0.00259 |
| 1% w/v | | | | | |
| Sodium Nitros0-N-Acetyl- | 0.0 | 0.00059 | 0.00117 | 0.00180 | 0.00249 |
| Penicillamine Sodium | | | | | |
| (0.5% w/v) | | | | | |
| Sodium L-Alpha- | 0.0 | 0.00009 | 0.00020 | 0.00054 | 0.00191 |
| Phosphatidylcholine | | | | | |
| Diedecanoyl (3.5% w/v) | | | | | |
| Palmotoyl-Dl-Carnitine | 0.0 | 0.00008 | 0.00030 | 0.00042 | 0.00059 |
| (0.02% w/v) | | | | | |
| Poly-Arginine (0.5% w/v) | 0.0 | 0.00014 | 0.00032 | 0.00058 | 0.00165 |
| Alpha Cyclodextrin (5% w/v) | 0.0 | 0.00327 | 0.00689 | 0.02713 | 0.08434 |
| Chitosan (0.5% w/v) | 0.0 | 0.00018 | 0.00037 | 0.00322 | 0.05584 |
| Poly (Gud) 0.5% w/v pH = | 0.0 | 0.00004 | 0.00028 | 0.00053 | 0.01162 |
| 4.2 | | | | | |
| Sodium Nitroprusside | 0.0 | 0.00017 | 0.00044 | 0.00144 | 0.00221 |
| (0.3% w/v) | | | | | |

TABLE 7
Influence of Pharmaceutical Formulations Comprising Interferon-β-1a and
Intranasal Delivery-Enhancing Agents on Permeation of Interferon-β-1a
through EpiAirway ™ Cell Membrane.

| | | | | | | Fold |
|---|---|---|---|---|---|---|
| Pharmaceutical | % Permeation at Time Points (min) | | | | | Increase in |
| Formulation | 0 | 15 | 30 | 60 | 120 | Permeation |
| Avonex ® Control-1 | 0 | 0.0016 | 0.0054 | 0.0139 | 0.0254 | 1 |
| (Interferon-β-1a) | | | | | | |
| F2 | 0 | 0.0016 | 0.0043 | 0.0337 | 0.3818 | 15 |
| (HPC, Alpha-CD, | | | | | | |
| Arginine, Na-TC, | | | | | | |
| EDTA, BC) | | | | | | |
| F7 | 0 | 0.0024 | 0.0046 | 0.0164 | 0.0439 | 2 |
| (Chitosan, EDTA, M- | | | | | | |
| beta-CD, SGC, BC) | | | | | | |
| F8 | 0 | 0.0024 | 0.0035 | 0.0105 | 0.0179 | 1 |
| (HPMC, HPCD, Na TC, BC) | | | | | | |
| Avonex ® Control-2 | 0 | 0.0002 | 0.0007 | 0.0025 | 0.0048 | 1 |
| (Interferon-β-1a) | | | | | | |
| F3 | 0 | 0.0079 | 0.0234 | 0.0596 | 0.1212 | 25 |
| (HPC, Gamma-CD, | | | | | | |
| Arginine, Na-TC, | | | | | | |
| EDTA, BC) | | | | | | |
| F6 | 0 | 0.0052 | 0.0292 | 0.1072 | 0.3318 | 69 |
| (HPC, Arginine, Na-TC, | | | | | | |
| EDTA, BC) | | | | | | |
| F4 | 0 | 0.0035 | 0.0138 | 0.0584 | 0.4082 | 85 |
| (Chitosan, SDC, Meth- | | | | | | |
| B-CD, EDTA, BC) | | | | | | |
| F5 | 0 | 0.0196 | 0.1810 | 0.5958 | 1.5141 | 316 |
| (Gelatin, DDPC, MB- | | | | | | |
| CD, EDTA, BC) | | | | | | |

The Avonex control contains human serum albumin and a phosphate buffer, essentially the composition described in U.S. patent application No. 20010043915 (Nov. 22, 2001). The formulations described herein, especially F5, have substantially larger permeation over control (for example, 316 fold).

Formulation F7 of the present invention, described herein, contains sodium glycocholate. A similar formulation containing a surfactant, sodium glycocholate, is described in Maitani, et al., Drug Des Deliv 1: 65-70. While the formulation containing sodium glycocholate of Maitani showed only a 2-fold increase over control, Formulation F7 shows a 316-fold increase over control.

MTT Assay: The MTT assays were performed using MTT-100, MatTek kits. 0.3 mL of the MTT solution was added into each well. Tissue inserts were gently rinsed with clean PBS and placed in the MTT solution. The samples were incubated at 37° C. for 3 hours. After incubation the cell culture inserts were then immersed with 2.0 mL of the extractant solution per well to completely cover each insert. The extraction plate was covered and sealed to reduce evaporation. Extraction proceeds overnight at RT in the dark. After the extraction period was complete, the extractant solution was mixed and pipetted into a 96-well microtiter plate. Triplicates of each sample were loaded, as well as extractant blanks. The optical density of the samples was then measured at 550 nm on an optical density plate reader (Molecular Devices).

The MTT assay on exemplary formulations of the present invention for enhanced mucosal delivery of interferon-β-1a are shown in Tables 8 and 9. The results for formulations comprising interferon-β-1a and one or more intranasal delivery enhancing agents, for example, Formulations F2, F3, F4, F5, F7, and F8, indicate that there is minimal toxic effect of this exemplary embodiment on viability of the mucosal epithelial tissue (Table 9).

TABLE 8
Influence of Pharmaceutical Formulations
Comprising Interferon-β-1a and
Intranasal Delivery-Enhancing Agents on
the Viability of EpiAirway^{MT} Cell
Membrane as shown by % MTT

| Formulation with Interferon-β-1a | % MTT |
|---|---|
| No TreatmentControl-1 | 100.00 |
| N-Caproic Acid Sodium (0.138% w/v) | 96.93 |
| Pluronic 127 (0.3% w/v) | 98.02 |
| Alpha-Cyclodextrin (5% w/v, Inf. Conc. 33 ug/mL) | 97.97 |
| Sodium-salt of Chenodeoxycholic Acid | 43.03 |
| (0.5% w/v, Inf. Conc. 165 ug/mL) | |
| Na-Nitroprusside (0.3% w/v, Inf. Conc. 165 ug/mL) | 77.28 |
| No TreatmentControl-2 | 100.00 |
| Chitosan 0.5% w/v | 96.24 |
| Arginine 10% w/v | 95.06 |
| Gamma-CD 1% w/v | 100.00 |
| SNAP | 98.46 |
| Avonex Control-1 (Interferon-β-1a) | 100.00 |
| No TreatmentControl-3 | 100.00 |
| Diedecanoyl-1-alpha-phosphatidylcholine | 92.37 |
| palmotoyl-Dl-Carnitine | 99.29 |
| Poly-Arginine | 100.58 |
| No TreatmentControl-4 | 100.00 |
| Avonex ® Control-2 (Interferon-β-1a) | 96.56 |
| Alpha-Cyclodextrin (5% w/v)-2 | 99.34 |
| Chitosan 0.5% w/v-2 | 100.00 |
| Poly (Gud) 0.5% w/v | 100.00 |
| Sodium Nitroprusside (0.3% w/v)-2 | 100.00 |

TABLE 9
Influence of Pharmaceutical Formulations
Comprising Interferon-β-1a and
Intranasal Delivery-Enhancing Agents on
the Viability of EpiAirway ™ Cell
Membrane as shown by % MTT

| Formulations | |
|---|---|
| with Interferon-β-1a | % MTT |
| No Treatmentl-1 | 100.0 |
| Avonex ® Control-1 (Interferon-β-1a) | 96.4 |
| F2 (HPC, Alpha-CD, Arginine, Na-TC, EDTA, BC) | 51.8 |
| F7 (Chitosan, EDTA, M-beta-CD, SGC, BC) | 98.7 |
| F8 (HPMC, HPCD, Na TC, BC) | 101.6 |
| No Treatment-2 | 100 |
| Avonex ® Control-2 (Interferon-β-1a) | 97 |
| F3 (HPC, Gamma-CD, Arginine, STC, EDTA, BC) | 39 |
| F6 (HPC, EDTA, Arginine, STC, BC) | 13 |
| F4 (Chitosan, BC, SDC, EDTA, Methyl-β-CD) | 97 |
| F5 (DDPC, M-B-CD, EDTA, Gelatin) | 51 |

LDH Assay: The LDH assay on exemplary formulations of the present invention for enhanced mucosal delivery of interferon-β-1a are shown in Tables 10 and 11. The results indicate that there is minimal toxic effect of exemplary embodiments, for example, Formulations F2, F3, F4, F5, F7, and F8, on viability of the mucosal epithelial tissue (Table 11).

TABLE 10
Influence of Pharmaceutical Formulations Comprising
Interferon-β-1a and Intranasal Delivery-Enhancing
Agents on the Viability of EpiAirway^{MT} cell
membrane as shown by % Dead Cells (LDH Assay)

| Formulation with | % Dead Cells at Time points (min) | | | | |
|---|---|---|---|---|---|
| Interferon-β-1a | 0 | 15 | 30 | 60 | 120 |
| N-Caproic Acid Sodium | 0 | 0.28 | 0.28 | 0.28 | 0.28 |
| (0.138% w/v, Inf. Conc. | | | | | |
| 33 ug/mL) | | | | | |
| Pluronic-127 (0.3% w/v, Inf. | 0 | 0.30 | 0.30 | 0.30 | 0.30 |
| Conc. 33 ug/mL) | | | | | |
| Alpha-Cyclodextrin (5% w/v, | 0 | 0.14 | 0.14 | 0.14 | 0.14 |
| Inf. Conc. 33 ug/mL) | | | | | |
| Sodium-salt of | 0 | 4.18 | 18.15 | 37.28 | 63.77 |
| Chenodeoxycholic Acid (0.5% | | | | | |
| w/v, Inf. Conc. 165 ug/mL) | | | | | |
| Na-Nitroprusside (0.3% w/v, | 0 | 2.717 | 7.326 | 12.759 | 20.654 |
| Inf. Conc. 165 ug/mL) | | | | | |
| Chitosan 0.5% w/v | 0 | 0.046 | 0.046 | 0.100 | 0.378 |
| Arginine 10% w/v | 0 | 0.232 | 0.255 | 0.302 | 0.372 |
| Gamma-CD 1% | 0 | 0.372 | 0.488 | 0.604 | 0.789 |
| SNAP 0.5% | 0 | 0.743 | 0.813 | 0.906 | 1.068 |
| Avonex ® Control | 0 | 0.441 | 0.534 | 0.650 | 0.789 |
| (Interferon-β-1a) | | | | | |
| Diedecanoyl-1-alpha- | 0 | 0.372 | 0.488 | 0.906 | 2.276 |
| phosphatidylcholine 3.5% | | | | | |
| Palmotoyl-Dl-Carnitine 0.02% | 0 | 0.627 | 0.697 | 0.766 | 0.882 |
| Poly-Arginine 0.5% | 0 | 0.836 | 0.998 | 1.161 | 1.486 |
| No treatment | 0 | 0.000 | 0.000 | 0.000 | 0.046 |
| Avonex ® Control | 0 | 0.000 | 0.000 | 0.000 | 1.370 |
| (Interferon-β-1a)-2 | | | | | |
| Alpha-Cyclodextrin | 0 | 0.000 | 0.000 | 0.000 | 0.093 |
| (5% w/v)-2 | | | | | |
| Chitosan 0.5% (w/v)-2 | 0 | 0.000 | 0.000 | 0.000 | 0.139 |
| Poly (Gud) 0.5% w/v | 0 | 0.070 | 0.163 | 0.279 | 0.720 |
| Na-Nitroprusside (0.3% w/v)-2 | 0 | 0.163 | 0.209 | 0.255 | 0.488 |

TABLE 11
Influence of Pharmaceutical Formulations Comprising
Interferon-β-1a and Intranasal Delivery-Enhancing
Agents on the Viability of EpiAirway ™ cell
membrane as shown by % Dead Cells (LDH Assay)

| Formulations | % Dead Cells at Time points (min) | | | | |
|---|---|---|---|---|---|
| with Interferon-β-1a | 0 | 15 | 30 | 60 | 120 |
| Control (No Treatment) | 0.000 | 0.000 | 0.000 | 0.023 | 0.023 |
| Avonex ® Control-2 | 0.000 | 0.000 | 0.000 | 0.000 | 0.000 |
| (Interferon-β-1a) | | | | | |
| F2 | 0.000 | 0.464 | 1.045 | 3.135 | 8.034 |
| (HPC, Alpha-CD, Arginine, | | | | | |
| Na-TC, EDTA, BC) | | | | | |
| F7 | 0.000 | 0.000 | 0.116 | 0.255 | 0.372 |
| (Chitosan, EDTA, M-beta- | | | | | |
| CD, SGC, BC) | | | | | |
| F8 | 0.000 | 0.000 | 0.070 | 0.163 | 0.232 |
| (HPMC, HPCD, Na TC, BC) | | | | | |
| Avonex ® Control-2 | 0.000 | 0.163 | 0.163 | 0.163 | 0.186 |
| (Interferon-β-1a) | | | | | |
| F3 | 0.000 | 0.720 | 2.554 | 5.387 | 10.797 |
| (HPC, Gamma-CD, | | | | | |
| Arginine, STC, EDTA, BC) | | | | | |
| F6 | 0.000 | 1.138 | 6.618 | 15.766 | 30.580 |
| (HPC, EDTA, Arginine, STC, | | | | | |
| BC) | | | | | |
| F4 | 0.000 | 0.070 | 0.139 | 0.279 | 0.859 |
| (Chitosan, BC, SDC, EDTA, | | | | | |
| Methyl-β-CD) | | | | | |
| F5 | 0.000 | 0.255 | 1.045 | 2.415 | 4.992 |
| (DDPC, M-β-CD, EDTA, | | | | | |
| Gelatin) | | | | | |

### EXAMPLE 5

### Combinatorial Formulations of a Cytokine and Steroid for Treating Multiple Sclerosis

The current standards of care for multiple sclerosis include injections, either intravenously, subcutaneously or intramuscularly, of interferon beta, glatiramer, or steroids, including corticosteroids like methylprednisolone and prednisolone. All of these have the disadvantage of being injections with some local adverse reactions associated with them. According to the methods and formulations of the invention, all of these important pharmaceuticals can be effectively delivered intranasally to for the treatment of target diseases and conditions such as multiple sclerosis.

COPAXONE® (glatiramer acetate for injection) is indicated for the reduction of relapses in relapsing-remitting multiple sclerosis. Glatiramer acetate (GA) is a mixture of synthetic polypeptides composed of four amino acids, L-glutamic acid, L-alanine, L-tyrosine, and L-lysine, with an average molecular weight of 4,700 to 11,000. GA is very effective in suppression of experimental autoimmune encephalomyelitis (EAE), the animal model of multiple sclerosis (MS). Various mechanisms of action of GA have been proposed, but the most important is probably the induction of antigen-specific suppressor T cells.

The most common side effects of COPAXONE® are redness, pain, swelling, itching, or a lump at the site of injection, flushing, chest pain, weakness, infection, pain, nausea, joint pain, anxiety, and muscle stiffness. These reactions are usually mild and seldom require professional treatment. Some patients report a short-term reaction right after injecting COPAXONE®. This reaction can involve flushing (feeling of warmth and/or redness), chest tightness or pain with heart palpitations, anxiety, and trouble breathing. These symptoms generally appear within minutes of an injection, last about 15 minutes, and go away by themselves without further problems.

Formulation of Glatiramer

| | |
|---|---|
| Glatiramer acetate | 200 mg |
| Mannitol | 400 mg |
| Water | 1.0 mL |

**One or more delivery enhancing agents as disclosed above

0.1 mL of the above formulation is administered in a fine spray to one nostril every day, alternating from left nostril to right.

Formulation of Corticisteroids

| Corticosteroid: | |
|---|---|
| Bethamethasone | 6.0 mg or |
| Dexamethasone | 7.5 mg or |
| Methylprednisolone | 40.0 mg or |
| Triamcinolone | 40.0 mg |
| Water | 1.0 mL |

**One or more delivery enhancing agents as disclosed above

0.1 mL of the above formulation is administered in a fine spray to one nostril every day, alternating from left nostril to right. Cortisone, hydrocortisone, prednisone and prednisolone, clobetasol, desonide, fluocinolone, fluocinonide, and mometasone can be substituted in the formulation above at doses that provide benefit in multiple sclerosis.

The following steroids exemplify useful steroids that can be employed within the formulations and methods herein to treat multiple sclerosis

1. Amcinonide

2. Beclomethasone

3. Betamethasone

4. Clobetasol

5. Clobetasone

6. Desoximetasone

7. Diflorasone

8. Diflucortolone

9. Fluocinolone

10. Fluocinonide

11. Flurandrenolide (except Drenison-1/4)

12. Fluticasone

13. Halcinonide

14. Halobetasol

15. Hydrocortisone butyrate

16. Hydrocortisone valerate

17. Mometasone

18. Triamcinolone

### EXAMPLE 6

### Formulation F5 of the Present Invention in Combination with Triamcinolone Acetonide Corticosteroid Improves Cell Viability

The present example provides an in vitro study to determine the permeability and reduction in epithelial mucosal inflammation of an intranasally administered cytokine, for example, interferon-β, in combination with a steroid composition, for example, triamcinolone acetonide, and further in combination with one or more intranasal delivery-enhancing agents. The study involves determination of epithelial cell permeability by TER assay and reduction in epithelial mucosal inflammation as measured by cell viability in an MTT assay by application of an embodiment comprising interferon-β and triamcinolone acetonide.

Formulation F5 (Interferon β-1a, 60 μg+DDPC, M-β-CD, EDTA, Gelatin; See Table 3 above) is combined in a formulation with triamcinolone acetonide at a dosage of 0.5, 2.0, 5.0, or 50 μg. Normal dose of, (Nasacort®, Aventis Pharmaceuticals) for seasonal allergic rhinitis, is 55 μg per spray. Formulation F5 in combination with triamcinolone acetonide corticosteroid improves cell viability as measured by the MTT assay, while maintaining epithelial cell permeability as measured by TER and ELISA assays.

Measurement of permeability of Formulation F5 in the presence or absence of triamcinolone acetonide was performed by transepithelial electrical resistance (TER) assays in an EpiAirway™ cell membrane. TER assays of Formulation F5 plus triamcinolone acetonide at a concentration of 0.5, 2.0, 5.0, or 50 μg per spray indicate that interferon β permeability did not decrease and was equal to permeability of Formulation F5 alone. Formulation F5 plus triamcinolone acetonide at a triamcinolone acetonide concentration between 0 and 50 μg per spray is at least 10-fold greater than permeability of interferon β in an Avonex® control. See Table 12.

Measurement of permeability of Formulation F5 in the presence or absence of triamcinolone acetonide was performed by ELISA assay in an EpiAirway™ cell membrane. Similar to the TER assay above, ELISA assay of Formulation F5 plus triamcinolone acetonide at a concentration of 0.5, 2.0, 5.0, or 50 μg per spray indicate that interferon β permeability did not decrease and was equal to permeability of Formulation F5 alone. Formulation F5 plus triamcinolone acetonide at a triamcinolone acetonide concentration between 0 and 50 μg per spray is greater than permeability of interferon β in an Avonex control. See Table 13.

MTT assay measured cell viability of Formulation F5 in the presence or absence of triamcinolone acetonide. These results indicate that addition of triamcinolone acetonide (at a concentration of 0.5, 2.0, 5.0, or 50 μg per spray) to Formulation F5 improves cell viability by approximately 18% to 50% compared to Formulation F5 in the absence of triamcinolone acetonide. See Table 14.

Addition of triamcinolone acetonide to Formulation F5 increases cell viability and maintains epithelial permeability as measured by TER assay comparable to Formulation F5 in the absence of triamcinolone acetonide.

TABLE 12
Influence of Pharmaceutical Formulations
Comprising Interferon-β-1a, Intranasal
Delivery-Enhancing Agents, and
Triamcinolone Acetonide on TER of
EpiAirway ™ Cell Membrane

| Formulations | % TER |
|---|---|
| No Treatment-1 | 100 |
| Avonex ® (Control) | 22 |
| Formulation F5 | 2 |
| (DDPC, MBCD, EDTA, Gelatin) | |
| F5 + 50 μg Triamcinolone Acetonide | 2 |
| F5 + 5 μg Triamcinolone Acetonide | 2 |
| F5 + 2 μg Triamcinolone Acetonide | 1 |
| F5 + 0.5 μg Triamcinolone Acetonide | 2 |

TABLE 13
Influence of Pharmaceutical Formulations Comprising Interferon-
β-1a, Intranasal Delivery-Enhancing Agents, and Triamcinolone
Acetonide on Permeation of Interferon-β-1a through
EpiAirway ™ Cell Membrane as measured by ELISA assay.

| | % Permeation at Time points (min) | | | | |
|---|---|---|---|---|---|
| Formulations | 0 | 15 | 30 | 60 | 120 |
| No Treatment-1 | 0.0 | 0.0 | 0.0 | 0.0 | 0.0 |
| Avonex ® | 0.0 | 0.0083 | 0.0333 | 0.113 | 0.3567 |
| (Control) | | | | | |
| F5 (DDPC, | 0.0 | 0.2092 | 0.5783 | 0.7867 | 0.8617 |
| MBCD, EDTA, | | | | | |
| Gelatin) | | | | | |
| F5 + 50 μg | 0.0 | 0.1133 | 0.4583 | 0.8080 | 0.9417 |
| Triamcinolone | | | | | |
| Acetonide | | | | | |
| F5 + 5 μg | 0.0 | 0.1583 | 0.5658 | 0.7808 | 0.8617 |
| Triamcinolone | | | | | |
| Acetonide | | | | | |
| F5 + 2 μg | 0.0 | 0.2092 | 0.5900 | 0.8208 | 0.8883 |
| Triamcinolone | | | | | |
| Acetonide | | | | | |
| F5 + 0.5 μg | 0.0 | 0.1300 | 0.5383 | 0.7542 | 0.8342 |
| Triamcinolone | | | | | |
| Acetonide | | | | | |

TABLE 14
Influence of Pharmaceutical Formulations Comprising
Interferon-β-1a, Intranasal Delivery-Enhancing
Agents, and Triamcinolone Acetonide on the
Viability of EpiAirway ™
Cell Membrane as shown by % MTT

| Formulation with Interferon-β-1a | % MTT |
|---|---|
| No Treatment-1 | 100 |
| Avonex ® (IFN-β Control) | 82 |
| F5 (IFN-β + DDPC, MBCD, EDTA, Gelatin) | 61 |
| F5 + 50 μg Triamcinolone Acetonide | 72 |
| F5 + 5 μg Triamcinolone Acetonide | 90 |
| F5 + 2 μg Triamcinolone Acetonide | 75 |
| F5 + 0.5 μg Triamcinolone Acetonide | 71 |

Treatment of MS disease is accomplished with an intranasal formulation of interferon-β in combination with one or more steroid or corticosteroid compound(s) typically high potency compounds or formulations, but also in certain cases medium potency, or low potency compounds or formulations. Overall potency (equivalent dosages) of high, medium, and low potency steroids are given. In one embodiment, an intranasal formulation of interferon-β in combination with a high potency steroid composition includes, but is not limited to, betamethasone (0.6 to 0.75 mg dosage), or dexamethasone (0.75 mg dosage). In a further embodiment, an intranasal formulation of interferon-β in combination with a medium potency steroid composition includes, but is not limited to, methylprednisolone (4 mg dosage), triamcinolone (4 mg dosage), or prednisolone (5 mg dosage). In a further embodiment, an intranasal formulation of interferon-β in combination with a low potency steroid composition includes, but is not limited to hydrocortisone (20 mg dosage) or cortisone (25 mg dosage).

### EXAMPLE 7

### Bioavailability and Bioactivity of Intranasal Administration of Interferon-β (IFN-β) Formulations of the Present Invention Administered to Healthy Male Volunteers

Study Synopses.

The present example provides a non-blinded study to determine the uptake of intranasally administered interferon-β in combination with one or more intranasal delivery-enhancing agents into the blood serum in healthy male volunteers. The study involves administration of an intranasal effective amount of an exemplary formulation of the invention, Formulation F9, as described above, to evaluate the absorption and tolerance of the interferon-β intranasal formulation by the subjects. The study is a single dose, parallel group pharmacokinetic/pharmacodynamic study to evaluate absorption and tolerance of interferon-β-1a by two routes of administration: intramuscular and intranasal. The objective of the study is to evaluate the absorption, tolerance and pharmacodynamic parameters of equimolar doses of a exemplary formulation of interferon-β-1a in combination with one or more intranasal delivery-enhancing agents of the present invention, administered intranasally, versus interferon-β-1a, (Avonex®, Biogen, Inc.) recombinant for injection administered intramuscularly.

Protocol: Twelve healthy male subjects, age 18-50, are enrolled in the study. Six subjects receive a single intranasal dose of 60 μg (6.0 MIU) delivered as two 0.1 ml sprays, each containing 30 μg/0.1 ml and six subjects receive a single intramuscular dose of 60 μg delivered intramuscularly.

The study was conducted in compliance with Good Clinical Practice regulations and all necessary regulatory and Institutional Review Board approvals were in place prior to start of the study. Dosage is also evaluated during the clinical testing phase for Avonex®. Each subject visits the clinical site ten times in a six-month period. These visits consist of a screening visit, one dosing visit and eight safety monitoring visits.

A complete medical history and physical examination is performed at the pre-study screening visit. Blood pressure, pulse, respiration rate, and body temperature are measured. Clinical laboratory evaluations are performed during the pre-study period and on Visit 6 (96 hours post dosing).

On the day of dosing, vital signs (blood pressure, pulse, respiration rate, and body temperature) are measured before dosing and post dosing at 15, 30, 45, 60, 75, 90, 120, 240, 480 minutes and 12, 15 (prior to discharge), hours and 1, 2, 3, and 4 days after dosing. Vital signs are also be measured at 30, 60, 90 and 180 days after dosing.

Serial blood samples (7 mL each) are drawn into appropriate vacutainers for interferon-β, neopterin/β-2-microglobulin/2′,5′-oligoadenylate synthetase [OAS], and neutralizing antibodies at various time points. Nasal examinations are performed at the pre-study screening period, immediately before intranasal dosing and at 15, 30, 45, 75, 90, 120, 240, 480 minutes, 12, and 15 hours and 1, 2, 3, and 4 days after dosing for patients in the intranasal group.

The results of the study are evaluated for safety and bioavailability (Cₘₐₓ, tₘₐₓ, AUC). If administration of the product results in a grading scale of 3 (based on the Common Toxicity Criteria [CTC]) for any of the parameters observed, the study is discontinued.

Interferon-β-1a is currently marketed as Avonex® (Biogen, Inc.) for the treatment of relapsing-remitting multiple sclerosis. The commercial product, Avonex® (Biogen, Inc.), is reconstituted as Interferon-β-1a, recombinant powder for injection, 33 μg (6.6 MIU) with a single use 10 cc vial of diluent. When reconstituted according to its approved labeling, the resulting solution is 30 μg/mL.

The objective of the study is to evaluate the absorption, tolerance and pharmacodynamic parameters (neopterin, β-2-microglobulinin, and 2′,5′-oligoadenylate synthetase [OAS]) by intranasal administration of interferon-β-1a utilizing pharmaceutical formulations and methods of the present invention compared to intramuscular administration of interferon-β-1a (Avonex®, Biogen, Inc.) in formulations known in the art.

Reference Product: Reference product is interferon-β-1a (Avonex®) 60 μg for intramuscular injection. Interferon-β-1a is supplied as lyophilized powder in a single use vial containing 33 μg of interferon β-1a, albumin human, sodium chloride, dibasic sodium phosphate, and monobasic sodium phospate. Diluent is supplied in a single-use vial (Sterile Water for Injection, preservative free).

The Avonex® Interferon β-1a injection is purchased from a commercial supplier and administered to the subject by the principal investigator.

The Avonex® is hand delivered on ice to clinical site on the day of dosing. Once delivered to the site, the product is stored in the refrigerator with the original packaging and labeling until reconstituted. Once reconstituted, any unused medication is discarded.

Test Formulation (F9) Product: The test formulation, Formulation F9, is a nasal spray containing 60 μg interferon-β-1a=(2×30 μg/0.1 ml spray unit dose vials). Each unit dose vial contains 30 μg/0.1 ml spray in each nostril for a dose of 60 μg. Formulation F9 contains: interferon-β-1a (300 μg; 60 MIU), dibasic sodium phosphate, monobasic sodium phosphate, albumin human, sodium chloride, benzalkonium chloride, L-alpha-phosphatidylcholine didecanoyl, methyl-β-cyclodextrin, EDTA disodium, gelatin and purified water per 1.0 ml.

The intranasal product formulation is manufactured under GMP conditions. Storage conditions is at 5° C.

Trial Design:

This is a single dose, parallel group study to evaluate absorption, tolerance and pharmacodynamic parameters of Interferon-β-1a by two routes of administration: intramuscular and intranasally. The study involves twelve healthy male subjects randomly assigned six per group (6 subjects intramuscular and 6 intranasal). The objective of the study is to evaluate the absorption, tolerance and pharmacodynamic parameters; neopterin, β-2-microglobulin, and 2′,5′-oligoadenylate synthetase of intranasal administration of Interferon β-1a by formulations of the present invention versus intramuscular administration of interferon-β-1a (Avonex®, Biogen, Inc.). Each subject visits the clinical site ten times within a 6 month period. These visits consist of a screening visit, one dosing visit and eight safety monitoring visits.

Subjects: This study involves twelve healthy male subjects for the initial screening of a potential intranasal formulation. Only male subjects participate in this study as the aim is to have as homogenous cohort as possible. Future studies will include women when the optimal nasal formulation is obtained. Subjects are twelve healthy non-smoking male subjects, age 18-50.

Clinical and Laboratory Testing.

Serum β-Interferon Samples: Serial blood collections are made to measure serum β-interferon levels. A total of 133 mL is collected. Each blood sample is prepared and separated into two aliquots. One sample is analyzed and the second is stored for repeat analysis, if necessary. All blood samples are analyzed for levels of interferon-β-1a using a commercially available ELISA method conducted by an accredited, certified laboratory.

Serum Neopterin and β-2-microglobulin Samples: Serial blood collections are made to measure serum neopterin and β-2-microglobulin levels. A total of 56 mL are collected. Each blood sample is prepared and separated into two aliquots. One sample is analyzed and the second is stored for repeat analysis, if necessary. All blood samples are analyzed for levels of interferon using a commercially available method conducted by an accredited, certified laboratory.

Whole blood for 2′5′-oligoadenylate synthetase in peripheral blood mononuclear Cells: Activity of 2′5′-oligoadenylate synthetase (OAS) is measured in peripheral blood mononuclear cells. A total of 56 mL is collected. Each blood sample is collected using EDTA as anticoagulant and a peripheral mononuclear cell medium. The blood and the medium is centrifuged together. The activity of OAS is measured by incorporation of [3H]-ATP into oligoadenylate.

Serum Neutralizing Antibodies: Serial blood collections are used to measure serum neutralizing antibodies. A total of 56 mL is collected. Each blood sample is prepared and separated into two aliquots. One sample is analyzed and the second is stored for repeat analysis, if necessary. All blood samples are analyzed for levels of interferon using viral Cytopathic Effect Inhibition (CPE) assay (bioassay) conducted by an accredited, certified laboratory.

Bioanalytics.

Neopterin/β-2 microglobin/Interferon-β and Neutralizing Antibodies: Blood samples are collected in 7 mL vacutainers and centrifuged at room temperature for not less than 8 minutes at 1,500 rpm after at least 30 minutes have elapsed from the time of the blood draw. At least 1.2 mL of serum is pipetted into the first of two prelabeled polypropylene tubes, with the remainder pipetted into the second tube. Both tubes are frozen promptly and stored at −10° C. for no more than 30 days until shipped for analysis. When instructed by the study monitor, the first samples (containing at least 1.2 mL of serum) are placed in test tube racks packed on dry ice sufficient for 2 days and shipped (with a complete inventory of samples sent) for delivery via overnight mail to Lofstrand Labs Limited or Lab Corp.

2′5′-oligoadenylate synthetase: Blood samples are collected in 7 mL ETDA vacutainers. Each blood sample is collected using EDTA as anticoagulant and a peripheral mononuclear cell medium. The blood and the medium is centrifuged together at 450-500×g for 35 minutes. Centrifugal force or the time is decreased when the second or lower band of cells is too close to the RBC pellet. Centrifugal force or the time is increased when the two bands are close together. After centrifugation, two cell bands are visible. The top band at the sample medium interface consists of mononuclear cells and the lower of polymorphonuclear cells: the RBC are pelleted. The cell band may be harvested using a Pasteur pipette. The polymorphonuclear cells is diluted by adding an equal volume of 0.45% NACL or 0.5 normal culture media. The polymorphonuclear cells is transferred to a 10 mL tube and the tube filled with 0.9% saline or 1N culture medium. The tube is centrifuged at approximately 400×g for 10 minutes at room temperature. The RBC contamination is usually between 2-6% of the total cells. The activity of OAS is measured by incorporation of [³H]-ATP into oligoadenylate. When instructed by the study monitor, the sample is placed in test tube racks packed on dry ice sufficient for 2 days and shipped (with a complete inventory of samples sent) for delivery via overnight mail to Stony Brook University.

Absorption and Pharmcodynamic Data Evaluation.

All absorption data are plotted for individual subjects as well as for the averaged data. The Cₘₐₓ, tₘₐₓ, AUC (bioavailability), t_{1/2}, Kₑₗ, Vₛₛ and Cl values of the reference and test products, based on the best fit PK model, are evaluated with the goal of comparing the aforementioned pharmacokinetic parameters to those in the medical literature.

For neopterin and β-2-microglobulin, arithmetic means by time, the maximum change from baseline, Cₘₐₓ, AUC and induction ratio of peak to baseline are calculated for reference and test products.

OAS activity in the peripheral blood mononuclear cells, arithmetic means by time, function of dose and change from baseline are calculated for reference and test products.

Serum neutralizing activity data. Data from anti-interferon-β-1a serum neutralizing activity evaluations is recorded. Serum neutralizing activity does not appear to be associated with accelerated disease progression in multiple sclerosis patients, however, if subjects have serum neutralizing antibodies, they are notified.

Tolerance data evaluation: All tolerance data collected for the test product is tabulated and evaluated to determine if the test product is tolerated by the subjects studied. A special emphasis is placed on the nasal tolerance data.

Institutional review board (IRB): The intent of the research program, the study protocol and the Informed Consent Form to be used in the study is approved (in writing) by an appropriate IRB prior to the start of the study. A copy of the approval is forwarded to the study sponsor. When necessary, an extension or renewal for the IRB approval is obtained and a copy also forwarded to the study sponsor.

Subject informed consent: All prospective volunteers have the study explained by a member of the research team or a member of their staff. The nature of the drug substance to be evaluated is explained together with the potential hazards involving drug allergies and possible adverse reactions. An acknowledgment of the receipt of this information and the participant's freely-tendered offer to volunteer is obtained in writing from each participant in the study.

Nasal tolerance-symptoms questionnaire (intranasal group only): In order to test the nasal tolerance of each of the intranasal administration, subjects complete a questionnaire at 5, 10, 15, 30, 45 and 60 minutes after dosing. A member of the study staff provides assistance if needed by any subject so that the questionnaire can be filled out properly.

Results: Due to its unique characteristics, the intranasal administration of pharmaceutical formulations of the present invention comprising interferon-β and one or more intranasal delivery-enhancing agents offers many advantages in terms of providing absorption of macromolecular drugs which are either not absorbed or variably absorbed after oral administration or absorbed more slowly following intramuscular or subcutaneous injection. No non-injectable products of interferon-β-1a are currently available. Pulmonary administration has achieved some success but has disadvantages including patient inconvenience and questionable pulmonary safety.

TABLE 14
Pharmacokinetic and pharmacodynamic parameters^{a}

| | Rebif ®, | Rebif ®, | Avonex ®, | |
|---|---|---|---|---|
| | SC | IM | IM | Intranasal |
| | 12 MIU | 12 MIU | 12 MIU | Formulation F9 |
| | (60 μg) | (60 μg) | (60 μg) | 12 MIU (60 μg) |
| | dose | dose | dose | dose |
| Serum | | | | |
| neopterin: | | | | |
| AUC_{0-144 h} | 2700 | 2930 | 2974 | 161.7 (0-96 h, |
| (nmol h/l) | | | | ng · h/ml) |
| | | | | (= 610 nmol · h/l |
| Cₘₐₓ (nmol/l) | 32 | 35 | 36 | 2.07 (ng/ml) |
| | | | | (= 7.8 nmol/l) |
| tₘₐₓ (h) | 36 | 36 | 36 | 28.7 |
| Serum β₂- | | | | |
| microglobulin: | | | | |
| AUC_{0-24 h} | 271 | 277 | 270 | 197.5 (0-96 h, |
| (mg h/l) | | | | μIU · h/ml) |
| Cₘₐₓ (mg/l) | 2.3 | 2.4 | 2.3 | 1.74 |
| tₘₐₓ (h) | 24 | 36 | 36 | 41.7 |

^{a}Per hour and 10⁴ cells.
*P = 0.015, Avonex ® IM > Rebif ® SC.
Data on Avonex ® and Rebif ®: Munafo, et al., Eur.J. Neurology, 5: 187-193, 1998.

Results: Table 14 provides pharmacokinetic data for intranasal delivery of interferon-β-1a in a pharmaceutical formulation of the present invention (e.g., Formulation F-9) compared to subcutaneous or intramuscular delivery of interferon-β-1a (Avonex® or Rebif®).

The results exemplify bioavailability of interferon-β as measured by interferon-β markers, for example, β-2 microglobulin and neopterin, achieved by the methods and formulations herein, e.g., as measured by area under the concentration curve (AUC) in blood serum, CNS, CSF or in another selected physiological compartment or target tissue. Bioavailability of interferon-β as measured by interferon-β markers will be, for example, AUC_{0-96 hr} for β-2 microglobulin of approximately 200 μIU·hr/ml of blood plasma or CSF, or AUC_{0-96 hr} for β-2 microglobulin up to approximately 500 μIU·hr/ml of blood plasma or CSF; AUC_{0-96 hr} for neopterin of approximately 200 ng·hr/ml of blood plasma or CSF, or AUC_{0-96 hr} for neopterin up to approximately 500 ng·hr/ml of blood plasma or CSF.

Although the foregoing invention has been described in detail by way of example for purposes of clarity of understanding, it will be apparent to the artisan that certain changes and modifications are comprehended by the disclosure and may be practiced without undue experimentation within the scope of the appended claims, which are presented by way of illustration not limitation.

### EXAMPLE 8

### Preparation of Intranasal Beta Interferon-β (IFN-β)

A preferred embodiment of the present invention was prepared according to the following procedure.

A vial of AVONEX® containing a lyophilized formulation of interferon beta-1a was purchased from the Biogen, Inc, Cambridge, Mass. The lyophilized formulation in the vial cotained 30 μg of interferon beta-1a, 15 mg of human albumin, USP, 5.8 mg of sodium chloride, USP, 5.7 mg of dibasic sodium phosphate and 1.2 mg monobasic sodium phosphate. The lyophilized product was dissolved into a 1 mL aqueous diluent preparation comprised of the following:

Composition of Interferon Beta-1a Diluent

| Item | | |
|---|---|---|
| No | Ingredients | % W/V |
| 1 | Gelatin, NF | 0.5 |
| 2 | Methyl Beta Cyclodextrin | 3.0 |
| 3 | L-Alpha-phosphatidylcholine | 0.5 |
| | Didecanoyl | |
| 4 | Edetate Disodium, USP | 0.1 |
| 5 | Benzalkonium Chloride, NF (50%) | 0.1 |
| 6 | Purified Water, USP q.s. to | 100 |

What is claimed is:

1. A stable pharmaceutical composition comprising one or more interferon-β compound(s) formulated for mucosal delivery to a mammalian subject in combination with one or more mucosal delivery-enhancing agent(s), wherein said composition following mucosal administration to said subject yields enhanced mucosal delivery of said one or more interferon-β compound(s) characterized by: (i) a peak concentration (Cₘₐₓ) of said interferon-β compound(s) in a CNS tissue or fluid or in a blood plasma of said subject that is 15% or greater as compared to a peak concentration of said interferon-β compounds in CNS or blood plasma following intramuscular injection of an equivalent concentration or dose of said interferon-β compound(s) to said subject; (ii) an area under concentration curve (AUC) of said interferon-β compound(s) in a central nervous system (CNS) tissue or fluid or in a blood plasma of the subject that is 25% or greater compared to an AUC of interferon-β in CNS or blood plasma following intramuscular injection of an equivalent concentration or dose of said interferon-β compound(s) to said subject; or (iii) a time to maximal concentration (tₘₐₓ) of said interferon-β in a central nervous system (CNS) tissue or fluid or in a blood plasma of the subject between about 0.1 to 1.0 hours.

2. The pharmaceutical composition of claim 1, wherein said composition following mucosal administration to said subject yields a peak concentration (Cₘₐₓ) of said interferon-β compound(s) in said CNS tissue or fluid or in a blood plasma of said subject that is 25% or greater as compared to a peak concentration of said interferon-β compound(s) in said CNS tissue or fluid or blood plasma following intramuscular injection of an equivalent concentration or dose of said interferon-β compound(s) to said subject.

3. The pharmaceutical composition of claim 1, wherein said composition following mucosal administration to said subject yields a peak concentration (Cₘₐₓ) of said interferon-β compound(s) in said CNS tissue or fluid or in a blood plasma of said subject that is 50% or greater as compared to a peak concentration of said interferon-β compound(s) in said CNS or blood plasma following intramuscular injection of an equivalent concentration or dose of said interferon-β compound(s) to said subject.

4. The pharmaceutical composition of claim 1, wherein said composition following mucosal administration to said subject yields an area under concentration curve (AUC) of said interferon-β compound(s) in said CNS tissue or fluid or in a blood plasma of the subject that is 25% or greater compared to an AUC of said interferon-β compound(s) in said CNS or blood plasma following intramuscular injection of an equivalent concentration or dose of said interferon-β compound(s) to said subject.

5. The pharmaceutical composition of claim 1, wherein said composition following mucosal administration to said subject yields an area under concentration curve (AUC) of said interferon-β compound(s) in said CNS tissue or fluid or in a blood plasma of the subject that is 50% or greater compared to an AUC of said interferon-β compound(s) in said CNS or blood plasma following intramuscular injection of an equivalent concentration or dose of said interferon-β compound(s) to said subject.

6. The pharmaceutical composition of claim 1, wherein said composition following mucosal administration to said subject yields a time to maximal plasma concentration (tₘₐₓ) of said interferon-β compound(s) in said CNS tissue or fluid or in a blood plasma of the subject between about 0.1 to 1.0 hours.

7. The pharmaceutical composition of claim 1, wherein said composition following mucosal administration to said subject yields a time to maximal plasma concentration (tₘₐₓ) of said interferon-β compound(s) in said CNS tissue or fluid or in a blood plasma of the subject between about 0.2 to 0.5 hours.

8. The pharmaceutical composition of claim 1, wherein said composition following mucosal administration to said subject yields an area under concentration curve (AUC) of neopterin or β2-microglobulin in said CNS tissue or fluid or in a blood plasma of the subject that is 25% or greater compared to an AUC of neopterin or β2-microglobulin in said CNS tissue or fluid or blood plasma following intramuscular injection of an equivalent concentration or dose of said interferon-β compound(s) to said subject.

9. The pharmaceutical composition of claim 1, wherein said composition following mucosal administration to said subject yields an area under concentration curve (AUC) of neopterin or β2-microglobulin in said CNS tissue or fluid or in a blood plasma of the subject that is 50% or greater compared to an AUC of neopterin or β2-microglobulin in said CNS tissue or fluid or blood plasma following intramuscular injection of an equivalent concentration or dose of said interferon-β compound(s) to said subject.

10. The pharmaceutical composition of claim 1, wherein said composition following mucosal administration to said subject yields a peak concentration (Cₘₐₓ) for neopterin or β2-microglobulin in said CNS tissue or fluid or in a blood plasma of the subject that is 25% or greater as compared to a peak concentration of neopterin or β2-microglobulin in said CNS tissue or fluid or blood plasma following intramuscular injection of an equivalent concentration or dose of said interferon-β compound(s) to said subject.

11. The pharmaceutical composition of claim 1, wherein said composition following mucosal administration to said subject yields a peak concentration (Cₘₐₓ) for neopterin or β2-microglobulin in said CNS tissue or fluid or in a blood plasma of the subject that is 50% or greater as compared to a peak concentration of neopterin or β2-microglobulin in said CNS tissue or fluid or blood plasma following intramuscular injection of an equivalent concentration or dose of said interferon-β compound(s) to said subject.

12. The pharmaceutical composition of claim 1, wherein said composition following mucosal administration to said subject yields a time to maximal plasma concentration (tₘₐₓ) of neopterin or β2-microglobulin in said CNS tissue or fluid or in a blood plasma of the subject between about 25 to 35 hours.

13. The pharmaceutical composition of claim 1, wherein said composition following mucosal administration to said subject yields a peak concentration of said human interferon-β compound(s) in said CNS tissue or fluid of the subject that is 10% or greater compared to a peak concentration of said human interferon-β compound(s) in a blood plasma of the subject.

14. The pharmaceutical composition of claim 1, wherein said composition following mucosal administration to said subject yields a peak concentration of said human interferon-β compound(s) in said CNS tissue or fluid of the subject that is 20% or greater compared to a peak concentration of said human interferon-β compound(s) in a blood plasma of the subject.

15. The pharmaceutical composition of claim 14, wherein said composition following mucosal administration to said subject yields a peak concentration of said human interferon-β compound(s) in said CNS tissue or fluid of the subject that is 40% or greater compared to a peak concentration of said human interferon-β compound(s) in a blood plasma of the subject.

16. The pharmaceutical composition of claim 1, wherein said interferon-β compound(s) formulated for intranasal delivery to said subject in combination with said one or more intranasal delivery-enhancing agent(s) is effective following intranasal administration to alleviate one or more symptom(s) of autoimmune disease or viral infection in said subject without unacceptable adverse side effects.

17. The pharmaceutical composition of claim 1, wherein said mucosal delivery-enhancing agent(s) is/are selected from:
(a) an aggregation inhibitory agent;
(b) a charge-modifying agent;
(c) a pH control agent;
(d) a degradative enzyme inhibitory agent;
(e) a mucolytic or mucus clearing agent;
(f) a ciliostatic agent;
(g) a membrane penetration-enhancing agent selected from (i) a surfactant, (ii) a bile salt, (ii) a phospholipid additive, mixed micelle, liposome, or carrier, (iii) an alcohol, (iv) an enamine, (v) an NO donor compound, (vi) a long-chain amphipathic molecule (vii) a small hydrophobic penetration enhancer; (viii) sodium or a salicylic acid derivative; (ix) a glycerol ester of acetoacetic acid (x) a cyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, or (x) an inhibitor of cholesterol synthesis; or (xi) any combination of the membrane penetration enhancing agents recited in (i)-(x);
(h) a modulatory agent of epithelial junction physiology;
(i) a vasodilator agent;
(j) a selective transport-enhancing agent; and
(k) a stabilizing delivery vehicle, carrier, support or complex-forming species with which the interferon-β is effectively combined, associated, contained, encapsulated or bound resulting in stabilization of the interferon-β for enhanced nasal mucosal delivery, wherein the formulation of said interferon-β with said one or more intranasal delivery-enhancing agents provides for increased bioavailability of the interferon-β in a blood plasma of said subject.

18. The pharmaceutical composition of claim 17, further comprising a plurality of mucosal delivery-enhancing agents.

19. The pharmaceutical composition of claim 17, comprising one or more intranasal delivery-enhancing agents.

20. The pharmaceutical composition of claim 1, wherein said mucosal delivery-enhancing agent(s) is/are selected from the group consisting of citric acid, sodium citrate, propylene glycol, glycerin, L-ascorbic acid, sodium metabisulfite, EDTA disodium, benzalkonium chloride, sodium hydroxide and mixtures thereof.

21. The pharmaceutical composition of claim 1, further comprising one or more sustained release-enhancing agent(s).

22. The pharmaceutical composition of claim 21, wherein the sustained release-enhancing agent is polyethylene glycol (PEG) in combination with interferon-β.

23. The pharmaceutical composition of claim 1, wherein the interferon-β is human interferon-β or a biologically active analog, fragment, or derivative thereof.

24. The pharmaceutical composition of claim 1, wherein said interferon-β is formulated in an effective dosage unit of between about 30 and 250 μg.

30. The pharmaceutical composition of claim 1, further comprising one or more steroid or corticosteroid compound(s), wherein said composition is effective following mucosal administration to alleviate one or more symptom(s) of inflammation, nasal irritation, rhinitis, or allergy without unacceptable adverse side effects.

31. The pharmaceutical composition of claim 1, further comprising one or more steroid or corticosteroid compound(s), wherein said composition is effective following mucosal administration to alleviate one or more symptom(s) of an autoimmune disease or viral infection in said subject without unacceptable adverse side effects.

33. A method for treating or preventing an autoimmune or viral disease or condition in a mammalian subject amenable to treatment by therapeutic administration of a interferon-βcompound comprising administering to a mucosal surface of said subject a pharmaceutical composition comprising an effective amount of one or more interferon-β compound(s) formulated for mucosal delivery in combination with one or more mucosal delivery-enhancing agent(s) in an effective dosage regimen to alleviate one or more symptom(s) of said autoimmune disease or viral infection in said subject without unacceptable adverse side effects.

34. The method of claim 33, wherein said interferon-β compound(s) is/are formulated for intranasal delivery to said subject in combination with one or more intranasal delivery-enhancing agent(s), and wherein said method employs an intranasal effective dosage regimen to alleviate one or more symptom(s) of said autoimmune disease or viral infection in said subject without unacceptable adverse side effects.

35. The method of claim 34, wherein said interferon-β compound(s) is/are provided in a multiple dosage unit kit or container for repeated self-dosing by said subject.

36. The method of claim 33, wherein said interferon-β compound(s) is/are repeatedly administered through an intranasal effective dosage regimen that involves multiple administrations said interferon-β compound(s) to said subject during a daily or weekly schedule to maintain a therapeutically effective baseline level of interferon-βduring an extended dosing period.

37. The method of claim 36, wherein said interferon-β compound(s) is/are self-administered by said subject in a nasal formulation between two and six times daily to maintain a therapeutically effective baseline level of interferon-βduring an 8 hour to 24 hour extended dosing period.

38. The method of claim 33, which yields a peak concentration (Cₘₐₓ) of said interferon-β in a blood plasma or cerebral spinal fluid (CNS) of said subject following mucosal administration that is 25% or greater as compared to a peak concentration of interferon-β in blood plasma or CNS following intramuscular injection of an equivalent concentration or dose of interferon-β to said subject.

39. The method of claim 38, which yields a peak concentration (Cₘₐₓ) of said interferon-β in a blood plasma or cerebral spinal fluid (CNS) of said subject following mucosal administration that is 50% or greater as compared to a peak concentration of interferon-β in blood plasma or CNS following intramuscular injection of an equivalent concentration or dose of interferon-β to said subject.

40. The method of claim 33, which yields an area under concentration curve (AUC) of said interferon-β in a blood plasma or cerebral spinal fluid (CNS) of the subject following mucosal administration that is 25% or greater compared to an AUC of interferon-β in blood plasma or CNS following intramuscular injection of an equivalent concentration or dose of interferon-β to said subject.

41. The method of claim 40, which yields an area under concentration curve (AUC) of said interferon-β in a blood plasma or cerebral spinal fluid (CNS) of the subject following mucosal administration that is 50% or greater compared to an AUC of interferon-β in blood plasma or CNS following intramuscular injection of an equivalent concentration or dose of interferon-β to said subject.

42. The method of claim 33, which yields a time to maximal plasma concentration (tₘₐₓ) of said interferon-β in a blood plasma or cerebral spinal fluid (CNS) of the subject following mucosal administration of between about 0.1 to 1.0 hours.

43. The method of claim 42, which yields a time to maximal plasma concentration (tₘₐₓ) of said interferon-β in a blood plasma or cerebral spinal fluid (CNS) of the subject following mucosal administration of between 0.2 to 0.5 hours.

44. The method of claim 33, which yields a peak concentration of said human interferon-β in a central nervous system (CNS) tissue or fluid of the subject following mucosal administration that is 10% or greater compared to a peak concentration of said human interferon-β in a blood plasma of the subject.

45. The method of claim 44, which yields a peak concentration of said human interferon-β in a central nervous system (CNS) tissue or fluid of the subject following mucosal administration that is 20% or greater compared to a peak concentration of said human interferon-β in a blood plasma of the subject.

46. The method of claim 44, which yields a peak concentration of said human interferon-β in a central nervous system (CNS) tissue or fluid of the subject following mucosal administration that is 40% or greater compared to a peak concentration of said human interferon-β in a blood plasma of the subject.

47. The method of claim 60, wherein said mucosal delivery-enhancing agent(s) is/are selected from:
(a) an aggregation inhibitory agent;
(b) a charge-modifying agent;
(c) a pH control agent;
(d) a degradative enzyme inhibitory agent;
(e) a mucolytic or mucus clearing agent;
(f) a ciliostatic agent;
(g) a membrane penetration-enhancing agent selected from (i) a surfactant, (ii) a bile salt, (ii) a phospholipid additive, mixed micelle, liposome, or carrier, (iii) an alcohol, (iv) an enamine, (v) an NO donor compound, (vi) a long-chain amphipathic molecule (vii) a small hydrophobic penetration enhancer; (viii) sodium or a salicylic acid derivative; (ix) a glycerol ester of acetoacetic acid (x) a cyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, or (x) an inhibitor of cholesterol synthesis; or (xi) any combination of the membrane penetration enhancing agents recited in (i)-(x);
(h) a modulatory agent of epithelial junction physiology;
(i) a vasodilator agent;
(j) a selective transport-enhancing agent; and
(k) a stabilizing delivery vehicle, carrier, support or complex-forming species with which the interferon-β is effectively combined, associated, contained, encapsulated or bound resulting in stabilization of the interferon-β for enhanced nasal mucosal delivery, wherein the formulation of said interferon-β with said one or more intranasal delivery-enhancing agents provides for increased bioavailability of the interferon-β in a blood plasma of said subject.

48. The method of claim 47, wherein said pharmaceutical composition further comprises a plurality of mucosal delivery-enhancing agents.

49. The method of claim 33, wherein said pharmaceutical composition comprises one or more intranasal delivery-enhancing agents.

50. The method of claim 33, wherein said mucosal delivery-enhancing agent(s) is/are selected from the group consisting of citric acid, sodium citrate, propylene glycol, glycerin, L-ascorbic acid, sodium metabisulfite, EDTA disodium, benzalkonium chloride, sodium hydroxide and mixtures thereof.

79. The method of claim 60, wherein said pharmaceutical composition further comprises one or more sustained release-enhancing agent(s).

80. The method of claim 79, wherein the sustained release-enhancing agent is polyethylene glycol (PEG).

81. The method of claim 60, wherein the interferon-β is human interferon-β or a biologically active analog, fragment, or derivative thereof.

82. The method of claim 60, wherein said interferon-β is formulated in an effective dosage unit of between about 30 and 250 μg.

83. The method of claim 60, which is effective to alleviate one or more symptom(s) of multiple sclerosis (MS) in said subject without unacceptable adverse side effects.

84. The method of claim 60, which is effective to alleviate one or more symptom(s) of chronic hepatitis B, condyloma acuminata, papillomavirus warts of the larynx or skin, or childhood viral encephalitis in said subject without unacceptable adverse side effects.

85. The method of claim 60, wherein said pharmaceutical composition comprises a plurality of different interferon-βcompounds.

86. The method of claim 60, wherein said pharmaceutical composition further comprises an interferon- [symbol] compound.

87. The pharmaceutical formulation of claim 1, which is pH adjusted to between about pH 3.0-6.0.

88. The pharmaceutical formulation of claim 1, which is pH adjusted to between about pH 3.0-5.0.

89. The pharmaceutical formulation of claim 1, which is pH adjusted to between about pH 4.0-5.0.

90. The pharmaceutical formulation of claim 1, which is pH adjusted to about pH 4.0-4.5.

91. The pharmaceutical formulation of claim 1, wherein said mucosal delivery-enhancing agent is a permeabilizing peptide that reversibly enhances mucosal epithelial paracellular transport by modulating epithelial junctional structure and/or physiology in a mammalian subject, wherein said peptide effectively inhibits homotypic binding of an epithelial membrane adhesive protein selected from ajunctional adhesion molecule (JAM), occludin, or claudin.


SEQUENCE LISTING (790 sequences)

SEQ ID NO: 1 - protein, 4 aa, Homo sapiens
  VARIANT 3: Xaa = Ile, Val or Ala
VRXP

SEQ ID NO: 2 - protein, 7 aa, Homo sapiens
  VARIANT 1: Xaa = Val, Ala, or Ile
  VARIANT 4: Xaa = Ser or Thr
XKLXCAY

SEQ ID NO: 3 - protein, 9 aa, Homo sapiens
  VARIANT 3: Xaa = Thr or Ser
  VARIANT 7: Xaa = Thr or Arg
  VARIANT 9: Xaa = Met or Glu
EDXGTYXCX

SEQ ID NO: 4 - protein, 4 aa, Homo sapiens
VRIP

SEQ ID NO: 5 - protein, 7 aa, Homo sapiens
VKLSCAY

SEQ ID NO: 6 - protein, 9 aa, Homo sapiens
TGITFKSVT

SEQ ID NO: 7 - protein, 4 aa, Homo sapiens
ITAS

SEQ ID NO: 8 - protein, 4 aa, Homo sapiens
SVTR

SEQ ID NO: 9 - protein, 9 aa, Homo sapiens
EDTGTYTCM

SEQ ID NO: 10 - protein, 9 aa, Homo sapiens
GFSSPRVEW

SEQ ID NO: 11 - protein, 10 aa, Homo sapiens
SVTVHSSEPE

SEQ ID NO: 12 - protein, 10 aa, Homo sapiens
VRIPENNPVK

SEQ ID NO: 13 - protein, 10 aa, Homo sapiens
LSCAYSGFSS

SEQ ID NO: 14 - protein, 10 aa, Homo sapiens
PRVEWKFDQG

SEQ ID NO: 15 - protein, 10 aa, Homo sapiens
DTTRLVCYNN

SEQ ID NO: 16 - protein, 10 aa, Homo sapiens
KITASYEDRV

SEQ ID NO: 17 - protein, 10 aa, Homo sapiens
TFLPTGITFK

SEQ ID NO: 18 - protein, 10 aa, Homo sapiens
SVTREDTGTY

SEQ ID NO: 19 - protein, 10 aa, Homo sapiens
TCMVSEEGGN

SEQ ID NO: 20 - protein, 10 aa, Homo sapiens
SYGEVKVKLI

SEQ ID NO: 21 - protein, 10 aa, Homo sapiens
VLVPPSKPTV

SEQ ID NO: 22 - protein, 10 aa, Homo sapiens
NIPSSATIGN

SEQ ID NO: 23 - protein, 10 aa, Homo sapiens
RAVLTCSEQD

SEQ ID NO: 24 - protein, 10 aa, Homo sapiens
GSPPSEYTWF

SEQ ID NO: 25 - protein, 10 aa, Homo sapiens
KDGIVMPTNP

SEQ ID NO: 26 - protein, 10 aa, Homo sapiens
KSTRAFSNSS

SEQ ID NO: 27 - protein, 10 aa, Homo sapiens
YVLNPTTGEL

SEQ ID NO: 28 - protein, 10 aa, Homo sapiens
VFDPLSASDT

SEQ ID NO: 29 - protein, 10 aa, Homo sapiens
GEYSCEARNG

SEQ ID NO: 30 - protein, 10 aa, Homo sapiens
YGTPMTSNAV

SEQ ID NO: 31 - protein, 10 aa, Homo sapiens
RMEAVERNVG

SEQ ID NO: 32 - protein, 5 aa, Homo sapiens
SVTVH

SEQ ID NO: 33 - protein, 10 aa, Homo sapiens
SSEPEVRIPE

SEQ ID NO: 34 - protein, 10 aa, Homo sapiens
NNPVKLSCAY

SEQ ID NO: 35 - protein, 10 aa, Homo sapiens
SGFSSPRVEW

SEQ ID NO: 36 - protein, 10 aa, Homo sapiens
KFDQGDTTRL

SEQ ID NO: 37 - protein, 10 aa, Homo sapiens
VCYNNKITAS

SEQ ID NO: 38 - protein, 10 aa, Homo sapiens
YEDRVTFLPT

SEQ ID NO: 39 - protein, 10 aa, Homo sapiens
GITFKSVTRE

SEQ ID NO: 40 - protein, 10 aa, Homo sapiens
DTGTYTCMVS

SEQ ID NO: 41 - protein, 10 aa, Homo sapiens
EEGGNSYGEV

SEQ ID NO: 42 - protein, 10 aa, Homo sapiens
KVKLIVLVPP

SEQ ID NO: 43 - protein, 10 aa, Homo sapiens
SKPTVNIPSS

SEQ ID NO: 44 - protein, 10 aa, Homo sapiens
ATIGNRAVLT

SEQ ID NO: 45 - protein, 10 aa, Homo sapiens
CSEQDGSPPS

SEQ ID NO: 46 - protein, 10 aa, Homo sapiens
EYTWFKDGIV

SEQ ID NO: 47 - protein, 10 aa, Homo sapiens
MPTNPKSTRA

SEQ ID NO: 48 - protein, 10 aa, Homo sapiens
FSNSSYVLNP

SEQ ID NO: 49 - protein, 10 aa, Homo sapiens
TTGELVFDPL

SEQ ID NO: 50 - protein, 10 aa, Homo sapiens
SASDTGEYSC

SEQ ID NO: 51 - protein, 10 aa, Homo sapiens
EARNGYGTPM

SEQ ID NO: 52 - protein, 10 aa, Homo sapiens
TSNAVRMEAV

SEQ ID NO: 53 - protein, 7 aa, Homo sapiens
ERNVGVI

SEQ ID NO: 54 - protein, 8 aa, Homo sapiens
SVTVHSSE

SEQ ID NO: 55 - protein, 8 aa, Homo sapiens
PEVRIPEN

SEQ ID NO: 56 - protein, 8 aa, Homo sapiens
NPVKLSCA

SEQ ID NO: 57 - protein, 8 aa, Homo sapiens
YSGFSSPR

SEQ ID NO: 58 - protein, 8 aa, Homo sapiens
VEWKFDQG

SEQ ID NO: 59 - protein, 8 aa, Homo sapiens
DTTRLVCY

SEQ ID NO: 60 - protein, 8 aa, Homo sapiens
NNKITASY

SEQ ID NO: 61 - protein, 8 aa, Homo sapiens
EDRVTFLP

SEQ ID NO: 62 - protein, 8 aa, Homo sapiens
TGITFKSV

SEQ ID NO: 63 - protein, 8 aa, Homo sapiens
TREDTGTY

SEQ ID NO: 64 - protein, 8 aa, Homo sapiens
TCMVSEEG

SEQ ID NO: 65 - protein, 8 aa, Homo sapiens
GNSYGEVK

SEQ ID NO: 66 - protein, 8 aa, Homo sapiens
VKLIVLVP

SEQ ID NO: 67 - protein, 8 aa, Homo sapiens
PSKPTVNI

SEQ ID NO: 68 - protein, 8 aa, Homo sapiens
PSSATIGN

SEQ ID NO: 69 - protein, 8 aa, Homo sapiens
RAVLTCSE

SEQ ID NO: 70 - protein, 8 aa, Homo sapiens
QDGSPPSE

SEQ ID NO: 71 - protein, 8 aa, Homo sapiens
YTWFKDGI

SEQ ID NO: 72 - protein, 8 aa, Homo sapiens
VMPTNPKS

SEQ ID NO: 73 - protein, 8 aa, Homo sapiens
TRAFSNSS

SEQ ID NO: 74 - protein, 8 aa, Homo sapiens
YVLNPTTG

SEQ ID NO: 75 - protein, 8 aa, Homo sapiens
ELVFDPLS

SEQ ID NO: 76 - protein, 8 aa, Homo sapiens
ASDTGEYS

SEQ ID NO: 77 - protein, 8 aa, Homo sapiens
CEARNGYG

SEQ ID NO: 78 - protein, 8 aa, Homo sapiens
TPMTSNAV

SEQ ID NO: 79 - protein, 8 aa, Homo sapiens
RMEAVERN

SEQ ID NO: 80 - protein, 4 aa, Homo sapiens
VGVI

SEQ ID NO: 81 - protein, 4 aa, Homo sapiens
SVTV

SEQ ID NO: 82 - protein, 8 aa, Homo sapiens
HSSEPEVR

SEQ ID NO: 83 - protein, 8 aa, Homo sapiens
IPENNPVK

SEQ ID NO: 84 - protein, 8 aa, Homo sapiens
LSCAYSGF

SEQ ID NO: 85 - protein, 8 aa, Homo sapiens
SSPRVEWK

SEQ ID NO: 86 - protein, 8 aa, Homo sapiens
FDQGDTTR

SEQ ID NO: 87 - protein, 8 aa, Homo sapiens
LVCYNNKI

SEQ ID NO: 88 - protein, 8 aa, Homo sapiens
TASYEDRV

SEQ ID NO: 89 - protein, 8 aa, Homo sapiens
TFLPTGIT

SEQ ID NO: 90 - protein, 8 aa, Homo sapiens
FKSVTRED

SEQ ID NO: 91 - protein, 8 aa, Homo sapiens
TGTYTCMV

SEQ ID NO: 92 - protein, 8 aa, Homo sapiens
SEEGGNSY

SEQ ID NO: 93 - protein, 8 aa, Homo sapiens
GEVKVKLI

SEQ ID NO: 94 - protein, 8 aa, Homo sapiens
VLVPPSKP

SEQ ID NO: 95 - protein, 16 aa, Homo sapiens
TVNIPSSATIGNRAVL

SEQ ID NO: 96 - protein, 8 aa, Homo sapiens
TCSEQDGS

SEQ ID NO: 97 - protein, 8 aa, Homo sapiens
PPSEYTWF

SEQ ID NO: 98 - protein, 8 aa, Homo sapiens
KDGIVMPT

SEQ ID NO: 99 - protein, 8 aa, Homo sapiens
NPKSTRAF

SEQ ID NO: 100 - protein, 8 aa, Homo sapiens
SNSSYVLN

SEQ ID NO: 101 - protein, 8 aa, Homo sapiens
PTTGELVF

SEQ ID NO: 102 - protein, 8 aa, Homo sapiens
DPLSASDT

SEQ ID NO: 103 - protein, 8 aa, Homo sapiens
GEYSCEAR

SEQ ID NO: 104 - protein, 8 aa, Homo sapiens
NGYGTPMT

SEQ ID NO: 105 - protein, 8 aa, Homo sapiens
SNAVRMEA

SEQ ID NO: 106 - protein, 8 aa, Homo sapiens
VERNVGVI

SEQ ID NO: 107 - protein, 6 aa, Homo sapiens
PVRIPE

SEQ ID NO: 108 - protein, 10 aa, Homo sapiens
EPEVRIPENN

SEQ ID NO: 109 - protein, 12 aa, Homo sapiens
SEPEVRIPENNP

SEQ ID NO: 110 - protein, 14 aa, Homo sapiens
SSEPEVRIPENNPV

SEQ ID NO: 111 - protein, 16 aa, Homo sapiens
HSSEPEVRIPENNPVK

SEQ ID NO: 112 - protein, 18 aa, Homo sapiens
VHSSEPEVRIPENNPVKL

SEQ ID NO: 113 - protein, 20 aa, Homo sapiens
TVHSSEPEVRIPENNPVKLS

SEQ ID NO: 114 - protein, 5 aa, Homo sapiens
VRIPE

SEQ ID NO: 115 - protein, 6 aa, Homo sapiens
VRIPEN

SEQ ID NO: 116 - protein, 7 aa, Homo sapiens
VRIPENN

SEQ ID NO: 117 - protein, 8 aa, Homo sapiens
VRIPENNP

SEQ ID NO: 118 - protein, 9 aa, Homo sapiens
VRIPENNPV

SEQ ID NO: 119 - protein, 10 aa, Homo sapiens
VRIPENNPVK

SEQ ID NO: 120 - protein, 11 aa, Homo sapiens
VRIPENNPVKL

SEQ ID NO: 121 - protein, 12 aa, Homo sapiens
VRIPENNPVKLS

SEQ ID NO: 122 - protein, 5 aa, Homo sapiens
EVRIP

SEQ ID NO: 123 - protein, 6 aa, Homo sapiens
PEVRIP

SEQ ID NO: 124 - protein, 7 aa, Homo sapiens
EPEVRIP

SEQ ID NO: 125 - protein, 8 aa, Homo sapiens
SEPEVRIP

SEQ ID NO: 126 - protein, 9 aa, Homo sapiens
SSEPEVRIP

SEQ ID NO: 127 - protein, 10 aa, Homo sapiens
HSSEPEVRIP

SEQ ID NO: 128 - protein, 11 aa, Homo sapiens
VHSSEPEVRIP

SEQ ID NO: 129 - protein, 12 aa, Homo sapiens
TVHSSEPEVRIP

SEQ ID NO: 130 - protein, 4 aa, Homo sapiens
VRVP

SEQ ID NO: 131 - protein, 6 aa, Homo sapiens
PVRVPE

SEQ ID NO: 132 - protein, 8 aa, Homo sapiens
PEVRVPEN

SEQ ID NO: 133 - protein, 10 aa, Homo sapiens
EPEVRVPENN

SEQ ID NO: 134 - protein, 12 aa, Homo sapiens
SEPEVRVPENNP

SEQ ID NO: 135 - protein, 14 aa, Homo sapiens
SSEPEVRVPENNPV

SEQ ID NO: 136 - protein, 16 aa, Homo sapiens
HSSEPEVRVPENNPVK

SEQ ID NO: 137 - protein, 18 aa, Homo sapiens
VHSSEPEVRVPENNPVKL

SEQ ID NO: 138 - protein, 20 aa, Homo sapiens
TVHSSEPEVRVPENNPVKLS

SEQ ID NO: 139 - protein, 5 aa, Homo sapiens
VRVPE

SEQ ID NO: 140 - protein, 6 aa, Homo sapiens
VRVPEN

SEQ ID NO: 141 - protein, 7 aa, Homo sapiens
VRVPENN

SEQ ID NO: 142 - protein, 8 aa, Homo sapiens
VRVPENNP

SEQ ID NO: 143 - protein, 9 aa, Homo sapiens
VRVPENNPV

SEQ ID NO: 144 - protein, 10 aa, Homo sapiens
VRVPENNPVK

SEQ ID NO: 145 - protein, 11 aa, Homo sapiens
VRVPENNPVKL

SEQ ID NO: 146 - protein, 12 aa, Homo sapiens
VRVPENNPVKLS

SEQ ID NO: 147 - protein, 5 aa, Homo sapiens
EVRVP

SEQ ID NO: 148 - protein, 6 aa, Homo sapiens
PEVRVP

SEQ ID NO: 149 - protein, 7 aa, Homo sapiens
EPEVRVP

SEQ ID NO: 150 - protein, 8 aa, Homo sapiens
SEPEVRVP

SEQ ID NO: 151 - protein, 9 aa, Homo sapiens
SSEPEVRVP

SEQ ID NO: 152 - protein, 10 aa, Homo sapiens
HSSEPEVRVP

SEQ ID NO: 153 - protein, 11 aa, Homo sapiens
VHSSEPEVRVP

SEQ ID NO: 154 - protein, 12 aa, Homo sapiens
TVHSSEPEVRVP

SEQ ID NO: 155 - protein, 4 aa, Homo sapiens
VRAP

SEQ ID NO: 156 - protein, 6 aa, Homo sapiens
PVRAPE

SEQ ID NO: 157 - protein, 8 aa, Homo sapiens
PEVRAPEN

SEQ ID NO: 158 - protein, 10 aa, Homo sapiens
EPEVRAPENN

SEQ ID NO: 159 - protein, 12 aa, Homo sapiens
SEPEVRAPENNP

SEQ ID NO: 160 - protein, 14 aa, Homo sapiens
SSEPEVRAPENNPV

SEQ ID NO: 161 - protein, 16 aa, Homo sapiens
HSSEPEVRAPENNPVK

SEQ ID NO: 162 - protein, 18 aa, Homo sapiens
VHSSEPEVRAPENNPVKL

SEQ ID NO: 163 - protein, 20 aa, Homo sapiens [sequence omitted]

SEQ ID NO: 164 - protein, 5 aa, Homo sapiens
VRAPE

SEQ ID NO: 165 - protein, 6 aa, Homo sapiens
VRAPEN

SEQ ID NO: 166 - protein, 7 aa, Homo sapiens
VRAPENN

SEQ ID NO: 167 - protein, 8 aa, Homo sapiens
VRAPENNP

SEQ ID NO: 168 - protein, 9 aa, Homo sapiens
VRAPENNPV

SEQ ID NO: 169 - protein, 10 aa, Homo sapiens
VRAPENNPVK

SEQ ID NO: 170 - protein, 11 aa, Homo sapiens
VRAPENNPVKL

SEQ ID NO: 171 - protein, 12 aa, Homo sapiens
VRAPENNPVKLS

SEQ ID NO: 172 - protein, 5 aa, Homo sapiens
EVRAP

SEQ ID NO: 173 - protein, 6 aa, Homo sapiens
PEVRAP

SEQ ID NO: 174 - protein, 7 aa, Homo sapiens
EPEVRAP

SEQ ID NO: 175 - protein, 8 aa, Homo sapiens
SEPEVRAP

SEQ ID NO: 176 - protein, 9 aa, Homo sapiens
SSEPEVRAP

SEQ ID NO: 177 - protein, 10 aa, Homo sapiens
HSSEPEVRAP

SEQ ID NO: 178 - protein, 11 aa, Homo sapiens
VHSSEPEVRAP

SEQ ID NO: 179 - protein, 12 aa, Homo sapiens
TVHSSEPEVRAP

SEQ ID NO: 180 - protein, 9 aa, Homo sapiens
PVKLSCAYS

SEQ ID NO: 181 - protein, 11 aa, Homo sapiens
NPVKLSCAYSG

SEQ ID NO: 182 - protein, 13 aa, Homo sapiens
NNPVKLSCAYSGF

SEQ ID NO: 183 - protein, 15 aa, Homo sapiens
ENNPVKLSCAYSGFS

SEQ ID NO: 184 - protein, 17 aa, Homo sapiens
PENNPVKLSCAYSGFSS

SEQ ID NO: 185 - protein, 19 aa, Homo sapiens
IPENNPVKLSCAYSGFSSP

SEQ ID NO: 186 - protein, 21 aa, Homo sapiens
RIPENNPVKLSCAYSGFSSPR

SEQ ID NO: 187 - protein, 8 aa, Homo sapiens
PVKLSCAY

SEQ ID NO: 188 - protein, 9 aa, Homo sapiens
NPVKLSCAY

SEQ ID NO: 189 - protein, 10 aa, Homo sapiens
NNPVKLSCAY

SEQ ID NO: 190 - protein, 11 aa, Homo sapiens
ENNPVKLSCAY

SEQ ID NO: 191 - protein, 12 aa, Homo sapiens
PENNPVKLSCAY

SEQ ID NO: 192 - protein, 13 aa, Homo sapiens
IPENNPVKLSCAY

SEQ ID NO: 193 - protein, 14 aa, Homo sapiens
RIPENNPVKLSCAY

SEQ ID NO: 194 - protein, 8 aa, Homo sapiens
VKLSCAYS

SEQ ID NO: 195 - protein, 9 aa, Homo sapiens
VKLSCAYSG

SEQ ID NO: 196 - protein, 10 aa, Homo sapiens
VKLSCAYSGF

SEQ ID NO: 197 - protein, 11 aa, Homo sapiens
VKLSCAYSGFS

SEQ ID NO: 198 - protein, 12 aa, Homo sapiens
VKLSCAYSGFSS

SEQ ID NO: 199 - protein, 13 aa, Homo sapiens
VKLSCAYSGFSSP

SEQ ID NO: 200 - protein, 14 aa, Homo sapiens
VKLSCAYSGFSSPR

SEQ ID NO: 201 - protein, 7 aa, Homo sapiens
VKLTCAY

SEQ ID NO: 202 - protein, 7 aa, Homo sapiens
VKLTCAY

SEQ ID NO: 203 - protein, 9 aa, Homo sapiens
PVKLTCAYS

SEQ ID NO: 204 - protein, 11 aa, Homo sapiens
NPVKLTCAYSG

SEQ ID NO: 205 - protein, 13 aa, Homo sapiens
NNPVKLTCAYSGF

SEQ ID NO: 206 - protein, 15 aa, Homo sapiens
ENNPVKLTCAYSGFS

SEQ ID NO: 207 - protein, 17 aa, Homo sapiens
PENNPVKLTCAYSGFSS

SEQ ID NO: 208 - protein, 19 aa, Homo sapiens
IPENNPVKLTCAYSGFSSP

SEQ ID NO: 209 - protein, 21 aa, Homo sapiens
RIPENNPVKLTCAYSGFSSPR

SEQ ID NO: 210 - protein, 8 aa, Homo sapiens
PVKLTCAY

SEQ ID NO: 211 - protein, 9 aa, Homo sapiens
NPVKLTCAY

SEQ ID NO: 212 - protein, 10 aa, Homo sapiens
NNPVKLTCAY

SEQ ID NO: 213 - protein, 11 aa, Homo sapiens
ENNPVKLTCAY

SEQ ID NO: 214 - protein, 12 aa, Homo sapiens
PENNPVKLTCAY

SEQ ID NO: 215 - protein, 13 aa, Homo sapiens
IPENNPVKLTCAY

SEQ ID NO: 216 - protein, 14 aa, Homo sapiens
RIPENNPVKLTCAY

SEQ ID NO: 217 - protein, 8 aa, Homo sapiens
VKLTCAYS

SEQ ID NO: 218 - protein, 9 aa, Homo sapiens
VKLTCAYSG

SEQ ID NO: 219 - protein, 10 aa, Homo sapiens
VKLTCAYSGF

SEQ ID NO: 220 - protein, 11 aa, Homo sapiens
VKLTCAYSGFS

SEQ ID NO: 221 - protein, 12 aa, Homo sapiens
VKLTCAYSGFSS

SEQ ID NO: 222 - protein, 13 aa, Homo sapiens
VKLTCAYSGFSSP

SEQ ID NO: 223 - protein, 14 aa, Homo sapiens
VKLTCAYSGFSSPR

SEQ ID NO: 224 - protein, 7 aa, Homo sapiens
AKLSCAY

SEQ ID NO: 225 - protein, 7 aa, Homo sapiens
AKLSCAY

SEQ ID NO: 226 - protein, 9 aa, Homo sapiens
PAKLSCAYS

SEQ ID NO: 227 - protein, 11 aa, Homo sapiens
NPAKLSCAYSG

SEQ ID NO: 228 - protein, 13 aa, Homo sapiens
NNPAKLSCAYSGF

SEQ ID NO: 229 - protein, 15 aa, Homo sapiens
ENNPAKLSCAYSGFS

SEQ ID NO: 230 - protein, 17 aa, Homo sapiens
PENNPAKLSCAYSGFSS

SEQ ID NO: 231 - protein, 19 aa, Homo sapiens
IPENNPAKLSCAYSGFSSP

SEQ ID NO: 232 - protein, 21 aa, Homo sapiens [sequence omitted]

SEQ ID NO: 233 - protein, 8 aa, Homo sapiens
PAKLSCAY

SEQ ID NO: 234 - protein, 9 aa, Homo sapiens
NPAKLSCAY

SEQ ID NO: 235 - protein, 10 aa, Homo sapiens
NNPAKLSCAY

SEQ ID NO: 236 - protein, 11 aa, Homo sapiens
ENNPAKLSCAY

SEQ ID NO: 237 - protein, 12 aa, Homo sapiens
PENNPAKLSCAY

SEQ ID NO: 238 - protein, 13 aa, Homo sapiens
IPENNPAKLSCAY

SEQ ID NO: 239 - protein, 14 aa, Homo sapiens
RIPENNPAKLSCAY

SEQ ID NO: 240 - protein, 8 aa, Homo sapiens
AKLSCAYS

SEQ ID NO: 241 - protein, 9 aa, Homo sapiens
AKLSCAYSG

SEQ ID NO: 242 - protein, 10 aa, Homo sapiens
AKLSCAYSGF

SEQ ID NO: 243 - protein, 11 aa, Homo sapiens
AKLSCAYSGFS

SEQ ID NO: 244 - protein, 12 aa, Homo sapiens
AKLSCAYSGFSS

SEQ ID NO: 245 - protein, 13 aa, Homo sapiens
AKLSCAYSGFSSP

SEQ ID NO: 246 - protein, 14 aa, Homo sapiens
AKLSCAYSGFSSPR

SEQ ID NO: 247 - protein, 7 aa, Homo sapiens
AKLTCAY

SEQ ID NO: 248 - protein, 7 aa, Homo sapiens
AKLTCAY

SEQ ID NO: 249 - protein, 9 aa, Homo sapiens
PAKLTCAYS

SEQ ID NO: 250 - protein, 11 aa, Homo sapiens
NPAKLTCAYSG

SEQ ID NO: 251 - protein, 13 aa, Homo sapiens
NNPAKLTCAYSGF

SEQ ID NO: 252 - protein, 15 aa, Homo sapiens
ENNPAKLTCAYSGFS

SEQ ID NO: 253 - protein, 17 aa, Homo sapiens
PENNPAKLTCAYSGFSS

SEQ ID NO: 254 - protein, 19 aa, Homo sapiens
IPENNPAKLTCAYSGFSSP

SEQ ID NO: 255 - protein, 21 aa, Homo sapiens
RIPENNPAKLTCAYSGFSSPR

SEQ ID NO: 256 - protein, 8 aa, Homo sapiens
PAKLTCAY

SEQ ID NO: 257 - protein, 9 aa, Homo sapiens
NPAKLTCAY

SEQ ID NO: 258 - protein, 10 aa, Homo sapiens
NNPAKLTCAY

SEQ ID NO: 259 - protein, 11 aa, Homo sapiens
ENNPAKLTCAY

SEQ ID NO: 260 - protein, 12 aa, Homo sapiens
PENNPAKLTCAY

SEQ ID NO: 261 - protein, 13 aa, Homo sapiens
IPENNPAKLTCAY

SEQ ID NO: 262 - protein, 14 aa, Homo sapiens
RIPENNPAKLTCAY

SEQ ID NO: 263 - protein, 8 aa, Homo sapiens
AKLTCAYS

SEQ ID NO: 264 - protein, 9 aa, Homo sapiens
AKLTCAYSG

SEQ ID NO: 265 - protein, 10 aa, Homo sapiens
AKLTCAYSGF

SEQ ID NO: 266 - protein, 11 aa, Homo sapiens
AKLTCAYSGFS

SEQ ID NO: 267 - protein, 12 aa, Homo sapiens
AKLTCAYSGFSS

SEQ ID NO: 268 - protein, 13 aa, Homo sapiens
AKLTCAYSGFSSP

SEQ ID NO: 269 - protein, 14 aa, Homo sapiens
AKLTCAYSGFSSPR

SEQ ID NO: 270 - protein, 11 aa, Homo sapiens
REDTGTYTCMV

SEQ ID NO: 271 - protein, 13 aa, Homo sapiens
TREDTGTYTCMVS

SEQ ID NO: 272 - protein, 15 aa, Homo sapiens
VTREDTGTYTCMVSE

SEQ ID NO: 273 - protein, 17 aa, Homo sapiens
SVTREDTGTYTCMVSEE

SEQ ID NO: 274 - protein, 19 aa, Homo sapiens
KSVTREDTGTYTCMVSEEG

SEQ ID NO: 275 - protein, 10 aa, Homo sapiens
REDTGTYTCM

SEQ ID NO: 276 - protein, 11 aa, Homo sapiens
TREDTGTYTCM

SEQ ID NO: 277 - protein, 12 aa, Homo sapiens
VTREDTGTYTCM

SEQ ID NO: 278 - protein, 13 aa, Homo sapiens
SVTREDTGTYTCM

SEQ ID NO: 279 - protein, 14 aa, Homo sapiens
KSVTREDTGTYTCM

SEQ ID NO: 280 - protein, 10 aa, Homo sapiens
EDTGTYTCMV

SEQ ID NO: 281 - protein, 11 aa, Homo sapiens
EDTGTYTCMVS

SEQ ID NO: 282 - protein, 12 aa, Homo sapiens
EDTGTYTCMVSE

SEQ ID NO: 283 - protein, 13 aa, Homo sapiens
EDTGTYTCMVSEE

SEQ ID NO: 284 - protein, 14 aa, Homo sapiens
EDTGTYTCMVSEEG

SEQ ID NO: 285 - protein, 9 aa, Homo sapiens
EDTGTYTCE

SEQ ID NO: 286 - protein, 11 aa, Homo sapiens
REDTGTYTCEV

SEQ ID NO: 287 - protein, 13 aa, Homo sapiens
TREDTGTYTCEVS

SEQ ID NO: 288 - protein, 15 aa, Homo sapiens
VTREDTGTYTCEVSE

SEQ ID NO: 289 - protein, 17 aa, Homo sapiens
SVTREDTGTYTCEVSEE

SEQ ID NO: 290 - protein, 19 aa, Homo sapiens
KSVTREDTGTYTCEVSEEG

SEQ ID NO: 291 - protein, 10 aa, Homo sapiens
REDTGTYTCE

SEQ ID NO: 292 - protein, 11 aa, Homo sapiens
TREDTGTYTCE

SEQ ID NO: 293 - protein, 12 aa, Homo sapiens
VTREDTGTYTCE

SEQ ID NO: 294 - protein, 13 aa, Homo sapiens
SVTREDTGTYTCE

SEQ ID NO: 295 - protein, 14 aa, Homo sapiens
KSVTREDTGTYTCE

SEQ ID NO: 296 - protein, 10 aa, Homo sapiens
EDTGTYTCEV

SEQ ID NO: 297 - protein, 11 aa, Homo sapiens
EDTGTYTCEVS

SEQ ID NO: 298 - protein, 12 aa, Homo sapiens
EDTGTYTCEVSE

SEQ ID NO: 299 - protein, 13 aa, Homo sapiens
EDTGTYTCEVSEE

SEQ ID NO: 300 - protein, 14 aa, Homo sapiens
EDTGTYTCEVSEEG

SEQ ID NO: 301 - protein, 9 aa, Homo sapiens
EDTGTYRCM

SEQ ID NO: 302 - protein, 11 aa, Homo sapiens
REDTGTYTCMV

SEQ ID NO: 303 - protein, 13 aa, Homo sapiens
TREDTGTYTCMVS

SEQ ID NO: 304 - protein, 15 aa, Homo sapiens
VTREDTGTYTCMVSE

SEQ ID NO: 305 - protein, 17 aa, Homo sapiens
SVTREDTGTYTCMVSEE

SEQ ID NO: 306 - protein, 19 aa, Homo sapiens
KSVTREDTGTYTCMVSEEG

SEQ ID NO: 307 - protein, 10 aa, Homo sapiens
REDTGTYTCM

SEQ ID NO: 308 - protein, 11 aa, Homo sapiens
TREDTGTYTCM

SEQ ID NO: 309 - protein, 12 aa, Homo sapiens
VTREDTGTYTCM

SEQ ID NO: 310 - protein, 13 aa, Homo sapiens
SVTREDTGTYTCM

SEQ ID NO: 311 - protein, 14 aa, Homo sapiens
KSVTREDTGTYTCM

SEQ ID NO: 312 - protein, 10 aa, Homo sapiens
EDTGTYTCMV

SEQ ID NO: 313 - protein, 11 aa, Homo sapiens
EDTGTYTCMVS

SEQ ID NO: 314 - protein, 12 aa, Homo sapiens
EDTGTYTCMVSE

SEQ ID NO: 315 - protein, 13 aa, Homo sapiens
EDTGTYTCMVSEE

SEQ ID NO: 316 - protein, 14 aa, Homo sapiens
EDTGTYTCMVSEEG

SEQ ID NO: 317 - protein, 9 aa, Homo sapiens
EDTGTYRCE

SEQ ID NO: 318 - protein, 11 aa, Homo sapiens
REDTGTYTCMV

SEQ ID NO: 319 - protein, 13 aa, Homo sapiens
TREDTGTYTCMVS

SEQ ID NO: 320 - protein, 15 aa, Homo sapiens
VTREDTGTYTCMVSE

SEQ ID NO: 321 - protein, 17 aa, Homo sapiens
SVTREDTGTYTCMVSEE

SEQ ID NO: 322 - protein, 19 aa, Homo sapiens
KSVTREDTGTYTCMVSEEG

SEQ ID NO: 323 - protein, 10 aa, Homo sapiens
REDTGTYTCM

SEQ ID NO: 324 - protein, 11 aa, Homo sapiens
TREDTGTYTCM

SEQ ID NO: 325 - protein, 12 aa, Homo sapiens
VTREDTGTYTCM

SEQ ID NO: 326 - protein, 13 aa, Homo sapiens
SVTREDTGTYTCM

SEQ ID NO: 327 - protein, 14 aa, Homo sapiens
KSVTREDTGTYTCM

SEQ ID NO: 328 - protein, 10 aa, Homo sapiens
EDTGTYTCMV

SEQ ID NO: 329 - protein, 11 aa, Homo sapiens
EDTGTYTCMVS

SEQ ID NO: 330 - protein, 12 aa, Homo sapiens
EDTGTYTCMVSE

SEQ ID NO: 331 - protein, 13 aa, Homo sapiens
EDTGTYTCMVSEE

SEQ ID NO: 332 - protein, 14 aa, Homo sapiens
EDTGTYTCMVSEEG

SEQ ID NO: 333 - protein, 9 aa, Homo sapiens
EDSGTYTCM

SEQ ID NO: 334 - protein, 11 aa, Homo sapiens
REDTGTYTCMV

SEQ ID NO: 335 - protein, 13 aa, Homo sapiens
TREDTGTYTCMVS

SEQ ID NO: 336 - protein, 15 aa, Homo sapiens
VTREDTGTYTCMVSE

SEQ ID NO: 337 - protein, 17 aa, Homo sapiens
SVTREDTGTYTCMVSEE

SEQ ID NO: 338 - protein, 19 aa, Homo sapiens
KSVTREDTGTYTCMVSEEG

SEQ ID NO: 339 - protein, 10 aa, Homo sapiens
REDTGTYTCM

SEQ ID NO: 340 - protein, 11 aa, Homo sapiens
TREDTGTYTCM

SEQ ID NO: 341 - protein, 12 aa, Homo sapiens
VTREDTGTYTCM

SEQ ID NO: 342 - protein, 13 aa, Homo sapiens
SVTREDTGTYTCM

SEQ ID NO: 343 - protein, 14 aa, Homo sapiens
KSVTREDTGTYTCM

SEQ ID NO: 344 - protein, 10 aa, Homo sapiens
EDTGTYTCMV

SEQ ID NO: 345 - protein, 11 aa, Homo sapiens
EDTGTYTCMVS

SEQ ID NO: 346 - protein, 12 aa, Homo sapiens
EDTGTYTCMVSE

SEQ ID NO: 347 - protein, 13 aa, Homo sapiens
EDTGTYTCMVSEE

SEQ ID NO: 348 - protein, 14 aa, Homo sapiens
EDTGTYTCMVSEEG

SEQ ID NO: 349 - protein, 9 aa, Homo sapiens
EDSGTYTCE

SEQ ID NO: 350 - protein, 11 aa, Homo sapiens
REDSGTYTCEV

SEQ ID NO: 351 - protein, 13 aa, Homo sapiens
TREDSGTYTCEVS

SEQ ID NO: 352 - protein, 15 aa, Homo sapiens
VTREDSGTYTCEVSE

SEQ ID NO: 353 - protein, 17 aa, Homo sapiens
SVTREDSGTYTCEVSEE

SEQ ID NO: 354 - protein, 19 aa, Homo sapiens
KSVTREDSGTYTCEVSEEG

SEQ ID NO: 355 - protein, 10 aa, Homo sapiens
REDSGTYTCE

SEQ ID NO: 356 - protein, 11 aa, Homo sapiens
TREDSGTYTCE

SEQ ID NO: 357 - protein, 12 aa, Homo sapiens
VTREDSGTYTCE

SEQ ID NO: 358 - protein, 13 aa, Homo sapiens
SVTREDSGTYTCE

SEQ ID NO: 359 - protein, 14 aa, Homo sapiens
KSVTREDSGTYTCE

SEQ ID NO: 360 - protein, 10 aa, Homo sapiens
EDSGTYTCEV

SEQ ID NO: 361 - protein, 11 aa, Homo sapiens
EDSGTYTCEVS

SEQ ID NO: 362 - protein, 12 aa, Homo sapiens
EDSGTYTCEVSE

SEQ ID NO: 363 - protein, 13 aa, Homo sapiens
EDSGTYTCEVSEE

SEQ ID NO: 364 - protein, 14 aa, Homo sapiens
EDSGTYTCEVSEEG

SEQ ID NO: 365 - protein, 9 aa, Homo sapiens
EDSGTYRCM

SEQ ID NO: 366 - protein, 11 aa, Homo sapiens
REDSGTYRCMV

SEQ ID NO: 367 - protein, 13 aa, Homo sapiens
TREDSGTYRCMVS

SEQ ID NO: 368 - protein, 15 aa, Homo sapiens
VTREDSGTYRCMVSE

SEQ ID NO: 369 - protein, 17 aa, Homo sapiens
SVTREDSGTYRCMVSEE

SEQ ID NO: 370 - protein, 19 aa, Homo sapiens
KSVTREDSGTYRCMVSEEG

SEQ ID NO: 371 - protein, 10 aa, Homo sapiens
REDSGTYRCM

SEQ ID NO: 372 - protein, 11 aa, Homo sapiens
TREDSGTYRCM

SEQ ID NO: 373 - protein, 12 aa, Homo sapiens
VTREDSGTYRCM

SEQ ID NO: 374 - protein, 13 aa, Homo sapiens
SVTREDSGTYRCM

SEQ ID NO: 375 - protein, 14 aa, Homo sapiens
KSVTREDSGTYRCM

SEQ ID NO: 376 - protein, 10 aa, Homo sapiens
EDSGTYRCMV

SEQ ID NO: 377 - protein, 11 aa, Homo sapiens
EDSGTYRCMVS

SEQ ID NO: 378 - protein, 12 aa, Homo sapiens
EDSGTYRCMVSE

SEQ ID NO: 379 - protein, 13 aa, Homo sapiens
EDSGTYRCMVSEE

SEQ ID NO: 380 - protein, 14 aa, Homo sapiens
EDSGTYRCMVSEEG

SEQ ID NO: 381 - protein, 9 aa, Homo sapiens
EDSGTYRCE

SEQ ID NO: 382 - protein, 11 aa, Homo sapiens
REDSGTYRCEV

SEQ ID NO: 383 - protein, 13 aa, Homo sapiens
TREDSGTYRCEVS

SEQ ID NO: 384 - protein, 15 aa, Homo sapiens
VTREDSGTYRCEVSE

SEQ ID NO: 385 - protein, 17 aa, Homo sapiens
SVTREDSGTYRCEVSEE

SEQ ID NO: 386 - protein, 19 aa, Homo sapiens
KSVTREDSGTYRCEVSEEG

SEQ ID NO: 387 - protein, 10 aa, Homo sapiens
REDSGTYRCE

SEQ ID NO: 388 - protein, 11 aa, Homo sapiens
TREDSGTYRCE

SEQ ID NO: 389 - protein, 12 aa, Homo sapiens
VTREDSGTYRCE

SEQ ID NO: 390 - protein, 13 aa, Homo sapiens
SVTREDSGTYRCE

SEQ ID NO: 391 - protein, 14 aa, Homo sapiens
KSVTREDSGTYRCE

SEQ ID NO: 392 - protein, 10 aa, Homo sapiens
EDSGTYRCEV

SEQ ID NO: 393 - protein, 11 aa, Homo sapiens
EDSGTYRCEVS

SEQ ID NO: 394 - protein, 12 aa, Homo sapiens
EDSGTYRCEVSE

SEQ ID NO: 395 - protein, 13 aa, Homo sapiens
EDSGTYRCEVSEE

SEQ ID NO: 396 - protein, 14 aa, Homo sapiens
EDSGTYRCEVSEEG

SEQ ID NO: 397 - protein, 10 aa, Homo sapiens
AVNLKSSNRT

SEQ ID NO: 398 - protein, 10 aa, Homo sapiens
PVVQEFESVE

SEQ ID NO: 399 - protein, 10 aa, Homo sapiens
LSCIITDSQT

SEQ ID NO: 400 - protein, 10 aa, Homo sapiens
SDPRIEWKKI

SEQ ID NO: 401 - protein, 10 aa, Homo sapiens
QDEQTTYVFF

SEQ ID NO: 402 - protein, 10 aa, Homo sapiens
DNKIQGDLAG

SEQ ID NO: 403 - protein, 10 aa, Homo sapiens
RAEILGKTSL

SEQ ID NO: 404 - protein, 10 aa, Homo sapiens
KIWNVTRRDS

SEQ ID NO: 405 - protein, 10 aa, Homo sapiens
ALYRCEVVAR

SEQ ID NO: 406 - protein, 10 aa, Homo sapiens
NDRKEIDEIV

SEQ ID NO: 407 - protein, 10 aa, Homo sapiens
IELTVQVKPV

SEQ ID NO: 408 - protein, 10 aa, Homo sapiens
TPVCRVPKAV

SEQ ID NO: 409 - protein, 10 aa, Homo sapiens
PVGKMATLHC

SEQ ID NO: 410 - protein, 10 aa, Homo sapiens
QESEGHPRPH

SEQ ID NO: 411 - protein, 10 aa, Homo sapiens
YSWYRNDVPL

SEQ ID NO: 412 - protein, 10 aa, Homo sapiens
PTDSRANPRF

SEQ ID NO: 413 - protein, 10 aa, Homo sapiens
RNSSFHLNSE

SEQ ID NO: 414 - protein, 10 aa, Homo sapiens
TGTLVFTAVH

SEQ ID NO: 415 - protein, 10 aa, Homo sapiens
KDDSGQYYCI

SEQ ID NO: 416 - protein, 10 aa, Homo sapiens
ASNDAGSARC

SEQ ID NO: 417 - protein, 11 aa, Homo sapiens
EEQEMEVYDLN

SEQ ID NO: 418 - protein, 5 aa, Homo sapiens
AVNLK

SEQ ID NO: 419 - protein, 10 aa, Homo sapiens
SSNRTPVVQE

SEQ ID NO: 420 - protein, 10 aa, Homo sapiens
FESVELSCII

SEQ ID NO: 421 - protein, 10 aa, Homo sapiens
TDSQTSDPRI

SEQ ID NO: 422 - protein, 10 aa, Homo sapiens
EWKKIQDEQT

SEQ ID NO: 423 - protein, 10 aa, Homo sapiens
TYVFFDNKIQ

SEQ ID NO: 424 - protein, 10 aa, Homo sapiens
GDLAGRAEIL

SEQ ID NO: 425 - protein, 10 aa, Homo sapiens
GKTSLKIWNV

SEQ ID NO: 426 - protein, 10 aa, Homo sapiens
TRRDSALYRC

SEQ ID NO: 427 - protein, 10 aa, Homo sapiens
EVVARNDRKE

SEQ ID NO: 428 - protein, 10 aa, Homo sapiens
IDEIVIELTV

SEQ ID NO: 429 - protein, 10 aa, Homo sapiens
QVKPVTPVCR

SEQ ID NO: 430 - protein, 10 aa, Homo sapiens
VPKAVPVGKM

SEQ ID NO: 431 - protein, 10 aa, Homo sapiens
ATLHCQESEG

SEQ ID NO: 432 - protein, 10 aa, Homo sapiens
HPRPHYSWYR

SEQ ID NO: 433 - protein, 10 aa, Homo sapiens
NDVPLPTDSR

SEQ ID NO: 434 - protein, 10 aa, Homo sapiens
ANPRFRNSSF

SEQ ID NO: 435 - protein, 10 aa, Homo sapiens
HLNSETGTLV

SEQ ID NO: 436 - protein, 10 aa, Homo sapiens
FTAVHKDDSG

SEQ ID NO: 437 - protein, 10 aa, Homo sapiens
QYYCIASNDA

SEQ ID NO: 438 - protein, 10 aa, Homo sapiens
GSARCEEQEM

SEQ ID NO: 439 - protein, 6 aa, Homo sapiens
EVYDLN

SEQ ID NO: 440 - protein, 8 aa, Homo sapiens
AVNLKSSN

SEQ ID NO: 441 - protein, 8 aa, Homo sapiens
RTPVVQEF

SEQ ID NO: 442 - protein, 8 aa, Homo sapiens
ESVELSCI

SEQ ID NO: 443 - protein, 8 aa, Homo sapiens
PRIEWKKI

SEQ ID NO: 444 - protein, 8 aa, Homo sapiens
QDEQTTYV

SEQ ID NO: 445 - protein, 8 aa, Homo sapiens
FFDNKIQG

SEQ ID NO: 446 - protein, 8 aa, Homo sapiens
DLAGRAEI

SEQ ID NO: 447 - protein, 8 aa, Homo sapiens
LGKTSLKI

SEQ ID NO: 448 - protein, 8 aa, Homo sapiens
WNVTRRDS

SEQ ID NO: 449 - protein, 8 aa, Homo sapiens
ALYRCEVV

SEQ ID NO: 450 - protein, 8 aa, Homo sapiens
ARNDRKEI

SEQ ID NO: 451 - protein, 8 aa, Homo sapiens
DEIVIELT

SEQ ID NO: 452 - protein, 8 aa, Homo sapiens
VQVKPVTP

SEQ ID NO: 453 - protein, 8 aa, Homo sapiens
VCRVPKAV

SEQ ID NO: 454 - protein, 8 aa, Homo sapiens
PVGKMATL

SEQ ID NO: 455 - protein, 8 aa, Homo sapiens
HCQESEGH

SEQ ID NO: 456 - protein, 8 aa, Homo sapiens
PRPHYSWY

SEQ ID NO: 457 - protein, 8 aa, Homo sapiens
RNDVPLPT

SEQ ID NO: 458 - protein, 8 aa, Homo sapiens
DSRANPRF

SEQ ID NO: 459 - protein, 8 aa, Homo sapiens
RNSSFHLN

SEQ ID NO: 460 - protein, 8 aa, Homo sapiens
SETGTLVF

SEQ ID NO: 461 - protein, 8 aa, Homo sapiens
TAVHKDDS

SEQ ID NO: 462 - protein, 8 aa, Homo sapiens
GQYYCIAS

SEQ ID NO: 463 - protein, 8 aa, Homo sapiens
NDAGSARC

SEQ ID NO: 464 - protein, 8 aa, Homo sapiens
EEQEMEVY

SEQ ID NO: 465 - protein, 3 aa, Homo sapiens
DLN

SEQ ID NO: 466 - protein, 4 aa, Homo sapiens
AVNL

SEQ ID NO: 467 - protein, 8 aa, Homo sapiens
KSSNRTPV

SEQ ID NO: 468 - protein, 8 aa, Homo sapiens
VQEFESVE

SEQ ID NO: 469 - protein, 8 aa, Homo sapiens
LSCIITDS

SEQ ID NO: 470 - protein, 8 aa, Homo sapiens
QTSDPRIE

SEQ ID NO: 471 - protein, 8 aa, Homo sapiens
WKKIQDEQ

SEQ ID NO: 472 - protein, 8 aa, Homo sapiens
TTYVFFDN

SEQ ID NO: 473 - protein, 8 aa, Homo sapiens
KIQGDLAG

SEQ ID NO: 474 - protein, 8 aa, Homo sapiens
RAEILGKT

SEQ ID NO: 475 - protein, 8 aa, Homo sapiens
SLKIWNVT

SEQ ID NO: 476 - protein, 8 aa, Homo sapiens
RRDSALYR

SEQ ID NO: 477 - protein, 8 aa, Homo sapiens
CEVVARND

SEQ ID NO: 478 - protein, 8 aa, Homo sapiens
RKEIDEIV

SEQ ID NO: 479 - protein, 8 aa, Homo sapiens
IELTVQVK

SEQ ID NO: 480 - protein, 8 aa, Homo sapiens
PVTPVCRV

SEQ ID NO: 481 - protein, 8 aa, Homo sapiens
PKAVPVGK

SEQ ID NO: 482 - protein, 8 aa, Homo sapiens
MATLHCQE

SEQ ID NO: 483 - protein, 8 aa, Homo sapiens
SEGHPRPH

SEQ ID NO: 484 - protein, 8 aa, Homo sapiens
YSWYRNDV

SEQ ID NO: 485 - protein, 8 aa, Homo sapiens
PLPTDSRA

SEQ ID NO: 486 - protein, 8 aa, Homo sapiens
NPRFRNSS

SEQ ID NO: 487 - protein, 8 aa, Homo sapiens
FHLNSETG

SEQ ID NO: 488 - protein, 8 aa, Homo sapiens
TLVFTAVH

SEQ ID NO: 489 - protein, 8 aa, Homo sapiens
KDDSGQYY

SEQ ID NO: 490 - protein, 8 aa, Homo sapiens
CIASNDAG

SEQ ID NO: 491 - protein, 8 aa, Homo sapiens
SARCEEQE

SEQ ID NO: 492 - protein, 7 aa, Homo sapiens
MEVYDLN

SEQ ID NO: 493 - protein, 10 aa, Homo sapiens
GFSAPKDQQV

SEQ ID NO: 494 - protein, 10 aa, Homo sapiens
VTAVEYQEAI

SEQ ID NO: 495 - protein, 10 aa, Homo sapiens
LACKTPKKTV

SEQ ID NO: 496 - protein, 10 aa, Homo sapiens
SSRLEWKKLG

SEQ ID NO: 497 - protein, 10 aa, Homo sapiens
RSVSFVYYQQ

SEQ ID NO: 498 - protein, 10 aa, Homo sapiens
TLQGDFKNRA

SEQ ID NO: 499 - protein, 10 aa, Homo sapiens
EMIDFNIRIK

SEQ ID NO: 500 - protein, 10 aa, Homo sapiens
NVTRSDAGKY

SEQ ID NO: 501 - protein, 10 aa, Homo sapiens
RCEVSAPSEQ

SEQ ID NO: 502 - protein, 10 aa, Homo sapiens
GQNLEEDTVT

SEQ ID NO: 503 - protein, 10 aa, Homo sapiens
LEVLVAPAVP

SEQ ID NO: 504 - protein, 10 aa, Homo sapiens
SCEVPSSALS

SEQ ID NO: 505 - protein, 10 aa, Homo sapiens
GTVVELRCQD

SEQ ID NO: 506 - protein, 10 aa, Homo sapiens
KEGNPAPEYT

SEQ ID NO: 507 - protein, 10 aa, Homo sapiens
WFKDGIRLLE

SEQ ID NO: 508 - protein, 10 aa, Homo sapiens
NPRLGSQSTN

SEQ ID NO: 509 - protein, 10 aa, Homo sapiens
SSYTMNTKTG

SEQ ID NO: 510 - protein, 10 aa, Homo sapiens
TLQFNTVSKL

SEQ ID NO: 511 - protein, 10 aa, Homo sapiens
DTGEYSCEAR

SEQ ID NO: 512 - protein, 10 aa, Homo sapiens
NSVGYRRCPG

SEQ ID NO: 513 - protein, 9 aa, Homo sapiens
KRMQVDDLN

SEQ ID NO: 514 - protein, 5 aa, Homo sapiens
GFSAP

SEQ ID NO: 515 - protein, 10 aa, Homo sapiens
KDQQVVTAVE

SEQ ID NO: 516 - protein, 10 aa, Homo sapiens
YQEAILACKT

SEQ ID NO: 517 - protein, 10 aa, Homo sapiens
PKKTVSSRLE

SEQ ID NO: 518 - protein, 10 aa, Homo sapiens
WKKLGRSVSF

SEQ ID NO: 519 - protein, 10 aa, Homo sapiens
VYYQQTLQGD

SEQ ID NO: 520 - protein, 10 aa, Homo sapiens
FKNRAEMIDF

SEQ ID NO: 521 - protein, 10 aa, Homo sapiens
NIRIKNVTRS

SEQ ID NO: 522 - protein, 10 aa, Homo sapiens
DAGKYRCEVS

SEQ ID NO: 523 - protein, 10 aa, Homo sapiens
APSEQGQNLE

SEQ ID NO: 524 - protein, 10 aa, Homo sapiens
EDTVTLEVLV

SEQ ID NO: 525 - protein, 10 aa, Homo sapiens
APAVPSCEVP

SEQ ID NO: 526 - protein, 10 aa, Homo sapiens
SSALSGTVVE

SEQ ID NO: 527 - protein, 10 aa, Homo sapiens
LRCQDKEGNP

SEQ ID NO: 528 - protein, 10 aa, Homo sapiens
APEYTWFKDG

SEQ ID NO: 529 - protein, 10 aa, Homo sapiens
IRLLENPRLG

SEQ ID NO: 530 - protein, 10 aa, Homo sapiens
SQSTNSSYTM

SEQ ID NO: 531 - protein, 10 aa, Homo sapiens
NTKTGTLQFN

SEQ ID NO: 532 - protein, 10 aa, Homo sapiens
TVSKLDTGEY

SEQ ID NO: 533 - protein, 10 aa, Homo sapiens
SCEARNSVGY

SEQ ID NO: 534 - protein, 10 aa, Homo sapiens
RRCPGKRMQV

SEQ ID NO: 535 - protein, 4 aa, Homo sapiens
DDLN

SEQ ID NO: 536 - protein, 8 aa, Homo sapiens
GFSAPKDQ

SEQ ID NO: 537 - protein, 8 aa, Homo sapiens
QVVTAVEY

SEQ ID NO: 538 - protein, 8 aa, Homo sapiens
QEAILACK

SEQ ID NO: 539 - protein, 8 aa, Homo sapiens
TPKKTVSS

SEQ ID NO: 540 - protein, 8 aa, Homo sapiens
RLEWKKLG

SEQ ID NO: 541 - protein, 8 aa, Homo sapiens
RSVSFVYY

SEQ ID NO: 542 - protein, 8 aa, Homo sapiens
QQTLQGDF

SEQ ID NO: 543 - protein, 8 aa, Homo sapiens
KNRAEMID

SEQ ID NO: 544 - protein, 8 aa, Homo sapiens
FNIRIKNV

SEQ ID NO: 545 - protein, 8 aa, Homo sapiens
TRSDAGKY

SEQ ID NO: 546 - protein, 8 aa, Homo sapiens
RCEVSAPS

SEQ ID NO: 547 - protein, 8 aa, Homo sapiens
EQGQNLEE

SEQ ID NO: 548 - protein, 8 aa, Homo sapiens
DTVTLEVL

SEQ ID NO: 549 - protein, 8 aa, Homo sapiens
VAPAVPSC

SEQ ID NO: 550 - protein, 8 aa, Homo sapiens
EVPSSALS

SEQ ID NO: 551 - protein, 8 aa, Homo sapiens
GTVVELRC

SEQ ID NO: 552 - protein, 8 aa, Homo sapiens
QDKEGNPA

SEQ ID NO: 553 - protein, 8 aa, Homo sapiens
PEYTWFKD

SEQ ID NO: 554 - protein, 8 aa, Homo sapiens
GIRLLENP

SEQ ID NO: 555 - protein, 8 aa, Homo sapiens
RLGSQSTN

SEQ ID NO: 556 - protein, 8 aa, Homo sapiens
SSYTMNTK

SEQ ID NO: 557 - protein, 8 aa, Homo sapiens
TGTLQFNT

SEQ ID NO: 558 - protein, 8 aa, Homo sapiens
VSKLDTGE

SEQ ID NO: 559 - protein, 8 aa, Homo sapiens
YSCEARNS

SEQ ID NO: 560 - protein, 8 aa, Homo sapiens
VGYRRCPG

SEQ ID NO: 561 - protein, 9 aa, Homo sapiens
KRMQVDDLN

SEQ ID NO: 562 - protein, 4 aa, Homo sapiens
GFSA

SEQ ID NO: 563 - protein, 8 aa, Homo sapiens
PKDQQVVT

SEQ ID NO: 564 - protein, 8 aa, Homo sapiens
AVEYQEAI

SEQ ID NO: 565 - protein, 8 aa, Homo sapiens
LACKTPKK

SEQ ID NO: 566 - protein, 8 aa, Homo sapiens
TVSSRLEW

SEQ ID NO: 567 - protein, 8 aa, Homo sapiens
KKLGRSVS

SEQ ID NO: 568 - protein, 8 aa, Homo sapiens
FVYYQQTL

SEQ ID NO: 569 - protein, 8 aa, Homo sapiens
QGDFKNRA

SEQ ID NO: 570 - protein, 8 aa, Homo sapiens
EMIDFNIR

SEQ ID NO: 571 - protein, 8 aa, Homo sapiens
IKNVTRSD

SEQ ID NO: 572 - protein, 8 aa, Homo sapiens
AGKYRCEV

SEQ ID NO: 573 - protein, 8 aa, Homo sapiens
SAPSEQGQ

SEQ ID NO: 574 - protein, 8 aa, Homo sapiens
NLEEDTVT

SEQ ID NO: 575 - protein, 8 aa, Homo sapiens
LEVLVAPA

SEQ ID NO: 576 - protein, 8 aa, Homo sapiens
VPSCEVPS

SEQ ID NO: 577 - protein, 8 aa, Homo sapiens
SALSGTVV

SEQ ID NO: 578 - protein, 8 aa, Homo sapiens
ELRCQDKE

SEQ ID NO: 579 - protein, 8 aa, Homo sapiens
GNPAPEYT

SEQ ID NO: 580 - protein, 8 aa, Homo sapiens
WFKDGIRL

SEQ ID NO: 581 - protein, 8 aa, Homo sapiens
LENPRLGS

SEQ ID NO: 582 - protein, 8 aa, Homo sapiens
QSTNSSYT

SEQ ID NO: 583 - protein, 8 aa, Homo sapiens
MNTKTGTL

SEQ ID NO: 584 - protein, 8 aa, Homo sapiens
QFNTVSKL

SEQ ID NO: 585 - protein, 8 aa, Homo sapiens
DTGEYSCE

SEQ ID NO: 586 - protein, 8 aa, Homo sapiens
ARNSVGYR

SEQ ID NO: 587 - protein, 8 aa, Homo sapiens
RCPGKRMQ

SEQ ID NO: 588 - protein, 5 aa, Homo sapiens
VDDLN

SEQ ID NO: 589 - protein, 10 aa, Homo sapiens
RIYSYAGDNI

SEQ ID NO: 590 - protein, 10 aa, Homo sapiens
VTAQAMYEGL

SEQ ID NO: 591 - protein, 10 aa, Homo sapiens
WMSCVSQSTG

SEQ ID NO: 592 - protein, 10 aa, Homo sapiens
QIQCKVFDSL

SEQ ID NO: 593 - protein, 11 aa, Homo sapiens
LNLSSTLQATR

SEQ ID NO: 594 - protein, 5 aa, Homo sapiens
RIYSY

SEQ ID NO: 595 - protein, 10 aa, Homo sapiens
AGDNIVTAQA

SEQ ID NO: 596 - protein, 10 aa, Homo sapiens
MYEGLWMSCV

SEQ ID NO: 597 - protein, 10 aa, Homo sapiens
SQSTGQIQCK

SEQ ID NO: 598 - protein, 10 aa, Homo sapiens
VFDSLLNLSS

SEQ ID NO: 599 - protein, 6 aa, Homo sapiens
TLQATR

SEQ ID NO: 600 - protein, 8 aa, Homo sapiens
QEFYDPMT

SEQ ID NO: 601 - protein, 7 aa, Homo sapiens
PVNARYE

SEQ ID NO: 602 - protein, 11 aa, Homo sapiens
QEFYDPMTPVN

SEQ ID NO: 603 - protein, 4 aa, Homo sapiens
ARYE

SEQ ID NO: 604 - protein, 10 aa, Homo sapiens
KTSSYVGASI

SEQ ID NO: 605 - protein, 10 aa, Homo sapiens
VTAVGFSKGL

SEQ ID NO: 606 - protein, 10 aa, Homo sapiens
WMECATHSTG

SEQ ID NO: 607 - protein, 10 aa, Homo sapiens
ITQCDIYSTL

SEQ ID NO: 608 - protein, 11 aa, Homo sapiens
LGLPADIQAAQ

SEQ ID NO: 609 - protein, 5 aa, Homo sapiens
KTSSY

SEQ ID NO: 610 - protein, 10 aa, Homo sapiens
VGASIVTAVG

SEQ ID NO: 611 - protein, 10 aa, Homo sapiens
FSKGLWMECA

SEQ ID NO: 612 - protein, 10 aa, Homo sapiens
THSTGITQCD

SEQ ID NO: 613 - protein, 10 aa, Homo sapiens
IYSTLLGLPA

SEQ ID NO: 614 - protein, 6 aa, Homo sapiens
DIQAAQ

SEQ ID NO: 615 - protein, 7 aa, Homo sapiens
RDFYSPL

SEQ ID NO: 616 - protein, 10 aa, Homo sapiens
RVSAFIGSNI

SEQ ID NO: 617 - protein, 10 aa, Homo sapiens
ITSQNIWEGL

SEQ ID NO: 618 - protein, 10 aa, Homo sapiens
WMNCVVQSTG

SEQ ID NO: 619 - protein, 10 aa, Homo sapiens
QMQCKVYDSL

SEQ ID NO: 620 - protein, 11 aa, Homo sapiens
LALPQDLQAAR

SEQ ID NO: 621 - protein, 5 aa, Homo sapiens
RVSAF

SEQ ID NO: 622 - protein, 10 aa, Homo sapiens
IGSNIITSQN

SEQ ID NO: 623 - protein, 10 aa, Homo sapiens
IWEGLWMNCV

SEQ ID NO: 624 - protein, 10 aa, Homo sapiens
VQSTGQMQCK

SEQ ID NO: 625 - protein, 10 aa, Homo sapiens
VYDSLLALPQ

SEQ ID NO: 626 - protein, 6 aa, Homo sapiens
DLQAAR

SEQ ID NO: 627 - protein, 8 aa, Homo sapiens
RDFYNPVV

SEQ ID NO: 628 - protein, 7 aa, Homo sapiens
PEAQKRE

SEQ ID NO: 629 - protein, 10 aa, Homo sapiens
RVTAFIGSNI

SEQ ID NO: 630 - protein, 10 aa, Homo sapiens
VTSQTIWEGL

SEQ ID NO: 631 - protein, 10 aa, Homo sapiens
WMNCVVQSTG

SEQ ID NO: 632 - protein, 10 aa, Homo sapiens
QMQCKVYDSL

SEQ ID NO: 633 - protein, 11 aa, Homo sapiens
LALPQDLQAAR

SEQ ID NO: 634 - protein, 5 aa, Homo sapiens
RVTAF

SEQ ID NO: 635 - protein, 10 aa, Homo sapiens
IGSNIVTSQT

SEQ ID NO: 636 - protein, 10 aa, Homo sapiens
IWEGLWMNCV

SEQ ID NO: 637 - protein, 10 aa, Homo sapiens
VQSTGQMQCK

SEQ ID NO: 638 - protein, 10 aa, Homo sapiens
VYDSLLALPQ

SEQ ID NO: 639 - protein, 6 aa, Homo sapiens
DLQAAR

SEQ ID NO: 640 - protein, 8 aa, Homo sapiens
QDFYNPLV

SEQ ID NO: 641 - protein, 7 aa, Homo sapiens
ASGQKRE

SEQ ID NO: 642 - protein, 10 aa, Homo sapiens
QVTAFLDHNI

SEQ ID NO: 643 - protein, 10 aa, Homo sapiens
VTAQTTWKGL

SEQ ID NO: 644 - protein, 10 aa, Homo sapiens
WMSCVVQSTG

SEQ ID NO: 645 - protein, 10 aa, Homo sapiens
HMQCKVYDSV

SEQ ID NO: 646 - protein, 11 aa, Homo sapiens
LALSTEVQAAR

SEQ ID NO: 647 - protein, 5 aa, Homo sapiens
QVTAF

SEQ ID NO: 648 - protein, 10 aa, Homo sapiens
LDHNIVTAQT

SEQ ID NO: 649 - protein, 10 aa, Homo sapiens
TWKGLWMSCV

SEQ ID NO: 650 - protein, 10 aa, Homo sapiens
VQSTGHMQCK

SEQ ID NO: 651 - protein, 10 aa, Homo sapiens
VYDSVLALST

SEQ ID NO: 652 - protein, 6 aa, Homo sapiens
EVQAAR

SEQ ID NO: 653 - protein, 8 aa, Homo sapiens
REFYDPSV

SEQ ID NO: 654 - protein, 10 aa, Homo sapiens
KVTAFIGNSI

SEQ ID NO: 655 - protein, 10 aa, Homo sapiens
VVAQVVWEGL

SEQ ID NO: 656 - protein, 10 aa, Homo sapiens
WMSCVVQSTG

SEQ ID NO: 657 - protein, 10 aa, Homo sapiens
QMQCKVYDSL

SEQ ID NO: 658 - protein, 11 aa, Homo sapiens
LALPQDLQAAR

SEQ ID NO: 659 - protein, 5 aa, Homo sapiens
KVTAF

SEQ ID NO: 660 - protein, 10 aa, Homo sapiens
IGNSIVVAQV

SEQ ID NO: 661 - protein, 10 aa, Homo sapiens
VWEGLWMSCV

SEQ ID NO: 662 - protein, 10 aa, Homo sapiens
VQSTGQMQCK

SEQ ID NO: 663 - protein, 10 aa, Homo sapiens
VYDSLLALPQ

SEQ ID NO: 664 - protein, 6 aa, Homo sapiens
DLQAAR

SEQ ID NO: 665 - protein, 8 aa, Homo sapiens
RDFYNPLV

SEQ ID NO: 666 - protein, 7 aa, Homo sapiens
AEAQKRE

SEQ ID NO: 667 - protein, 10 aa, Homo sapiens
QMSSYAGDNI

SEQ ID NO: 668 - protein, 10 aa, Homo sapiens
ITAQAMYKGL

SEQ ID NO: 669 - protein, 10 aa, Homo sapiens
WMDCVTQSTG

SEQ ID NO: 670 - protein, 10 aa, Homo sapiens
MMSCKMYDSV

SEQ ID NO: 671 - protein, 11 aa, Homo sapiens
LALSAALQATR

SEQ ID NO: 672 - protein, 5 aa, Homo sapiens
QMSSY

SEQ ID NO: 673 - protein, 10 aa, Homo sapiens
AGDNIITAQA

SEQ ID NO: 674 - protein, 10 aa, Homo sapiens
MYKGLWMDCV

SEQ ID NO: 675 - protein, 10 aa, Homo sapiens
TQSTGMMSCK

SEQ ID NO: 676 - protein, 10 aa, Homo sapiens
MYDSVLALSA

SEQ ID NO: 677 - protein, 6 aa, Homo sapiens
ALQATR

SEQ ID NO: 678 - protein, 8 aa, Homo sapiens
TDFYNPLI

SEQ ID NO: 679 - protein, 7 aa, Homo sapiens
PTNIKYE

SEQ ID NO: 680 - protein, 10 aa, Homo sapiens
RVSAFIENNI

SEQ ID NO: 681 - protein, 10 aa, Homo sapiens
VVFENFWEGL

SEQ ID NO: 682 - protein, 10 aa, Homo sapiens
WMNCVRQANI

SEQ ID NO: 683 - protein, 10 aa, Homo sapiens
RMQCKIYDSL

SEQ ID NO: 684 - protein, 11 aa, Homo sapiens
LALSPDLQAAR

SEQ ID NO: 685 - protein, 5 aa, Homo sapiens
RVSAF

SEQ ID NO: 686 - protein, 10 aa, Homo sapiens
IENNIVVFEN

SEQ ID NO: 687 - protein, 10 aa, Homo sapiens
FWEGLWMNCV

SEQ ID NO: 688 - protein, 10 aa, Homo sapiens
RQANIRMQCK

SEQ ID NO: 689 - protein, 10 aa, Homo sapiens
IYDSLLALSP

SEQ ID NO: 690 - protein, 6 aa, Homo sapiens
DLQAAR

SEQ ID NO: 691 - protein, 8 aa, Homo sapiens
RDFYNSIV

SEQ ID NO: 692 - protein, 7 aa, Homo sapiens
NVAQKRE

SEQ ID NO: 693 - protein, 10 aa, Homo sapiens
KVTAFIGNSI

SEQ ID NO: 694 - protein, 10 aa, Homo sapiens
VVAQVVWEGL

SEQ ID NO: 695 - protein, 10 aa, Homo sapiens
WMSCVVQSTG

SEQ ID NO: 696 - protein, 10 aa, Homo sapiens
QMQCKVYDSL

SEQ ID NO: 697 - protein, 11 aa, Homo sapiens
LALPQDLQAAR

SEQ ID NO: 698 - protein, 5 aa, Homo sapiens
KVTAF

SEQ ID NO: 699 - protein, 10 aa, Homo sapiens
IGNSIVVAQV

SEQ ID NO: 700 - protein, 10 aa, Homo sapiens
VWEGLWMSCV

SEQ ID NO: 701 - protein, 10 aa, Homo sapiens
VQSTGQMQCK

SEQ ID NO: 702 - protein, 10 aa, Homo sapiens
VYDSLLALPQ

SEQ ID NO: 703 - protein, 6 aa, Homo sapiens
DLQAAR

SEQ ID NO: 704 - protein, 8 aa, Homo sapiens
QDFYNPLV

SEQ ID NO: 705 - protein, 7 aa, Homo sapiens
AEALKRE

SEQ ID NO: 706 - protein, 10 aa, Homo sapiens
KVSTIDGTVI

SEQ ID NO: 707 - protein, 10 aa, Homo sapiens
TTATYWANLW

SEQ ID NO: 708 - protein, 10 aa, Homo sapiens
KACVTDSTGV

SEQ ID NO: 709 - protein, 10 aa, Homo sapiens
SNCKDFPSML

SEQ ID NO: 710 - protein, 10 aa, Homo sapiens
ALDGYIQACR

SEQ ID NO: 711 - protein, 5 aa, Homo sapiens
KVSTI

SEQ ID NO: 712 - protein, 10 aa, Homo sapiens
DGTVITTATY

SEQ ID NO: 713 - protein, 10 aa, Homo sapiens
WANLWKACVT

SEQ ID NO: 714 - protein, 10 aa, Homo sapiens
DSTGVSNCKD

SEQ ID NO: 715 - protein, 10 aa, Homo sapiens
FPSMLALDGY

SEQ ID NO: 716 - protein, 5 aa, Homo sapiens
IQACR

SEQ ID NO: 717 - protein, 7 aa, Homo sapiens
EFFDPLF

SEQ ID NO: 718 - protein, 6 aa, Homo sapiens
VEQKYE

SEQ ID NO: 719 - protein, 10 aa, Homo sapiens
DRGYGTSLLG

SEQ ID NO: 720 - protein, 10 aa, Homo sapiens
GSVGYPYGGS

SEQ ID NO: 721 - protein, 10 aa, Homo sapiens
GFGSYGSGYG

SEQ ID NO: 722 - protein, 10 aa, Homo sapiens
YGYGYGYGYG

SEQ ID NO: 723 - protein, 6 aa, Homo sapiens
GYTDPR

SEQ ID NO: 724 - protein, 5 aa, Homo sapiens
DRGYG

SEQ ID NO: 725 - protein, 10 aa, Homo sapiens
TSLLGGSVGY

SEQ ID NO: 726 - protein, 10 aa, Homo sapiens
PYGGSGFGSY

SEQ ID NO: 727 - protein, 10 aa, Homo sapiens
GSGYGYGYGY

SEQ ID NO: 728 - protein, 11 aa, Homo sapiens
GYGYGGYTDPR

SEQ ID NO: 729 - protein, 10 aa, Homo sapiens
GVNPTAQSSG

SEQ ID NO: 730 - protein, 10 aa, Homo sapiens
SLYGSQIYAL

SEQ ID NO: 731 - protein, 10 aa, Homo sapiens
CNQFYTPAAT

SEQ ID NO: 732 - protein, 10 aa, Homo sapiens
GLYVDQYLYH

SEQ ID NO: 733 - protein, 8 aa, Homo sapiens
YCVVDPQE

SEQ ID NO: 734 - protein, 5 aa, Homo sapiens
GVNPT

SEQ ID NO: 735 - protein, 10 aa, Homo sapiens
AQSSGSLYGS

SEQ ID NO: 736 - protein, 10 aa, Homo sapiens
QIYALCNQFY

SEQ ID NO: 737 - protein, 10 aa, Homo sapiens
TPAATGLYVD

SEQ ID NO: 738 - protein, 10 aa, Homo sapiens
QYLYHYCVVD

SEQ ID NO: 739 - protein, 3 aa, Homo sapiens
PQE

SEQ ID NO: 740 - protein, 9 aa, Homo sapiens
SVTVHSSEP

SEQ ID NO: 741 - protein, 9 aa, Homo sapiens
KFDQGDTTR

SEQ ID NO: 742 - protein, 9 aa, Homo sapiens
GEVKVKLIV

SEQ ID NO: 743 - protein, 9 aa, Homo sapiens
VSEEGGNSY

SEQ ID NO: 744 - protein, 9 aa, Homo sapiens
LVCYNNKIT

SEQ ID NO: 745 - protein, 5 aa, Homo sapiens
VLPPS

SEQ ID NO: 746 - protein, 7 aa, Homo sapiens
YEDRVTF

SEQ ID NO: 747 - protein, 5 aa, Homo sapiens
PRVEW

SEQ ID NO: 748 - protein, 10 aa, Homo sapiens
KVFDSLLNLS

SEQ ID NO: 749 - protein, 10 aa, Homo sapiens
NRIVQEFYDP

SEQ ID NO: 750 - protein, 10 aa, Homo sapiens
YAGDNIVTAQ

SEQ ID NO: 751 - protein, 10 aa, Homo sapiens
VSQSTGQIQC

SEQ ID NO: 752 - protein, 10 aa, Homo sapiens
MTPVNARYEF

SEQ ID NO: 753 - protein, 10 aa, Homo sapiens
AMYEGLWMSC

SEQ ID NO: 754 - protein, 10 aa, Homo sapiens
TTWLGLWMSC

SEQ ID NO: 755 - protein, 10 aa, Homo sapiens
YVGASIVTAV

SEQ ID NO: 756 - protein, 10 aa, Homo sapiens
GILRDFYSPL

SEQ ID NO: 757 - protein, 10 aa, Homo sapiens
VPDSMKFEIG

SEQ ID NO: 758 - protein, 10 aa, Homo sapiens
DIYSTLLGLP

SEQ ID NO: 759 - protein, 10 aa, Homo sapiens
GFSLGLWMEC

SEQ ID NO: 760 - protein, 10 aa, Homo sapiens
ATHSTGITQC

SEQ ID NO: 761 - protein, 10 aa, Homo sapiens
GFSKGLWMEC

SEQ ID NO: 762 - protein, 10 aa, Homo sapiens
KVYDSLLALP

SEQ ID NO: 763 - protein, 10 aa, Homo sapiens
NTIIRDFYNP

SEQ ID NO: 764 - protein, 10 aa, Homo sapiens
VVPEAQKREM

SEQ ID NO: 765 - protein, 10 aa, Homo sapiens
NIWEGLWMNC

SEQ ID NO: 766 - protein, 10 aa, Homo sapiens
VVQSTGQMQC

SEQ ID NO: 767 - protein, 10 aa, Homo sapiens
FIGSNIITSQ

SEQ ID NO: 768 - protein, 10 aa, Homo sapiens
VASGQKREMG

SEQ ID NO: 769 - protein, 10 aa, Homo sapiens
NIIQDFYNPL

SEQ ID NO: 770 - protein, 10 aa, Homo sapiens
FIGSNIVTSQ

SEQ ID NO: 771 - protein, 10 aa, Homo sapiens
TIWEGLWMNC

SEQ ID NO: 772 - protein, 10 aa, Homo sapiens
IVVREFYDPS

SEQ ID NO: 773 - protein, 10 aa, Homo sapiens
VVQSTGHMQC

SEQ ID NO: 774 - protein, 10 aa, Homo sapiens
FLDHNIVTAQ

SEQ ID NO: 775 - protein, 10 aa, Homo sapiens
VPVSQKYELG

SEQ ID NO: 776 - protein, 10 aa, Homo sapiens
KVYDSVLALS

SEQ ID NO: 777 - protein, 10 aa, Homo sapiens
TTWKGLWMSC

SEQ ID NO: 778 - protein, 9 aa, Homo sapiens
DRGYGTSLL

SEQ ID NO: 779 - protein, 9 aa, Homo sapiens
GYGYGYGYG

SEQ ID NO: 780 - protein, 9 aa, Homo sapiens
GSGFGSYGS

SEQ ID NO: 781 - protein, 9 aa, Homo sapiens
YGYGGYTDP

SEQ ID NO: 782 - protein, 9 aa, Homo sapiens
GVNPTAQSS

SEQ ID NO: 783 - protein, 9 aa, Homo sapiens
GSLYGSQIY

SEQ ID NO: 784 - protein, 9 aa, Homo sapiens
AATGLYVDQ

SEQ ID NO: 785 - protein, 9 aa, Homo sapiens
ALCNQFYTP

SEQ ID NO: 786 - protein, 9 aa, Homo sapiens
YLYHYCVVD

SEQ ID NO: 787 - protein, 9 aa, Homo sapiens
GGSVGYPYG

SEQ ID NO: 788 - skipped

SEQ ID NO: 789 - protein, 8 aa, Homo sapiens
PEVRIPEN

SEQ ID NO: 790 - protein, 7 aa, Homo sapiens
PRIEWKK